(19) United States
(12) Patent Application Publication
(10) Pub. No.: US 2018/0282310 A1
(43) Pub. Date: Oct. 4, 2018

(54) FORMS AND COMPOSITIONS OF BIARYL INHIBITORS OF BRUTON'S TYROSINE KINASE

(71) Applicant: BIOGEN MA INC., Cambridge, MA (US)

(72) Inventors: J. Michael MacPhee, Cambridge, MA (US); Michael Humora, Cambridge, MA (US)

(21) Appl. No.: 15/580,564

(22) PCT Filed: Jun. 10, 2016
(86) PCT No.: PCT/US2016/036963

§ 371 Date: Dec. 7, 2017

Related U.S. Application Data
(60) Provisional application No. 62/173,897, filed on Jun. 10, 2015.

(51) Int. Cl. C07D 403/14; C07C 309/04; C07C 309/05; C07C 309/08; C07C 309/29; C07C 309/30; C07C 309/35; C07C 55/07; C07C 57/15; C07C 59/255; C07C 59/265; C07C 59/245; C07C 55/10; C07C 233/83; C07C 57/145; C07C 229/24; C07C 55/28; C07C 59/11; C07C 55/08; C07C 57/44; C07C 237/12; C07C 229/26; C07C 229/36; C07D 207/16; C07C 229/22; C07D 213/82; C07D 213/80; C07D 275/06; C07D 207/28

(52) U.S. Cl. CPC C07D 403/14; C07B 2200/13; C07C 309/05; C07C 309/08; C07C 309/29; C07C 309/30; C07C 309/35; C07C 55/07; C07C 57/15; C07C 59/255; C07C 59/265; C07C 59/245; C07C 55/10; C07C 233/83; C07C 57/145; C07C 229/24; C07C 55/28; C07C 59/11; C07C 55/08; C07C 57/44; C07C 237/12; C07C 229/26; C07C 229/36; C07D 207/16; C07C 229/22; C07D 213/82; C07D 213/80; C07D 275/06; C07D 207/28; C07C 309/04

(57) ABSTRACT

The present invention provides compounds and compositions thereof which are useful as inhibitors of Bruton's tyrosine kinase and which exhibit desirable characteristics for the same.

### CROSS REFERENCE TO RELATED APPLICATIONS

The present application claims priority to U.S. provisional patent application No. 62/173,897, filed Jun. 10, 2015, the entire contents of which are hereby incorporated by reference.

### BACKGROUND OF THE INVENTION

Protein kinases are a large multigene family consisting of more than 500 proteins which play a critical role in the development and treatment of a number of human diseases in oncology, neurology and immunology. The Tec kinases are non-receptor tyrosine kinases which consists of five members (Tec (tyrosine kinase expressed in hepatocellular carcinoma), Btk (Bruton's tyrosine kinase), Itk (interleukin-2 (IL-2)-inducible T-cell kinase; also known as Emt or Tsk), Rlk (resting lymphocyte kinase; also known as Txk) and Bmx (bone-marrow tyrosine kinase gene on chromosome X; also known as Etk)) and are primarily expressed in haematopoietic cells, although expression of Bmx and Tec has been detected in endothelial and liver cells. Tec kinases (Itk, Rlk and Tec) are expressed in T cell and are all activated downstream of the T-cell receptor (TCR). Btk is a downstream mediator of B cell receptor (BCR) signaling which is involved in regulating B cell activation, proliferation, and differentiation. More specifically, Btk contains a PH domain that binds phosphatidylinositol (3,4,5)-trisphosphate (PIP3). PIP3 binding induces Btk to phosphorylate phospholipase C (PLCγ), which in turn hydrolyzes PIP2 to produce two secondary messengers, inositol triphosphate (IP3) and diacylglycerol (DAG), which activate protein kinase PKC, which then induces additional B-cell signaling. Mutations that disable Btk enzymatic activity result in XLA syndrome (X-linked agammaglobulinemia), a primary immunodeficiency. Given the critical roles which Tec kinases play in both B-cell and T-cell signaling, Tec kinases are targets of interest for autoimmune disorders.

Consequently, there is a great need in the art for effective inhibitors of Btk. The present invention fulfills these and other needs.

### SUMMARY OF THE INVENTION

It has now been found that novel forms of the present invention, and compositions thereof, are useful as inhibitors of one or more protein kinases and exhibit desirable characteristics for the same. In general, salt forms or freebase forms, and pharmaceutically acceptable compositions thereof, are useful for treating or lessening the severity of a variety of diseases or disorders as described in detail herein.

### BRIEF DESCRIPTION OF THE DRAWINGS

FIG. 1 provides the XRPD overlay of starting material and Compound 1 Type A reference.

FIG. 2 provides the TGA/DSC curves for Compound 1 Type A.

FIG. 3 provides XRPD patterns of two different samples of Compound 1 Type B (Samples A and A2).

FIG. 4 provides TGA/DSC curves of Compound 1 Type B.

FIG. 5 provides the XRPD pattern of Compound 1 Type C.

FIG. 6 provides the TGA/DSC curves of Compound 1 Type C.

FIG. 7 shows that no form change or decreased weight loss was observed by TGA after Compound 1 Type C was heated to 110° C.

FIG. 8 provides the XRPD pattern of Compound 1 Type D.

FIG. 9 provides the TGA/DSC curves of Compound 1 Type D.

FIG. 10 provides the XRPD overlay of Compound 1 Type D before and after heating.

FIG. 11 provides the XRPD pattern of Compound 1 Type E.

FIG. 12 provides the XRPD overlay of Compound 1 Type E before and after storage.

FIG. 13 provides the XRPD pattern of Compound 1 Type F.

FIG. 14 provides TGA and DSC curves of Compound 1 Type F.

FIG. 15 provides the XRPD pattern of Compound 1 Type G.

FIG. 16 provides the TGA/DSC curves of Compound 1 Type G.

FIG. 17 provides the XRPD patterns after slurry competition of Compound 1 anhydrates at room temperature and 50° C.

FIG. 18 provides XRPD patterns of Compound 1 Types A and B after slurry competition in acetone and water at room temperature.

FIG. 19 provides XRPD patterns of Compound 1 Types A and B after slurry competition in acetone and water at 50° C.

FIG. 20 provides the DVS plot of Compound 1 Type B.

FIG. 21 provides the XRPD pattern of Compound 2 Type A.

FIG. 22 provides the TGA and DSC curves of Compound 2 Type A.

FIG. 23 provides the XRPD pattern of the Compound 2 Type A solid form obtained by this scale-up procedure.

FIG. 24 provides the TGA/DSC curves of Compound 2 Type A obtained by the scale-up procedure.

FIG. 25 provides TGA studies showing bound water content.

FIG. 26 shows the water uptake of Compound 2 Type A.

FIG. 27 provides an overlay of XRPD data Compound 2 Type B before and after storage and as compared to Compound 2 Type A.

FIG. 28 shows XRPD data for new solid form Compound 2 Type C.

FIG. 29 provides DSC data for a sample comprising Compound 2 Type C (top) and compared to the DSC data for Compound 2 Type A (bottom).

FIG. 30 provides the XRPD pattern of the Compound 3 Type A, Compound 3 Type B, and Compound 3 Type C.

FIG. 31 provides the TGA/DSC curves of Compound 3 Type A.

FIG. 32 provides the TGA/DSC curves of Compound 3 Type B.

FIG. 33 provides the XRPD pattern of Compound 3 Type B obtained from the scale-up procedure.

FIG. 34 provides the TGA/DSC curves of Compound 3 Type B obtained from the scale-up procedure.

FIG. 35 provides the TGA/DSC curves of Compound 3 Type C.

FIG. 36 provides the XRPD pattern of Compound 4 Type A.

FIG. 37 provides the TGA/DSC curves of Compound 4 Type A.

FIG. 38 provides the XRPD pattern of Compound 5 Type A.

FIG. 39 provides the TGA/DSC curves of Compound 5 Type A.

FIG. 40 provides the XRPD pattern of Compound 6 Type A.

FIG. 41 provides the TGA/DSC curves of Compound 6 Type A.

FIG. 42 provides an overlay of XRPD patterns of Compound 7 Type A and Compound 7 Type B.

FIG. 43 provides TGA/DSC curves of Compound 7 Type A.

FIG. 44 provides TGA/DSC curves of Compound 7 Type B.

FIG. 45 provides an overlay of XRPD patterns of Compound 8 Types A-C, Compound 1 Type A, and toluenesulfonic acid.

FIG. 46 provides TGA/DSC curves of Compound 8 Type A.

FIG. 47 provides TGA/DSC curves of Compound 8 Type B.

FIG. 48 provides TGA/DSC curves of Compound 8 Type C.

FIG. 49 provides an overlay of XRPD patterns of Compound 9 Type A and Type B.

FIG. 50 provides TGA/DSC curves of Compound 9 Type A.

FIG. 51 provides TGA/DSC curves of Compound 9 Type B.

FIG. 52 provides an overlay of XRPD patterns of Compound 10 Type A, Type B, and Type C with Compound 1 Type A.

FIG. 53 provides TGA/DSC curves of Compound 10 Type A.

FIG. 54 provides TGA/DSC curves of Compound 10 Type B.

FIG. 55 provides TGA/DSC curves of Compound 10 Type C.

FIG. 56 provides an overlay of XRPD patterns of Compound 11 Type A, Type B, and Type C with Oxalic Acid and Compound 1 Type A.

FIG. 57 provides TGA/DSC curves of Compound 11 Type A.

FIG. 58 provides TGA/DSC curves of Compound 11 Type B.

FIG. 59 provides TGA/DSC curves of Compound 11 Type C.

FIG. 60 provides the XRPD pattern of Compound 12 Type A along with fumaric acid and Compound 1 Type A.

FIG. 61 provides TGA/DSC curves of Compound 12 Type A.

FIG. 62 provides the XRPD pattern of Compound 13 Type A.

FIG. 63 provides TGA/DSC curves of Compound 13 Type A.

FIG. 64 provides an overlay of XRPD patterns of Compound 14 Type A and Type B with citric acid and Compound 1 Type A.

FIG. 65 provides TGA/DSC curves of Compound 14 Type A.

FIG. 66 provides the XRPD pattern of Compound 14 Type A obtained from this scale-up protocol.

FIG. 67 provides TGA/DSC curves of Compound 14 Type A obtained from this scale-up protocol.

FIG. 68 provides TGA/DSC curves of Compound 14 Type B.

FIG. 69 provides the XRPD pattern of Compound 15 Type A.

FIG. 70 provides TGA/DSC curves of Compound 15 Type A.

FIG. 71 provides the XRPD pattern of Compound 16 Type A.

FIG. 72 provides TGA/DSC curves of Compound 16 Type A.

FIG. 73 provides an overlay of XRPD patterns of Compound 18 Type A and B with maleic acid and Compound 1 Type A.

FIG. 74 provides TGA/DSC curves of Compound 18 Type A.

FIG. 75 provides XRPD pattern of Compound 18 Type A as obtained from the scale-up procedure.

FIG. 76 provides TGA/DSC curves of Compound 18 Type A as obtained from the scale-up procedure.

FIG. 77 provides TGA/DSC curves of Compound 18 Type B.

FIG. 78 provides the XRPD pattern of Compound 21 Type A.

FIG. 79 provides the TGA/DSC curves of Compound 21 Type A.

FIG. 80 provides an overlay of XRPD patterns of Compound 32 Type A, Compound 32 Type B, Compound 32 Type C, and Compound 32 Type D, along with Compound 1 Type A and saccharin.

FIG. 81 provides TGA/DSC curves of Compound 32 Type A.

FIG. 82 provides TGA/DSC curves of Compound 32 Type B.

FIG. 83 provides TGA/DSC curves of Compound 32 Type C.

FIG. 84 provides TGA/DSC curves of Compound 32 Type D.

FIG. 85 provides stereochemical structure of Compound 1 single crystal.

FIG. 86 provides unit cell of Compound 1 single crystal.

### DETAILED DESCRIPTION OF THE INVENTION

### General Description of Certain Aspects of the Invention:

PCT patent publication WO2015/089337 (PCT application PCT/US14/69853, filed Dec. 11, 2014 (“the '853 application”)), the entirety of which is hereby incorporated herein by reference, describes certain Btk inhibitor compounds. Such compounds include 3-isopropoxy-N-(2-methyl-4-(2-((1-methyl-1H-pyrazol-4-yl)amino)pyrimidin-4-yl)benzyl) azetidine-1-carboxamide:

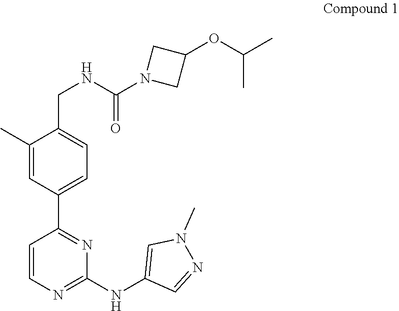 [SMILES: Cc1cc(-c2ccnc(Nc3cnn(C)c3)n2)ccc1CNC(=O)N1CC(OC(C)C)C1]

Compound 1, which is a freebase, is designated as compound number I-21 in the '853 application. The synthesis of Compound 1 is described in detail at Example 21 of the '853 application, which is reproduced herein for ease of reference.

Compound 1 has shown potency against BTK in in vitro and in vivo assays of BTK inhibition (see, e.g., Tables 1 and 2 of the '853 application). For example, the '853 application reports that Compound 1 has an IC₅₀<10 nM as measured in an in vitro Btk kinase assay and an IC₅₀<500 nM as measured in a pBTK assay. Accordingly, Compound 1 is useful for treating one or more disorders associated with activity of BTK.

It would be desirable to provide an acid addition product or solid form of compound 1 that imparts characteristics such as improved aqueous solubility, stability, absorption, bioavailability, and ease of formulation. Accordingly, the present invention provides both free base forms and acid addition forms of Compound 1.

### 1. Free Base Forms of Compound 1

It is contemplated that Compound 1 can exist in a variety of physical forms. For example, Compound 1 can be in solution, suspension, or in solid form. In certain embodiments, Compound 1 is in solid form. When Compound 1 is in solid form, said compound may be amorphous, crystalline, or a mixture thereof. Exemplary solid forms are described in more detail below.

In some embodiments, the present invention provides a form of Compound 1 substantially free of impurities. As used herein, the term “substantially free of impurities” means that the compound contains no significant amount of extraneous matter. Such extraneous matter may include different forms of Compound 1, residual solvents, or any other impurities that may result from the preparation of, and/or isolation of, Compound 1. In certain embodiments, at least about 95% by weight of a form of Compound 1 is present. In still other embodiments of the invention, at least about 99% by weight of a form of Compound 1 is present.

According to one embodiment, a form of Compound 1 is present in an amount of at least about 97, 97.5, 98.0, 98.5, 99, 99.5, 99.8 weight percent where the percentages are based on the total weight of the composition. According to another embodiment, a form of compound 1 contains no more than about 3.0 area percent HPLC of total organic impurities and, in certain embodiments, no more than about 1.5 area percent HPLC total organic impurities relative to the total area of the HPLC chromatogram. In other embodiments, a form of Compound 1 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for a form of Compound 1 is also meant to include all tautomeric forms of Compound 1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 1 can exist in a variety of solid forms. Exemplary such forms include polymorphs such as those described herein.

As used herein, the term “polymorph” refers to the different crystal structures into which a compound, or a salt or solvate thereof, can crystallize.

In certain embodiments, Compound 1 is a crystalline solid. In other embodiments, Compound 1 is a crystalline solid substantially free of amorphous Compound 1. As used herein, the term “substantially free of amorphous Compound 1” means that the compound contains no significant amount of amorphous Compound 1. In certain embodiments, at least about 95% by weight of crystalline Compound 1 is present. In still other embodiments of the invention, at least about 99% by weight of crystalline Compound 1 is present.

It has been found that Compound 1 can exist in at least seven distinct polymorphic forms. In certain embodiments, the present invention provides a polymorphic form of Compound 1 referred to herein as Type A. In certain embodiments, the present invention provides a polymorphic form of Compound 1 referred to herein as Type B. In certain embodiments, the present invention provides a polymorphic form of Compound 1 referred to herein as Type C. In certain embodiments, the present invention provides a polymorphic form of Compound 1 referred to herein as Type D. In certain embodiments, the present invention provides a polymorphic form of Compound 1 referred to herein as Type E. In certain embodiments, the present invention provides a polymorphic form of Compound 1 referred to herein as Type F. In certain embodiments, the present invention provides a polymorphic form of Compound 1 referred to herein as Type G.

In some embodiments, Compound 1 is amorphous. In some embodiments, compound 1 is amorphous, and is substantially free of crystalline compound 1.

In some embodiments, Compound 1 is an anhydrate. In other embodiments, Compound 1 is a hydrate.

### Compound 1 Type A

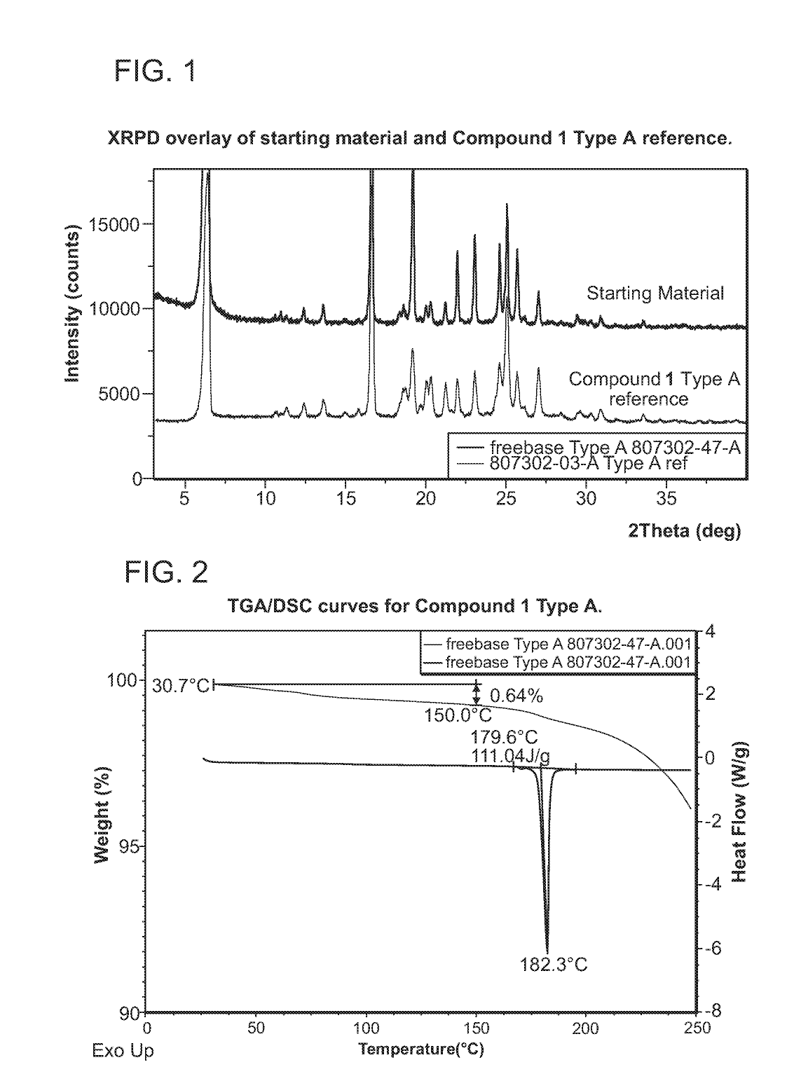

In certain embodiments, the X-ray powder diffraction pattern is substantially similar to the XRPD provided in FIG. 1.

Methods for preparing Compound 1 Type A are described infra.

### Compound 1 Type B

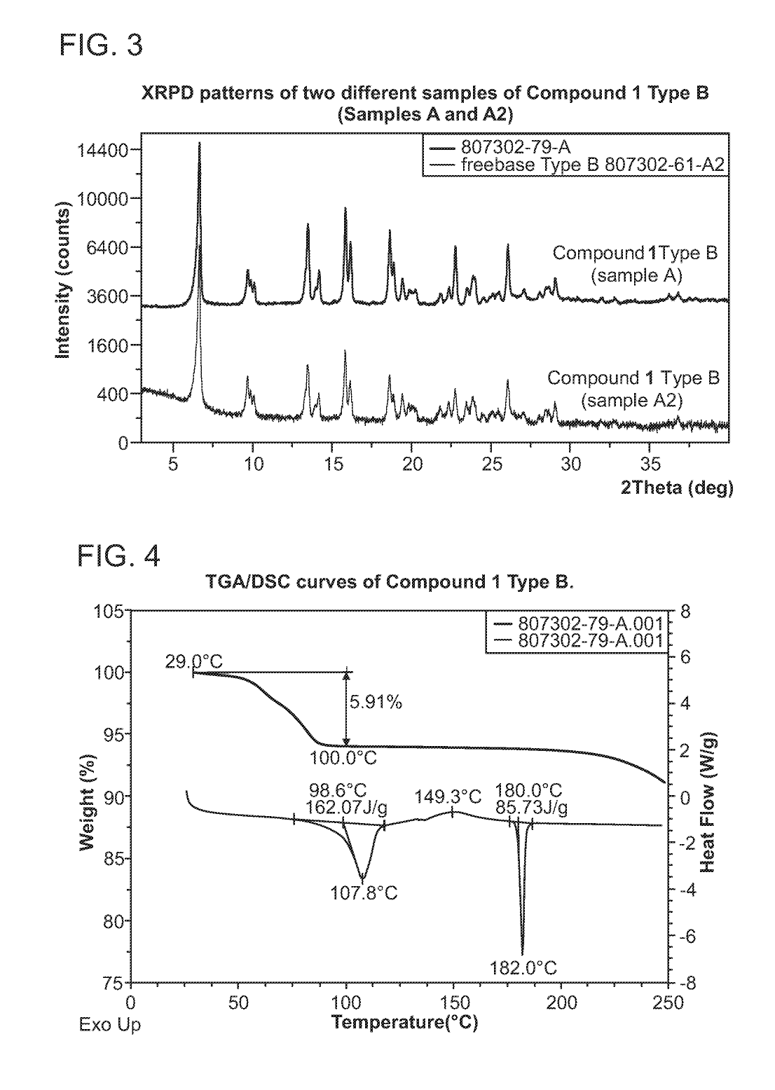

In certain embodiments, the X-ray powder diffraction pattern is substantially similar to the XRPD provided in FIG. 3.

Methods for preparing Compound 1 Type B are described infra.

### Compound 1 Type C

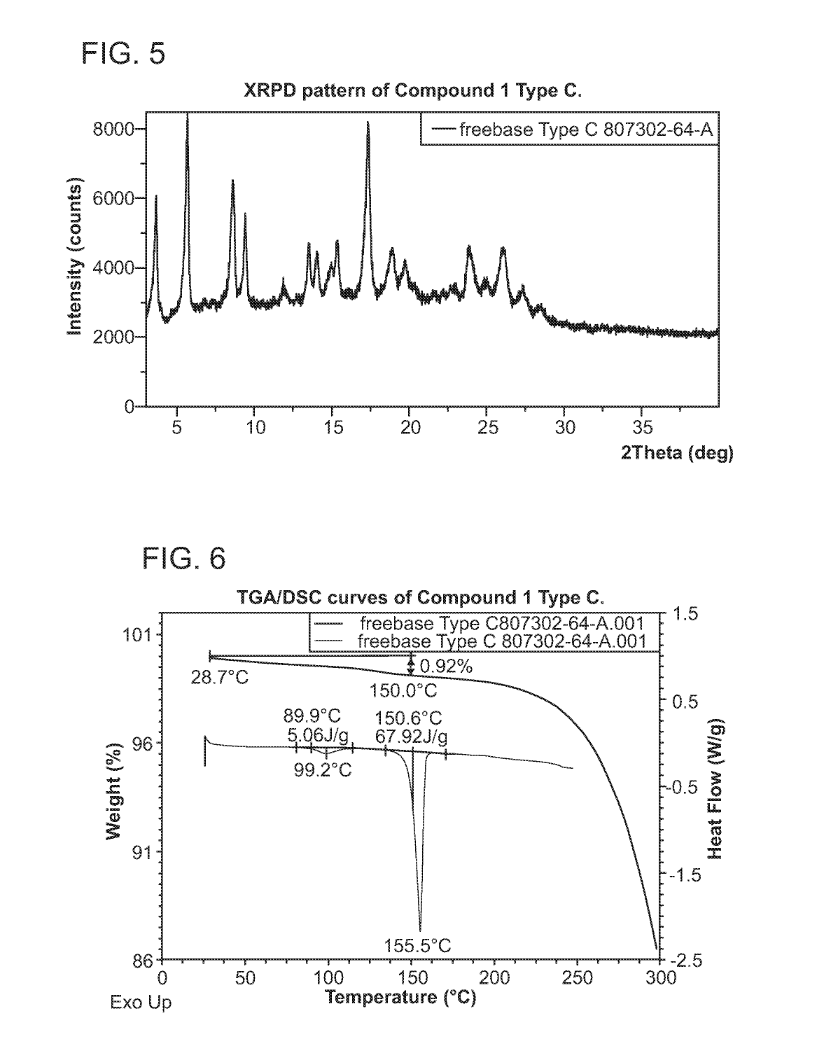

In certain embodiments, the X-ray powder diffraction pattern is substantially similar to the XRPD provided in FIG. 5.

Methods for preparing Compound 1 Type C are described infra.

### Compound 1 Type D

In certain embodiments, the X-ray powder diffraction pattern is substantially similar to the XRPD provided in FIG. 8.

Methods for preparing Compound 1 Type D are described infra.

### Compound 1 Type E

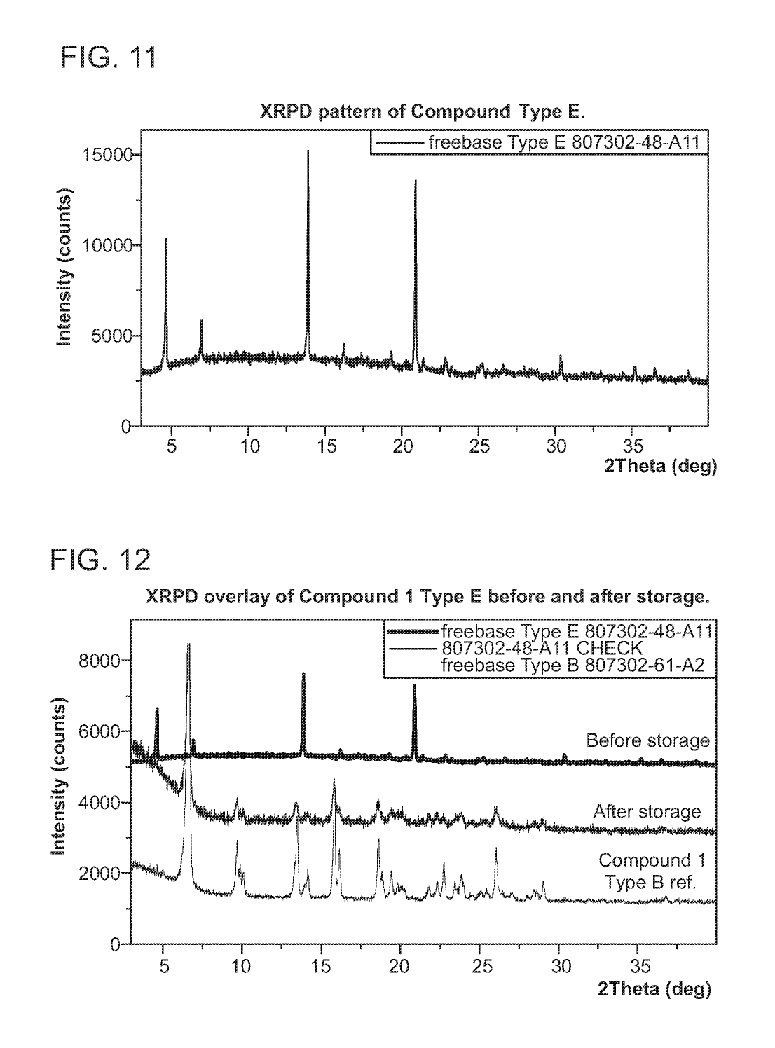

In certain embodiments, the X-ray powder diffraction pattern is substantially similar to the XRPD provided in FIG. 11.

Methods for preparing Compound 1 Type E are described infra.

### Compound 1 Type F

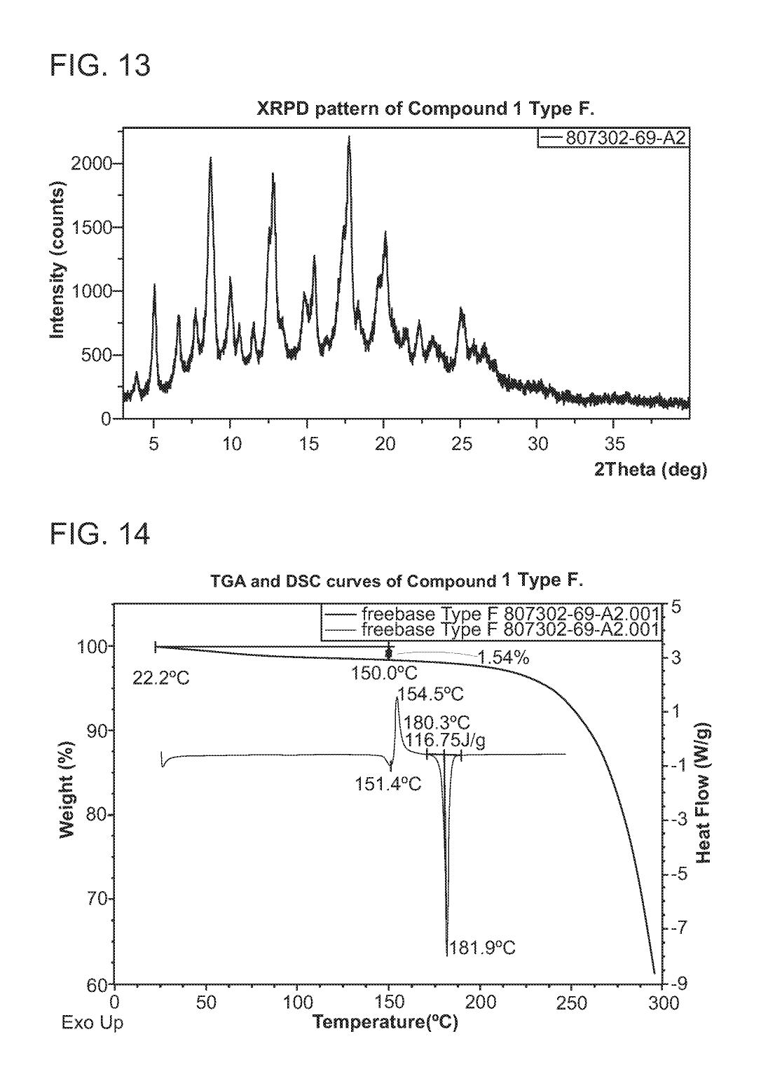

In certain embodiments, the X-ray powder diffraction pattern is substantially similar to the XRPD provided in FIG. 13.

Methods for preparing Compound 1 Type F are described infra.

### Compound 1 Type G

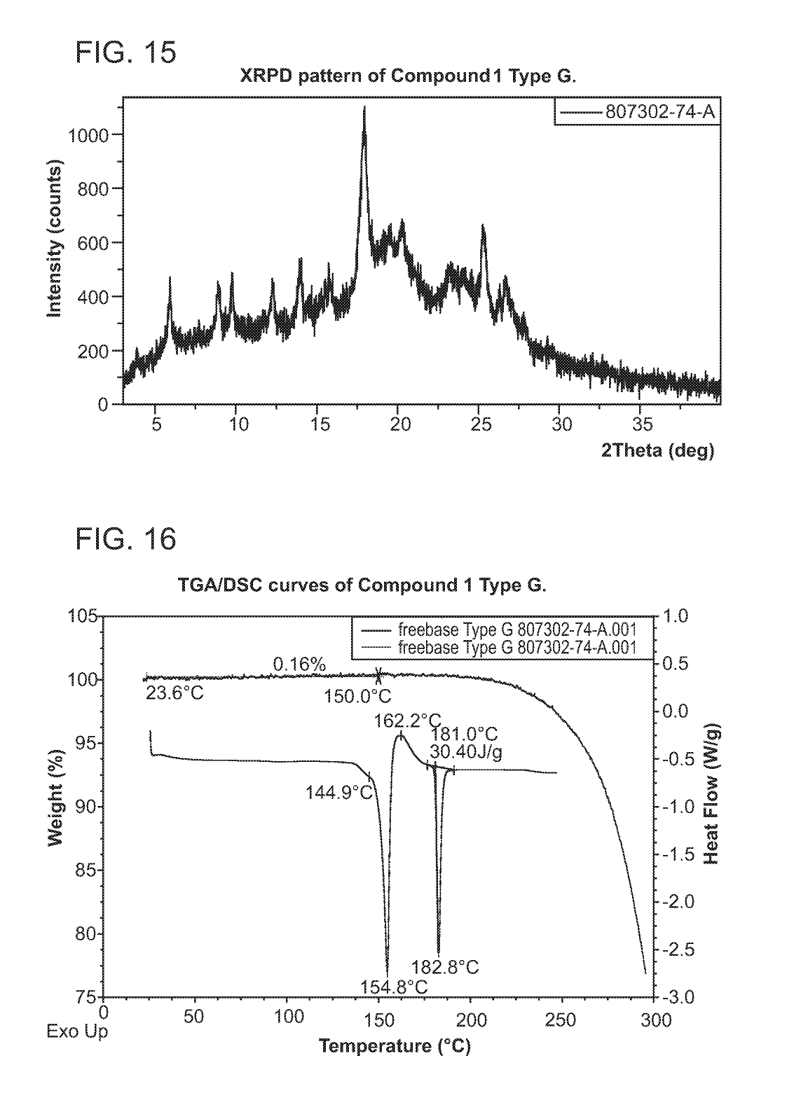

In certain embodiments, the X-ray powder diffraction pattern is substantially similar to the XRPD provided in FIG. 15.

Methods for preparing Compound 1 Type G are described infra.

Combinations of Co-Formers with Compound 1

In some embodiments, Compound 1 and a co-former (e.g., an acid) are combined to provide a species where Compound 1 and the acid are, e.g., ionically bonded or are hydrogen bonded to form one of Compounds 2 through 33, described below. It is contemplated that Compounds 2 through 33 can exist in a variety of physical forms. For example, Compounds 2 through 33 can be in solution, suspension, or in solid form. In certain embodiments, Compounds 2 through 33 are in solid form. When Compounds 2 through 33 are in solid form, said compounds may be amorphous, crystalline, or a mixture thereof. Exemplary solid forms of compounds 2 through 33 are described in more detail below.

### 2. Compound 2 (Hydrochloric Acid×Compound 1)

According to one embodiment, the present invention provides a chemical species Compound 2 comprising Compound 1 and hydrochloric acid:

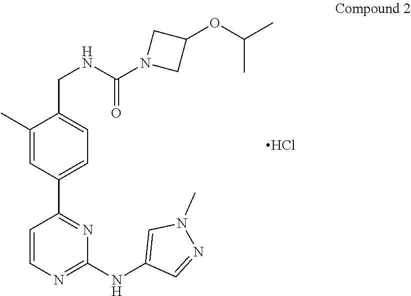 [SMILES: Cc1cc(-c2ccnc(Nc3cnn(C)c3)n2)ccc1CNC(=O)N1CC(OC(C)C)C1 ; Cl]

It is contemplated that Compound 2 can exist in a variety of physical forms. For example, Compound 2 can be in solution, suspension, or in solid form. In certain embodiments, Compound 2 is in solid form. When Compound 2 is in solid form, said compound may be amorphous, crystalline, or a mixture thereof. Exemplary solid forms are described in more detail below.

In one embodiment, the solid form of Compound 2 has a stoichiometry of (Compound 1):(hydrochloric acid) that is about 1:1.

In some embodiments, the present invention provides Compound 2 substantially free of impurities. As used herein, the term “substantially free of impurities” means that the compound contains no significant amount of extraneous matter. Such extraneous matter may include excess hydrochloric acid, excess Compound 1, residual solvents, or any other impurities that may result from the preparation of, and/or isolation of, Compound 2. In certain embodiments, at least about 95% by weight of Compound 2 is present. In still other embodiments of the invention, at least about 99% by weight of Compound 2 is present.

According to one embodiment, Compound 2 is present in an amount of at least about 97, 97.5, 98.0, 98.5, 99, 99.5, 99.8 weight percent where the percentages are based on the total weight of the composition. According to another embodiment, Compound 2 contains no more than about 3.0 area percent HPLC of total organic impurities and, in certain embodiments, no more than about 1.5 area percent HPLC total organic impurities relative to the total area of the HPLC chromatogram. In other embodiments, Compound 2 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 2 is also meant to include all tautomeric forms of Compound 2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 2 can exist in a variety of solid forms. Exemplary such forms include polymorphs such as those described herein.

In certain embodiments, Compound 2 is a crystalline solid. In other embodiments, Compound 2 is a crystalline solid substantially free of amorphous Compound 2. As used herein, the term “substantially free of amorphous Compound 2” means that the compound contains no significant amount of amorphous Compound 2. In certain embodiments, at least about 95% by weight of crystalline Compound 2 is present. In still other embodiments of the invention, at least about 99% by weight of crystalline Compound 2 is present.

It has been found that Compound 2 can exist in at least three distinct polymorphic forms. In some embodiments, the present invention provides a polymorphic form of Compound 2 referred to herein as Type A. In certain embodiments, the present invention provides a polymorphic form of Compound 2 referred to herein as Type B. In certain embodiments, the present invention provides a polymorphic form of Compound 2 referred to herein as Type C.

In some embodiments, Compound 2 is amorphous. In some embodiments, Compound 2 is amorphous, and is substantially free of crystalline Compound 2.

In some embodiments, Compound 2 is an anhydrate. In other embodiments, Compound 2 is a hydrate (e.g., a monohydrate).

### Compound 2 Type A

In some embodiments, Compound 2 Type A has at least 1, 2, 3, 4 or 5 spectral peak(s) selected from the peaks listed in Table 1 below.

TABLE 1
XRPD Peak Postions for Compound 2 Type A¹

| Position (°2θ) | Position (°2θ) |
|---|---|
| 5.23 | 21.27 |
| 8.27 | 21.55 |
| 9.11 | 21.91 |
| 12.39 | 23.83 |
| 13.58 | 25.01 |
| 14.40 | 25.58 |
| 14.73 | 26.57 |
| 15.78 | 27.24 |
| 16.70 | 27.69 |
| 17.30 | 28.59 |
| 17.96 | 29.21 |
| 18.29 | 30.41 |
| 18.75 | 31.67 |
| 20.60 | 33.21 |
| 21.08 | 33.86 |

¹this and all subsequent tables, the position 2θ is within ± 0.2.

In some embodiments, Compound 2 Type A is characterized in that it has one or more peaks in its X-ray powder diffraction pattern selected from those at about 5.23, 9.11, 12.39, 14.40, 14.73, 25.58, and 26.57. In some embodiments, Compound 2 Type A is characterized in that it has two or more peaks in its X-ray powder diffraction pattern selected from those at about 5.23, 9.11, 12.39, 14.40, 14.73, 25.58, and 26.57. In some embodiments, Compound 2 Type A is characterized in that it has three or more peaks in its X-ray powder diffraction pattern selected from those at about 5.23, 9.11, 12.39, 14.40, 14.73, 25.58, and 26.57. In some embodiments, Compound 2 Type A is characterized in that it has four or more peaks in its X-ray powder diffraction pattern selected from those at about 5.23, 9.11, 12.39, 14.40, 14.73, 25.58, and 26.57. In some embodiments, Compound 2 Type A is characterized in that it has five or more peaks in its X-ray powder diffraction pattern selected from those at about 5.23, 9.11, 12.39, 14.40, 14.73, 25.58, and 26.57. In some embodiments, Compound 2 Type A is characterized in that it has six or more peaks in its X-ray powder diffraction pattern selected from those at about 5.23, 9.11, 12.39, 14.40, 14.73, 25.58, and 26.57. As used herein, the term “about”, when used in reference to a degree 2-theta value refers to the stated value±0.2 degree 2-theta.

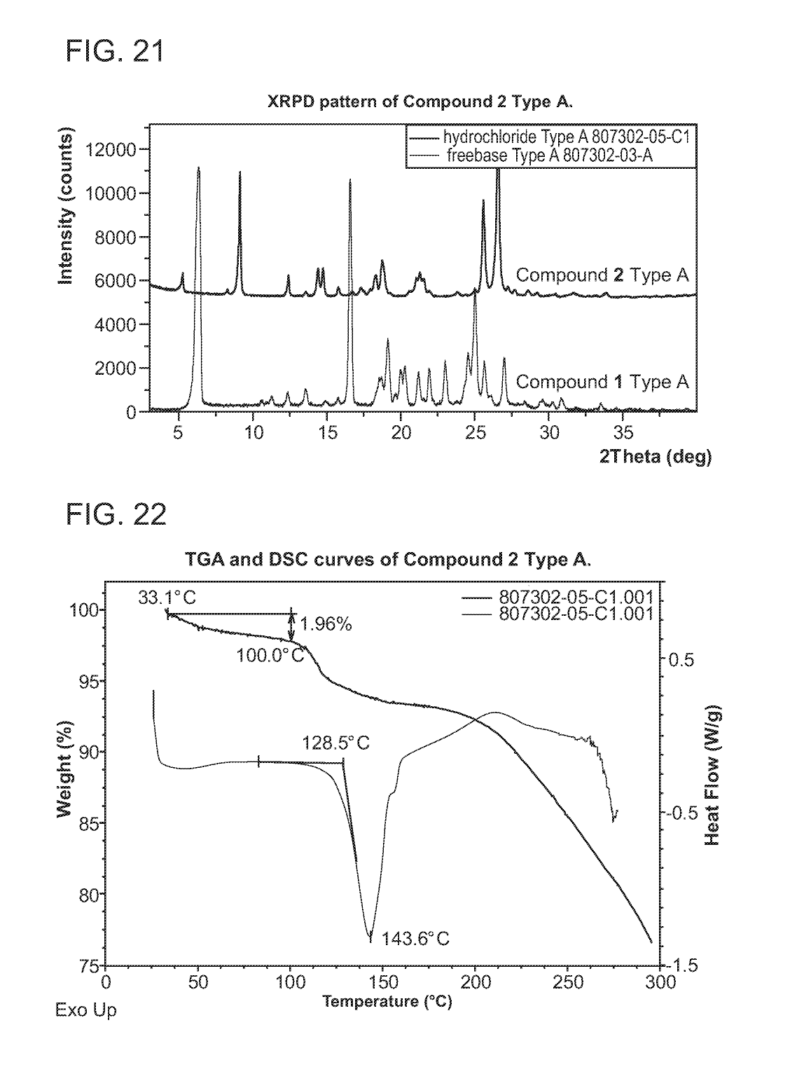

In certain embodiments, the X-ray powder diffraction pattern is substantially similar to the XRPD provided in FIG. 21.

Methods for preparing Compound 2 Type A are described infra.

### Compound 2 Type B

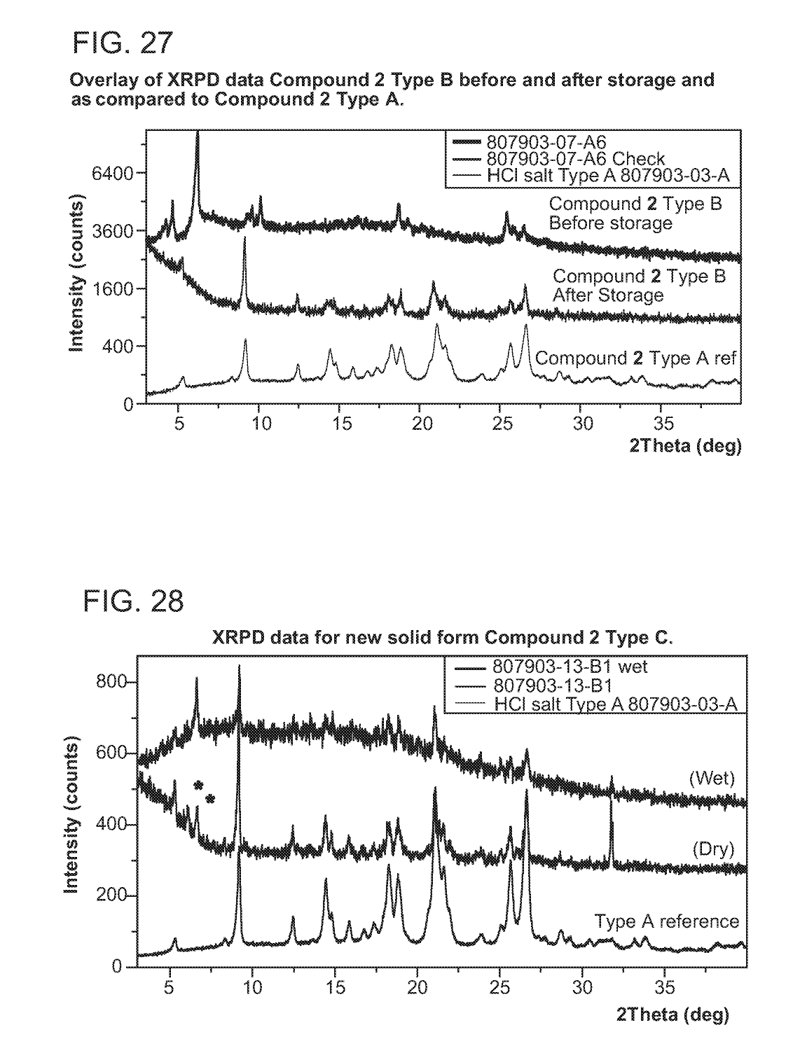

In certain embodiments, the X-ray powder diffraction pattern is substantially similar to the XRPD provided in FIG. 27.

Methods for preparing Compound 2 Type B are described infra.

### Compound 2 Type C

In certain embodiments, the X-ray powder diffraction pattern is substantially similar to the XRPD provided in FIG. 28.

Methods for preparing Compound 2 Type C are described infra.

### 3. Compound 3 (Sulfuric Acid×Compound 1)

According to one embodiment, the present invention provides a chemical species Compound 3 comprising Compound 1 and sulfuric acid:

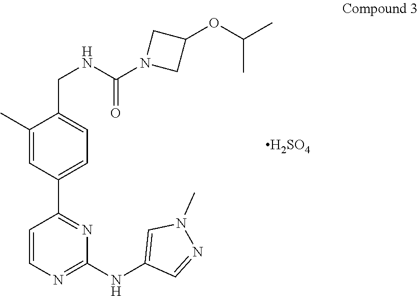

It is contemplated that Compound 3 can exist in a variety of physical forms. For example, Compound 3 can be in solution, suspension, or in solid form. In certain embodiments, Compound 3 is in solid form. When Compound 3 is in solid form, said compound may be amorphous, crystalline, or a mixture thereof. Exemplary solid forms are described in more detail below.

In one embodiment, the solid form of Compound 3 has a stoichiometry of (Compound 1):(sulfuric acid) that is about 1:1.

In some embodiments, the present invention provides Compound 3 substantially free of impurities. As used herein, the term “substantially free of impurities” means that the compound contains no significant amount of extraneous matter. Such extraneous matter may include excess sulfuric acid, excess Compound 1, residual solvents, or any other impurities that may result from the preparation of, and/or isolation of, Compound 3. In certain embodiments, at least about 95% by weight of Compound 3 is present. In still other embodiments of the invention, at least about 99% by weight of Compound 3 is present.

According to one embodiment, Compound 3 is present in an amount of at least about 97, 97.5, 98.0, 98.5, 99, 99.5, 99.8 weight percent where the percentages are based on the total weight of the composition. According to another embodiment, Compound 3 contains no more than about 3.0 area percent HPLC of total organic impurities and, in certain embodiments, no more than about 1.5 area percent HPLC total organic impurities relative to the total area of the HPLC chromatogram. In other embodiments, Compound 3 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 3 is also meant to include all tautomeric forms of Compound 3. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 3 can exist in a variety of solid forms. Exemplary such forms include polymorphs such as those described herein.

In certain embodiments, Compound 3 is a crystalline solid. In other embodiments, Compound 3 is a crystalline solid substantially free of amorphous Compound 3. As used herein, the term “substantially free of amorphous Compound 3” means that the compound contains no significant amount of amorphous Compound 3. In certain embodiments, at least about 95% by weight of crystalline Compound 3 is present. In still other embodiments of the invention, at least about 99% by weight of crystalline Compound 3 is present.

It has been found that Compound 3 can exist in at least three distinct polymorphic forms. In some embodiments, the present invention provides a polymorphic form of Compound 3 referred to herein as Type A. In certain embodiments, the present invention provides a polymorphic form of Compound 3 referred to herein as Type B. In certain embodiments, the present invention provides a polymorphic form of Compound 3 referred to herein as Type C.

In some embodiments, Compound 3 is amorphous. In some embodiments, Compound 3 is amorphous, and is substantially free of crystalline Compound 3.

### Compound 3 Type A

In some embodiments, Compound 3 Type A has at least 1, 2, 3, 4 or 5 spectral peak(s) selected from the peaks listed in Table 2 below.

TABLE 2
XRPD Peak Positions for Compound 3 Type A
Position (°2θ)

6.88
7.55
10.08
10.49
11.28
15.16
15.56
15.99
16.88
17.45
17.45
17.86
19.99
20.26
20.55
20.79
21.08
22.41
23.41
24.07
24.97
26.09
26.98
27.24
28.21
28.69
30.65

In some embodiments, Compound 3 Type A is characterized in that it has one or more peaks in its X-ray powder diffraction pattern selected from those at about 10.49, 15.99, 16.88, 17.86, and 21.96. In some embodiments, Compound 3 Type A is characterized in that it has two or more peaks in its X-ray powder diffraction pattern selected from those at about 10.49, 15.99, 16.88, 17.86, and 21.96. In some embodiments, Compound 3 Type A is characterized in that it has at least three peaks in its X-ray powder diffraction pattern selected from those at about 10.49, 15.99, 16.88, 17.86, and 21.96. In some embodiments, Compound 3 Type A is characterized in that it has at least four peaks in its X-ray powder diffraction pattern selected from those at about 10.49, 15.99, 16.88, 17.86, and 21.96. In some embodiments, Compound 3 Type A is characterized in that it has all five peaks in its X-ray powder diffraction pattern selected from those at about 10.49, 15.99, 16.88, 17.86, and 21.96.

In certain embodiments, the X-ray powder diffraction pattern is substantially similar to the XRPD provided in FIG. 30.

Methods for preparing Compound 3 Type A are described infra.

### Compound 3 Type B

In some embodiments, Compound 3 Type B has at least 1, 2, 3, 4 or 5 spectral peak(s) selected from the peaks listed in Table 3 below.

TABLE 3
XRPD Peak Positions for Compound 3 Type B
Position (°2θ)

6.11
8.97
10.70
11.49
12.93
15.38
16.20
16.50
17.23
18.05
18.25
19.02
19.64
20.04
21.54
22.55
23.11
24.15
25.08
26.08
26.54
27.08
27.59
33.93

In some embodiments, Compound 3 Type B is characterized in that it has one or more peaks in its X-ray powder diffraction pattern selected from those at about 6.11, 8.97, 11.49, 16.50, 21.54, and 26.54. In some embodiments, Compound 3 Type B is characterized in that it has two or more peaks in its X-ray powder diffraction pattern selected from those at about 6.11, 8.97, 11.49, 16.50, 21.54, and 26.54. In some embodiments, Compound 3 Type B is characterized in that it has at least three peaks in its X-ray powder diffraction pattern selected from those at about 6.11, 8.97, 11.49, 16.50, 21.54, and 26.54. In some embodiments, Compound 3 Type B is characterized in that it has at least four peaks in its X-ray powder diffraction pattern selected from those at about 6.11, 8.97, 11.49, 16.50, 21.54, and 26.54. In some embodiments, Compound 3 Type B is characterized in that it has at least five peaks in its X-ray powder diffraction pattern selected from those at about 6.11, 8.97, 11.49, 16.50, 21.54, and 26.54. In some embodiments, Compound 3 Type B is characterized in that it has at all six peaks in its X-ray powder diffraction pattern selected from those at about 6.11, 8.97, 11.49, 16.50, 21.54, and 26.54.

In certain embodiments, the X-ray powder diffraction pattern is substantially similar to the XRPD provided in FIG. 30.

Methods for preparing Compound 3 Type B are described infra.

### Compound 3 Type C

In some embodiments, Compound 3 Type C has at least 1, 2, 3, 4 or 5 spectral peak(s) selected from the peaks listed in Table 4 below.

TABLE 4
XRPD Peak Positions for Compound 3 Type C
Position (°2θ)

3.58
6.24
6.90
7.37
10.11
10.94
13.31
14.81
15.72
16.18
17.61
17.90
20.11
20.88
21.37
22.11
22.66
23.33
24.21
24.44
25.45
26.83
27.46
28.20
28.97
30.66

In some embodiments, Compound 3 Type C is characterized in that it has one or more peaks in its X-ray powder diffraction pattern selected from those at about 3.58, 10.94, 14.81, and 24.44. In some embodiments, Compound 3 Type C is characterized in that it has two or more peaks in its X-ray powder diffraction pattern selected from those at about 3.58, 10.94, 14.81, and 24.44. In some embodiments, Compound 3 Type C is characterized in that it has at least three peaks in its X-ray powder diffraction pattern selected from those at about 3.58, 10.94, 14.81, and 24.44. In some embodiments, Compound 3 Type C is characterized in that it has at all four peaks in its X-ray powder diffraction pattern selected from those at about 3.58, 10.94, 14.81, and 24.44.

In certain embodiments, the X-ray powder diffraction pattern is substantially similar to the XRPD provided in FIG. 30.

Methods for preparing Compound 3 Type C are described infra.

### 4. Compound 4 (Methanesulfonic Acid×Compound 1)

According to one embodiment, the present invention provides a chemical species Compound 4 comprising Compound 1 and methanesulfonic acid:

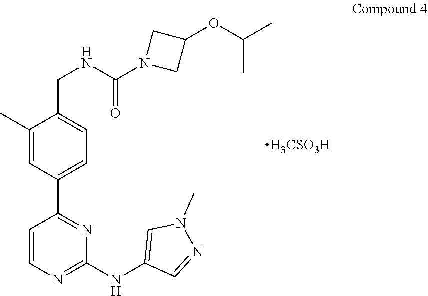

It is contemplated that Compound 4 can exist in a variety of physical forms. For example, Compound 4 can be in solution, suspension, or in solid form. In certain embodiments, Compound 4 is in solid form. When Compound 4 is in solid form, said compound may be amorphous, crystalline, or a mixture thereof. Exemplary solid forms are described in more detail below.

In one embodiment, the solid form of Compound 4 has a stoichiometry of (Compound 1):(methanesulfonic acid) that is about 1:1.

In some embodiments, the present invention provides Compound 4 substantially free of impurities. As used herein, the term “substantially free of impurities” means that the compound contains no significant amount of extraneous matter. Such extraneous matter may include excess methanesulfonic acid, excess compound 1, residual solvents, or any other impurities that may result from the preparation of, and/or isolation of, Compound 4. In certain embodiments, at least about 95% by weight of Compound 4 is present. In still other embodiments of the invention, at least about 99% by weight of Compound 4 is present.

According to one embodiment, Compound 4 is present in an amount of at least about 97, 97.5, 98.0, 98.5, 99, 99.5, 99.8 weight percent where the percentages are based on the total weight of the composition. According to another embodiment, Compound 4 contains no more than about 3.0 area percent HPLC of total organic impurities and, in certain embodiments, no more than about 1.5 area percent HPLC total organic impurities relative to the total area of the HPLC chromatogram. In other embodiments, Compound 4 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 4 is also meant to include all tautomeric forms of Compound 4. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 4 can exist in a variety of solid forms. Exemplary such forms include polymorphs such as those described herein.

In certain embodiments, Compound 4 is a crystalline solid. In other embodiments, Compound 4 is a crystalline solid substantially free of amorphous Compound 4. As used herein, the term “substantially free of amorphous Compound 4” means that the compound contains no significant amount of amorphous Compound 4. In certain embodiments, at least about 95% by weight of crystalline Compound 4 is present. In still other embodiments of the invention, at least about 99% by weight of crystalline Compound 4 is present.

It has been found that Compound 4 can exist in at least one distinct crystalline form. In some embodiments, the present invention provides a crystalline form of Compound 4 referred to herein as Type A.

In some embodiments, Compound 4 is amorphous. In some embodiments, Compound 4 is amorphous, and is substantially free of crystalline Compound 4.

### Compound 4 Type A

In some embodiments, Compound 4 Type A has at least 1, 2, 3, 4 or 5 spectral peak(s) selected from the peaks listed in Table 5 below.

TABLE 5
XRPD Peak Positions for Compound 4 Type A
Position (°2θ)

4.14
5.80
7.51
9.53
10.54
11.08
11.77
12.33
14.63
15.10
16.83
17.19
17.52
17.97
18.41
18.88
19.46
20.02
20.70
21.13
21.66
22.33
22.89
23.41
23.80
24.15
24.85
25.56
26.08
27.10
27.33
29.35
30.59
30.98
31.71
31.94
33.01
39.29

In some embodiments, Compound 4 Type A is characterized in that it has one or more peaks in its X-ray powder diffraction pattern selected from those at about 5.80, 7.51, 10.54, 11.77, 20.02, 21.66, and 25.56. In some embodiments, Compound 4 Type A is characterized in that it has two or more peaks in its X-ray powder diffraction pattern selected from those at about 5.80, 7.51, 10.54, 11.77, 20.02, 21.66, and 25.56. In some embodiments, Compound 4 Type A is characterized in that it has three or more peaks in its X-ray powder diffraction pattern selected from those at about 5.80, 7.51, 10.54, 11.77, 20.02, 21.66, and 25.56. In some embodiments, Compound 4 Type A is characterized in that it has four or more peaks in its X-ray powder diffraction pattern selected from those at about 5.80, 7.51, 10.54, 11.77, 20.02, 21.66, and 25.56. In some embodiments, Compound 4 Type A is characterized in that it has five or more peaks in its X-ray powder diffraction pattern selected from those at about 5.80, 7.51, 10.54, 11.77, 20.02, 21.66, and 25.56. In some embodiments, Compound 4 Type A is characterized in that it has six or more peaks in its X-ray powder diffraction pattern selected from those at about 5.80, 7.51, 10.54, 11.77, 20.02, 21.66, and 25.56. In some embodiments, Compound 4 Type A is characterized in that it has all seven peaks in its X-ray powder diffraction pattern selected from those at about 5.80, 7.51, 10.54, 11.77, 20.02, 21.66, and 25.56.

In certain embodiments, the X-ray powder diffraction pattern is substantially similar to the XRPD provided in FIG. 36.

Methods for preparing Compound 4 Type A are described infra.

### 5. Compound 5 (Ethanedisulfonic Acid×Compound 1)

According to one embodiment, the present invention provides a chemical species Compound 5 comprising Compound 1 and ethanedisulfonic acid:

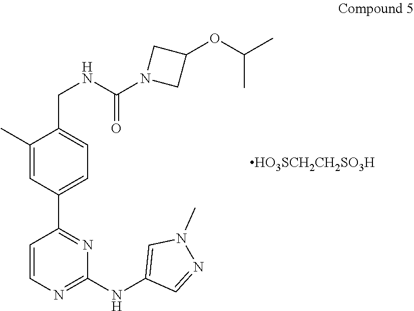

It is contemplated that Compound 5 can exist in a variety of physical forms. For example, Compound 5 can be in solution, suspension, or in solid form. In certain embodiments, Compound 5 is in solid form. When Compound 5 is in solid form, said compound may be amorphous, crystalline, or a mixture thereof. Exemplary solid forms are described in more detail below.

In one embodiment, the solid form of Compound 5 has a stoichiometry of (Compound 1):(ethanedisulfonic acid) that is about 1:1.

In some embodiments, the present invention provides Compound 5 substantially free of impurities. As used herein, the term “substantially free of impurities” means that the compound contains no significant amount of extraneous matter. Such extraneous matter may include excess ethanedisulfonic acid, excess Compound 1, residual solvents, or any other impurities that may result from the preparation of, and/or isolation of, Compound 5. In certain embodiments, at least about 95% by weight of Compound 5 is present. In still other embodiments of the invention, at least about 99% by weight of Compound 5 is present.

According to one embodiment, Compound 5 is present in an amount of at least about 97, 97.5, 98.0, 98.5, 99, 99.5, 99.8 weight percent where the percentages are based on the total weight of the composition. According to another embodiment, Compound 5 contains no more than about 3.0 area percent HPLC of total organic impurities and, in certain embodiments, no more than about 1.5 area percent HPLC total organic impurities relative to the total area of the HPLC chromatogram. In other embodiments, Compound 5 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 5 is also meant to include all tautomeric forms of Compound 5. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 5 can exist in a variety of solid forms. Exemplary such forms include polymorphs such as those described herein.

In certain embodiments, Compound 5 is a crystalline solid. In other embodiments, Compound 5 is a crystalline solid substantially free of amorphous Compound 5. As used herein, the term “substantially free of amorphous Compound 5” means that the compound contains no significant amount of amorphous Compound 5. In certain embodiments, at least about 95% by weight of crystalline Compound 5 is present. In still other embodiments of the invention, at least about 99% by weight of crystalline Compound 5 is present.

It has been found that Compound 5 can exist in at least one distinct crystalline form. In some embodiments, the present invention provides a crystalline form of Compound 5 referred to herein as Type A.

In some embodiments, Compound 5 is amorphous. In some embodiments, Compound 5 is amorphous, and is substantially free of crystalline Compound 5.

### Compound 5 Type A

In some embodiments, Compound 5 Type A has at least 1, 2, 3, 4 or 5 spectral peak(s) selected from the peaks listed in Table 6 below.

TABLE 6
XRPD Peak Positions for Compound 5 Type A
Position (°2θ)

5.47
6.18
7.98
8.55
12.26
14.46
14.72
15.17
15.35
15.70
16.37
17.21
17.69
18.10
18.64
19.04
19.15
19.81
20.10
21.07
21.48
21.69
22.58
23.43
24.52
24.67
25.00
25.86
26.30
26.77
27.47
28.80
29.36
29.88
30.73
31.43
32.43
32.93
33.34

In some embodiments, Compound 5 Type A is characterized in that it has one or more peaks in its X-ray powder diffraction pattern selected from those at about 5.47, 8.55, 17.69, 18.10, 23.43, and 25.86. In some embodiments, Compound 5 Type A is characterized in that it has two or more peaks in its X-ray powder diffraction pattern selected from those at about 5.47, 8.55, 17.69, 18.10, 23.43, and 25.86. In some embodiments, Compound 5 Type A is characterized in that it has three or more peaks in its X-ray powder diffraction pattern selected from those at about 5.47, 8.55, 17.69, 18.10, 23.43, and 25.86. In some embodiments, Compound 5 Type A is characterized in that it has four or more peaks in its X-ray powder diffraction pattern selected from those at about 5.47, 8.55, 17.69, 18.10, 23.43, and 25.86. In some embodiments, Compound 5 Type A is characterized in that it has five or more peaks in its X-ray powder diffraction pattern selected from those at about 5.47, 8.55, 17.69, 18.10, 23.43, and 25.86. In some embodiments, Compound 5 Type A is characterized in that it has all six peaks in its X-ray powder diffraction pattern selected from those at about 5.47, 8.55, 17.69, 18.10, 23.43, and 25.86.

In certain embodiments, the X-ray powder diffraction pattern is substantially similar to the XRPD provided in FIG. 38.

Methods for preparing Compound 5 Type A are described infra.

### 6. Compound 6 (2-Hydroxyethanesulfonic Acid×Compound 1)

According to one embodiment, the present invention provides a chemical species Compound 6 comprising Compound 1 and 2-hydroxyethanesulfonic acid:

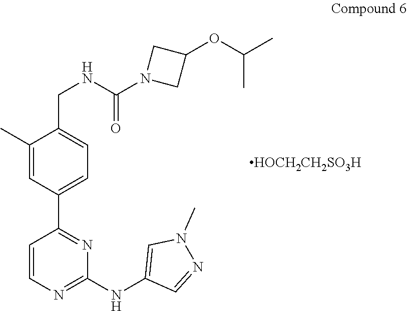

It is contemplated that Compound 6 can exist in a variety of physical forms. For example, Compound 6 can be in solution, suspension, or in solid form. In certain embodiments, Compound 6 is in solid form. When Compound 6 is in solid form, said compound may be amorphous, crystalline, or a mixture thereof. Exemplary solid forms are described in more detail below.

In one embodiment, the solid form of Compound 6 has a stoichiometry of (Compound 1):(2-hydroxyethanesulfonic acid) that is about 1:1.

In some embodiments, the present invention provides Compound 6 substantially free of impurities. As used herein, the term “substantially free of impurities” means that the compound contains no significant amount of extraneous matter. Such extraneous matter may include excess 2-hydroxyethanesulfonic acid, excess Compound 1, residual solvents, or any other impurities that may result from the preparation of, and/or isolation of, Compound 6. In certain embodiments, at least about 95% by weight of Compound 6 is present. In still other embodiments of the invention, at least about 99% by weight of Compound 6 is present.

According to one embodiment, Compound 6 is present in an amount of at least about 97, 97.5, 98.0, 98.5, 99, 99.5, 99.8 weight percent where the percentages are based on the total weight of the composition. According to another embodiment, Compound 6 contains no more than about 3.0 area percent HPLC of total organic impurities and, in certain embodiments, no more than about 1.5 area percent HPLC total organic impurities relative to the total area of the HPLC chromatogram. In other embodiments, Compound 6 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 6 is also meant to include all tautomeric forms of Compound 6. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 6 can exist in a variety of solid forms. Exemplary such forms include polymorphs such as those described herein.

In certain embodiments, Compound 6 is a crystalline solid. In other embodiments, Compound 6 is a crystalline solid substantially free of amorphous Compound 6. As used herein, the term “substantially free of amorphous Compound 6” means that the compound contains no significant amount of amorphous Compound 6. In certain embodiments, at least about 95% by weight of crystalline Compound 6 is present. In still other embodiments of the invention, at least about 99% by weight of crystalline Compound 6 is present.

It has been found that Compound 6 can exist in at least one distinct crystalline form. In some embodiments, the present invention provides a crystalline form of Compound 6 referred to herein as Type A.

In some embodiments, Compound 6 is amorphous. In some embodiments, Compound 6 is amorphous, and is substantially free of crystalline Compound 6.

### Compound 6 Type A

In some embodiments, Compound 6 Type A has at least 1, 2, 3, 4 or 5 spectral peak(s) selected from the peaks listed in Table 7 below.

TABLE 7
XRPD Peak Positions for Compound 6 Type A
Position (°2θ)

3.43
8.05
9.32
9.65
14.93
15.99
16.19
16.44
17.15
17.82
18.52
18.68
19.46
19.77
20.65
20.71
21.54
21.99
23.01
24.01
24.66
25.29
25.73
26.42
27.03
28.85
29.11
32.23
33.47
33.87
34.58
35.70
36.13

In some embodiments, Compound 6 Type A is characterized in that it has one or more peaks in its X-ray powder diffraction pattern selected from those at about 3.43, 8.05, 9.32, 17.15, 17.82, and 24.66. In some embodiments, Compound 6 Type A is characterized in that it has two or more peaks in its X-ray powder diffraction pattern selected from those at about 3.43, 8.05, 9.32, 17.15, 17.82, and 24.66. In some embodiments, Compound 6 Type A is characterized in that it has three or more peaks in its X-ray powder diffraction pattern selected from those at about 3.43, 8.05, 9.32, 17.15, 17.82, and 24.66. In some embodiments, Compound 6 Type A is characterized in that it has four or more peaks in its X-ray powder diffraction pattern selected from those at about 3.43, 8.05, 9.32, 17.15, 17.82, and 24.66. In some embodiments, Compound 6 Type A is characterized in that it has five or more peaks in its X-ray powder diffraction pattern selected from those at about 3.43, 8.05, 9.32, 17.15, 17.82, and 24.66. In some embodiments, Compound 6 Type A is characterized in that it has all six peaks in its X-ray powder diffraction pattern selected from those at about 3.43, 8.05, 9.32, 17.15, 17.82, and 24.66.

In certain embodiments, the X-ray powder diffraction pattern is substantially similar to the XRPD provided in FIG. 40.

Methods for preparing Compound 6 Type A are described infra.

### 7. Compound 7 (Benzenesulfonic Acid×Compound 1)

According to one embodiment, the present invention provides a chemical species Compound 7 comprising Compound 1 and benzenesulfonic acid:

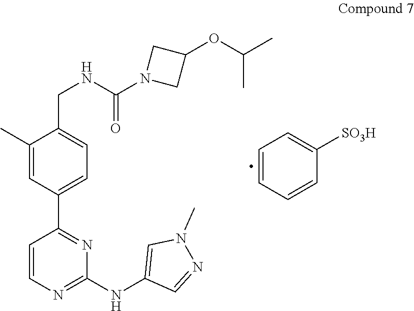 [SMILES: Cc1cc(-c2ccnc(Nc3cnn(C)c3)n2)ccc1CNC(=O)N1CC(OC(C)C)C1 ; O=S(=O)(O)c1ccccc1]

It is contemplated that Compound 7 can exist in a variety of physical forms. For example, Compound 7 can be in solution, suspension, or in solid form. In certain embodiments, Compound 7 is in solid form. When Compound 7 is in solid form, said compound may be amorphous, crystalline, or a mixture thereof. Exemplary solid forms are described in more detail below.

In one embodiment, the solid form of Compound 7 has a stoichiometry of (Compound 1):(benzenesulfonic acid) that is about 1:1.

In some embodiments, the present invention provides Compound 7 substantially free of impurities. As used herein, the term “substantially free of impurities” means that the compound contains no significant amount of extraneous matter. Such extraneous matter may include excess benzenesulfonic acid, excess Compound 1, residual solvents, or any other impurities that may result from the preparation of, and/or isolation of, Compound 7. In certain embodiments, at least about 95% by weight of Compound 7 is present. In still other embodiments of the invention, at least about 99% by weight of Compound 7 is present.

According to one embodiment, Compound 7 is present in an amount of at least about 97, 97.5, 98.0, 98.5, 99, 99.5, 99.8 weight percent where the percentages are based on the total weight of the composition. According to another embodiment, Compound 7 contains no more than about 3.0 area percent HPLC of total organic impurities and, in certain embodiments, no more than about 1.5 area percent HPLC total organic impurities relative to the total area of the HPLC chromatogram. In other embodiments, Compound 7 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 7 is also meant to include all tautomeric forms of Compound 7. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 7 can exist in a variety of solid forms. Exemplary such forms include polymorphs such as those described herein.

In certain embodiments, Compound 7 is a crystalline solid. In other embodiments, Compound 7 is a crystalline solid substantially free of amorphous Compound 7. As used herein, the term “substantially free of amorphous Compound 7” means that the compound contains no significant amount of amorphous Compound 7. In certain embodiments, at least about 95% by weight of crystalline Compound 7 is present. In still other embodiments of the invention, at least about 99% by weight of crystalline Compound 7 is present.

It has been found that Compound 7 can exist in at least two distinct polymorphic forms. In some embodiments, the present invention provides a polymorphic form of Compound 7 referred to herein as Type A. In some embodiments, the present invention provides a polymorphic form of Compound 7 referred to herein as Type B.

In some embodiments, Compound 7 is amorphous. In some embodiments, Compound 7 is amorphous, and is substantially free of crystalline Compound 7.

### Compound 7 Type A

In some embodiments, Compound 7 Type A has at least 1, 2, 3, 4 or 5 spectral peak(s) selected from the peaks listed in Table 8 below.

TABLE 8
XRPD Peak Positions for Compound 7 Type A
Position (°2θ)

4.56
5.29
6.61
7.14
9.56
10.28
10.54
10.93
11.52
11.56
12.17
13.22
14.34
14.88
15.32
16.04
17.08
17.15
17.95
18.29
18.91
19.48
19.68
20.28
21.23
22.00
22.77
23.35
23.93
24.68
25.17
25.89
26.42
27.23
28.11
28.47
30.41
30.96
32.21
32.48
37.43

In some embodiments, Compound 7 Type A is characterized in that it has one or more peaks in its X-ray powder diffraction pattern selected from those at about 4.56, 5.29, 10.28, 18.29, 21.23, and 23.35. In some embodiments, Compound 7 Type A is characterized in that it has two or more peaks in its X-ray powder diffraction pattern selected from those at about 4.56, 5.29, 10.28, 18.29, 21.23, and 23.35. In some embodiments, Compound 7 Type A is characterized in that it has three or more peaks in its X-ray powder diffraction pattern selected from those at about 4.56, 5.29, 10.28, 18.29, 21.23, and 23.35. In some embodiments, Compound 7 Type A is characterized in that it has four or more peaks in its X-ray powder diffraction pattern selected from those at about 4.56, 5.29, 10.28, 18.29, 21.23, and 23.35. In some embodiments, Compound 7 Type A is characterized in that it has five or more peaks in its X-ray powder diffraction pattern selected from those at about 4.56, 5.29, 10.28, 18.29, 21.23, and 23.35. In some embodiments, Compound 7 Type A is characterized in that it has all six peaks in its X-ray powder diffraction pattern selected from those at about 4.56, 5.29, 10.28, 18.29, 21.23, and 23.35.

In certain embodiments, the X-ray powder diffraction pattern is substantially similar to the XRPD provided in FIG. 42.

Methods for preparing Compound 7 Type A are described infra.

### Compound 7 Type B

In some embodiments, Compound 7 Type B has at least 1, 2, 3, 4 or 5 spectral peak(s) selected from the peaks listed in Table 9 below.

TABLE 9
XRPD Peak Positions for Compound 7 Type B
Position (°2θ)

4.30
8.66
13.99
17.80
25.83

In some embodiments, Compound 7 Type B is characterized in that it has one or more peaks in its X-ray powder diffraction pattern selected from those at about 4.30 and 8.66. In some embodiments, Compound 7 Type B is characterized in that it has both peaks in its X-ray powder diffraction pattern selected from those at about 4.30 and 8.66.

In certain embodiments, the X-ray powder diffraction pattern is substantially similar to the XRPD provided in FIG. 42.

Methods for preparing Compound 7 Type B are described infra.

### 8. Compound 8 (Toluenesulfonic Acid×Compound 1)

According to one embodiment, the present invention provides a chemical species Compound 8 comprising Compound 1 and toluenesulfonic acid:

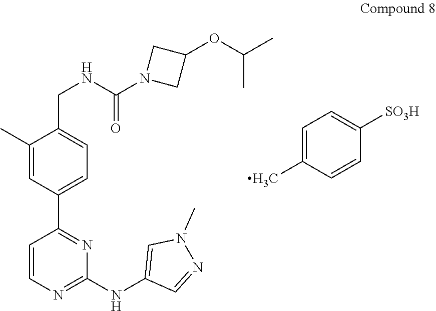

It is contemplated that Compound 8 can exist in a variety of physical forms. For example, Compound 8 can be in solution, suspension, or in solid form. In certain embodiments, Compound 8 is in solid form. When Compound 8 is in solid form, said compound may be amorphous, crystalline, or a mixture thereof. Exemplary solid forms are described in more detail below.

In one embodiment, the solid form of Compound 8 has a stoichiometry of (Compound 1):(toluenesulfonic acid) that is about 1:1.

In some embodiments, the present invention provides Compound 8 substantially free of impurities. As used herein, the term “substantially free of impurities” means that the compound contains no significant amount of extraneous matter. Such extraneous matter may include excess toluenesulfonic acid, excess Compound 1, residual solvents, or any other impurities that may result from the preparation of, and/or isolation of, Compound 8. In certain embodiments, at least about 95% by weight of Compound 8 is present. In still other embodiments of the invention, at least about 99% by weight of Compound 8 is present.

According to one embodiment, Compound 8 is present in an amount of at least about 97, 97.5, 98.0, 98.5, 99, 99.5, 99.8 weight percent where the percentages are based on the total weight of the composition. According to another embodiment, Compound 8 contains no more than about 3.0 area percent HPLC of total organic impurities and, in certain embodiments, no more than about 1.5 area percent HPLC total organic impurities relative to the total area of the HPLC chromatogram. In other embodiments, Compound 8 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 8 is also meant to include all tautomeric forms of Compound 8. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 8 can exist in a variety of solid forms. Exemplary such forms include polymorphs such as those described herein.

In certain embodiments, Compound 8 is a crystalline solid. In other embodiments, Compound 8 is a crystalline solid substantially free of amorphous Compound 8. As used herein, the term “substantially free of amorphous Compound 8” means that the compound contains no significant amount of amorphous Compound 8. In certain embodiments, at least about 95% by weight of crystalline Compound 8 is present. In still other embodiments of the invention, at least about 99% by weight of crystalline Compound 8 is present.

It has been found that Compound 8 can exist in at least three distinct polymorphic forms. In some embodiments, the present invention provides a polymorphic form of Compound 8 referred to herein as Type A. In some embodiments, the present invention provides a polymorphic form of Compound 8 referred to herein as Type B. In some embodiments, the present invention provides a polymorphic form of Compound 8 referred to herein as Type C.

In some embodiments, Compound 8 is amorphous. In some embodiments, Compound 8 is amorphous, and is substantially free of crystalline Compound 8.

### Compound 8 Type A

In some embodiments, Compound 8 Type A has at least 1, 2, 3, 4 or 5 spectral peak(s) selected from the peaks listed in Table 10 below.

TABLE 10
XRPD Peak Positions for Compound 8 Type A
Position (°2θ)

5.01
6.93
9.56
10.28
10.83
11.21
13.22
13.94
14.32
15.21
17.06
17.62
18.24
18.65
19.27
19.93
20.10
20.99
21.77
22.56
23.51
24.61
25.92
27.02
28.10
29.24
30.45

In some embodiments, Compound 8 Type A is characterized in that it has one or more peaks in its X-ray powder diffraction pattern selected from those at about 11.21, 17.06, 20.10, and 30.45. In some embodiments, Compound 8 Type A is characterized in that it has two or more peaks in its X-ray powder diffraction pattern selected from those at about 11.21, 17.06, 20.10, and 30.45. In some embodiments, Compound 8 Type A is characterized in that it has three or more peaks in its X-ray powder diffraction pattern selected from those at about 11.21, 17.06, 20.10, and 30.45. In some embodiments, Compound 8 Type A is characterized in that it has all four peaks in its X-ray powder diffraction pattern selected from those at about 11.21, 17.06, 20.10, and 30.45.

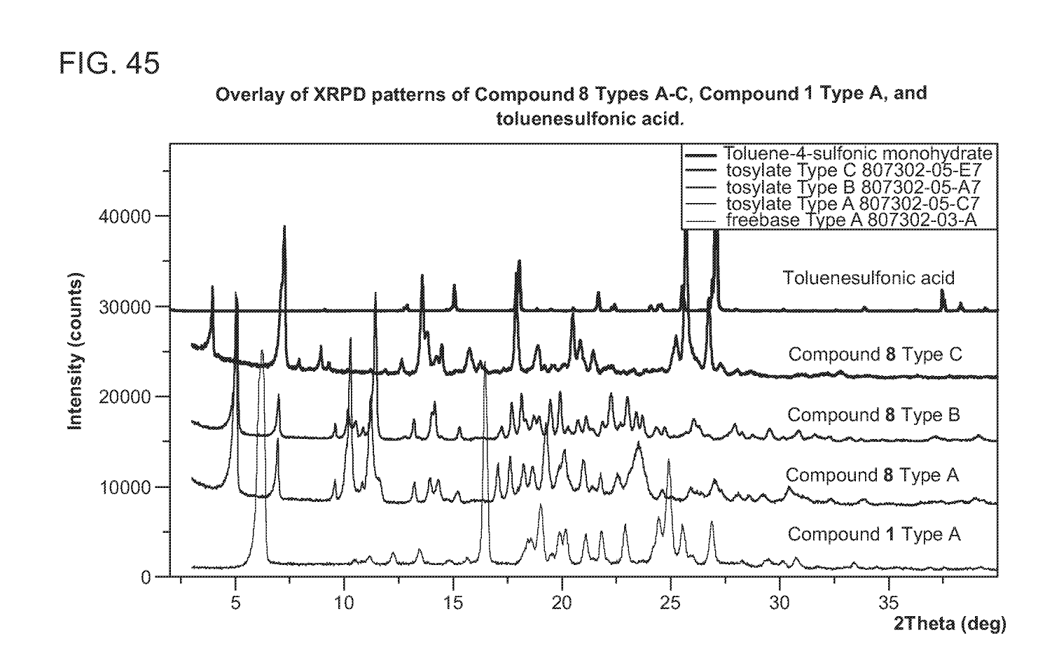

In certain embodiments, the X-ray powder diffraction pattern is substantially similar to the XRPD provided in FIG. 45.

Methods for preparing Compound 8 Type A are described infra.

### Compound 8 Type B

In some embodiments, Compound 8 Type B has at least 1, 2, 3, 4 or 5 spectral peak(s) selected from the peaks listed in Table 11 below.

TABLE 11
XRPD Peak Positions for Compound 8 Type B
Position (°2θ)

5.06
6.97
9.59
10.19
10.51
10.86
11.43
13.20
14.07
14.13
15.30
17.22
17.70
18.15
18.72
18.95
19.47
19.92
20.27
20.74
21.11
21.88
22.25
23.01
23.41
23.70
24.35
24.72
26.06
27.94
29.54
30.87
37.13

In some embodiments, Compound 8 Type B is characterized in that it has one or more peaks in its X-ray powder diffraction pattern selected from those at about 11.43, 14.13, 22.25, 24.35, and 27.94. In some embodiments, Compound 8 Type B is characterized in that it has two or more peaks in its X-ray powder diffraction pattern selected from those at about 11.43, 14.13, 22.25, 24.35, and 27.94. In some embodiments, Compound 8 Type B is characterized in that it has three or more peaks in its X-ray powder diffraction pattern selected from those at about 11.43, 14.13, 22.25, 24.35, and 27.94. In some embodiments, Compound 8 Type B is characterized in that it has four or more peaks in its X-ray powder diffraction pattern selected from those at about 11.43, 14.13, 22.25, 24.35, and 27.94. In some embodiments, Compound 8 Type B is characterized in that it has all five peaks in its X-ray powder diffraction pattern selected from those at about 11.43, 14.13, 22.25, 24.35, and 27.94.

In certain embodiments, the X-ray powder diffraction pattern is substantially similar to the XRPD provided in FIG. 45.

Methods for preparing Compound 8 Type B are described infra.

### Compound 8 Type C

In some embodiments, Compound 8 Type C has at least 1, 2, 3, 4 or 5 spectral peak(s) selected from the peaks listed in Table 12 below.

TABLE 12
XRPD Peak Positions for Compound 8 Type C
Position (°2θ)

3.93
7.23
7.92
8.92
9.29
12.64
13.58
14.25
14.48
15.75
16.25
17.90
18.89
19.60
20.12
20.49
20.84
21.44
25.24
25.70
26.74
27.28
28.04
18.89

In some embodiments, Compound 8 Type C is characterized in that it has one or more peaks in its X-ray powder diffraction pattern selected from those at about 3.93, 7.92, 8.92, 13.58, 15.75, 17.90, and 25.70. In some embodiments, Compound 8 Type C is characterized in that it has two or more peaks in its X-ray powder diffraction pattern selected from those at about 3.93, 7.92, 8.92, 13.58, 15.75, 17.90, and 25.70. In some embodiments, Compound 8 Type C is characterized in that it has three or more peaks in its X-ray powder diffraction pattern selected from those at about 3.93, 7.92, 8.92, 13.58, 15.75, 17.90, and 25.70. In some embodiments, Compound 8 Type C is characterized in that it has four or more peaks in its X-ray powder diffraction pattern selected from those at about 3.93, 7.92, 8.92, 13.58, 15.75, 17.90, and 25.70. In some embodiments, Compound 8 Type C is characterized in that it has five or more peaks in its X-ray powder diffraction pattern selected from those at about 3.93, 7.92, 8.92, 13.58, 15.75, 17.90, and 25.70. In some embodiments, Compound 8 Type C is characterized in that it has six or more peaks in its X-ray powder diffraction pattern selected from those at about 3.93, 7.92, 8.92, 13.58, 15.75, 17.90, and 25.70. In some embodiments, Compound 8 Type C is characterized in that it all seven peaks in its X-ray powder diffraction pattern selected from those at about 3.93, 7.92, 8.92, 13.58, 15.75, 17.90, and 25.70.

In certain embodiments, the X-ray powder diffraction pattern is substantially similar to the XRPD provided in FIG. 45.

Methods for preparing Compound 8 Type C are described infra.

### 9. Compound 9 (2-Naphthalenesulfonic Acid×Compound 1)

According to one embodiment, the present invention provides a chemical species Compound 9 comprising Compound 1 and 2-naphthalenesulfonic acid:

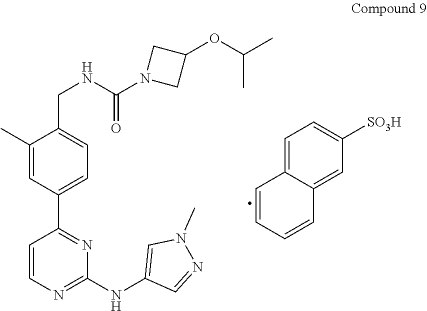 [SMILES: Cc1cc(-c2ccnc(Nc3cnn(C)c3)n2)ccc1CNC(=O)N1CC(OC(C)C)C1 ; O=S(=O)(O)c1ccc2ccccc2c1]

It is contemplated that Compound 9 can exist in a variety of physical forms. For example, Compound 9 can be in solution, suspension, or in solid form. In certain embodiments, Compound 9 is in solid form. When Compound 9 is in solid form, said compound may be amorphous, crystalline, or a mixture thereof. Exemplary solid forms are described in more detail below.

In one embodiment, the solid form of Compound 9 has a stoichiometry of (Compound 1):(2-naphthalenesulfonic acid) that is about 1:1.

In some embodiments, the present invention provides Compound 9 substantially free of impurities. As used herein, the term “substantially free of impurities” means that the compound contains no significant amount of extraneous matter. Such extraneous matter may include excess 2-naphthalenesulfonic acid, excess Compound 1, residual solvents, or any other impurities that may result from the preparation of, and/or isolation of, Compound 9. In certain embodiments, at least about 95% by weight of Compound 9 is present. In still other embodiments of the invention, at least about 99% by weight of Compound 9 is present.

According to one embodiment, Compound 9 is present in an amount of at least about 97, 97.5, 98.0, 98.5, 99, 99.5, 99.8 weight percent where the percentages are based on the total weight of the composition. According to another embodiment, Compound 9 contains no more than about 3.0 area percent HPLC of total organic impurities and, in certain embodiments, no more than about 1.5 area percent HPLC total organic impurities relative to the total area of the HPLC chromatogram. In other embodiments, Compound 9 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 9 is also meant to include all tautomeric forms of Compound 9. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 9 can exist in a variety of solid forms. Exemplary such forms include polymorphs such as those described herein.

In certain embodiments, Compound 9 is a crystalline solid. In other embodiments, Compound 9 is a crystalline solid substantially free of amorphous Compound 9. As used herein, the term “substantially free of amorphous Compound 9” means that the compound contains no significant amount of amorphous Compound 9. In certain embodiments, at least about 95% by weight of crystalline Compound 9 is present. In still other embodiments of the invention, at least about 99% by weight of crystalline Compound 9 is present.

It has been found that Compound 9 can exist in at least two distinct polymorphic forms. In some embodiments, the present invention provides a polymorphic form of Compound 9 referred to herein as Type A. In some embodiments, the present invention provides a polymorphic form of Compound 9 referred to herein as Type B.

In some embodiments, Compound 9 is amorphous. In some embodiments, Compound 9 is amorphous, and is substantially free of crystalline Compound 9.

### Compound 9 Type A

In some embodiments, of Compound 9 Type A has at least 1, 2, 3, 4 or 5 spectral peak(s) selected from the peaks listed in Table 13 below.

TABLE 13
XRPD Peak Positions for Compound 9 Type A
Position (°2θ)

3.89
7.22
8.83
9.22
13.41
13.79
14.19
15.12
15.57
15.77
16.17
17.10
17.68
17.93
18.49
18.67
18.91
19.41
20.41
21.00
21.37
21.93
22.46
22.98
23.78
24.10
24.34
25.07
25.36
25.71
26.69

In some embodiments, Compound 9 Type A is characterized in that it has one or more peaks in its X-ray powder diffraction pattern selected from those at about 3.89, 7.22, 8.83, 15.57, 17.93, 25.71, and 26.69. In some embodiments, Compound 9 Type A is characterized in that it has two or more peaks in its X-ray powder diffraction pattern selected from those at about 3.89, 7.22, 8.83, 15.57, 17.93, 25.71, and 26.69 In some embodiments, Compound 9 Type A is characterized in that it has three or more peaks in its X-ray powder diffraction pattern selected from those at about 3.89, 7.22, 8.83, 15.57, 17.93, 25.71, and 26.69. In some embodiments, Compound 9 Type A is characterized in that it has four or more peaks in its X-ray powder diffraction pattern selected from those at about 3.89, 7.22, 8.83, 15.57, 17.93, 25.71, and 26.69. In some embodiments, Compound 9 Type A is characterized in that it has five or more peaks in its X-ray powder diffraction pattern selected from those at about 3.89, 7.22, 8.83, 15.57, 17.93, 25.71, and 26.69. In some embodiments, Compound 9 Type A is characterized in that it has six or more peaks in its X-ray powder diffraction pattern selected from those at about 3.89, 7.22, 8.83, 15.57, 17.93, 25.71, and 26.69. In some embodiments, Compound 9 Type A is characterized in that it has all seven peaks in its X-ray powder diffraction pattern selected from those at about 3.89, 7.22, 8.83, 15.57, 17.93, 25.71, and 26.69.

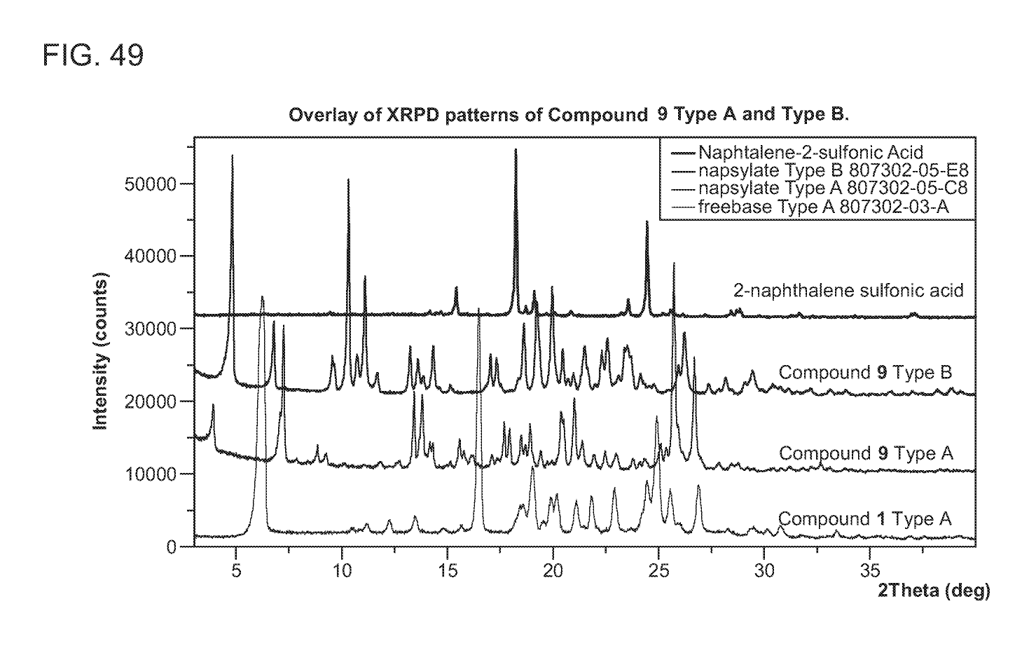

In certain embodiments, the X-ray powder diffraction pattern is substantially similar to the XRPD provided in FIG. 49.

Methods for preparing Compound 9 Type A are described infra.

### Compound 9 Type B

In some embodiments, Compound 9 Type B has at least 1, 2, 3, 4 or 5 spectral peak(s) selected from the peaks listed in Table 14 below.

TABLE 14
XRPD Peak Positions for Compound 9 Type B
Position (°2θ)

4.79
6.76
9.57
10.30
10.73
11.08
11.66
13.22
13.60
13.84
14.32
15.14
17.13
17.03
17.32
18.39
18.61
19.24
19.95
20.45
20.71
20.96
21.48
21.52
22.00
22.31
22.57
23.09
23.48
24.14
25.46
26.22
27.37
27.83
28.17
29.09
29.44

In some embodiments, Compound 9 Type B is characterized in that it has one or more peaks in its X-ray powder diffraction pattern selected from those at about 4.79, 6.76, 10.30, 11.08, 19.24, 19.95, and 26.22. In some embodiments, Compound 9 Type B is characterized in that it has two or more peaks in its X-ray powder diffraction pattern selected from those at about 4.79, 6.76, 10.30, 11.08, 19.24, 19.95, and 26.22. In some embodiments, Compound 9 Type B is characterized in that it has three or more peaks in its X-ray powder diffraction pattern selected from those at about 4.79, 6.76, 10.30, 11.08, 19.24, 19.95, and 26.22. In some embodiments, Compound 9 Type B is characterized in that it has four or more peaks in its X-ray powder diffraction pattern selected from those at about 4.79, 6.76, 10.30, 11.08, 19.24, 19.95, and 26.22. In some embodiments, Compound 9 Type B is characterized in that it has five or more peaks in its X-ray powder diffraction pattern selected from those at about 4.79, 6.76, 10.30, 11.08, 19.24, 19.95, and 26.22. In some embodiments, Compound 9 Type B is characterized in that it has six or more peaks in its X-ray powder diffraction pattern selected from those at about 4.79, 6.76, 10.30, 11.08, 19.24, 19.95, and 26.22. In some embodiments, Compound 9 Type B is characterized in that it has all seven peaks in its X-ray powder diffraction pattern selected from those at about 4.79, 6.76, 10.30, 11.08, 19.24, 19.95, and 26.22.

In certain embodiments, the X-ray powder diffraction pattern is substantially similar to the XRPD provided in FIG. 49.

Methods for preparing Compound 9 Type B are described infra.

### 10. Compound 10 (Nitric Acid×Compound 1)

According to one embodiment, the present invention provides a chemical species Compound 10 comprising Compound 1 and nitric acid:

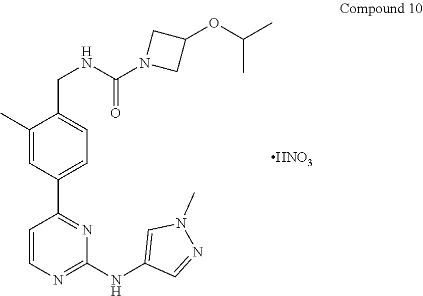 [SMILES: Cc1cc(-c2ccnc(Nc3cnn(C)c3)n2)ccc1CNC(=O)N1CC(OC(C)C)C1 ; "·HNO3"]

It is contemplated that Compound 10 can exist in a variety of physical forms. For example, Compound 10 can be in solution, suspension, or in solid form. In certain embodiments, Compound 10 is in solid form. When Compound 10 is in solid form, said compound may be amorphous, crystalline, or a mixture thereof. Exemplary solid forms are described in more detail below.

In one embodiment, the solid form of Compound 10 has a stoichiometry of (Compound 1):(nitric acid) that is about 1:1.

In some embodiments, the present invention provides Compound 10 substantially free of impurities. As used herein, the term “substantially free of impurities” means that the compound contains no significant amount of extraneous matter. Such extraneous matter may include excess nitric acid, excess Compound 1, residual solvents, or any other impurities that may result from the preparation of, and/or isolation of, Compound 10. In certain embodiments, at least about 95% by weight of Compound 10 is present. In still other embodiments of the invention, at least about 99% by weight of Compound 10 is present.

According to one embodiment, Compound 10 is present in an amount of at least about 97, 97.5, 98.0, 98.5, 99, 99.5, 99.8 weight percent where the percentages are based on the total weight of the composition. According to another embodiment, Compound 10 contains no more than about 3.0 area percent HPLC of total organic impurities and, in certain embodiments, no more than about 1.5 area percent HPLC total organic impurities relative to the total area of the HPLC chromatogram. In other embodiments, Compound 10 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 10 is also meant to include all tautomeric forms of Compound 10. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 10 can exist in a variety of solid forms. Exemplary such forms include polymorphs such as those described herein.

In certain embodiments, Compound 10 is a crystalline solid. In other embodiments, Compound 10 is a crystalline solid substantially free of amorphous Compound 10. As used herein, the term “substantially free of amorphous Compound 10” means that the compound contains no significant amount of amorphous Compound 10. In certain embodiments, at least about 95% by weight of crystalline Compound 10 is present. In still other embodiments of the invention, at least about 99% by weight of crystalline Compound 10 is present.

It has been found that Compound 10 can exist in at least three distinct polymorphic forms. In some embodiments, the present invention provides a polymorphic form of Compound 10 referred to herein as Type A. In some embodiments, the present invention provides a polymorphic form of Compound 10 referred to herein as Type B. In some embodiments, the present invention provides a polymorphic form of Compound 10 referred to herein as Type C.

In some embodiments, Compound 10 is amorphous. In some embodiments, Compound 10 is amorphous, and is substantially free of crystalline Compound 10.

### Compound 10 Type A

In some embodiments, Compound 10 Type A has at least 1, 2, 3, 4 or 5 spectral peak(s) selected from the peaks listed in Table 15 below.

TABLE 15
XRPD Peak Positions for Compound 10 Type A
Position (°2θ)

7.52
8.14
9.09
10.63
11.03
12.57
13.15
13.71
14.31
15.11
16.21
16.60
18.27
18.68
19.59
20.70
21.58
22.20
22.77
22.99
23.28
23.91
25.21
25.30
25.68
26.51
27.61
28.08
29.17
29.90
30.55
30.71
30.91
31.22
32.80
33.06
33.34
33.64
34.81
36.44
37.50
37.90

In some embodiments, Compound 10 Type A is characterized in that it has one or more peaks in its X-ray powder diffraction pattern selected from those at about 7.52, 11.03, 18.68, 20.70, 22.20, and 29.17. In some embodiments, Compound 10 Type A is characterized in that it has two or more peaks in its X-ray powder diffraction pattern selected from those at about 7.52, 11.03, 18.68, 20.70, 22.20, and 29.17. In some embodiments, Compound 10 Type A is characterized in that it has three or more peaks in its X-ray powder diffraction pattern selected from those at about 7.52, 11.03, 18.68, 20.70, 22.20, and 29.17. In some embodiments, Compound 10 Type A is characterized in that it has four or more peaks in its X-ray powder diffraction pattern selected from those at about 7.52, 11.03, 18.68, 20.70, 22.20, and 29.17. In some embodiments, Compound 10 Type A is characterized in that it has five or more peaks in its X-ray powder diffraction pattern selected from those at about 7.52, 11.03, 18.68, 20.70, 22.20, and 29.17. In some embodiments, Compound 10 Type A is characterized in that it has all six peaks in its X-ray powder diffraction pattern selected from those at about 7.52, 11.03, 18.68, 20.70, 22.20, and 29.17.

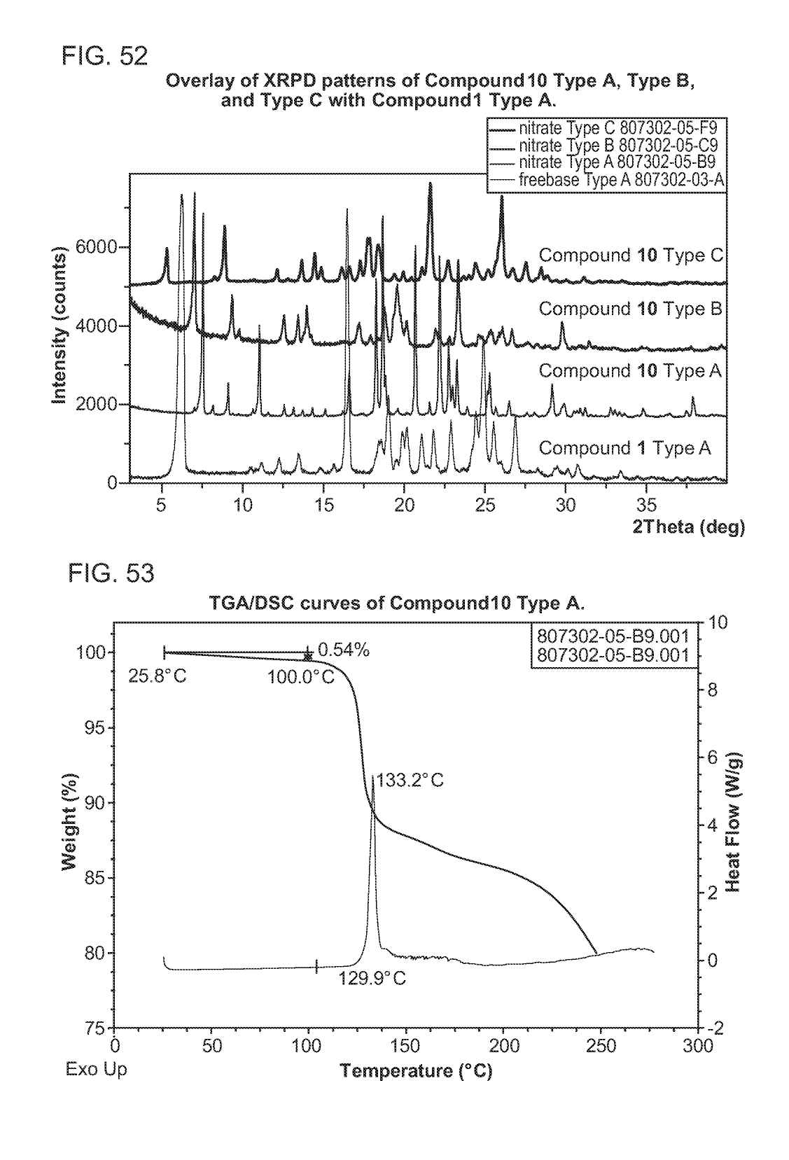

In certain embodiments, the X-ray powder diffraction pattern is substantially similar to the XRPD provided in FIG. 52.

Methods for preparing Compound 10 Type A are described infra.

### Compound 10 Type B

In some embodiments, Compound 10 Type B has at least 1, 2, 3, 4 or 5 spectral peak(s) selected from the peaks listed in Table 16 below.

TABLE 16
XRPD Peak Positions for Compound 10 Type B
Position (°2θ)

7.00
9.34
9.77
12.55
13.43
13.97
17.20
17.92
18.79
19.55
20.15
21.96
22.79
23.35
24.64
25.37
26.08
26.69
29.80
31.48

In some embodiments, Compound 10 Type B is characterized in that it has one or more peaks in its X-ray powder diffraction pattern selected from those at about 7.00, 9.34, 13.97, and 20.15. In some embodiments, Compound 10 Type B is characterized in that it has two or more peaks in its X-ray powder diffraction pattern selected from those at about 7.00, 9.34, 13.97, and 20.15. In some embodiments, Compound 10 Type B is characterized in that it has three or more peaks in its X-ray powder diffraction pattern selected from those at about 7.00, 9.34, 13.97, and 20.15. In some embodiments, Compound 10 Type B is characterized in that it has all four peaks in its X-ray powder diffraction pattern selected from those at about 7.00, 9.34, 13.97, and 20.15.

In certain embodiments, the X-ray powder diffraction pattern is substantially similar to the XRPD provided in FIG. 52.

Methods for preparing Compound 10 Type B are described infra.

### Compound 10 Type C

In some embodiments, Compound 10 Type C has at least 1, 2, 3, 4 or 5 spectral peak(s) selected from the peaks listed in Table 17 below.

TABLE 17
XRPD Peak Positions for Compound 10 Type C
Position (°2θ)

5.29
8.26
8.87
10.72
12.13
12.80
13.65
14.46
14.86
16.15
16.60
17.28
17.77
18.40
19.40
19.94
20.44
21.12
21.62
22.72
23.68
24.02
24.43
25.21
25.78
26.05
26.74
27.54
28.48
28.86
29.42
30.05
31.14

In some embodiments, Compound 10 Type C is characterized in that it has one or more peaks in its X-ray powder diffraction pattern selected from those at about 5.29, 8.87, 12.13, 14.86, 17.77, and 26.05. In some embodiments, Compound 10 Type C is characterized in that it has two or more peaks in its X-ray powder diffraction pattern selected from those at about 5.29, 8.87, 12.13, 14.86, 17.77, and 26.05. In some embodiments, Compound 10 Type C is characterized in that it has three or more peaks in its X-ray powder diffraction pattern selected from those at about 5.29, 8.87, 12.13, 14.86, 17.77, and 26.05. In some embodiments, Compound 10 Type C is characterized in that it has four or more peaks in its X-ray powder diffraction pattern selected from those at about 5.29, 8.87, 12.13, 14.86, 17.77, and 26.05. In some embodiments, Compound 10 Type C is characterized in that it has five or more peaks in its X-ray powder diffraction pattern selected from those at about 5.29, 8.87, 12.13, 14.86, 17.77, and 26.05. In some embodiments, Compound 10 Type C is characterized in that it has all six peaks in its X-ray powder diffraction pattern selected from those at about 5.29, 8.87, 12.13, 14.86, 17.77, and 26.05.

In certain embodiments, the X-ray powder diffraction pattern is substantially similar to the XRPD provided in FIG. 52.

Methods for preparing Compound 10 Type C are described infra.

### 11. Compound 11 (Oxalic Acid×Compound 1)

According to one embodiment, the present invention provides a chemical species Compound 11 comprising Compound 1 and oxalic acid:

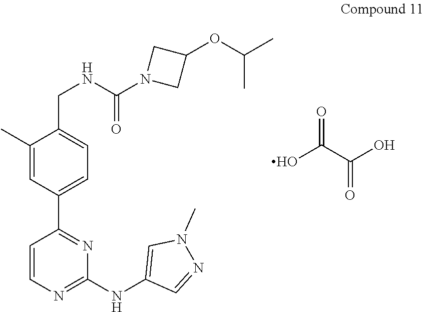

It is contemplated that Compound 11 can exist in a variety of physical forms. For example, Compound 11 can be in solution, suspension, or in solid form. In certain embodiments, Compound 11 is in solid form. When Compound 11 is in solid form, said compound may be amorphous, crystalline, or a mixture thereof. Exemplary solid forms are described in more detail below.

In one embodiment, the solid form of Compound 11 has a stoichiometry of (Compound 1):(oxalic acid) that is about 1:1.

In some embodiments, the present invention provides Compound 11 substantially free of impurities. As used herein, the term “substantially free of impurities” means that the compound contains no significant amount of extraneous matter. Such extraneous matter may include excess oxalic acid, excess compound 1, residual solvents, or any other impurities that may result from the preparation of, and/or isolation of, Compound 11. In certain embodiments, at least about 95% by weight of Compound 11 is present. In still other embodiments of the invention, at least about 99% by weight of Compound 11 is present.

According to one embodiment, Compound 11 is present in an amount of at least about 97, 97.5, 98.0, 98.5, 99, 99.5, 99.8 weight percent where the percentages are based on the total weight of the composition. According to another embodiment, Compound 11 contains no more than about 3.0 area percent HPLC of total organic impurities and, in certain embodiments, no more than about 1.5 area percent HPLC total organic impurities relative to the total area of the HPLC chromatogram. In other embodiments, Compound 11 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 11 is also meant to include all tautomeric forms of Compound 11. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 11 can exist in a variety of solid forms. Exemplary such forms include polymorphs such as those described herein.

In certain embodiments, Compound 11 is a crystalline solid. In other embodiments, Compound 11 is a crystalline solid substantially free of amorphous Compound 11. As used herein, the term “substantially free of amorphous Compound 11” means that the compound contains no significant amount of amorphous Compound 11. In certain embodiments, at least about 95% by weight of crystalline Compound 11 is present. In still other embodiments of the invention, at least about 99% by weight of crystalline Compound 11 is present.

It has been found that Compound 11 can exist in at least three distinct polymorphic forms. In some embodiments, the present invention provides a polymorphic form of Compound 11 referred to herein as Type A. In some embodiments, the present invention provides a polymorphic form of Compound 11 referred to herein as Type B. In some embodiments, the present invention provides a polymorphic form of Compound 11 referred to herein as Type C.

In some embodiments, Compound 11 is amorphous. In some embodiments, Compound 11 is amorphous, and is substantially free of crystalline Compound 11.

### Compound 11 Type A

In some embodiments, Compound 11 Type A has at least 1, 2, 3, 4 or 5 spectral peak(s) selected from the peaks listed in Table 18 below.

TABLE 18
XRPD Peak Positions for Compound 11 Type A
Position (°2θ)

3.98
6.36
7.27
8.06
10.59
11.25
12.16
13.62
13.86
15.87
16.54
17.54
18.38
19.02
20.05
20.22
20.65
21.48
22.63
23.36
24.54
25.11
25.35
25.49
26.17
27.10
30.11
30.74
31.33
36.58
37.58

In some embodiments, Compound 11 Type A is characterized in that it has one or more peaks in its X-ray powder diffraction pattern selected from those at about 3.98, 7.27, 12.16, 18.38, and 20.65. In some embodiments, Compound 11 Type A is characterized in that it has two or more peaks in its X-ray powder diffraction pattern selected from those at about 3.98, 7.27, 12.16, 18.38, and 20.65. In some embodiments, Compound 11 Type A is characterized in that it has three or more peaks in its X-ray powder diffraction pattern selected from those at about 3.98, 7.27, 12.16, 18.38, and 20.65. In some embodiments, Compound 11 Type A is characterized in that it has four or more peaks in its X-ray powder diffraction pattern selected from those at about 3.98, 7.27, 12.16, 18.38, and 20.65. In some embodiments, Compound 11 Type A is characterized in that it has all five peaks in its X-ray powder diffraction pattern selected from those at about 3.98, 7.27, 12.16, 18.38, and 20.65.

In certain embodiments, the X-ray powder diffraction pattern is substantially similar to the XRPD provided in FIG. 56.

Methods for preparing Compound 11 Type A are described infra.

### Compound 11 Type B

In some embodiments, Compound 11 Type B has at least 1, 2, 3, 4 or 5 spectral peak(s) selected from the peaks listed in Table 19 below.

TABLE 19
XRPD Peak Positions for Compound 11 Type B
Position (°2θ)

5.77
6.86
7.97
8.16
9.70
10.04
10.90
11.33
11.60
13.37
13.98
14.32
15.20
15.91
16.42
16.71
17.04
17.61
18.73
18.94
19.48
19.87
20.32
20.81
21.33
21.95
22.31
23.18
24.48
24.63
25.00
25.64
26.30
27.02
27.33
27.94
28.57
29.29
30.03
30.90
33.09
34.92
37.87

In some embodiments, Compound 11 Type B is characterized in that it has one or more peaks in its X-ray powder diffraction pattern selected from those at about 5.77, 6.86, 13.37, 15.20, 17.61, and 30.90. In some embodiments, Compound 11 Type B is characterized in that it has two or more peaks in its X-ray powder diffraction pattern selected from those at about 5.77, 6.86, 13.37, 15.20, 17.61, and 30.90. In some embodiments, Compound 11 Type B is characterized in that it has three or more peaks in its X-ray powder diffraction pattern selected from those at about 5.77, 6.86, 13.37, 15.20, 17.61, and 30.90. In some embodiments, Compound 11 Type B is characterized in that it has four or more peaks in its X-ray powder diffraction pattern selected from those at about 5.77, 6.86, 13.37, 15.20, 17.61, and 30.90. In some embodiments, Compound 11 Type B is characterized in that it has five or more peaks in its X-ray powder diffraction pattern selected from those at about 5.77, 6.86, 13.37, 15.20, 17.61, and 30.90. In some embodiments, Compound 11 Type B is characterized in that it has all six peaks in its X-ray powder diffraction pattern selected from those at about 5.77, 6.86, 13.37, 15.20, 17.61, and 30.90.

In certain embodiments, the X-ray powder diffraction pattern is substantially similar to the XRPD provided in FIG. 56.

Methods for preparing Compound 11 Type B are described infra.

### Compound 11 Type C

In some embodiments, Compound 11 Type C has at least 1, 2, 3, 4 or 5 spectral peak(s) selected from the peaks listed in Table 20 below.

TABLE 20
XRPD Peak Positions for Compound 11 Type C
Position (°2θ)

3.15
7.82
9.66
10.56
11.62
12.07
13.62
14.22
15.81
16.72
17.70
18.90
19.51
20.04
21.33
21.61
22.42
22.69
23.49
24.45
24.62
25.26
25.41
26.15
27.68
29.98
32.56
32.93
33.91
36.36
37.49

In some embodiments, Compound 11 Type C is characterized in that it has one or more peaks in its X-ray powder diffraction pattern selected from those at about 3.15, 10.56, 11.62, 13.62, 20.04, 27.68, and 32.56. In some embodiments, Compound 11 Type C is characterized in that it has two or more peaks in its X-ray powder diffraction pattern selected from those at about 3.15, 10.56, 11.62, 13.62, 20.04, 27.68, and 32.56. In some embodiments, Compound 11 Type C is characterized in that it has three or more peaks in its X-ray powder diffraction pattern selected from those at about 3.15, 10.56, 11.62, 13.62, 20.04, 27.68, and 32.56. In some embodiments, Compound 11 Type C is characterized in that it has four or more peaks in its X-ray powder diffraction pattern selected from those at about 3.15, 10.56, 11.62, 13.62, 20.04, 27.68, and 32.56. In some embodiments, Compound 11 Type C is characterized in that it has five or more peaks in its X-ray powder diffraction pattern selected from those at about 3.15, 10.56, 11.62, 13.62, 20.04, 27.68, and 32.56. In some embodiments, Compound 11 Type C is characterized in that it has six or more peaks in its X-ray powder diffraction pattern selected from those at about 3.15, 10.56, 11.62, 13.62, 20.04, 27.68, and 32.56. In some embodiments, Compound 11 Type C is characterized in that it has all seven peaks in its X-ray powder diffraction pattern selected from those at about 3.15, 10.56, 11.62, 13.62, 20.04, 27.68, and 32.56.

In certain embodiments, the X-ray powder diffraction pattern is substantially similar to the XRPD provided in FIG. 56.

Methods for preparing Compound 11 Type C are described infra.

### 12. Compound 12 (Fumaric Acid×Compound 1)

According to one embodiment, the present invention provides a chemical species Compound 12 comprising Compound 1 and fumaric acid:

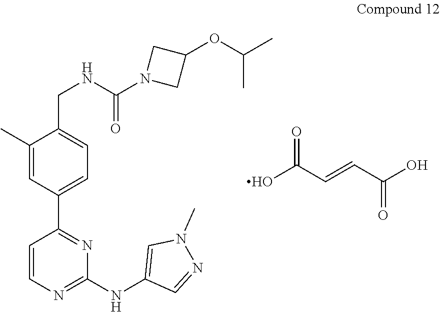

It is contemplated that Compound 12 can exist in a variety of physical forms. For example, Compound 12 can be in solution, suspension, or in solid form. In certain embodiments, Compound 12 is in solid form. When Compound 12 is in solid form, said compound may be amorphous, crystalline, or a mixture thereof. Exemplary solid forms are described in more detail below.

In one embodiment, the solid form of Compound 12 has a stoichiometry of (Compound 1):(fumaric acid) that is about 1:1.

In some embodiments, the present invention provides Compound 12 substantially free of impurities. As used herein, the term “substantially free of impurities” means that the compound contains no significant amount of extraneous matter. Such extraneous matter may include excess fumaric acid, excess Compound 1, residual solvents, or any other impurities that may result from the preparation of, and/or isolation of, Compound 12. In certain embodiments, at least about 95% by weight of Compound 12 is present. In still other embodiments of the invention, at least about 99% by weight of Compound 12 is present.

According to one embodiment, Compound 12 is present in an amount of at least about 97, 97.5, 98.0, 98.5, 99, 99.5, 99.8 weight percent where the percentages are based on the total weight of the composition. According to another embodiment, Compound 12 contains no more than about 3.0 area percent HPLC of total organic impurities and, in certain embodiments, no more than about 1.5 area percent HPLC total organic impurities relative to the total area of the HPLC chromatogram. In other embodiments, Compound 12 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 12 is also meant to include all tautomeric forms of Compound 12. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 12 can exist in a variety of solid forms. Exemplary such forms include polymorphs such as those described herein.

In certain embodiments, Compound 12 is a crystalline solid. In other embodiments, Compound 12 is a crystalline solid substantially free of amorphous Compound 12. As used herein, the term “substantially free of amorphous Compound 12” means that the compound contains no significant amount of amorphous Compound 12. In certain embodiments, at least about 95% by weight of crystalline Compound 12 is present. In still other embodiments of the invention, at least about 99% by weight of crystalline Compound 12 is present.

It has been found that Compound 12 can exist in at least one distinct crystalline form. In some embodiments, the present invention provides a crystalline form of Compound 12 referred to herein as Type A.

In some embodiments, Compound 12 is amorphous. In some embodiments, Compound 12 is amorphous, and is substantially free of crystalline Compound 12.

### Compound 12 Type A

In some embodiments, Compound 12 Type A has at least 1, 2, 3, 4 or 5 spectral peak(s) selected from the peaks listed in Table 21 below.

TABLE 21
XRPD Peak Positions for Compound 12 Type A
Position (°2θ)

5.68
6.53
7.12
8.65
10.74
13.07
13.43
14.00
14.27
14.64
15.67
16.37
17.10
18.30
19.23
19.45
19.75
20.47
21.00
21.45
22.50
22.86
23.60
24.20
25.90
26.49
27.55
28.15
28.89
31.56
33.20

In some embodiments, Compound 12 Type A is characterized in that it has one or more peaks in its X-ray powder diffraction pattern selected from those at about 5.68, 7.12, 8.65, 16.37, 20.47, and 22.86. In some embodiments, Compound 12 Type A is characterized in that it has two or more peaks in its X-ray powder diffraction pattern selected from those at about 5.68, 7.12, 8.65, 16.37, 20.47, and 22.86. In some embodiments, Compound 12 Type A is characterized in that it has three or more peaks in its X-ray powder diffraction pattern selected from those at about 5.68, 7.12, 8.65, 16.37, 20.47, and 22.86. In some embodiments, Compound 12 Type A is characterized in that it has four or more peaks in its X-ray powder diffraction pattern selected from those at about 5.68, 7.12, 8.65, 16.37, 20.47, and 22.86. In some embodiments, Compound 12 Type A is characterized in that it has five or more peaks in its X-ray powder diffraction pattern selected from those at about 5.68, 7.12, 8.65, 16.37, 20.47, and 22.86. In some embodiments, Compound 12 Type A is characterized in that it has all six peaks in its X-ray powder diffraction pattern selected from those at about 5.68, 7.12, 8.65, 16.37, 20.47, and 22.86.

In certain embodiments, the X-ray powder diffraction pattern is substantially similar to the XRPD provided in FIG. 60.

Methods for preparing Compound 12 Type A are described infra.

### 13. Compound 13 (L-Tartaric Acid×Compound 1)

According to one embodiment, the present invention provides a chemical species Compound 13 comprising Compound 1 and L-tartaric acid:

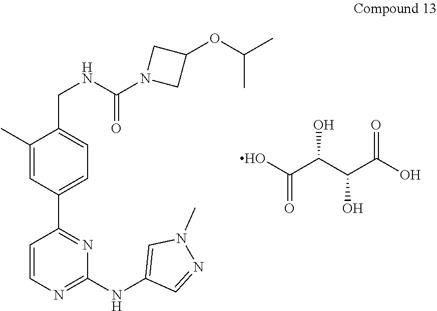

It is contemplated that Compound 13 can exist in a variety of physical forms. For example, Compound 13 can be in solution, suspension, or in solid form. In certain embodiments, Compound 13 is in solid form. When Compound 13 is in solid form, said compound may be amorphous, crystalline, or a mixture thereof. Exemplary solid forms are described in more detail below.

In one embodiment, the solid form of Compound 13 has a stoichiometry of (Compound 1):(L-tartaric acid) that is about 1:1.

In some embodiments, the present invention provides Compound 13 substantially free of impurities. As used herein, the term “substantially free of impurities” means that the compound contains no significant amount of extraneous matter. Such extraneous matter may include excess L-tartaric acid, excess Compound 1, residual solvents, or any other impurities that may result from the preparation of, and/or isolation of, Compound 13. In certain embodiments, at least about 95% by weight of Compound 13 is present. In still other embodiments of the invention, at least about 99% by weight of Compound 13 is present.

According to one embodiment, Compound 13 is present in an amount of at least about 97, 97.5, 98.0, 98.5, 99, 99.5, 99.8 weight percent where the percentages are based on the total weight of the composition. According to another embodiment, Compound 13 contains no more than about 3.0 area percent HPLC of total organic impurities and, in certain embodiments, no more than about 1.5 area percent HPLC total organic impurities relative to the total area of the HPLC chromatogram. In other embodiments, Compound 13 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 13 is also meant to include all tautomeric forms of Compound 13. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 13 can exist in a variety of solid forms. Exemplary such forms include polymorphs such as those described herein.

In certain embodiments, Compound 13 is a crystalline solid. In other embodiments, Compound 13 is a crystalline solid substantially free of amorphous Compound 13. As used herein, the term “substantially free of amorphous Compound 13” means that the compound contains no significant amount of amorphous Compound 13. In certain embodiments, at least about 95% by weight of crystalline Compound 13 is present. In still other embodiments of the invention, at least about 99% by weight of crystalline Compound 13 is present.

It has been found that Compound 13 can exist in at least one distinct crystalline form. In some embodiments, the present invention provides a crystalline form of Compound 13 referred to herein as Type A.

In some embodiments, Compound 13 is amorphous. In some embodiments, Compound 13 is amorphous, and is substantially free of crystalline Compound 13.

### Compound 13 Type A

In some embodiments, Compound 13 Type A has at least 1, 2, 3, 4 or 5 spectral peak(s) selected from the peaks listed in Table 22 below.

TABLE 22
XRPD Peak Positions for Compound 13 Type A
Position (°2θ)

5.61
6.51
7.08
8.59
10.64
13.36
13.88
14.24
14.55
15.37
16.28
16.88
18.11
19.24
19.61
20.40
21.40
22.15
22.53
23.48
25.52
25.88
26.38
27.42
27.98

In some embodiments, Compound 13 Type A is characterized in that it has one or more peaks in its X-ray powder diffraction pattern selected from those at about 6.51, 7.08, 13.36, 16.28, 19.24, and 25.88. In some embodiments, Compound 13 Type A is characterized in that it has two or more peaks in its X-ray powder diffraction pattern selected from those at about 6.51, 7.08, 13.36, 16.28, 19.24, and 25.88. In some embodiments, Compound 13 Type A is characterized in that it has three or more peaks in its X-ray powder diffraction pattern selected from those at about 6.51, 7.08, 13.36, 16.28, 19.24, and 25.88. In some embodiments, Compound 13 Type A is characterized in that it has four or more peaks in its X-ray powder diffraction pattern selected from those at about 6.51, 7.08, 13.36, 16.28, 19.24, and 25.88. In some embodiments, Compound 13 Type A is characterized in that it has five or more peaks in its X-ray powder diffraction pattern selected from those at about 6.51, 7.08, 13.36, 16.28, 19.24, and 25.88. In some embodiments, Compound 13 Type A is characterized in that it has all six peaks in its X-ray powder diffraction pattern selected from those at about 6.51, 7.08, 13.36, 16.28, 19.24, and 25.88.

In certain embodiments, the X-ray powder diffraction pattern is substantially similar to the XRPD provided in FIG. 62.

Methods for preparing Compound 13 Type A are described infra.

### 14. Compound 14 (Citric Acid×Compound 1)

According to one embodiment, the present invention provides a chemical species Compound 14 comprising Compound 1 and citric acid:

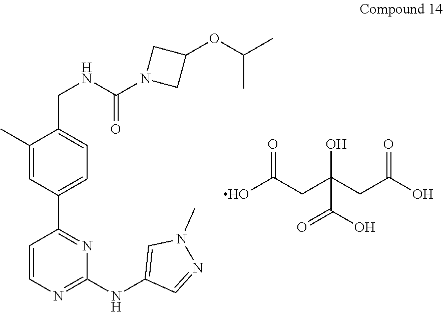

It is contemplated that Compound 14 can exist in a variety of physical forms. For example, Compound 14 can be in solution, suspension, or in solid form. In certain embodiments, Compound 14 is in solid form. When Compound 14 is in solid form, said compound may be amorphous, crystalline, or a mixture thereof. Exemplary solid forms are described in more detail below.

In one embodiment, the solid form of Compound 14 has a stoichiometry of (Compound 1):(citric acid) that is about 1:1.

In some embodiments, the present invention provides Compound 14 substantially free of impurities. As used herein, the term “substantially free of impurities” means that the compound contains no significant amount of extraneous matter. Such extraneous matter may include excess citric acid, excess Compound 1, residual solvents, or any other impurities that may result from the preparation of, and/or isolation of, Compound 14. In certain embodiments, at least about 95% by weight of Compound 14 is present. In still other embodiments of the invention, at least about 99% by weight of Compound 14 is present.

According to one embodiment, Compound 14 is present in an amount of at least about 97, 97.5, 98.0, 98.5, 99, 99.5, 99.8 weight percent where the percentages are based on the total weight of the composition. According to another embodiment, Compound 14 contains no more than about 3.0 area percent HPLC of total organic impurities and, in certain embodiments, no more than about 1.5 area percent HPLC total organic impurities relative to the total area of the HPLC chromatogram. In other embodiments, Compound 14 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 14 is also meant to include all tautomeric forms of Compound 14. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 14 can exist in a variety of solid forms. Exemplary such forms include polymorphs such as those described herein.

In certain embodiments, Compound 14 is a crystalline solid. In other embodiments, Compound 14 is a crystalline solid substantially free of amorphous Compound 14. As used herein, the term “substantially free of amorphous Compound 14” means that the compound contains no significant amount of amorphous Compound 14. In certain embodiments, at least about 95% by weight of crystalline Compound 14 is present. In still other embodiments of the invention, at least about 99% by weight of crystalline Compound 14 is present.

It has been found that Compound 14 can exist in at least two distinct polymorphic forms. In some embodiments, the present invention provides a polymorphic form of Compound 14 referred to herein as Type A. In some embodiments, the present invention provides a polymorphic form of Compound 14 referred to herein as Type B.

In some embodiments, Compound 14 is amorphous. In some embodiments, Compound 14 is amorphous, and is substantially free of crystalline Compound 14.

### Compound 14 Type A

In some embodiments, Compound 14 Type A has at least 1, 2, 3, 4 or 5 spectral peak(s) selected from the peaks listed in Table 23 below.

TABLE 23
XRPD Peak Positions for Compound 14 Type A
Position (°2θ)

3.97
7.16
8.05
8.72
11.58
14.48
15.02
15.58
15.97
16.26
16.87
17.82
18.03
18.28
18.76
20.43
20.70
21.54
22.05
22.41
22.80
23.40
24.58
25.01
26.79
27.16
28.69
30.72
31.32
32.80
33.29
34.23
34.88
35.57
36.99
38.18
39.08

In some embodiments, Compound 14 Type A is characterized in that it has one or more peaks in its X-ray powder diffraction pattern selected from those at about 8.05, 11.58, 16.87, 24.58, and 30.72. In some embodiments, Compound 14 Type A is characterized in that it has two or more peaks in its X-ray powder diffraction pattern selected from those at about 8.05, 11.58, 16.87, 24.58, and 30.72. In some embodiments, Compound 14 Type A is characterized in that it has three or more peaks in its X-ray powder diffraction pattern selected from those at about 8.05, 11.58, 16.87, 24.58, and 30.72. In some embodiments, Compound 14 Type A is characterized in that it has four or more peaks in its X-ray powder diffraction pattern selected from those at about 8.05, 11.58, 16.87, 24.58, and 30.72. In some embodiments, Compound 14 Type A is characterized in that it has all five peaks in its X-ray powder diffraction pattern selected from those at about 8.05, 11.58, 16.87, 24.58, and 30.72.

In certain embodiments, the X-ray powder diffraction pattern is substantially similar to the XRPD provided in FIG. 64.

Methods for preparing Compound 14 Type A are described infra.

### Compound 14 Type B

In some embodiments, Compound 14 Type B has at least 1, 2, 3, 4 or 5 spectral peak(s) selected from the peaks listed in Table 24 below.

TABLE 24
XRPD Peak Positions for Compound 14 Type B
Position (°2θ)

4.10
7.07
8.64
9.39
10.29
10.87
12.28
12.45
13.19
13.92
14.23
14.87
16.35
16.66
17.36
17.94
18.27
18.75
19.02
20.93
21.71
21.94
22.84
23.23
24.57
25.47
25.92
27.38
28.94
31.71
33.06
38.65

In some embodiments, Compound 14 Type B is characterized in that it has one or more peaks in its X-ray powder diffraction pattern selected from those at about 10.29, 12.45, 13.19, 19.02, 25.47, and 25.92. In some embodiments, Compound 14 Type B is characterized in that it has two more peaks in its X-ray powder diffraction pattern selected from those at about 10.29, 12.45, 13.19, 19.02, 25.47, and 25.92. In some embodiments, Compound 14 Type B is characterized in that it has three or more peaks in its X-ray powder diffraction pattern selected from those at about 10.29, 12.45, 13.19, 19.02, 25.47, and 25.92. In some embodiments, Compound 14 Type B is characterized in that it has four or more peaks in its X-ray powder diffraction pattern selected from those at about 10.29, 12.45, 13.19, 19.02, 25.47, and 25.92. In some embodiments, Compound 14 Type B is characterized in that it has five or more peaks in its X-ray powder diffraction pattern selected from those at about 10.29, 12.45, 13.19, 19.02, 25.47, and 25.92. In some embodiments, Compound 14 Type B is characterized in that it has all six peaks in its X-ray powder diffraction pattern selected from those at about 10.29, 12.45, 13.19, 19.02, 25.47, and 25.92.

In certain embodiments, the X-ray powder diffraction pattern is substantially similar to the XRPD provided in FIG. 64.

Methods for preparing Compound 14 Type B are described infra.

### 15. Compound 15 (L-Malic Acid×Compound 1)

According to one embodiment, the present invention provides a chemical species Compound 15 comprising Compound 1 and L-malic acid:

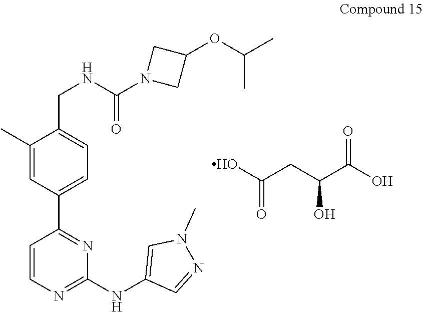

It is contemplated that Compound 15 can exist in a variety of physical forms. For example, Compound 15 can be in solution, suspension, or in solid form. In certain embodiments, Compound 15 is in solid form. When Compound 15 is in solid form, said compound may be amorphous, crystalline, or a mixture thereof. Exemplary solid forms are described in more detail below.

In one embodiment, the solid form of Compound 15 has a stoichiometry of (Compound 1):(L-malic acid) that is about 1:1.

In some embodiments, the present invention provides Compound 15 substantially free of impurities. As used herein, the term “substantially free of impurities” means that the compound contains no significant amount of extraneous matter. Such extraneous matter may include excess L-malic acid, excess Compound 1, residual solvents, or any other impurities that may result from the preparation of, and/or isolation of, Compound 15. In certain embodiments, at least about 95% by weight of Compound 15 is present. In still other embodiments of the invention, at least about 99% by weight of Compound 15 is present.

According to one embodiment, Compound 15 is present in an amount of at least about 97, 97.5, 98.0, 98.5, 99, 99.5, 99.8 weight percent where the percentages are based on the total weight of the composition. According to another embodiment, Compound 15 contains no more than about 3.0 area percent HPLC of total organic impurities and, in certain embodiments, no more than about 1.5 area percent HPLC total organic impurities relative to the total area of the HPLC chromatogram. In other embodiments, Compound 15 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 15 is also meant to include all tautomeric forms of Compound 15. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 15 can exist in a variety of solid forms. Exemplary such forms include polymorphs such as those described herein.

In certain embodiments, Compound 15 is a crystalline solid. In other embodiments, Compound 15 is a crystalline solid substantially free of amorphous Compound 15. As used herein, the term “substantially free of amorphous Compound 15” means that the compound contains no significant amount of amorphous Compound 15. In certain embodiments, at least about 95% by weight of crystalline Compound 15 is present. In still other embodiments of the invention, at least about 99% by weight of crystalline Compound 15 is present.

It has been found that Compound 15 can exist in at least one distinct crystalline form. In some embodiments, the present invention provides a crystalline form of Compound 15 referred to herein as Type A.

In some embodiments, Compound 15 is amorphous. In some embodiments, Compound 15 is amorphous, and is substantially free of crystalline Compound 15.

### Compound 15 Type A

In some embodiments, Compound 15 Type A has at least 1, 2, 3, 4 or 5 spectral peak(s) selected from the peaks listed in Table 25 below.

TABLE 25
XRPD Peak Positions for Compound 15 Type A
Position (°2θ)

5.58
6.44
7.02
8.55
10.64
12.96
13.30
13.98
14.12
15.51
16.28
16.93
18.16
19.32
19.63
20.31
20.87
21.30
22.31
22.64
23.52
25.67
25.87
26.45
27.48
28.07
28.81
31.38
32.97

In some embodiments, Compound 15 Type A is characterized in that it has one or more peaks in its X-ray powder diffraction pattern selected from those at 5.58, 7.02, 8.55, 16.28, 19.63, and 22.64. In some embodiments, Compound 15 Type A is characterized in that it has two or more peaks in its X-ray powder diffraction pattern selected from those at 5.58, 7.02, 8.55, 16.28, 19.63, and 22.64. In some embodiments, Compound 15 Type A is characterized in that it has three or more peaks in its X-ray powder diffraction pattern selected from those at 5.58, 7.02, 8.55, 16.28, 19.63, and 22.64. In some embodiments, Compound 15 Type A is characterized in that it has four or more peaks in its X-ray powder diffraction pattern selected from those at 5.58, 7.02, 8.55, 16.28, 19.63, and 22.64. In some embodiments, Compound 15 Type A is characterized in that it has five or more peaks in its X-ray powder diffraction pattern selected from those at 5.58, 7.02, 8.55, 16.28, 19.63, and 22.64. In some embodiments, Compound 15 Type A is characterized in that it has all six peaks in its X-ray powder diffraction pattern selected from those at 5.58, 7.02, 8.55, 16.28, 19.63, and 22.64.

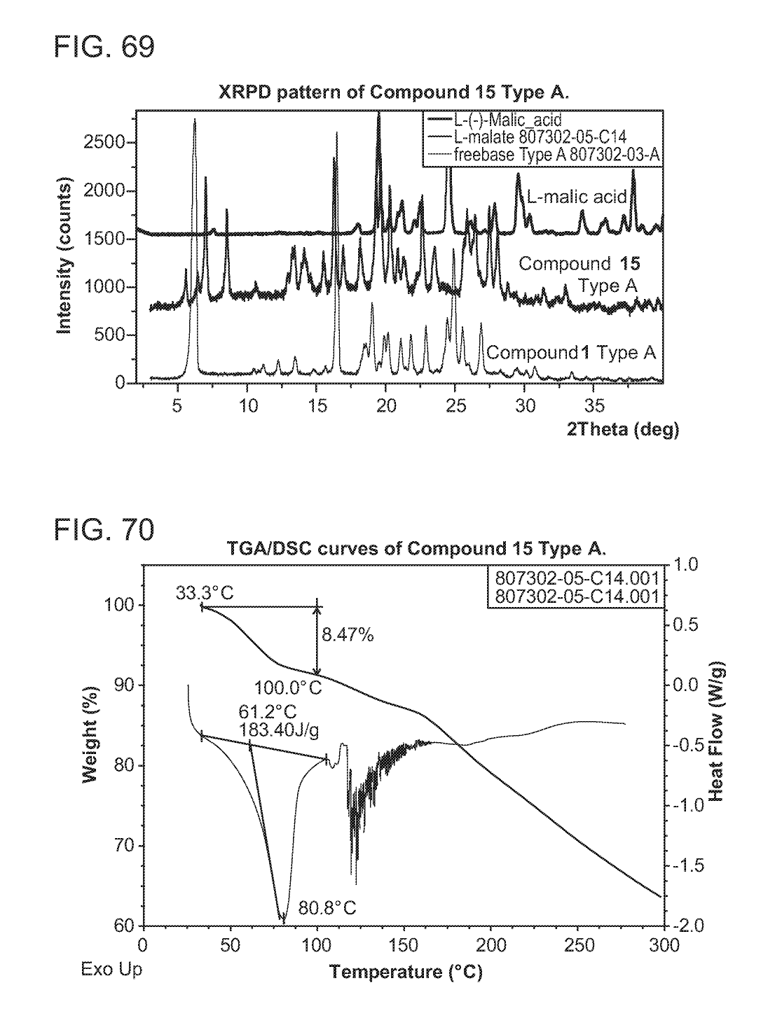

In certain embodiments, the X-ray powder diffraction pattern is substantially similar to the XRPD provided in FIG. 69.

Methods for preparing Compound 15 Type A are described infra.

### 16. Compound 16 (Succinic Acid×Compound 1)

According to one embodiment, the present invention provides a chemical species Compound 16 comprising Compound 1 and succinic acid:

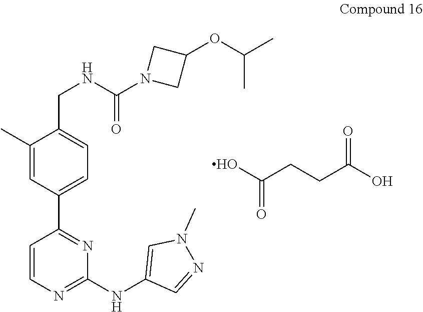

It is contemplated that Compound 16 can exist in a variety of physical forms. For example, Compound 16 can be in solution, suspension, or in solid form. In certain embodiments, Compound 16 is in solid form. When Compound 16 is in solid form, said compound may be amorphous, crystalline, or a mixture thereof. Exemplary solid forms are described in more detail below.

In one embodiment, the solid form of Compound 16 has a stoichiometry of (Compound 1):(succinic acid) that is about 1:1.

In some embodiments, the present invention provides Compound 16 substantially free of impurities. As used herein, the term “substantially free of impurities” means that the compound contains no significant amount of extraneous matter. Such extraneous matter may include excess succinic acid, excess Compound 1, residual solvents, or any other impurities that may result from the preparation of, and/or isolation of, Compound 16. In certain embodiments, at least about 95% by weight of Compound 16 is present. In still other embodiments of the invention, at least about 99% by weight of Compound 16 is present.

According to one embodiment, Compound 16 is present in an amount of at least about 97, 97.5, 98.0, 98.5, 99, 99.5, 99.8 weight percent where the percentages are based on the total weight of the composition. According to another embodiment, Compound 16 contains no more than about 3.0 area percent HPLC of total organic impurities and, in certain embodiments, no more than about 1.5 area percent HPLC total organic impurities relative to the total area of the HPLC chromatogram. In other embodiments, Compound 16 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 16 is also meant to include all tautomeric forms of Compound 16. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 16 can exist in a variety of solid forms. Exemplary such forms include polymorphs such as those described herein.

In certain embodiments, Compound 16 is a crystalline solid. In other embodiments, Compound 16 is a crystalline solid substantially free of amorphous Compound 16. As used herein, the term “substantially free of amorphous Compound 16” means that the compound contains no significant amount of amorphous Compound 16. In certain embodiments, at least about 95% by weight of crystalline Compound 16 is present. In still other embodiments of the invention, at least about 99% by weight of crystalline Compound 16 is present.

It has been found that Compound 16 can exist in at least one distinct crystalline form. In some embodiments, the present invention provides a crystalline form of Compound 16 referred to herein as Type A.

In some embodiments, Compound 16 is amorphous. In some embodiments, Compound 16 is amorphous, and is substantially free of crystalline Compound 16.

### Compound 16 Type A

In some embodiments, Compound 16 Type A has at least 1, 2, 3, 4 or 5 spectral peak(s) selected from the peaks listed in Table 26 below.

TABLE 26
XRPD Peak Positions for Compound 16 Type A
Position (°2θ)

5.62
7.04
8.55
10.78
11.32
12.36
13.97
15.47
16.02
16.58
18.24
18.79
19.89
20.65
21.18
21.99
22.80
25.25
25.66
26.28
26.86
28.10
30.60

In some embodiments, Compound 16 Type A is characterized in that it has one or more peaks in its X-ray powder diffraction pattern selected from those at about 5.62, 7.04, 8.55, 22.80, and 26.28. In some embodiments, Compound 16 Type A is characterized in that it has two or more peaks in its X-ray powder diffraction pattern selected from those at about 5.62, 7.04, 8.55, 22.80, and 26.28. In some embodiments, Compound 16 Type A is characterized in that it has three or more peaks in its X-ray powder diffraction pattern selected from those at about 5.62, 7.04, 8.55, 22.80, and 26.28. In some embodiments, Compound 16 Type A is characterized in that it has four or more peaks in its X-ray powder diffraction pattern selected from those at about 5.62, 7.04, 8.55, 22.80, and 26.28. In some embodiments, Compound 16 Type A is characterized in that it has all five peaks in its X-ray powder diffraction pattern selected from those at about 5.62, 7.04, 8.55, 22.80, and 26.28.

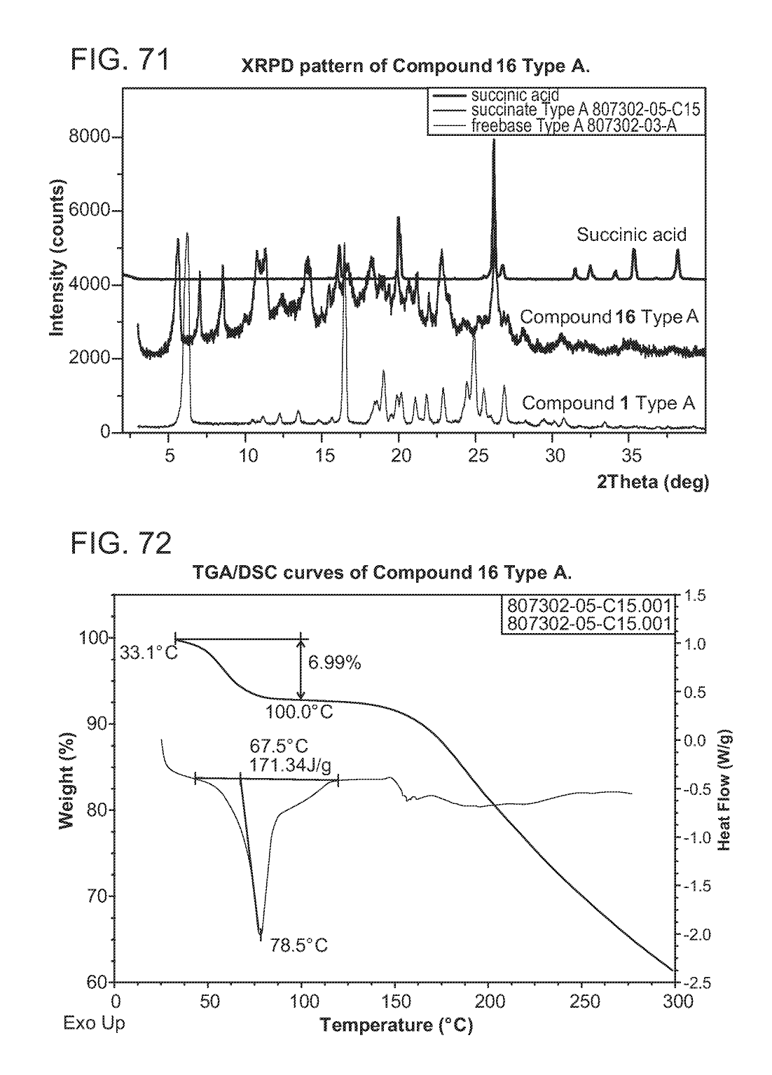

In certain embodiments, the X-ray powder diffraction pattern is substantially similar to the XRPD provided in FIG. 71.

Methods for preparing Compound 16 Type A are described infra.

### 17. Compound 17 (Hippuric Acid×Compound 1)

According to one embodiment, the present invention provides a chemical species Compound 17 comprising Compound 1 and hippuric acid:

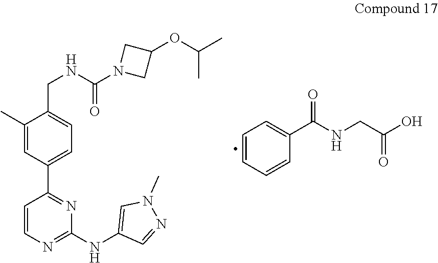 [SMILES: Cc1cc(-c2ccnc(Nc3cnn(C)c3)n2)ccc1CNC(=O)N1CC(OC(C)C)C1 ; O=C(O)CNC(=O)c1ccccc1]

It is contemplated that Compound 17 can exist in a variety of physical forms. For example, Compound 17 can be in solution, suspension, or in solid form. In certain embodiments, Compound 17 is in solid form. When Compound 17 is in solid form, said compound may be amorphous, crystalline, or a mixture thereof. Exemplary solid forms are described in more detail below.

In one embodiment, the solid form of Compound 17 has a stoichiometry of (Compound 1):(hippuric acid) that is about 1:1.

In some embodiments, the present invention provides Compound 17 substantially free of impurities. As used herein, the term “substantially free of impurities” means that the compound contains no significant amount of extraneous matter. Such extraneous matter may include excess hippuric acid, excess Compound 1, residual solvents, or any other impurities that may result from the preparation of, and/or isolation of, Compound 17. In certain embodiments, at least about 95% by weight of Compound 17 is present. In still other embodiments of the invention, at least about 99% by weight of Compound 17 is present.

According to one embodiment, Compound 17 is present in an amount of at least about 97, 97.5, 98.0, 98.5, 99, 99.5, 99.8 weight percent where the percentages are based on the total weight of the composition. According to another embodiment, Compound 17 contains no more than about 3.0 area percent HPLC of total organic impurities and, in certain embodiments, no more than about 1.5 area percent HPLC total organic impurities relative to the total area of the HPLC chromatogram. In other embodiments, Compound 17 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 17 is also meant to include all tautomeric forms of Compound 17. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 17 can exist in a variety of solid forms. Exemplary such forms include polymorphs such as those described herein.

In certain embodiments, Compound 17 is a crystalline solid. In other embodiments, Compound 17 is a crystalline solid substantially free of amorphous Compound 17. As used herein, the term “substantially free of amorphous Compound 17” means that the compound contains no significant amount of amorphous Compound 17. In certain embodiments, at least about 95% by weight of crystalline Compound 17 is present. In still other embodiments of the invention, at least about 99% by weight of crystalline Compound 17 is present.

In some embodiments, Compound 17 is amorphous. In some embodiments, Compound 17 is amorphous, and is substantially free of crystalline Compound 17.

### 18. Compound 18 (Maleic Acid×Compound 1)

According to one embodiment, the present invention provides a chemical species Compound 18 comprising Compound 1 and maleic acid:

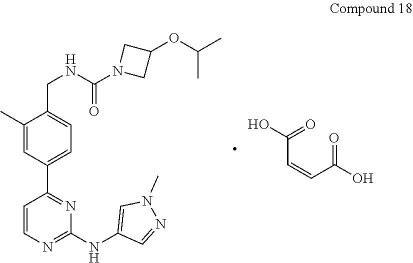 [SMILES: Cc1cc(-c2ccnc(Nc3cnn(C)c3)n2)ccc1CNC(=O)N1CC(OC(C)C)C1 ; O=C(O)/C=C\C(=O)O]

It is contemplated that Compound 18 can exist in a variety of physical forms. For example, Compound 18 can be in solution, suspension, or in solid form. In certain embodiments, Compound 18 is in solid form. When Compound 18 is in solid form, said compound may be amorphous, crystalline, or a mixture thereof. Exemplary solid forms are described in more detail below.

In one embodiment, the solid form of Compound 18 has a stoichiometry of (Compound 1):(maleic acid) that is about 1:1.

In some embodiments, the present invention provides Compound 18 substantially free of impurities. As used herein, the term “substantially free of impurities” means that the compound contains no significant amount of extraneous matter. Such extraneous matter may include excess maleic acid, excess Compound 1, residual solvents, or any other impurities that may result from the preparation of, and/or isolation of, Compound 18. In certain embodiments, at least about 95% by weight of Compound 18 is present. In still other embodiments of the invention, at least about 99% by weight of Compound 18 is present.

According to one embodiment, Compound 18 is present in an amount of at least about 97, 97.5, 98.0, 98.5, 99, 99.5, 99.8 weight percent where the percentages are based on the total weight of the composition. According to another embodiment, Compound 18 contains no more than about 3.0 area percent HPLC of total organic impurities and, in certain embodiments, no more than about 1.5 area percent HPLC total organic impurities relative to the total area of the HPLC chromatogram. In other embodiments, Compound 18 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 18 is also meant to include all tautomeric forms of Compound 18. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 18 can exist in a variety of solid forms. Exemplary such forms include polymorphs such as those described herein.

In certain embodiments, Compound 18 is a crystalline solid. In other embodiments, Compound 18 is a crystalline solid substantially free of amorphous Compound 18. As used herein, the term “substantially free of amorphous Compound 18” means that the compound contains no significant amount of amorphous Compound 18. In certain embodiments, at least about 95% by weight of crystalline Compound 18 is present. In still other embodiments of the invention, at least about 99% by weight of crystalline Compound 18 is present.

It has been found that Compound 18 can exist in at least two distinct polymorphic forms. In some embodiments, the present invention provides a polymorphic form of Compound 18 referred to herein as Type A. In some embodiments, the present invention provides a polymorphic form of Compound 18 referred to herein as Type B.

In some embodiments, Compound 18 is amorphous. In some embodiments, Compound 18 is amorphous, and is substantially free of crystalline Compound 18.

### Compound 18 Type A

In some embodiments, Compound 18 Type A has at least 1, 2, 3, 4 or 5 spectral peak(s) selected from the peaks listed in Table 27 below.

TABLE 27
XRPD Peak Positions for Compound 18 Type A
Position (°2θ)

8.50
9.48
11.83
13.33
14.81
15.77
16.88
17.11
17.84
18.34
18.90
19.14
19.32
19.93
20.24
20.82
21.11
21.50
22.11
22.67
23.13
23.70
23.85
24.36
24.96
25.51
26.49
27.12
27.49
27.99
28.78
29.32
29.75
31.84
32.01
34.52
36.15
37.19

In some embodiments, Compound 18 Type A is characterized in that it has one or more peaks in its X-ray powder diffraction pattern selected from those at about 8.50, 9.48, 11.83, 13.33, 17.84, 22.67, and 26.49. In some embodiments, Compound 18 Type A is characterized in that it has two or more peaks in its X-ray powder diffraction pattern selected from those at about 8.50, 9.48, 11.83, 13.33, 17.84, 22.67, and 26.49. In some embodiments, Compound 18 Type A is characterized in that it has three or more peaks in its X-ray powder diffraction pattern selected from those at about 8.50, 9.48, 11.83, 13.33, 17.84, 22.67, and 26.49. In some embodiments, Compound 18 Type A is characterized in that it has four or more peaks in its X-ray powder diffraction pattern selected from those at about 8.50, 9.48, 11.83, 13.33, 17.84, 22.67, and 26.49. In some embodiments, Compound 18 Type A is characterized in that it has five or more peaks in its X-ray powder diffraction pattern selected from those at about 8.50, 9.48, 11.83, 13.33, 17.84, 22.67, and 26.49. In some embodiments, Compound 18 Type A is characterized in that it has six or more peaks in its X-ray powder diffraction pattern selected from those at about 8.50, 9.48, 11.83, 13.33, 17.84, 22.67, and 26.49. In some embodiments, Compound 18 Type A is characterized in that it has all seven peaks in its X-ray powder diffraction pattern selected from those at about 8.50, 9.48, 11.83, 13.33, 17.84, 22.67, and 26.49.

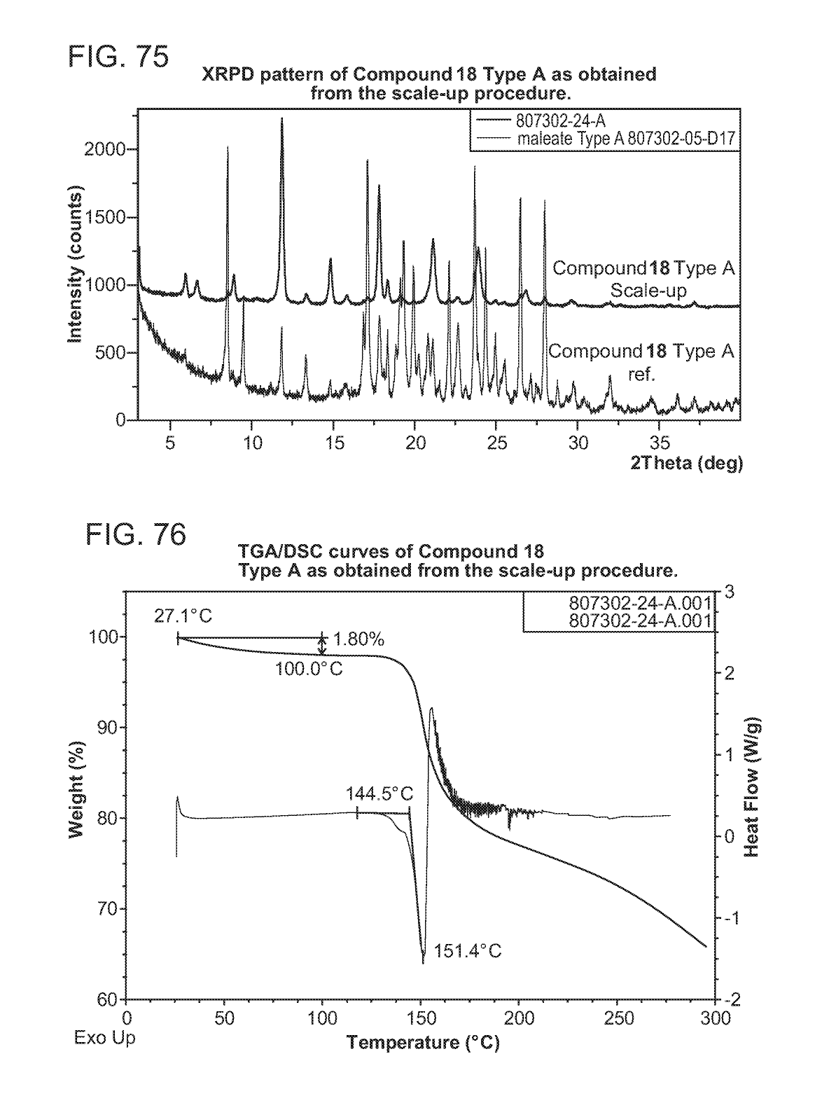

In certain embodiments, the X-ray powder diffraction pattern is substantially similar to the XRPD provided in FIG. 75.

Methods for preparing Compound 18 Type A are described infra.

### Compound 18 Type B

In some embodiments, Compound 18 Type B has at least 1, 2, 3, 4 or 5 spectral peak(s) selected from the peaks listed in Table 28 below.

TABLE 28
XRPD Peak Positions for Form B of Compound 18
Position (°2θ)

3.48
6.20
6.36
6.88
7.62
8.98
10.32
11.28
13.76
15.25
16.61
17.22
19.20
20.52
21.90
23.80
25.70
27.27
27.37

In some embodiments, Compound 18 Type B is characterized in that it has one or more peaks in its X-ray powder diffraction pattern selected from those at about 3.48, 6.20, 6.36, 7.62, 10.32, and 20.52. In some embodiments, Compound 18 Type B is characterized in that it has two or more peaks in its X-ray powder diffraction pattern selected from those at about 3.48, 6.20, 6.36, 7.62, 10.32, and 20.52. In some embodiments, Compound 18 Type B is characterized in that it has three or more peaks in its X-ray powder diffraction pattern selected from those at about 3.48, 6.20, 6.36, 7.62, 10.32, and 20.52. In some embodiments, Compound 18 Type B is characterized in that it has four or more peaks in its X-ray powder diffraction pattern selected from those at about 3.48, 6.20, 6.36, 7.62, 10.32, and 20.52. In some embodiments, Compound 18 Type B is characterized in that it has five or more peaks in its X-ray powder diffraction pattern selected from those at about 3.48, 6.20, 6.36, 7.62, 10.32, and 20.52. In some embodiments, Compound 18 Type B is characterized in that it has all six peaks in its X-ray powder diffraction pattern selected from those at about 3.48, 6.20, 6.36, 7.62, 10.32, and 20.52.

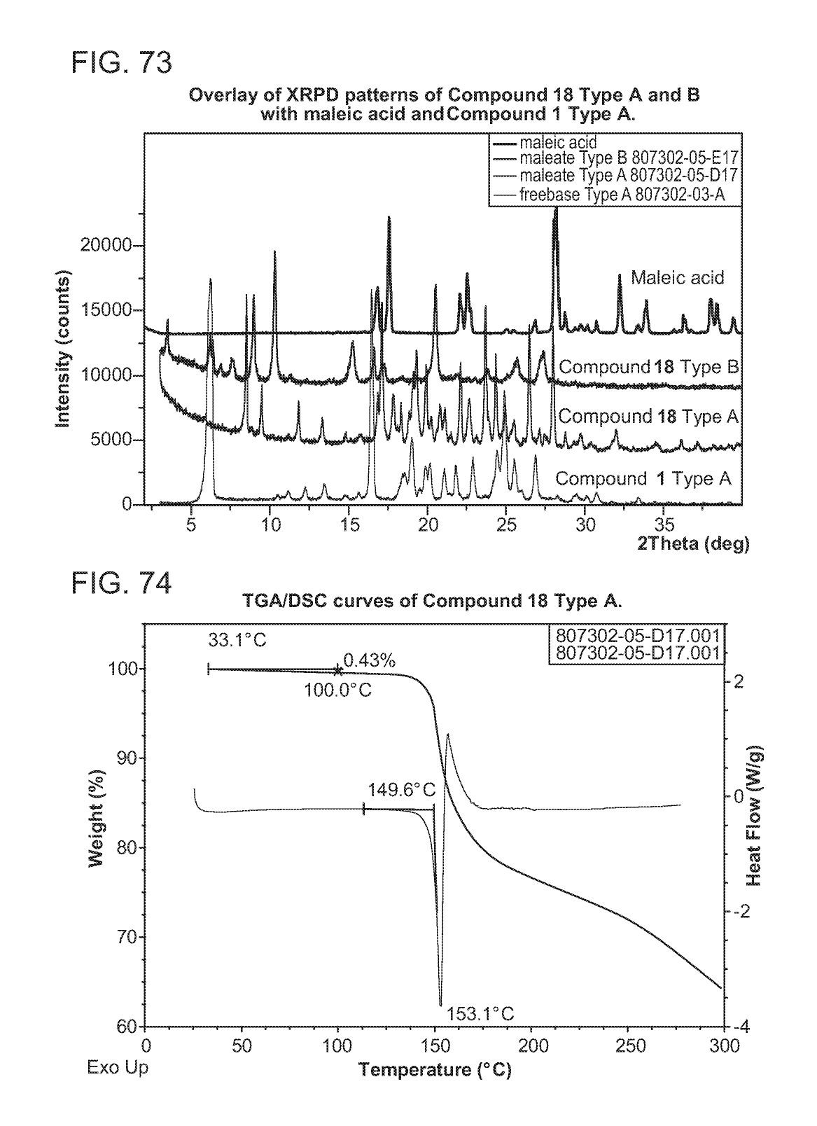

In certain embodiments, the X-ray powder diffraction pattern is substantially similar to the XRPD provided in FIG. 73.

Methods for preparing Compound 18 Type B are described infra.

### 19. Compound 19 (Glutamic Acid×Compound 1)

According to one embodiment, the present invention provides a chemical species Compound 19 comprising Compound 1 and glutamic acid:

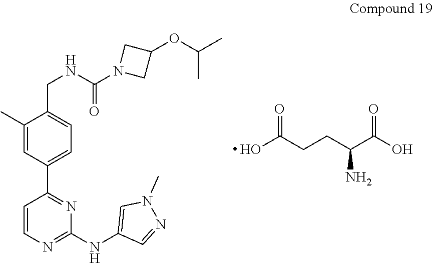 [SMILES: Cc1cc(-c2ccnc(Nc3cnn(C)c3)n2)ccc1CNC(=O)N1CC(OC(C)C)C1 ; N[C@@H](CCC(=O)O)C(=O)O]

It is contemplated that Compound 19 can exist in a variety of physical forms. For example, Compound 19 can be in solution, suspension, or in solid form. In certain embodiments, Compound 19 is in solid form. When Compound 19 is in solid form, said compound may be amorphous, crystalline, or a mixture thereof. Exemplary solid forms are described in more detail below.

In one embodiment, the solid form of Compound 19 has a stoichiometry of (Compound 1):(glutamic acid) that is about 1:1.

In some embodiments, the present invention provides Compound 19 substantially free of impurities. As used herein, the term “substantially free of impurities” means that the compound contains no significant amount of extraneous matter. Such extraneous matter may include excess glutamic acid, excess Compound 1, residual solvents, or any other impurities that may result from the preparation of, and/or isolation of, Compound 19. In certain embodiments, at least about 95% by weight of Compound 19 is present. In still other embodiments of the invention, at least about 99% by weight of Compound 19 is present.

According to one embodiment, Compound 19 is present in an amount of at least about 97, 97.5, 98.0, 98.5, 99, 99.5, 99.8 weight percent where the percentages are based on the total weight of the composition. According to another embodiment, Compound 19 contains no more than about 3.0 area percent HPLC of total organic impurities and, in certain embodiments, no more than about 1.5 area percent HPLC total organic impurities relative to the total area of the HPLC chromatogram. In other embodiments, Compound 19 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 19 is also meant to include all tautomeric forms of Compound 19. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 19 can exist in a variety of solid forms. Exemplary such forms include polymorphs such as those described herein.

In certain embodiments, Compound 19 is a crystalline solid. In other embodiments, Compound 19 is a crystalline solid substantially free of amorphous Compound 19. As used herein, the term “substantially free of amorphous Compound 19” means that the compound contains no significant amount of amorphous Compound 19. In certain embodiments, at least about 95% by weight of crystalline Compound 19 is present. In still other embodiments of the invention, at least about 99% by weight of crystalline Compound 19 is present.

In some embodiments, Compound 19 is amorphous. In some embodiments, Compound 19 is amorphous, and is substantially free of crystalline Compound 19.

### 20. Compound 20 (Benzoic Acid×Compound 1)

According to one embodiment, the present invention provides a chemical species Compound 20 comprising Compound 1 and benzoic acid:

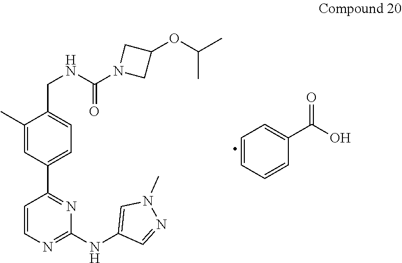 [SMILES: Cc1cc(-c2ccnc(Nc3cnn(C)c3)n2)ccc1CNC(=O)N1CC(OC(C)C)C1 ; O=C(O)c1ccccc1]

It is contemplated that Compound 20 can exist in a variety of physical forms. For example, Compound 20 can be in solution, suspension, or in solid form. In certain embodiments, Compound 20 is in solid form. When Compound 20 is in solid form, said compound may be amorphous, crystalline, or a mixture thereof. Exemplary solid forms are described in more detail below.

In one embodiment, the solid form of Compound 20 has a stoichiometry of (Compound 1):(benzoic acid) that is about 1:1.

In some embodiments, the present invention provides Compound 20 substantially free of impurities. As used herein, the term “substantially free of impurities” means that the compound contains no significant amount of extraneous matter. Such extraneous matter may include excess benzoic acid, excess Compound 1, residual solvents, or any other impurities that may result from the preparation of, and/or isolation of, Compound 20. In certain embodiments, at least about 95% by weight of Compound 20 is present. In still other embodiments of the invention, at least about 99% by weight of Compound 20 is present.

According to one embodiment, Compound 20 is present in an amount of at least about 97, 97.5, 98.0, 98.5, 99, 99.5, 99.8 weight percent where the percentages are based on the total weight of the composition. According to another embodiment, Compound 20 contains no more than about 3.0 area percent HPLC of total organic impurities and, in certain embodiments, no more than about 1.5 area percent HPLC total organic impurities relative to the total area of the HPLC chromatogram. In other embodiments, Compound 20 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 20 is also meant to include all tautomeric forms of Compound 20. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 20 can exist in a variety of solid forms. Exemplary such forms include polymorphs such as those described herein.

In certain embodiments, Compound 20 is a crystalline solid. In other embodiments, Compound 20 is a crystalline solid substantially free of amorphous Compound 20. As used herein, the term “substantially free of amorphous Compound 20” means that the compound contains no significant amount of amorphous Compound 20. In certain embodiments, at least about 95% by weight of crystalline Compound 20 is present. In still other embodiments of the invention, at least about 99% by weight of crystalline Compound 20 is present.

In some embodiments, Compound 20 is amorphous. In some embodiments, Compound 20 is amorphous, and is substantially free of crystalline Compound 20.

### 21. Compound 21 (Gentisic Acid×Compound 1)

According to one embodiment, the present invention provides a chemical species Compound 21 comprising Compound 1 and gentisic acid:

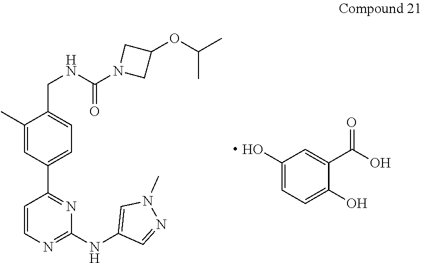 [SMILES: Cc1cc(-c2ccnc(Nc3cnn(C)c3)n2)ccc1CNC(=O)N1CC(OC(C)C)C1 ; O=C(O)c1cc(O)ccc1O]

It is contemplated that Compound 21 can exist in a variety of physical forms. For example, Compound 21 can be in solution, suspension, or in solid form. In certain embodiments, Compound 21 is in solid form. When Compound 21 is in solid form, said compound may be amorphous, crystalline, or a mixture thereof. Exemplary solid forms are described in more detail below.

In one embodiment, the solid form of Compound 21 has a stoichiometry of (Compound 1):(gentisic acid) that is about 1:1.

In some embodiments, the present invention provides Compound 21 substantially free of impurities. As used herein, the term “substantially free of impurities” means that the compound contains no significant amount of extraneous matter. Such extraneous matter may include excess gentisic acid, excess Compound 1, residual solvents, or any other impurities that may result from the preparation of, and/or isolation of, Compound 21. In certain embodiments, at least about 95% by weight of Compound 21 is present. In still other embodiments of the invention, at least about 99% by weight of Compound 21 is present.

According to one embodiment, Compound 21 is present in an amount of at least about 97, 97.5, 98.0, 98.5, 99, 99.5, 99.8 weight percent where the percentages are based on the total weight of the composition. According to another embodiment, Compound 21 contains no more than about 3.0 area percent HPLC of total organic impurities and, in certain embodiments, no more than about 1.5 area percent HPLC total organic impurities relative to the total area of the HPLC chromatogram. In other embodiments, Compound 21 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 21 is also meant to include all tautomeric forms of Compound 21. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 21 can exist in a variety of solid forms. Exemplary such forms include polymorphs such as those described herein.

In certain embodiments, Compound 21 is a crystalline solid. In other embodiments, Compound 21 is a crystalline solid substantially free of amorphous Compound 21. As used herein, the term “substantially free of amorphous Compound 21” means that the compound contains no significant amount of amorphous Compound 21. In certain embodiments, at least about 95% by weight of crystalline Compound 21 is present. In still other embodiments of the invention, at least about 99% by weight of crystalline Compound 21 is present.

It has been found that Compound 21 can exist in at least one distinct crystalline form. In some embodiments, the present invention provides a crystalline form of Compound 21 referred to herein as Form A.

In some embodiments, Compound 21 is amorphous. In some embodiments, Compound 21 is amorphous, and is substantially free of crystalline Compound 21.

### Compound 21 Type A

In some embodiments, Compound 21 Type A has at least 1, 2, 3, 4 or 5 spectral peak(s) selected from the peaks listed in Table 29 below.

TABLE 29
XRPD Peak Positions for Compound 21 Type A
Position (°2θ)

7.57
8.08
12.29
15.44
16.18
17.54
18.06
19.30
20.71
23.03
23.56
24.91
26.82
31.19
31.81

In some embodiments, Compound 21 Type A is characterized in that it has one or more peaks in its X-ray powder diffraction pattern selected from those at about 7.57, 8.08, 15.44, 16.18, 19.30, and 20.71. In some embodiments, Compound 21 Type A is characterized in that it has two or more peaks in its X-ray powder diffraction pattern selected from those at about 7.57, 8.08, 15.44, 16.18, 19.30, and 20.71. In some embodiments, Compound 21 Type A is characterized in that it has three or more peaks in its X-ray powder diffraction pattern selected from those at about 7.57, 8.08, 15.44, 16.18, 19.30, and 20.71. In some embodiments, Compound 21 Type A is characterized in that it has four or more peaks in its X-ray powder diffraction pattern selected from those at about 7.57, 8.08, 15.44, 16.18, 19.30, and 20.71. In some embodiments, Compound 21 Type A is characterized in that it has five or more peaks in its X-ray powder diffraction pattern selected from those at about 7.57, 8.08, 15.44, 16.18, 19.30, and 20.71. In some embodiments, Compound 21 Type A is characterized in that it has all six peaks in its X-ray powder diffraction pattern selected from those at about 7.57, 8.08, 15.44, 16.18, 19.30, and 20.71.

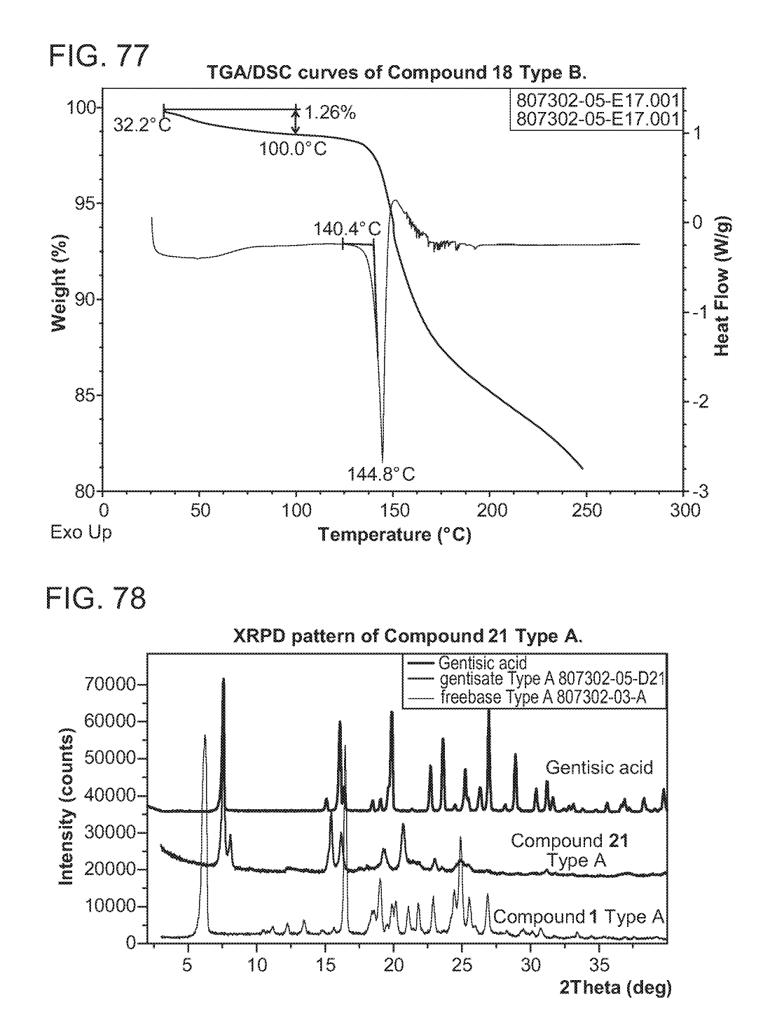

In certain embodiments, the X-ray powder diffraction pattern is substantially similar to the XRPD provided in FIG. 78.

Methods for preparing Compound 21 Type A are described infra.

### 22. Compound 22 (Malonic Acid×Compound 1)

According to one embodiment, the present invention provides a chemical species Compound 22 comprising Compound 1 and malonic acid:

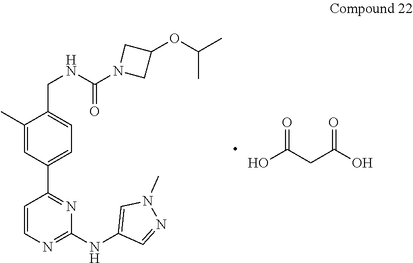 [SMILES: Cc1cc(-c2ccnc(Nc3cnn(C)c3)n2)ccc1CNC(=O)N1CC(OC(C)C)C1 ; O=C(O)CC(=O)O]

It is contemplated that Compound 22 can exist in a variety of physical forms. For example, Compound 22 can be in solution, suspension, or in solid form. In certain embodiments, Compound 22 is in solid form. When Compound 22 is in solid form, said compound may be amorphous, crystalline, or a mixture thereof. Exemplary solid forms are described in more detail below.

In one embodiment, the solid form of Compound 22 has a stoichiometry of (Compound 1):(malonic acid) that is about 1:1.

In some embodiments, the present invention provides Compound 22 substantially free of impurities. As used herein, the term “substantially free of impurities” means that the compound contains no significant amount of extraneous matter. Such extraneous matter may include excess malonic acid, excess Compound 1, residual solvents, or any other impurities that may result from the preparation of, and/or isolation of, Compound 22. In certain embodiments, at least about 95% by weight of Compound 22 is present. In still other embodiments of the invention, at least about 99% by weight of Compound 22 is present.

According to one embodiment, Compound 22 is present in an amount of at least about 97, 97.5, 98.0, 98.5, 99, 99.5, 99.8 weight percent where the percentages are based on the total weight of the composition. According to another embodiment, Compound 22 contains no more than about 3.0 area percent HPLC of total organic impurities and, in certain embodiments, no more than about 1.5 area percent HPLC total organic impurities relative to the total area of the HPLC chromatogram. In other embodiments, Compound 22 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 22 is also meant to include all tautomeric forms of Compound 22. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 22 can exist in a variety of solid forms. Exemplary such forms include polymorphs such as those described herein.

In certain embodiments, Compound 22 is a crystalline solid. In other embodiments, Compound 22 is a crystalline solid substantially free of amorphous Compound 22. As used herein, the term “substantially free of amorphous Compound 22” means that the compound contains no significant amount of amorphous Compound 22. In certain embodiments, at least about 95% by weight of crystalline Compound 22 is present. In still other embodiments of the invention, at least about 99% by weight of crystalline Compound 22 is present.

In some embodiments, Compound 22 is amorphous. In some embodiments, Compound 22 is amorphous, and is substantially free of crystalline Compound 22.

### 23. Compound 23 (Cinnamic Acid×Compound 1)

According to one embodiment, the present invention provides a chemical species Compound 23 comprising Compound 1 and cinnamic acid:

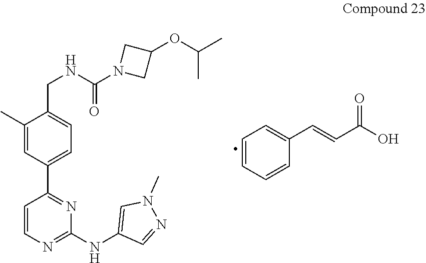 [SMILES: Cc1cc(-c2ccnc(Nc3cnn(C)c3)n2)ccc1CNC(=O)N1CC(OC(C)C)C1 ; O=C(O)/C=C/c1ccccc1]

It is contemplated that Compound 23 can exist in a variety of physical forms. For example, Compound 23 can be in solution, suspension, or in solid form. In certain embodiments, Compound 23 is in solid form. When Compound 23 is in solid form, said compound may be amorphous, crystalline, or a mixture thereof. Exemplary solid forms are described in more detail below.

In one embodiment, the solid form of Compound 23 has a stoichiometry of (Compound 1):(cinnamic acid) that is about 1:1.

In some embodiments, the present invention provides Compound 23 substantially free of impurities. As used herein, the term “substantially free of impurities” means that the compound contains no significant amount of extraneous matter. Such extraneous matter may include excess cinnamic acid, excess Compound 1, residual solvents, or any other impurities that may result from the preparation of, and/or isolation of, Compound 23. In certain embodiments, at least about 95% by weight of Compound 23 is present. In still other embodiments of the invention, at least about 99% by weight of Compound 23 is present.

According to one embodiment, Compound 23 is present in an amount of at least about 97, 97.5, 98.0, 98.5, 99, 99.5, 99.8 weight percent where the percentages are based on the total weight of the composition. According to another embodiment, Compound 23 contains no more than about 3.0 area percent HPLC of total organic impurities and, in certain embodiments, no more than about 1.5 area percent HPLC total organic impurities relative to the total area of the HPLC chromatogram. In other embodiments, Compound 23 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 23 is also meant to include all tautomeric forms of Compound 23. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 23 can exist in a variety of solid forms. Exemplary such forms include polymorphs such as those described herein.

In certain embodiments, Compound 23 is a crystalline solid. In other embodiments, Compound 23 is a crystalline solid substantially free of amorphous Compound 23. As used herein, the term “substantially free of amorphous Compound 23” means that the compound contains no significant amount of amorphous Compound 23. In certain embodiments, at least about 95% by weight of crystalline Compound 23 is present. In still other embodiments of the invention, at least about 99% by weight of crystalline Compound 23 is present.

In some embodiments, Compound 23 is amorphous. In some embodiments, Compound 23 is amorphous, and is substantially free of crystalline Compound 23.

### 24. Compound 24 (L-Glutamine×Compound 1)

According to one embodiment, the present invention provides a chemical species Compound 24 comprising Compound 1 and L-glutamine:

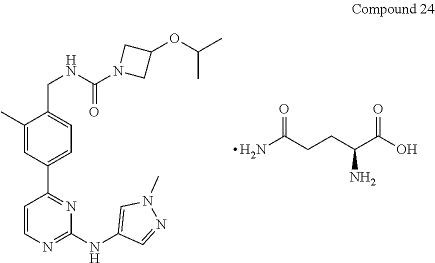 [SMILES: Cc1cc(-c2ccnc(Nc3cnn(C)c3)n2)ccc1CNC(=O)N1CC(OC(C)C)C1 ; NC(=O)CC[C@H](N)C(=O)O]

It is contemplated that Compound 24 can exist in a variety of physical forms. For example, Compound 24 can be in solution, suspension, or in solid form. In certain embodiments, Compound 24 is in solid form. When Compound 24 is in solid form, said compound may be amorphous, crystalline, or a mixture thereof. Exemplary solid forms are described in more detail below.

In one embodiment, the solid form of Compound 24 has a stoichiometry of (Compound 1):(L-glutamine) that is about 1:1.

In some embodiments, the present invention provides Compound 24 substantially free of impurities. As used herein, the term “substantially free of impurities” means that the compound contains no significant amount of extraneous matter. Such extraneous matter may include excess L-glutamine, excess Compound 1, residual solvents, or any other impurities that may result from the preparation of, and/or isolation of, Compound 24. In certain embodiments, at least about 95% by weight of Compound 24 is present. In still other embodiments of the invention, at least about 99% by weight of Compound 24 is present.

According to one embodiment, Compound 24 is present in an amount of at least about 97, 97.5, 98.0, 98.5, 99, 99.5, 99.8 weight percent where the percentages are based on the total weight of the composition. According to another embodiment, Compound 24 contains no more than about 3.0 area percent HPLC of total organic impurities and, in certain embodiments, no more than about 1.5 area percent HPLC total organic impurities relative to the total area of the HPLC chromatogram. In other embodiments, Compound 24 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 24 is also meant to include all tautomeric forms of Compound 24. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 24 can exist in a variety of solid forms. Exemplary such forms include polymorphs such as those described herein.

In certain embodiments, Compound 24 is a crystalline solid. In other embodiments, Compound 24 is a crystalline solid substantially free of amorphous Compound 24. As used herein, the term “substantially free of amorphous Compound 24” means that the compound contains no significant amount of amorphous Compound 24. In certain embodiments, at least about 95% by weight of crystalline Compound 24 is present. In still other embodiments of the invention, at least about 99% by weight of crystalline Compound 24 is present.

In some embodiments, Compound 24 is amorphous. In some embodiments, Compound 24 is amorphous, and is substantially free of crystalline Compound 24.

### 25. Compound 25 (L-Lysine×Compound 1)

According to one embodiment, the present invention provides a chemical species Compound 25 comprising Compound 1 and L-lysine:

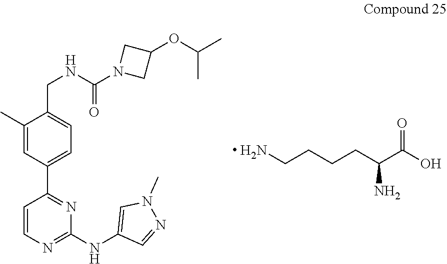 [SMILES: Cc1cc(-c2ccnc(Nc3cnn(C)c3)n2)ccc1CNC(=O)N1CC(OC(C)C)C1 ; NCCCC[C@H](N)C(=O)O]

It is contemplated that Compound 25 can exist in a variety of physical forms. For example, Compound 25 can be in solution, suspension, or in solid form. In certain embodiments, Compound 25 is in solid form. When Compound 25 is in solid form, said compound may be amorphous, crystalline, or a mixture thereof. Exemplary solid forms are described in more detail below.

In one embodiment, the solid form of Compound 25 has a stoichiometry of (Compound 1):(L-lysine) that is about 1:1.

In some embodiments, the present invention provides Compound 25 substantially free of impurities. As used herein, the term “substantially free of impurities” means that the compound contains no significant amount of extraneous matter. Such extraneous matter may include excess L-lysine, excess Compound 1, residual solvents, or any other impurities that may result from the preparation of, and/or isolation of, Compound 25. In certain embodiments, at least about 95% by weight of Compound 25 is present. In still other embodiments of the invention, at least about 99% by weight of Compound 25 is present.

According to one embodiment, Compound 25 is present in an amount of at least about 97, 97.5, 98.0, 98.5, 99, 99.5, 99.8 weight percent where the percentages are based on the total weight of the composition. According to another embodiment, Compound 25 contains no more than about 3.0 area percent HPLC of total organic impurities and, in certain embodiments, no more than about 1.5 area percent HPLC total organic impurities relative to the total area of the HPLC chromatogram. In other embodiments, Compound 25 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 25 is also meant to include all tautomeric forms of Compound 25. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 25 can exist in a variety of solid forms. Exemplary such forms include polymorphs such as those described herein.

In certain embodiments, Compound 25 is a crystalline solid. In other embodiments, Compound 25 is a crystalline solid substantially free of amorphous Compound 25. As used herein, the term “substantially free of amorphous Compound 25” means that the compound contains no significant amount of amorphous Compound 25. In certain embodiments, at least about 95% by weight of crystalline Compound 25 is present. In still other embodiments of the invention, at least about 99% by weight of crystalline Compound 25 is present.

In some embodiments, Compound 25 is amorphous. In some embodiments, Compound 25 is amorphous, and is substantially free of crystalline Compound 25.

### 26. Compound 26 (L-Phenylalanine×Compound 1)

According to one embodiment, the present invention provides a chemical species Compound 26 comprising Compound 1 and L-phenylalanine:

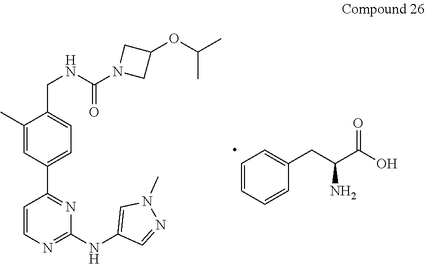 [SMILES: Cc1cc(-c2ccnc(Nc3cnn(C)c3)n2)ccc1CNC(=O)N1CC(OC(C)C)C1 ; N[C@@H](Cc1ccccc1)C(=O)O]

It is contemplated that Compound 26 can exist in a variety of physical forms. For example, Compound 26 can be in solution, suspension, or in solid form. In certain embodiments, Compound 26 is in solid form. When Compound 26 is in solid form, said compound may be amorphous, crystalline, or a mixture thereof. Exemplary solid forms are described in more detail below.

In one embodiment, the solid form of Compound 26 has a stoichiometry of (Compound 1):(L-phenylalanine) that is about 1:1.

In some embodiments, the present invention provides Compound 26 substantially free of impurities. As used herein, the term “substantially free of impurities” means that the compound contains no significant amount of extraneous matter. Such extraneous matter may include excess L-phenylalanine, excess Compound 1, residual solvents, or any other impurities that may result from the preparation of, and/or isolation of, Compound 26. In certain embodiments, at least about 95% by weight of Compound 26 is present. In still other embodiments of the invention, at least about 99% by weight of Compound 26 is present.

According to one embodiment, Compound 26 is present in an amount of at least about 97, 97.5, 98.0, 98.5, 99, 99.5, 99.8 weight percent where the percentages are based on the total weight of the composition. According to another embodiment, Compound 26 contains no more than about 3.0 area percent HPLC of total organic impurities and, in certain embodiments, no more than about 1.5 area percent HPLC total organic impurities relative to the total area of the HPLC chromatogram. In other embodiments, Compound 26 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 26 is also meant to include all tautomeric forms of Compound 26. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 26 can exist in a variety of solid forms. Exemplary such forms include polymorphs such as those described herein.

In certain embodiments, Compound 26 is a crystalline solid. In other embodiments, Compound 26 is a crystalline solid substantially free of amorphous Compound 26. As used herein, the term “substantially free of amorphous Compound 26” means that the compound contains no significant amount of amorphous Compound 26. In certain embodiments, at least about 95% by weight of crystalline Compound 26 is present. In still other embodiments of the invention, at least about 99% by weight of crystalline Compound 26 is present.

In some embodiments, Compound 26 is amorphous. In some embodiments, Compound 26 is amorphous, and is substantially free of crystalline Compound 26.

### 27. Compound 27 (L-Proline×Compound 1)

According to one embodiment, the present invention provides a chemical species Compound 27 comprising Compound 1 and L-proline:

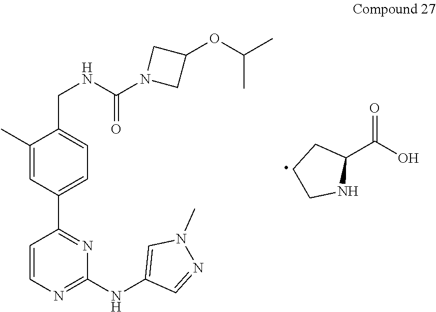 [SMILES: Cc1cc(-c2ccnc(Nc3cnn(C)c3)n2)ccc1CNC(=O)N1CC(OC(C)C)C1 ; O=C(O)[C@@H]1CCCN1]

It is contemplated that Compound 27 can exist in a variety of physical forms. For example, Compound 27 can be in solution, suspension, or in solid form. In certain embodiments, Compound 27 is in solid form. When Compound 27 is in solid form, said compound may be amorphous, crystalline, or a mixture thereof. Exemplary solid forms are described in more detail below.

In one embodiment, the solid form of Compound 27 has a stoichiometry of (Compound 1):(L-proline) that is about 1:1.

In some embodiments, the present invention provides Compound 27 substantially free of impurities. As used herein, the term “substantially free of impurities” means that the compound contains no significant amount of extraneous matter. Such extraneous matter may include excess L-proline, excess Compound 1, residual solvents, or any other impurities that may result from the preparation of, and/or isolation of, Compound 27. In certain embodiments, at least about 95% by weight of Compound 27 is present. In still other embodiments of the invention, at least about 99% by weight of Compound 27 is present.

According to one embodiment, Compound 27 is present in an amount of at least about 97, 97.5, 98.0, 98.5, 99, 99.5, 99.8 weight percent where the percentages are based on the total weight of the composition. According to another embodiment, Compound 27 contains no more than about 3.0 area percent HPLC of total organic impurities and, in certain embodiments, no more than about 1.5 area percent HPLC total organic impurities relative to the total area of the HPLC chromatogram. In other embodiments, Compound 27 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 27 is also meant to include all tautomeric forms of Compound 27. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 27 can exist in a variety of solid forms. Exemplary such forms include polymorphs such as those described herein.

In certain embodiments, Compound 27 is a crystalline solid. In other embodiments, Compound 27 is a crystalline solid substantially free of amorphous Compound 27. As used herein, the term “substantially free of amorphous Compound 27” means that the compound contains no significant amount of amorphous Compound 27. In certain embodiments, at least about 95% by weight of crystalline Compound 27 is present. In still other embodiments of the invention, at least about 99% by weight of crystalline Compound 27 is present.

In some embodiments, Compound 27 is amorphous. In some embodiments, Compound 27 is amorphous, and is substantially free of crystalline Compound 27.

### 28. Compound 28 (L-Serine×Compound 1)

According to one embodiment, the present invention provides a chemical species Compound 28 comprising Compound 1 and L-serine:

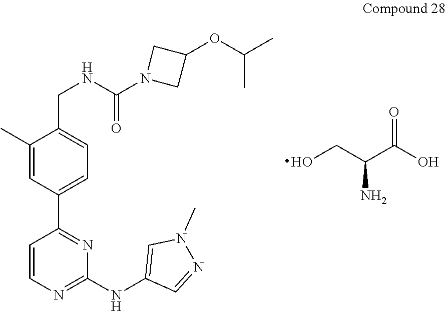 [SMILES: Cc1cc(-c2ccnc(Nc3cnn(C)c3)n2)ccc1CNC(=O)N1CC(OC(C)C)C1 ; N[C@@H](CO)C(=O)O]

It is contemplated that Compound 28 can exist in a variety of physical forms. For example, Compound 28 can be in solution, suspension, or in solid form. In certain embodiments, Compound 28 is in solid form. When Compound 28 is in solid form, said compound may be amorphous, crystalline, or a mixture thereof. Exemplary solid forms are described in more detail below.

In one embodiment, the solid form of Compound 3 has a stoichiometry of (Compound 1):(L-serine) that is about 1:1.

In some embodiments, the present invention provides Compound 28 substantially free of impurities. As used herein, the term “substantially free of impurities” means that the compound contains no significant amount of extraneous matter. Such extraneous matter may include excess L-serine, excess Compound 1, residual solvents, or any other impurities that may result from the preparation of, and/or isolation of, Compound 28. In certain embodiments, at least about 95% by weight of Compound 28 is present. In still other embodiments of the invention, at least about 99% by weight of Compound 28 is present.

According to one embodiment, Compound 28 is present in an amount of at least about 97, 97.5, 98.0, 98.5, 99, 99.5, 99.8 weight percent where the percentages are based on the total weight of the composition. According to another embodiment, Compound 28 contains no more than about 3.0 area percent HPLC of total organic impurities and, in certain embodiments, no more than about 1.5 area percent HPLC total organic impurities relative to the total area of the HPLC chromatogram. In other embodiments, Compound 28 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 28 is also meant to include all tautomeric forms of Compound 28. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 28 can exist in a variety of solid forms. Exemplary such forms include polymorphs such as those described herein.

In certain embodiments, Compound 28 is a crystalline solid. In other embodiments, Compound 28 is a crystalline solid substantially free of amorphous Compound 28. As used herein, the term “substantially free of amorphous Compound 28” means that the compound contains no significant amount of amorphous Compound 28. In certain embodiments, at least about 95% by weight of crystalline Compound 28 is present. In still other embodiments of the invention, at least about 99% by weight of crystalline Compound 28 is present.

In some embodiments, Compound 28 is amorphous. In some embodiments, Compound 28 is amorphous, and is substantially free of crystalline Compound 28.

### 29. Compound 29 (L-Tyrosine×Compound 1)

According to one embodiment, the present invention provides a chemical species Compound 29 comprising Compound 1 and L-tyrosine:

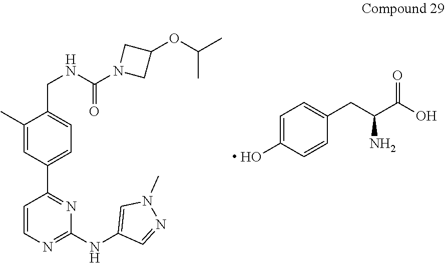 [SMILES: Cc1cc(-c2ccnc(Nc3cnn(C)c3)n2)ccc1CNC(=O)N1CC(OC(C)C)C1 ; N[C@@H](Cc1ccc(O)cc1)C(=O)O]

It is contemplated that Compound 29 can exist in a variety of physical forms. For example, Compound 29 can be in solution, suspension, or in solid form. In certain embodiments, Compound 29 is in solid form. When Compound 29 is in solid form, said compound may be amorphous, crystalline, or a mixture thereof. Exemplary solid forms are described in more detail below.

In one embodiment, the solid form of Compound 29 has a stoichiometry of (Compound 1):(L-tyrosine) that is about 1:1.

In some embodiments, the present invention provides Compound 29 substantially free of impurities. As used herein, the term “substantially free of impurities” means that the compound contains no significant amount of extraneous matter. Such extraneous matter may include excess L-tyrosine, excess Compound 1, residual solvents, or any other impurities that may result from the preparation of, and/or isolation of, Compound 29. In certain embodiments, at least about 95% by weight of Compound 29 is present. In still other embodiments of the invention, at least about 99% by weight of Compound 29 is present.

According to one embodiment, Compound 29 is present in an amount of at least about 97, 97.5, 98.0, 98.5, 99, 99.5, 99.8 weight percent where the percentages are based on the total weight of the composition. According to another embodiment, Compound 29 contains no more than about 3.0 area percent HPLC of total organic impurities and, in certain embodiments, no more than about 1.5 area percent HPLC total organic impurities relative to the total area of the HPLC chromatogram. In other embodiments, Compound 29 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 29 is also meant to include all tautomeric forms of Compound 29. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 29 can exist in a variety of solid forms. Exemplary such forms include polymorphs such as those described herein.

In certain embodiments, Compound 29 is a crystalline solid. In other embodiments, Compound 29 is a crystalline solid substantially free of amorphous Compound 29. As used herein, the term “substantially free of amorphous Compound 29” means that the compound contains no significant amount of amorphous Compound 29. In certain embodiments, at least about 95% by weight of crystalline Compound 29 is present. In still other embodiments of the invention, at least about 99% by weight of crystalline Compound 29 is present.

In some embodiments, Compound 29 is amorphous. In some embodiments, Compound 29 is amorphous, and is substantially free of crystalline Compound 29.

### 30. Compound 30 (Nicotinamide×Compound 1)

According to one embodiment, the present invention provides a chemical species Compound 30 comprising Compound 1 and nicotinamide:

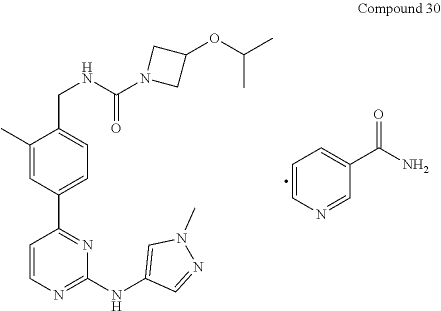 [SMILES: Cc1cc(-c2ccnc(Nc3cnn(C)c3)n2)ccc1CNC(=O)N1CC(OC(C)C)C1 ; NC(=O)c1cccnc1]

It is contemplated that Compound 30 can exist in a variety of physical forms. For example, Compound 30 can be in solution, suspension, or in solid form. In certain embodiments, Compound 30 is in solid form. When Compound 30 is in solid form, said compound may be amorphous, crystalline, or a mixture thereof. Exemplary solid forms are described in more detail below.

In one embodiment, the solid form of Compound 30 has a stoichiometry of (Compound 1):(nicotinamide) that is about 1:1.

In some embodiments, the present invention provides Compound 30 substantially free of impurities. As used herein, the term “substantially free of impurities” means that the compound contains no significant amount of extraneous matter. Such extraneous matter may include excess nicotinamide, excess Compound 1, residual solvents, or any other impurities that may result from the preparation of, and/or isolation of, Compound 30. In certain embodiments, at least about 95% by weight of Compound 30 is present. In still other embodiments of the invention, at least about 99% by weight of Compound 30 is present.

According to one embodiment, Compound 30 is present in an amount of at least about 97, 97.5, 98.0, 98.5, 99, 99.5, 99.8 weight percent where the percentages are based on the total weight of the composition. According to another embodiment, Compound 30 contains no more than about 3.0 area percent HPLC of total organic impurities and, in certain embodiments, no more than about 1.5 area percent HPLC total organic impurities relative to the total area of the HPLC chromatogram. In other embodiments, Compound 30 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 30 is also meant to include all tautomeric forms of Compound 30. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 30 can exist in a variety of solid forms. Exemplary such forms include polymorphs such as those described herein.

In certain embodiments, Compound 30 is a crystalline solid. In other embodiments, Compound 30 is a crystalline solid substantially free of amorphous Compound 30. As used herein, the term “substantially free of amorphous Compound 30” means that the compound contains no significant amount of amorphous Compound 30. In certain embodiments, at least about 95% by weight of crystalline Compound 30 is present. In still other embodiments of the invention, at least about 99% by weight of crystalline Compound 30 is present.

In some embodiments, Compound 30 is amorphous. In some embodiments, Compound 30 is amorphous, and is substantially free of crystalline Compound 30.

### 31. Compound 31 (Nicotinic Acid×Compound 1)

According to one embodiment, the present invention provides a chemical species Compound 31 comprising Compound 1 and nicotinic acid:

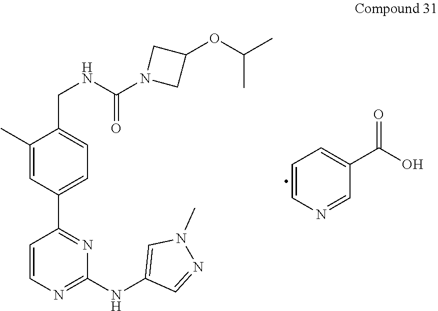 [SMILES: Cc1cc(-c2ccnc(Nc3cnn(C)c3)n2)ccc1CNC(=O)N1CC(OC(C)C)C1 ; O=C(O)c1cccnc1]

It is contemplated that Compound 31 can exist in a variety of physical forms. For example, Compound 31 can be in solution, suspension, or in solid form. In certain embodiments, Compound 31 is in solid form. When Compound 31 is in solid form, said compound may be amorphous, crystalline, or a mixture thereof. Exemplary solid forms are described in more detail below.

In one embodiment, the solid form of Compound 31 has a stoichiometry of (Compound 1):(nicotinic acid) that is about 1:1.

In some embodiments, the present invention provides Compound 31 substantially free of impurities. As used herein, the term “substantially free of impurities” means that the compound contains no significant amount of extraneous matter. Such extraneous matter may include excess nicotinic acid, excess Compound 1, residual solvents, or any other impurities that may result from the preparation of, and/or isolation of, Compound 31. In certain embodiments, at least about 95% by weight of Compound 31 is present. In still other embodiments of the invention, at least about 99% by weight of Compound 31 is present.

According to one embodiment, Compound 31 is present in an amount of at least about 97, 97.5, 98.0, 98.5, 99, 99.5, 99.8 weight percent where the percentages are based on the total weight of the composition. According to another embodiment, Compound 31 contains no more than about 3.0 area percent HPLC of total organic impurities and, in certain embodiments, no more than about 1.5 area percent HPLC total organic impurities relative to the total area of the HPLC chromatogram. In other embodiments, Compound 31 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 31 is also meant to include all tautomeric forms of Compound 31. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 31 can exist in a variety of solid forms. Exemplary such forms include polymorphs such as those described herein.

In certain embodiments, Compound 31 is a crystalline solid. In other embodiments, Compound 31 is a crystalline solid substantially free of amorphous Compound 31. As used herein, the term “substantially free of amorphous Compound 31” means that the compound contains no significant amount of amorphous Compound 31. In certain embodiments, at least about 95% by weight of crystalline Compound 31 is present. In still other embodiments of the invention, at least about 99% by weight of crystalline Compound 31 is present.

In some embodiments, Compound 31 is amorphous. In some embodiments, Compound 31 is amorphous, and is substantially free of crystalline Compound 31.

### 32. Compound 32 (Saccharin×Compound 1)

According to one embodiment, the present invention provides a chemical species Compound 32 comprising Compound 1 and saccharin:

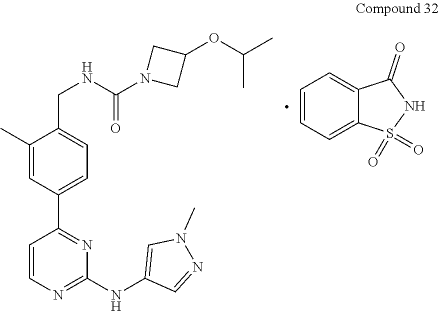 [SMILES: Cc1cc(-c2ccnc(Nc3cnn(C)c3)n2)ccc1CNC(=O)N1CC(OC(C)C)C1 ; O=C1NS(=O)(=O)c2ccccc21]

It is contemplated that Compound 32 can exist in a variety of physical forms. For example, Compound 32 can be in solution, suspension, or in solid form. In certain embodiments, Compound 32 is in solid form. When Compound 32 is in solid form, said compound may be amorphous, crystalline, or a mixture thereof. Exemplary solid forms are described in more detail below.

In one embodiment, the solid form of Compound 32 has a stoichiometry of (Compound 1):(saccharin) that is about 1:1.

In some embodiments, the present invention provides Compound 32 substantially free of impurities. As used herein, the term “substantially free of impurities” means that the compound contains no significant amount of extraneous matter. Such extraneous matter may include excess saccharin, excess Compound 1, residual solvents, or any other impurities that may result from the preparation of, and/or isolation of, Compound 32. In certain embodiments, at least about 95% by weight of Compound 32 is present. In still other embodiments of the invention, at least about 99% by weight of Compound 32 is present.

According to one embodiment, Compound 32 is present in an amount of at least about 97, 97.5, 98.0, 98.5, 99, 99.5, 99.8 weight percent where the percentages are based on the total weight of the composition. According to another embodiment, Compound 32 contains no more than about 3.0 area percent HPLC of total organic impurities and, in certain embodiments, no more than about 1.5 area percent HPLC total organic impurities relative to the total area of the HPLC chromatogram. In other embodiments, Compound 32 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 32 is also meant to include all tautomeric forms of Compound 32. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 32 can exist in a variety of solid forms. Exemplary such forms include polymorphs such as those described herein.

In certain embodiments, Compound 32 is a crystalline solid. In other embodiments, Compound 32 is a crystalline solid substantially free of amorphous Compound 32. As used herein, the term “substantially free of amorphous Compound 32” means that the compound contains no significant amount of amorphous Compound 32. In certain embodiments, at least about 95% by weight of crystalline Compound 32 is present. In still other embodiments of the invention, at least about 99% by weight of crystalline Compound 32 is present.

It has been found that Compound 32 can exist in at least four distinct polymorphic forms. In some embodiments, the present invention provides a polymorphic form of Compound 32 referred to herein as Type A. In some embodiments, the present invention provides a polymorphic form of Compound 32 referred to herein as Type B. In some embodiments, the present invention provides a polymorphic form of Compound 32 referred to herein as Type C. In some embodiments, the present invention provides a polymorphic form of Compound 32 referred to herein as Type D.

In some embodiments, Compound 32 is amorphous. In some embodiments, Compound 32 is amorphous, and is substantially free of crystalline Compound 32.

### Compound 32 Type A

In some embodiments, Compound 32 Type A has at least 1, 2, 3, 4 or 5 spectral peak(s) selected from the peaks listed in Table 30 below.

TABLE 30
XRPD Peak Positions for Compound 32 Type A
Position (°2θ)

4.51
6.52
7.32
8.32
9.07
9.42
9.74
10.74
10.98
11.32
11.54
12.37
13.26
14.02
14.57
14.87
15.26
15.59
15.73
16.09
16.58
16.84
17.15
17.41
17.79
18.21
18.92
19.57
20.17
20.58
21.01
21.26
21.53
21.77
22.33
22.83
23.08
23.30
23.74
23.99
24.93
25.23
26.28
26.86
27.77
28.19
29.04
29.38
29.97
30.57
30.77
31.06
31.77
32.22
33.09
33.62
33.95
35.23
36.34
36.64
37.25
37.97
38.41
39.51

In some embodiments, Compound 32 Type A is characterized in that it has one or more peaks in its X-ray powder diffraction pattern selected from those at about 7.32, 13.26, 15.59, 22.83, and 27.77. In some embodiments, Compound 32 Type A is characterized in that it has two or more peaks in its X-ray powder diffraction pattern selected from those at about 7.32, 13.26, 15.59, 22.83, and 27.77. In some embodiments, Compound 32 Type A is characterized in that it has three or more peaks in its X-ray powder diffraction pattern selected from those at about 7.32, 13.26, 15.59, 22.83, and 27.77. In some embodiments, Compound 32 Type A is characterized in that it has four or more peaks in its X-ray powder diffraction pattern selected from those at about 7.32, 13.26, 15.59, 22.83, and 27.77. In some embodiments, Compound 32 Type A is characterized in that it has all five peaks in its X-ray powder diffraction pattern selected from those at about 7.32, 13.26, 15.59, 22.83, and 27.77.

In certain embodiments, the X-ray powder diffraction pattern is substantially similar to the XRPD provided in FIG. 80.

Methods for preparing Compound 32 Type A are described infra.

### Compound 32 Type B

In some embodiments, Compound 32 Type B has at least 1, 2, 3, 4 or 5 spectral peak(s) selected from the peaks listed in Table 31 below.

TABLE 31
XRPD Peak Positions for Compound 32 Type B
Position (°2θ)

4.66
5.64
6.53
8.15
10.76
11.78
12.14
12.48
13.08
13.71
14.90
15.26
15.81
16.41
16.88
17.86
18.54
19.60
20.66
22.26
23.44
24.31
24.81
25.95
26.50
27.56

In some embodiments, Compound 32 Type B is characterized in that it has one or more peaks in its X-ray powder diffraction pattern selected from those at about 5.64, 18.54, and 24.31. In some embodiments, Compound 32 Type B is characterized in that it has two or more peaks in its X-ray powder diffraction pattern selected from those at about 5.64, 18.54, and 24.31. In some embodiments, Compound 32 Type B is characterized in that it has all three peaks in its X-ray powder diffraction pattern selected from those at about 5.64, 18.54, and 24.31.

In certain embodiments, the X-ray powder diffraction pattern is substantially similar to the XRPD provided in FIG. 80.

Methods for preparing Compound 32 Type B are described infra.

### Compound 32 Type C

In some embodiments, Compound 32 Type C has at least 1, 2, 3, 4 or 5 spectral peak(s) selected from the peaks listed in Table 32 below.

TABLE 32
XRPD Peak Positions for Compound 32 Type C
Position (°2θ)

4.31
6.46
8.59
12.88
13.81
14.74
15.15
16.13
16.66
18.63
19.66
19.98
21.35
23.69
25.06
25.71
28.42

In some embodiments, Compound 32 Type C is characterized in that it has one or more peaks in its X-ray powder diffraction pattern selected from those at about 8.59, 12.88, 19.98, and 25.71. In some embodiments, Compound 32 Type C is characterized in that it has two or more peaks in its X-ray powder diffraction pattern selected from those at about 8.59, 12.88, 19.98, and 25.71. In some embodiments, Compound 32 Type C is characterized in that it has three or more peaks in its X-ray powder diffraction pattern selected from those at about 8.59, 12.88, 19.98, and 25.71. In some embodiments, Compound 32 Type C is characterized in that it has all four peaks in its X-ray powder diffraction pattern selected from those at about 8.59, 12.88, 19.98, and 25.71.

In certain embodiments, the X-ray powder diffraction pattern is substantially similar to the XRPD provided in FIG. 80.

Methods for preparing Compound 32 Type C are described infra.

### Compound 32 Type D

In some embodiments, Compound 32 Type D has at least 1, 2, 3, 4 or 5 spectral peak(s) selected from the peaks listed in Table 33 below.

TABLE 33
XRPD Peak Positions for Compound 32 Type D
Position (°2θ)

4.66
9.28
10.75
11.53
13.94
15.68
16.88
17.24
17.53
17.85
18.60
19.19
20.89
21.47
21.65
23.30
24.47
24.62
24.85
25.78
27.76
29.02
32.80
33.68

In some embodiments, Compound 32 Type D is characterized in that it has one or more peaks in its X-ray powder diffraction pattern selected from those at about 9.28, 11.53, 13.94, and 19.19. In some embodiments, Compound 32 Type D is characterized in that it has two or more peaks in its X-ray powder diffraction pattern selected from those at about 9.28, 11.53, 13.94, and 19.19. In some embodiments, Compound 32 Type D is characterized in that it has three or more peaks in its X-ray powder diffraction pattern selected from those at about 9.28, 11.53, 13.94, and 19.19. In some embodiments, Compound 32 Type D is characterized in that it has all four peaks in its X-ray powder diffraction pattern selected from those at about 9.28, 11.53, 13.94, and 19.19.

In certain embodiments, the X-ray powder diffraction pattern is substantially similar to the XRPD provided in FIG. 80.

Methods for preparing Compound 32 Type D are described infra.

### 33. Compound 33 (L-Pyroglutamic Acid×Compound 1)

According to one embodiment, the present invention provides a chemical species Compound 33 comprising Compound 1 and L-pyroglutamic acid:

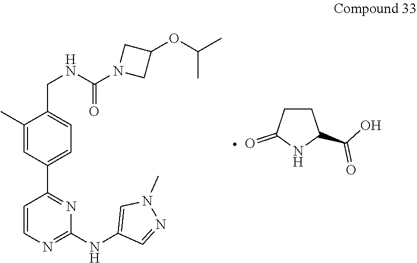

It is contemplated that Compound 33 can exist in a variety of physical forms. For example, Compound 33 can be in solution, suspension, or in solid form. In certain embodiments, Compound 33 is in solid form. When Compound 33 is in solid form, said compound may be amorphous, crystalline, or a mixture thereof. Exemplary solid forms are described in more detail below.

In one embodiment, the solid form of Compound 33 has a stoichiometry of (Compound 1):(L-pyroglutamic acid) that is about 1:1.

In some embodiments, the present invention provides Compound 33 substantially free of impurities. As used herein, the term “substantially free of impurities” means that the compound contains no significant amount of extraneous matter. Such extraneous matter may include excess L-pyroglutamic acid, excess Compound 1, residual solvents, or any other impurities that may result from the preparation of, and/or isolation of, Compound 33. In certain embodiments, at least about 95% by weight of Compound 33 is present. In still other embodiments of the invention, at least about 99% by weight of Compound 33 is present.

According to one embodiment, Compound 33 is present in an amount of at least about 97, 97.5, 98.0, 98.5, 99, 99.5, 99.8 weight percent where the percentages are based on the total weight of the composition. According to another embodiment, Compound 33 contains no more than about 3.0 area percent HPLC of total organic impurities and, in certain embodiments, no more than about 1.5 area percent HPLC total organic impurities relative to the total area of the HPLC chromatogram. In other embodiments, Compound 33 contains no more than about 1.0% area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for Compound 33 is also meant to include all tautomeric forms of Compound 33. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

It has been found that Compound 33 can exist in a variety of solid forms. Exemplary such forms include polymorphs such as those described herein.

In certain embodiments, Compound 33 is a crystalline solid. In other embodiments, Compound 33 is a crystalline solid substantially free of amorphous Compound 33. As used herein, the term “substantially free of amorphous Compound 33” means that the compound contains no significant amount of amorphous Compound 33. In certain embodiments, at least about 95% by weight of crystalline Compound 33 is present. In still other embodiments of the invention, at least about 99% by weight of crystalline Compound 33 is present.

In some embodiments, Compound 33 is amorphous. In some embodiments, Compound 33 is amorphous, and is substantially free of crystalline Compound 33.

Exemplary Compounds

The present invention includes the following 33 lists of embodiments (wherein each list is self-contained and any references to embodiment numbers refers to embodiments within the same list):

### Compound 1

1. Compound 1:

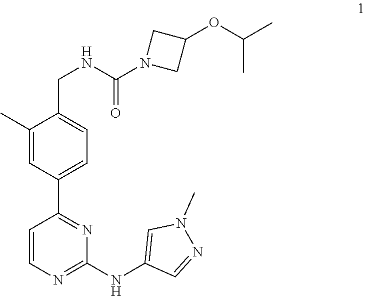 [SMILES: Cc1cc(-c2ccnc(Nc3cnn(C)c3)n2)ccc1CNC(=O)N1CC(OC(C)C)C1]

wherein said compound is crystalline.

2. The compound according to embodiment 1, wherein said compound is substantially free of amorphous Compound 1.

3. The compound according to embodiment 1, wherein said compound is substantially free of impurities.

4. The compound according to embodiment 1, wherein said compound is of Type A.

5. The compound according to embodiment 4, having a XRPD substantially similar to that depicted in FIG. 1.

6. The compound according to embodiment 4, wherein said compound is an anhydrate.

7. The compound according to embodiment 1, wherein said compound is of Type B.

8. The compound according to embodiment 7, having an XRPD substantially similar to that depicted in FIG. 3.

9. The compound according to embodiment 7, wherein said compound is a hydrate. 10. The compound according to embodiment 1, wherein said compound is of Type C.

11. The compound according to embodiment 10, having an XRPD substantially similar to that depicted in FIG. 5.

12. The compound according to embodiment 10, wherein said compound is an anhydrate.

13. The compound according to embodiment 1, wherein said compound is of Type D.

14. The compound according to embodiment 13, having an XRPD substantially similar to that depicted in FIG. 8.

15. The compound according to embodiment 13, wherein said compound is an anhydrate.

16. The compound according to embodiment 1, wherein said compound is of Type E.

17. The compound according to embodiment 16, having an XRPD substantially similar to that depicted in FIG. 11.

18. The compound according to embodiment 1, wherein said compound is of Type F.

19. The compound according to embodiment 18, having an XRPD substantially similar to that depicted in FIG. 13.

20. The compound according to embodiment 18, wherein said compound is an anhydrate.

21. The compound according to embodiment 1, wherein said compound is of Type G.

22. The compound according to embodiment 21, having an XRPD substantially similar to that depicted in FIG. 15.

23. The compound according to embodiment 21, wherein said compound is an anhydrate.

24. Compound 1:

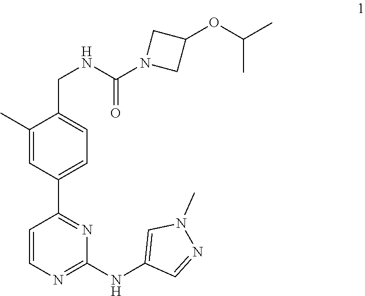 [SMILES: Cc1cc(-c2ccnc(Nc3cnn(C)c3)n2)ccc1CNC(=O)N1CC(OC(C)C)C1]

wherein said compound is amorphous.

25. The compound according to embodiment 24, wherein said compound is substantially free of crystalline Compound 1.

26. The compound according to embodiment 24, wherein said compound is substantially free of impurities.

27. A composition comprising the compound according to any one of embodiments 1-26 and a pharmaceutically acceptable carrier or excipient.

28. A method of decreasing the enzymatic activity of Bruton's tyrosine kinase comprising contacting Bruton's tyrosine kinase with an effective amount of a compound of any one of embodiments 1-26 or a composition thereof.

29. A method of treating a disorder responsive to inhibition of Bruton's tyrosine kinase comprising administering to a subject an effective amount of a compound of any one of embodiments 1-26 or a composition thereof.

30. A method of treating a disorder selected from the group consisting of autoimmune disorders, inflammatory disorders, and cancers comprising administering to a subject an effective amount of a compound of any one of embodiments 1-26 of a composition thereof.

31. The method according to embodiment 30, wherein the disorder is selected from rheumatoid arthritis, systemic lupus erythematosus, atopic dermatitis, leukemia and lymphoma.

### Compound 2

1. Compound 2, comprising Compound 1 and hydrochloric acid:

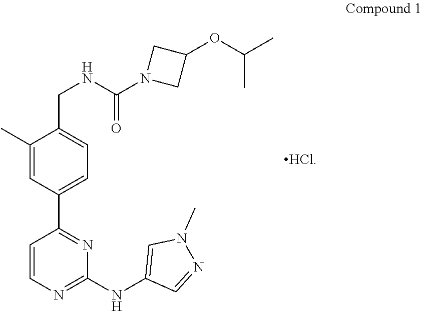 [SMILES: Cc1cc(-c2ccnc(Nc3cnn(C)c3)n2)ccc1CNC(=O)N1CC(OC(C)C)C1]

2. The compound according to embodiment 1, wherein the solid form of the compound has an acid:base ratio of about 1:1.

3. The compound according to embodiment 1, wherein said compound is crystalline.

4. The compound according to embodiment 3, wherein said compound is a crystalline solid substantially free of amorphous Compound 2.

5. The compound according to embodiment 1, wherein said compound is substantially free of impurities.

6. The compound according to embodiment 1, wherein said compound is of Type A.

7. The compound according to embodiment 6, having one or more peaks in its XRPD selected from those at about 5.23, about 9.11, about 12.39, about 14.40, about 14.73, about 25.58, and about 26.57 degrees 2-theta.

8. The compound according to embodiment 7, having at least two peaks in its XRPD selected from those at about 5.23, about 9.11, about 12.39, about 14.40, about 14.73, about 25.58, and about 26.57 degrees 2-theta.

9. The compound according to embodiment 6, having a XRPD substantially similar to that depicted in FIG. 21.

10. The compound according to embodiment 6, wherein said compound is a monohydrate.

11. The compound according to embodiment 1, wherein said compound is of Type B.

12. The compound according to embodiment 11, having an XRPD substantially similar to that depicted in FIG. 27.

13. The compound according to embodiment 1, wherein said compound is of Type C.

14. The compound according to embodiment 13, having an XRPD substantially similar to that depicted in FIG. 28.

15. The compound of embodiment 1, wherein said compound is amorphous.

16. The compound according to embodiment 15, wherein said compound is substantially free of crystalline Compound 2.

17. The compound according to embodiment 15, wherein said compound is substantially free of impurities.

18. A composition comprising the compound according to any one of embodiments 1-17 and a pharmaceutically acceptable carrier or excipient.

19. A method of decreasing the enzymatic activity of Bruton's tyrosine kinase comprising contacting Bruton's tyrosine kinase with an effective amount of a compound of any one of embodiments 1-17 or a composition thereof.

20. A method of treating a disorder responsive to inhibition of Bruton's tyrosine kinase comprising administering to a subject an effective amount of a compound of any one of embodiments 1-17 or a composition thereof.

21. A method of treating a disorder selected from the group consisting of autoimmune disorders, inflammatory disorders, and cancers comprising administering to a subject an effective amount of a compound of any one of embodiments 1-17 of a composition thereof.

22. The method according to embodiment 21, wherein the disorder is selected from rheumatoid arthritis, systemic lupus erythematosus, atopic dermatitis, leukemia and lymphoma.

### Compound 3

1. Compound 3, comprising Compound 1 and sulfuric acid,

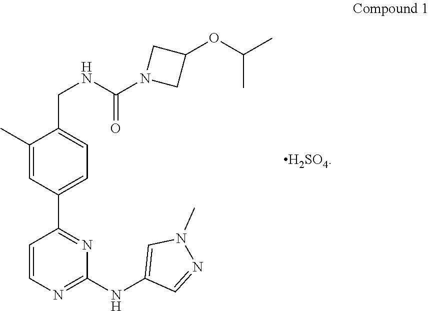 [SMILES: Cc1cc(-c2ccnc(Nc3cnn(C)c3)n2)ccc1CNC(=O)N1CC(OC(C)C)C1]

2. The compound according to embodiment 1, wherein the solid form of the compound has an acid:base ratio of about 1:1.

3. The compound according to embodiment 1, wherein said compound is crystalline.

4. The compound according to embodiment 3, wherein said compound is a crystalline solid substantially free of amorphous Compound 3.

5. The compound according to embodiment 1, wherein said compound is substantially free of impurities.

6. The compound according to embodiment 1, wherein said compound is of Type A.

7. The compound according to embodiment 6, having one or more peaks in its XRPD selected from those at about 10.49, about 15.99, about 16.88, about 17.86, and about 21.96 degrees 2-theta.

8. The compound according to embodiment 7, having at least two peaks in its XRPD selected from those at about 10.49, about 15.99, about 16.88, about 17.86, and about 21.96 degrees 2-theta.

9. The compound according to embodiment 6, having a XRPD substantially similar to that depicted in FIG. 30.

10. The compound according to embodiment 1, wherein said compound is of Type B.

11. The compound according to embodiment 10, having one or more peaks in its XRPD selected from those at about 6.11, about 8.97, about 11.49, about 16.50, about 21.54, and about 26.54 degrees 2-theta.

12. The compound according to embodiment 11, having at least two peaks in its XRPD selected from those at about 6.11, about 8.97, about 11.49, about 16.50, about 21.54, and about 26.54 degrees 2-theta.

13. The compound according to embodiment 10, having an XRPD substantially similar to that depicted in FIG. 30.

14. The compound according to embodiment 1, wherein said compound is of Type C.

15. The compound according to embodiment 14, having one or more peaks in its XRPD selected from those at about 3.58, about 10.94, about 14.81, and about 24.44 degrees 2-theta.

16. The compound according to embodiment 15, having at least two peaks in its XRPD selected from those at about 3.58, about 10.94, about 14.81, and about 24.44 degrees 2-theta.

17. The compound according to embodiment 14, having an XRPD substantially similar to that depicted in FIG. 30.

18. The compound of embodiment 1, wherein said compound is amorphous.

19. The compound according to embodiment 18, wherein said compound is substantially free of crystalline Compound 3.

20. The compound according to embodiment 18, wherein said compound is substantially free of impurities.

21. A composition comprising the compound according to any one of embodiments 1-20 and a pharmaceutically acceptable carrier or excipient.

22. A method of decreasing the enzymatic activity of Bruton's tyrosine kinase comprising contacting Bruton's tyrosine kinase with an effective amount of a compound of any one of embodiments 1-20 or a composition thereof.

23. A method of treating a disorder responsive to inhibition of Bruton's tyrosine kinase comprising administering to a subject an effective amount of a compound of any one of embodiments 1-20 or a composition thereof.

24. A method of treating a disorder selected from the group consisting of autoimmune disorders, inflammatory disorders, and cancers comprising administering to a subject an effective amount of a compound of any one of embodiments 1-20 of a composition thereof.

25. The method according to embodiment 24, wherein the disorder is selected from rheumatoid arthritis, systemic lupus erythematosus, atopic dermatitis, leukemia and lymphoma.

### Compound 4

1. Compound 4, comprising Compound 1 and methansulfonic acid:

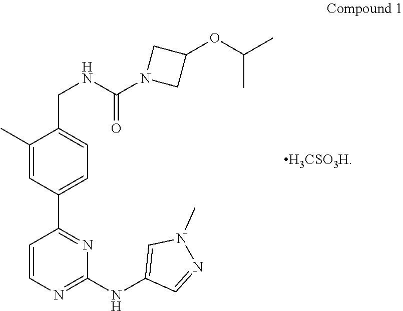 [SMILES: Cc1cc(-c2ccnc(Nc3cnn(C)c3)n2)ccc1CNC(=O)N1CC(OC(C)C)C1]

2. The compound according to embodiment 1, wherein said compound is crystalline.

3. The compound according to embodiment 2, wherein said compound is a crystalline solid substantially free of amorphous Compound 4.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 5.80, about 7.51, about 10.54, about 11.77, about 20.02, about 21.66, and about 25.56 degrees 2-theta.

7. The compound according to embodiment 6, having at least two peaks in its XRPD selected from those at about 5.80, about 7.51, about 10.54, about 11.77, about 20.02, about 21.66, and about 25.56 degrees 2-theta.

8. The solid form according to embodiment 5, having a XRPD substantially similar to that depicted in FIG. 36.

9. The compound of embodiment 1, wherein said compound is amorphous.

10. The compound according to embodiment 9, wherein said compound is substantially free of crystalline Compound 4.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 5

1. Compound 5, comprising Compound 1 and ethanedisulfonic acid:

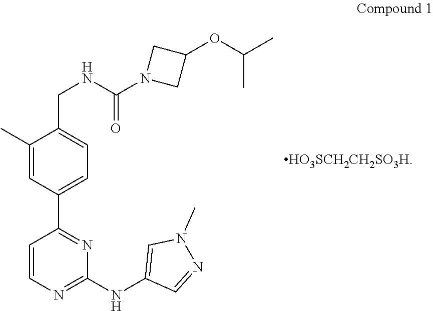 [SMILES: Cc1cc(-c2ccnc(Nc3cnn(C)c3)n2)ccc1CNC(=O)N1CC(OC(C)C)C1]

2. The compound according to embodiment 1, wherein said compound is crystalline.

3. The compound according to embodiment 2, wherein said compound is a crystalline solid substantially free of amorphous Compound 5.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 5.47, about 8.55, about 17.69, about 18.10, about 23.43, and about 25.86 degrees 2-theta.

7. The compound according to embodiment 6, having at least two peaks in its XRPD selected from those at about 5.47, about 8.55, about 17.69, about 18.10, about 23.43, and about 25.86 degrees 2-theta.

8. The solid form according to embodiment 5, having a XRPD substantially similar to that depicted in FIG. 38.

9. The compound of embodiment 1, wherein said compound is amorphous.

10. The compound according to embodiment 9, wherein said compound is substantially free of crystalline Compound 5.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 6

1. Compound 6, comprising Compound 1 and 2-hydroxyethanesulfonic acid:

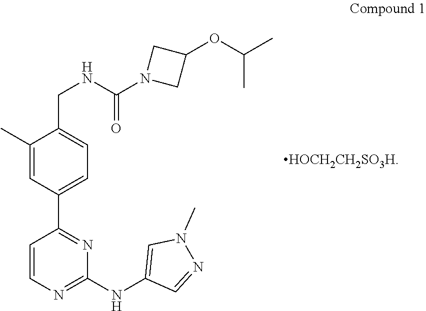 [SMILES: Cc1cc(-c2ccnc(Nc3cnn(C)c3)n2)ccc1CNC(=O)N1CC(OC(C)C)C1]

2. The compound according to embodiment 1, wherein said compound is crystalline.

3. The compound according to embodiment 2, wherein said compound is a crystalline solid substantially free of amorphous Compound 6.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 3.43, about 8.05, about 9.32, about 17.15, about 17.82, and about 24.66 degrees 2-theta.

7. The compound according to embodiment 6, having at least two peaks in its XRPD selected from those at about 3.43, about 8.05, about 9.32, about 17.15, about 17.82, and about 24.66 degrees 2-theta.

8. The solid form according to embodiment 5, having a XRPD substantially similar to that depicted in FIG. 40.

9. The compound of embodiment 1, wherein said compound is amorphous.

10. The compound according to embodiment 9, wherein said compound is substantially free of crystalline Compound 6.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 7

1. Compound 7, comprising Compound 1 and benzenesulfonic acid:

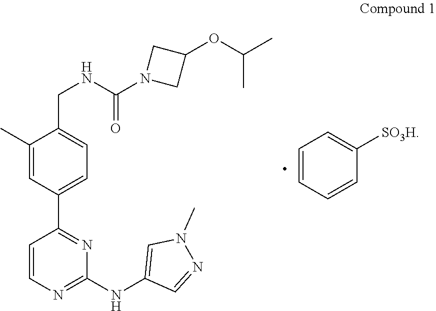 [SMILES: Cc1cc(-c2ccnc(Nc3cnn(C)c3)n2)ccc1CNC(=O)N1CC(OC(C)C)C1 ; O=S(=O)(O)c1ccccc1]

2. The compound according to embodiment 1, wherein said compound is crystalline.

3. The compound according to embodiment 2, wherein said compound is a crystalline solid substantially free of amorphous Compound 7.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 4.56, about 5.29, about 10.28, about 18.29, about 21.23, and about 23.35 degrees 2-theta.

7. The compound according to embodiment 6, having at least two peaks in its XRPD selected from those at about 4.56, about 5.29, about 10.28, about 18.29, about 21.23, and about 23.35 degrees 2-theta.

8. The solid form according to embodiment 5, having a XRPD substantially similar to that depicted in FIG. 42.

9. The compound according to embodiment 1, wherein said compound is of Type B.

10. The compound according to embodiment 9, having one or more peaks in its XRPD selected from those at about 4.30 and about 8.66 degrees 2-theta.

11. The compound according to embodiment 10, having at both peaks in its XRPD selected from those at about 4.30 and about 8.66 degrees 2-theta.

12. The solid form according to embodiment 9, having a XRPD substantially similar to that depicted in FIG. 42.

13. The compound of embodiment 1, wherein said compound is amorphous.

14. The compound according to embodiment 13, wherein said compound is substantially free of crystalline Compound 7.

15. The compound according to embodiment 13, wherein said compound is substantially free of impurities.

16. A composition comprising the compound according to any one of embodiments 1-15 and a pharmaceutically acceptable carrier or excipient.

17. A method of decreasing the enzymatic activity of Bruton's tyrosine kinase comprising contacting Bruton's tyrosine kinase with an effective amount of a compound of any one of embodiments 1-15 or a composition thereof.

18. A method of treating a disorder responsive to inhibition of Bruton's tyrosine kinase comprising administering to a subject an effective amount of a compound of any one of embodiments 1-15 or a composition thereof.

19. A method of treating a disorder selected from the group consisting of autoimmune disorders, inflammatory disorders, and cancers comprising administering to a subject an effective amount of a compound of any one of embodiments 1-15 of a composition thereof.

20. The method according to embodiment 19, wherein the disorder is selected from rheumatoid arthritis, systemic lupus erythematosus, atopic dermatitis, leukemia and lymphoma.

### Compound 8

1. Compound 8, comprising Compound 1 and toluenesulfonic acid:

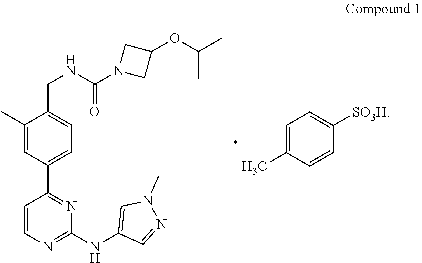 [SMILES: Cc1cc(-c2ccnc(Nc3cnn(C)c3)n2)ccc1CNC(=O)N1CC(OC(C)C)C1 ; Cc1ccc(S(=O)(=O)O)cc1]

2. The compound according to embodiment 1, wherein said compound is crystalline.

3. The compound according to embodiment 2, wherein said compound is a crystalline solid substantially free of amorphous Compound 8.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 11.21, about 17.06, about 20.10, and about 30.45 degrees 2-theta.

7. The compound according to embodiment 6, having at least two peaks in its XRPD selected from those at about 11.21, about 17.06, about 20.10, and about 30.45 degrees 2-theta.

8. The solid form according to embodiment 5, having a XRPD substantially similar to that depicted in FIG. 45.

9. The compound according to embodiment 1, wherein said compound is of Type B.

10. The compound according to embodiment 9, having one or more peaks in its XRPD selected from those at about 11.43, about 14.13, about 22.25, about 24.35, and about 27.94 degrees 2-theta.

11. The compound according to embodiment 10, having at least two peaks in its XRPD selected from those at about 11.43, about 14.13, about 22.25, about 24.35, and about 27.94 degrees 2-theta.

12. The solid form according to embodiment 9, having a XRPD substantially similar to that depicted in FIG. 45.

13. The compound according to embodiment 1, wherein said compound is of Type C.

14. The compound according to embodiment 13, having one or more peaks in its XRPD selected from those at about 3.93, about 7.92, about 8.92, about 13.58, about 15.75, about 17.90, and about 25.70 degrees 2-theta.

15. The compound according to embodiment 14, having at least two peaks in its XRPD selected from those at about 3.93, about 7.92, about 8.92, about 13.58, about 15.75, about 17.90, and about 25.70 degrees 2-theta.

16. The solid form according to embodiment 13, having a XRPD substantially similar to that depicted in FIG. 45.

17. The compound of embodiment 1, wherein said compound is amorphous.

18. The compound according to embodiment 17, wherein said compound is substantially free of crystalline Compound 8.

19. The compound according to embodiment 17, wherein said compound is substantially free of impurities.

20. A composition comprising the compound according to any one of embodiments 1-19 and a pharmaceutically acceptable carrier or excipient.

21. A method of decreasing the enzymatic activity of Bruton's tyrosine kinase comprising contacting Bruton's tyrosine kinase with an effective amount of a compound of any one of embodiments 1-19 or a composition thereof.

22. A method of treating a disorder responsive to inhibition of Bruton's tyrosine kinase comprising administering to a subject an effective amount of a compound of any one of embodiments 1-19 or a composition thereof.

23. A method of treating a disorder selected from the group consisting of autoimmune disorders, inflammatory disorders, and cancers comprising administering to a subject an effective amount of a compound of any one of embodiments 1-19 of a composition thereof.

24. The method according to embodiment 23, wherein the disorder is selected from rheumatoid arthritis, systemic lupus erythematosus, atopic dermatitis, leukemia and lymphoma.

### Compound 9

1. Compound 9, comprising Compound 1 and 2-naphthalenesulfonic acid:

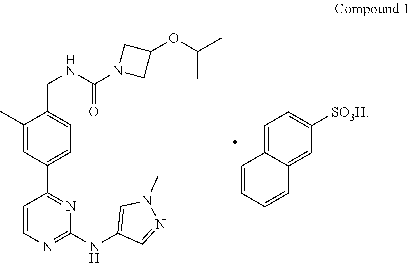 [SMILES: Cc1cc(-c2ccnc(Nc3cnn(C)c3)n2)ccc1CNC(=O)N1CC(OC(C)C)C1 ; O=S(=O)(O)c1ccc2ccccc2c1]

2. The compound according to embodiment 1, wherein said compound is crystalline.

3. The compound according to embodiment 2, wherein said compound is a crystalline solid substantially free of amorphous Compound 9.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 3.89, about 7.22, about 8.83, about 15.57, about 17.93, about 25.71, and about 26.69 degrees 2-theta.

7. The compound according to embodiment 6, having at least two peaks in its XRPD selected from those at about 3.89, about 7.22, about 8.83, about 15.57, about 17.93, about 25.71, and about 26.69 degrees 2-theta.

8. The solid form according to embodiment 5, having a XRPD substantially similar to that depicted in FIG. 49.

9. The compound according to embodiment 1, wherein said compound is of Type B.

10. The compound according to embodiment 9, having one or more peaks in its XRPD selected from those at about 4.79, about 6.76, about 10.30, about 11.08, about 19.24, about 19.95, and about 26.22 degrees 2-theta.

11. The compound according to embodiment 10, having at least two peaks in its XRPD selected from those at about 4.79, about 6.76, about 10.30, about 11.08, about 19.24, about 19.95, and about 26.22 degrees 2-theta.

12. The solid form according to embodiment 9, having a XRPD substantially similar to that depicted in FIG. 49.

13. The compound of embodiment 1, wherein said compound is amorphous.

14. The compound according to embodiment 13, wherein said compound is substantially free of crystalline Compound 9.

15. The compound according to embodiment 13, wherein said compound is substantially free of impurities.

16. A composition comprising the compound according to any one of embodiments 1-15 and a pharmaceutically acceptable carrier or excipient.

17. A method of decreasing the enzymatic activity of Bruton's tyrosine kinase comprising contacting Bruton's tyrosine kinase with an effective amount of a compound of any one of embodiments 1-15 or a composition thereof.

18. A method of treating a disorder responsive to inhibition of Bruton's tyrosine kinase comprising administering to a subject an effective amount of a compound of any one of embodiments 1-15 or a composition thereof.

19. A method of treating a disorder selected from the group consisting of autoimmune disorders, inflammatory disorders, and cancers comprising administering to a subject an effective amount of a compound of any one of embodiments 1-15 of a composition thereof.

20. The method according to embodiment 19, wherein the disorder is selected from rheumatoid arthritis, systemic lupus erythematosus, atopic dermatitis, leukemia and lymphoma.

### Compound 10

1. Compound 10, comprising Compound 1 and nitric acid:

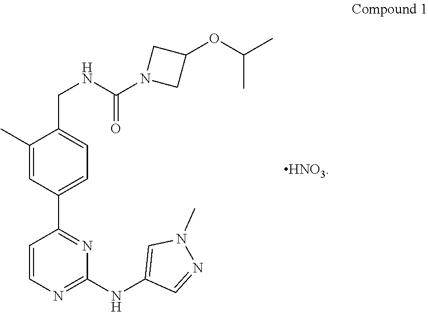 [SMILES: Cc1cc(-c2ccnc(Nc3cnn(C)c3)n2)ccc1CNC(=O)N1CC(OC(C)C)C1]

2. The compound according to embodiment 1, wherein said compound is crystalline.

3. The compound according to embodiment 2, wherein said compound is a crystalline solid substantially free of amorphous Compound 10.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 7.52, about 11.03, about 18.68, about 20.70, about 22.20, and about 29.17 degrees 2-theta.

7. The compound according to embodiment 6, having at least two peaks in its XRPD selected from those at about 7.52, about 11.03, about 18.68, about 20.70, about 22.20, and about 29.17 degrees 2-theta.

8. The solid form according to embodiment 5, having a XRPD substantially similar to that depicted in FIG. 52.

9. The compound according to embodiment 1, wherein said compound is of Type B.

10. The compound according to embodiment 9, having one or more peaks in its XRPD selected from those at about 7.00, about 9.34, about 13.97, and about 20.15 degrees 2-theta.

11. The compound according to embodiment 10, having at least two peaks in its XRPD selected from those at about 7.00, about 9.34, about 13.97, and about 20.15 degrees 2-theta.

12. The solid form according to embodiment 9, having a XRPD substantially similar to that depicted in FIG. 52.

13. The compound according to embodiment 1, wherein said compound is of Type C.

14. The compound according to embodiment 13, having one or more peaks in its XRPD selected from those at about 5.29, about 8.87, about 12.13, about 14.86, about 17.77, and about 26.05 degrees 2-theta.

15. The compound according to embodiment 14, having at least two peaks in its XRPD selected from those at about 5.29, about 8.87, about 12.13, about 14.86, about 17.77, and about 26.05 degrees 2-theta.

16. The solid form according to embodiment 13, having a XRPD substantially similar to that depicted in FIG. 52.

17. The compound of embodiment 1, wherein said compound is amorphous.

18. The compound according to embodiment 17, wherein said compound is substantially free of crystalline Compound 10.

19. The compound according to embodiment 17, wherein said compound is substantially free of impurities.

20. A composition comprising the compound according to any one of embodiments 1-19 and a pharmaceutically acceptable carrier or excipient.

21. A method of decreasing the enzymatic activity of Bruton's tyrosine kinase comprising contacting Bruton's tyrosine kinase with an effective amount of a compound of any one of embodiments 1-19 or a composition thereof.

22. A method of treating a disorder responsive to inhibition of Bruton's tyrosine kinase comprising administering to a subject an effective amount of a compound of any one of embodiments 1-19 or a composition thereof.

23. A method of treating a disorder selected from the group consisting of autoimmune disorders, inflammatory disorders, and cancers comprising administering to a subject an effective amount of a compound of any one of embodiments 1-19 of a composition thereof.

24. The method according to embodiment 23, wherein the disorder is selected from rheumatoid arthritis, systemic lupus erythematosus, atopic dermatitis, leukemia and lymphoma.

### Compound 11

1. Compound 11, comprising Compound 1 and oxalic acid:

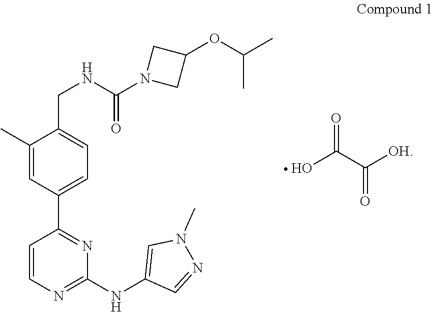 [SMILES: Cc1cc(-c2ccnc(Nc3cnn(C)c3)n2)ccc1CNC(=O)N1CC(OC(C)C)C1 ; O=C(O)C(=O)O]

2. The compound according to embodiment 1, wherein said compound is crystalline.

3. The compound according to embodiment 2, wherein said compound is a crystalline solid substantially free of amorphous Compound 11.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 3.98, about 7.27, about 12.16, about 18.38, and about 20.65 degrees 2-theta.

7. The compound according to embodiment 6, having at least two peaks in its XRPD selected from those at about 3.98, about 7.27, about 12.16, about 18.38, and about 20.65 degrees 2-theta.

8. The solid form according to embodiment 5, having a XRPD substantially similar to that depicted in FIG. 56.

9. The compound according to embodiment 1, wherein said compound is of Type B.

10. The compound according to embodiment 9, having one or more peaks in its XRPD selected from those at about 5.77, about 6.86, about 13.37, about 15.20, about 17.61, and about 30.90 degrees 2-theta.

11. The compound according to embodiment 10, having at least two peaks in its XRPD selected from those at about 5.77, about 6.86, about 13.37, about 15.20, about 17.61, and about 30.90 degrees 2-theta.

12. The solid form according to embodiment 9, having a XRPD substantially similar to that depicted in FIG. 56.

13. The compound according to embodiment 1, wherein said compound is of Type C.

14. The compound according to embodiment 13, having one or more peaks in its XRPD selected from those at about 3.15, about 10.56, about 11.62, about 13.62, about 20.04, about 27.68, and about 32.56 degrees 2-theta.

15. The compound according to embodiment 14, having at least two peaks in its XRPD selected from those at about 3.15, about 10.56, about 11.62, about 13.62, about 20.04, about 27.68, and about 32.56 degrees 2-theta.

16. The solid form according to embodiment 13, having a XRPD substantially similar to that depicted in FIG. 56.

17. The compound of embodiment 1, wherein said compound is amorphous.

18. The compound according to embodiment 17, wherein said compound is substantially free of crystalline Compound 11.

19. The compound according to embodiment 17, wherein said compound is substantially free of impurities.

20. A composition comprising the compound according to any one of embodiments 1-19 and a pharmaceutically acceptable carrier or excipient.

21. A method of decreasing the enzymatic activity of Bruton's tyrosine kinase comprising contacting Bruton's tyrosine kinase with an effective amount of a compound of any one of embodiments 1-19 or a composition thereof.

22. A method of treating a disorder responsive to inhibition of Bruton's tyrosine kinase comprising administering to a subject an effective amount of a compound of any one of embodiments 1-19 or a composition thereof.

23. A method of treating a disorder selected from the group consisting of autoimmune disorders, inflammatory disorders, and cancers comprising administering to a subject an effective amount of a compound of any one of embodiments 1-19 of a composition thereof.

24. The method according to embodiment 23, wherein the disorder is selected from rheumatoid arthritis, systemic lupus erythematosus, atopic dermatitis, leukemia and lymphoma.

### Compound 12

1. Compound 12, comprising Compound 1 and fumaric acid:

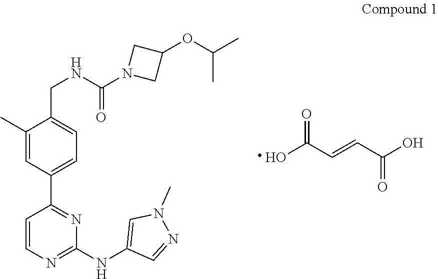 [SMILES: Cc1cc(-c2ccnc(Nc3cnn(C)c3)n2)ccc1CNC(=O)N1CC(OC(C)C)C1 ; O=C(O)/C=C/C(=O)O]

2. The compound according to embodiment 1, wherein said compound is crystalline.

3. The compound according to embodiment 2, wherein said compound is a crystalline solid substantially free of amorphous Compound 12.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 5.68, about 7.12, about 8.65, about 16.37, about 20.47, and about 22.86 degrees 2-theta.

7. The compound according to embodiment 6, having at least two peaks in its XRPD selected from those at about 5.68, about 7.12, about 8.65, about 16.37, about 20.47, and about 22.86 degrees 2-theta.

8. The solid form according to embodiment 5, having a XRPD substantially similar to that depicted in FIG. 60.

9. The compound of embodiment 1, wherein said compound is amorphous.

10. The compound according to embodiment 9, wherein said compound is substantially free of crystalline Compound 12.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 13

1. Compound 13, comprising Compound 1 and L-tartaric acid:

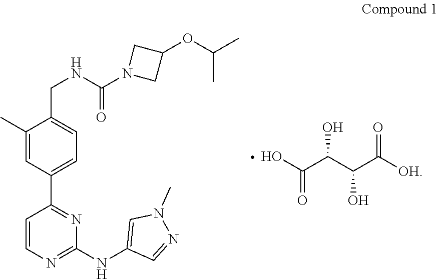 [SMILES: Cc1cc(-c2ccnc(Nc3cnn(C)c3)n2)ccc1CNC(=O)N1CC(OC(C)C)C1 ; O=C(O)[C@H](O)[C@@H](O)C(=O)O]

2. The compound according to embodiment 1, wherein said compound is crystalline.

3. The compound according to embodiment 2, wherein said compound is a crystalline solid substantially free of amorphous Compound 13.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 6.51, about 7.08, about 13.36, about 16.28, about 19.24, and about 25.88 degrees 2-theta.

7. The compound according to embodiment 6, having at least two peaks in its XRPD selected from those at about 6.51, about 7.08, about 13.36, about 16.28, about 19.24, and about 25.88 degrees 2-theta.

8. The solid form according to embodiment 5, having a XRPD substantially similar to that depicted in FIG. 62.

9. The compound of embodiment 1, wherein said compound is amorphous.

10. The compound according to embodiment 9, wherein said compound is substantially free of crystalline Compound 13.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 14

1. Compound 14, comprising Compound 1 and citric acid:

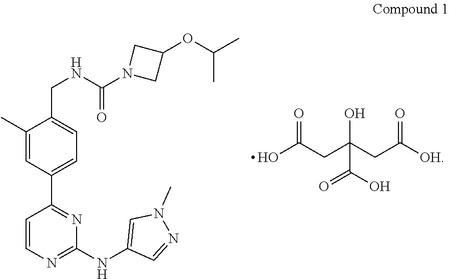 [SMILES: Cc1cc(-c2ccnc(Nc3cnn(C)c3)n2)ccc1CNC(=O)N1CC(OC(C)C)C1 ; O=C(O)CC(O)(CC(=O)O)C(=O)O]

2. The compound according to embodiment 1, wherein said compound is crystalline.

3. The compound according to embodiment 2, wherein said compound is a crystalline solid substantially free of amorphous Compound 14.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 8.05, about 11.58, about 16.87, about 24.58, and about 30.72 degrees 2-theta.

7. The compound according to embodiment 6, having at least two peaks in its XRPD selected from those at about 8.05, about 11.58, about 16.87, about 24.58, and about 30.72 degrees 2-theta.

8. The solid form according to embodiment 5, having a XRPD substantially similar to that depicted in FIG. 64.

9. The compound according to embodiment 1, wherein said compound is of Type B.

10. The compound according to embodiment 9, having one or more peaks in its XRPD selected from those at about 10.29, about 12.45, about 13.19, about 19.02, about 25.47, and about 25.92 degrees 2-theta.

11. The compound according to embodiment 10, having at least two peaks in its XRPD selected from those at about 10.29, about 12.45, about 13.19, about 19.02, about 25.47, and about 25.92 degrees 2-theta.

12. The solid form according to embodiment 9, having a XRPD substantially similar to that depicted in FIG. 64.

13. The compound of embodiment 1, wherein said compound is amorphous.

14. The compound according to embodiment 13, wherein said compound is substantially free of crystalline Compound 14.

15. The compound according to embodiment 13, wherein said compound is substantially free of impurities.

16. A composition comprising the compound according to any one of embodiments 1-15 and a pharmaceutically acceptable carrier or excipient.

17. A method of decreasing the enzymatic activity of Bruton's tyrosine kinase comprising contacting Bruton's tyrosine kinase with an effective amount of a compound of any one of embodiments 1-15 or a composition thereof.

18. A method of treating a disorder responsive to inhibition of Bruton's tyrosine kinase comprising administering to a subject an effective amount of a compound of any one of embodiments 1-15 or a composition thereof.

19. A method of treating a disorder selected from the group consisting of autoimmune disorders, inflammatory disorders, and cancers comprising administering to a subject an effective amount of a compound of any one of embodiments 1-15 of a composition thereof.

20. The method according to embodiment 19, wherein the disorder is selected from rheumatoid arthritis, systemic lupus erythematosus, atopic dermatitis, leukemia and lymphoma.

### Compound 15

1. Compound 15, comprising Compound 1 and L-malic acid:

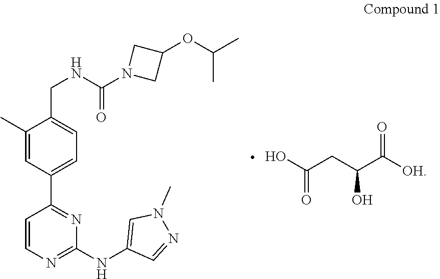 [SMILES: Cc1cc(-c2ccnc(Nc3cnn(C)c3)n2)ccc1CNC(=O)N1CC(OC(C)C)C1 ; O=C(O)C[C@H](O)C(=O)O]

2. The compound according to embodiment 1, wherein said compound is crystalline.

3. The compound according to embodiment 2, wherein said compound is a crystalline solid substantially free of amorphous Compound 15.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 5.58, about 7.02, about 8.55, about 16.28, about 19.63, and about 22.64 degrees 2-theta.

7. The compound according to embodiment 6, having at least two peaks in its XRPD selected from those at about 5.58, about 7.02, about 8.55, about 16.28, about 19.63, and about 22.64 degrees 2-theta.

8. The solid form according to embodiment 5, having a XRPD substantially similar to that depicted in FIG. 69.

9. The compound of embodiment 1, wherein said compound is amorphous.

10. The compound according to embodiment 9, wherein said compound is substantially free of crystalline Compound 15.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 16

1. Compound 16, comprising Compound 1 and succinic acid:

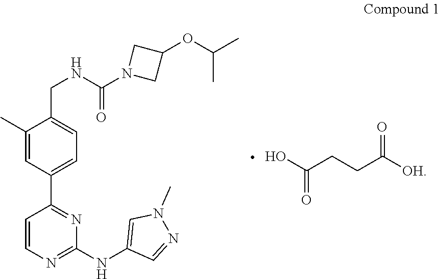 [SMILES: Cc1cc(-c2ccnc(Nc3cnn(C)c3)n2)ccc1CNC(=O)N1CC(OC(C)C)C1 ; O=C(O)CCC(=O)O]

2. The compound according to embodiment 1, wherein said compound is crystalline.

3. The compound according to embodiment 2, wherein said compound is a crystalline solid substantially free of amorphous Compound 16.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 5.62, about 7.04, about 8.55, about 22.80, and about 26.28 degrees 2-theta.

7. The compound according to embodiment 6, having at least two peaks in its XRPD selected from those at about 5.62, about 7.04, about 8.55, about 22.80, and about 26.28 degrees 2-theta.

8. The solid form according to embodiment 5, having a XRPD substantially similar to that depicted in FIG. 71.

9. The compound of embodiment 1, wherein said compound is amorphous.

10. The compound according to embodiment 9, wherein said compound is substantially free of crystalline Compound 16.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 17

1. Compound 17, comprising Compound 1 and hippuric acid:

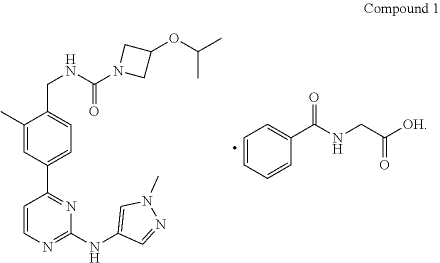 [SMILES: Cc1cc(-c2ccnc(Nc3cnn(C)c3)n2)ccc1CNC(=O)N1CC(OC(C)C)C1 ; O=C(O)CNC(=O)c1ccccc1]

2. The compound according to embodiment 1, wherein said compound is crystalline.

3. The compound according to embodiment 2, wherein said compound is a crystalline solid substantially free of amorphous Compound 17.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 17.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 18

1. Compound 18, comprising Compound 1 and maleic acid:

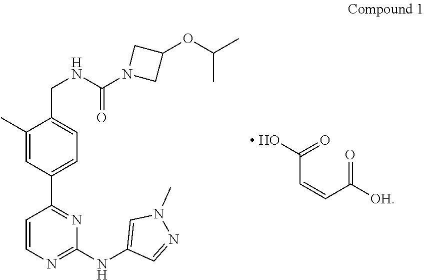 [SMILES: Cc1cc(-c2ccnc(Nc3cnn(C)c3)n2)ccc1CNC(=O)N1CC(OC(C)C)C1 ; O=C(O)/C=C\C(=O)O]

2. The compound according to embodiment 1, wherein said compound is crystalline.

3. The compound according to embodiment 2, wherein said compound is a crystalline solid substantially free of amorphous Compound 18.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 8.50, about 9.48, about 11.83, about 13.33, about 17.84, about 22.67, and about 26.49 degrees 2-theta.

7. The compound according to embodiment 6, having at least two peaks in its XRPD selected from those at about 8.50, about 9.48, about 11.83, about 13.33, about 17.84, about 22.67, and about 26.49 degrees 2-theta.

8. The solid form according to embodiment 5, having a XRPD substantially similar to that depicted in FIG. 75.

9. The compound according to embodiment 1, wherein said compound is of Type B.

10. The compound according to embodiment 9, having one or more peaks in its XRPD selected from those at about 3.48, about 6.20, about 6.36, about 7.62, about 10.32, and about 20.52 degrees 2-theta.

11. The compound according to embodiment 10, having at least two peaks in its XRPD selected from those at about 3.48, about 6.20, about 6.36, about 7.62, about 10.32, and about 20.52 degrees 2-theta.

12. The solid form according to embodiment 9, having a XRPD substantially similar to that depicted in FIG. 73.

13. The compound of embodiment 1, wherein said compound is amorphous.

14. The compound according to embodiment 13, wherein said compound is substantially free of crystalline Compound 18.

15. The compound according to embodiment 13, wherein said compound is substantially free of impurities.

16. A composition comprising the compound according to any one of embodiments 1-15 and a pharmaceutically acceptable carrier or excipient.

17. A method of decreasing the enzymatic activity of Bruton's tyrosine kinase comprising contacting Bruton's tyrosine kinase with an effective amount of a compound of any one of embodiments 1-15 or a composition thereof.

18. A method of treating a disorder responsive to inhibition of Bruton's tyrosine kinase comprising administering to a subject an effective amount of a compound of any one of embodiments 1-15 or a composition thereof.

19. A method of treating a disorder selected from the group consisting of autoimmune disorders, inflammatory disorders, and cancers comprising administering to a subject an effective amount of a compound of any one of embodiments 1-15 of a composition thereof.

20. The method according to embodiment 19, wherein the disorder is selected from rheumatoid arthritis, systemic lupus erythematosus, atopic dermatitis, leukemia and lymphoma.

### Compound 19

1. Compound 19, comprising Compound 1 and glutamic acid:

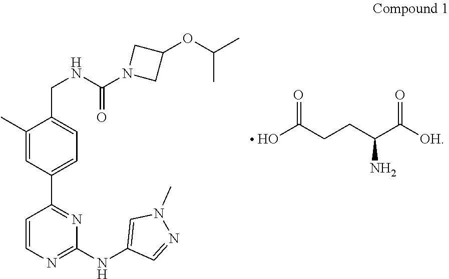 [SMILES: Cc1cc(-c2ccnc(Nc3cnn(C)c3)n2)ccc1CNC(=O)N1CC(OC(C)C)C1 ; N[C@@H](CCC(=O)O)C(=O)O]

2. The compound according to embodiment 1, wherein said compound is crystalline.

3. The compound according to embodiment 2, wherein said compound is a crystalline solid substantially free of amorphous Compound 19.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 19.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 20

1. Compound 20, comprising Compound 1 and benzoic acid:

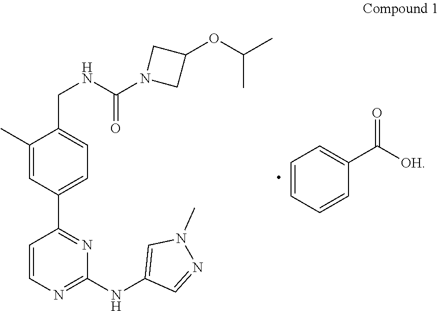 [SMILES: Cc1cc(-c2ccnc(Nc3cnn(C)c3)n2)ccc1CNC(=O)N1CC(OC(C)C)C1 ; O=C(O)c1ccccc1]

2. The compound according to embodiment 1, wherein said compound is crystalline.

3. The compound according to embodiment 2, wherein said compound is a crystalline solid substantially free of amorphous Compound 20.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 20.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 21

1. Compound 21, comprising Compound 1 and gentisic acid:

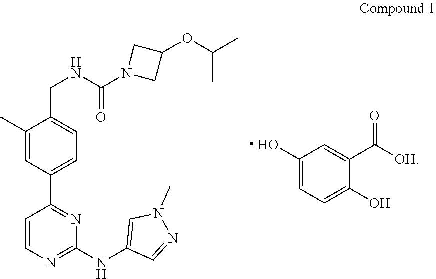 [SMILES: Cc1cc(-c2ccnc(Nc3cnn(C)c3)n2)ccc1CNC(=O)N1CC(OC(C)C)C1 ; O=C(O)c1cc(O)ccc1O]

2. The compound according to embodiment 1, wherein said compound is crystalline.

3. The compound according to embodiment 2, wherein said compound is a crystalline solid substantially free of amorphous Compound 21.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 7.57, about 8.08, about 15.44, about 16.18, about 19.30, and about 20.71 degrees 2-theta.

7. The compound according to embodiment 6, having at least two peaks in its XRPD selected from those at about 7.57, about 8.08, about 15.44, about 16.18, about 19.30, and about 20.71 degrees 2-theta.

8. The solid form according to embodiment 5, having a XRPD substantially similar to that depicted in FIG. 78.

9. The compound of embodiment 1, wherein said compound is amorphous.

10. The compound according to embodiment 9, wherein said compound is substantially free of crystalline Compound 21.

11. The compound according to embodiment 9, wherein said compound is substantially free of impurities.

12. A composition comprising the compound according to any one of embodiments 1-11 and a pharmaceutically acceptable carrier or excipient.

13. A method of decreasing the enzymatic activity of Bruton's tyrosine kinase comprising contacting Bruton's tyrosine kinase with an effective amount of a compound of any one of embodiments 1-11 or a composition thereof.

14. A method of treating a disorder responsive to inhibition of Bruton's tyrosine kinase comprising administering to a subject an effective amount of a compound of any one of embodiments 1-11 or a composition thereof.

15. A method of treating a disorder selected from the group consisting of autoimmune disorders, inflammatory disorders, and cancers comprising administering to a subject an effective amount of a compound of any one of embodiments 1-11 of a composition thereof.

16. The method according to embodiment 15, wherein the disorder is selected from rheumatoid arthritis, systemic lupus erythematosus, atopic dermatitis, leukemia and lymphoma.

### Compound 22

1. Compound 22, comprising Compound 1 and malonic acid:

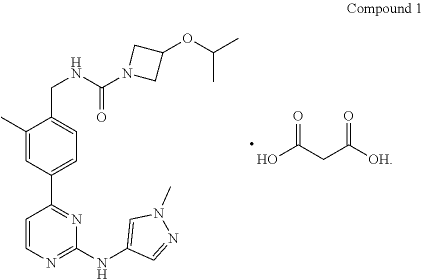 [SMILES: Cc1cc(-c2ccnc(Nc3cnn(C)c3)n2)ccc1CNC(=O)N1CC(OC(C)C)C1 ; O=C(O)CC(=O)O]

2. The compound according to embodiment 1, wherein said compound is crystalline.

3. The compound according to embodiment 2, wherein said compound is a crystalline solid substantially free of amorphous Compound 22.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 22.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 23

1. Compound 23, comprising Compound 1 and cinnamic acid:

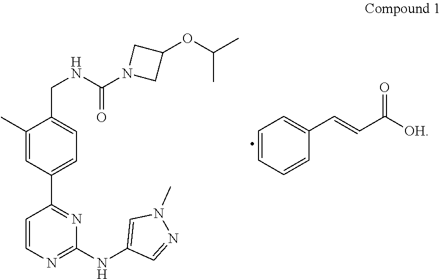 [SMILES: Cc1cc(-c2ccnc(Nc3cnn(C)c3)n2)ccc1CNC(=O)N1CC(OC(C)C)C1 ; O=C(O)/C=C/c1ccccc1]

2. The compound according to embodiment 1, wherein said compound is crystalline.

3. The compound according to embodiment 2, wherein said compound is a crystalline solid substantially free of amorphous Compound 23.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 23.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 24

1. Compound 24, comprising Compound 1 and L-glutamine:

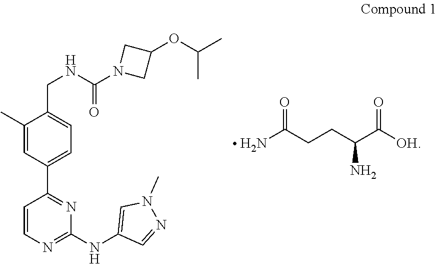 [SMILES: Cc1cc(-c2ccnc(Nc3cnn(C)c3)n2)ccc1CNC(=O)N1CC(OC(C)C)C1 ; NC(=O)CC[C@H](N)C(=O)O]

2. The compound according to embodiment 1, wherein said compound is crystalline.

3. The compound according to embodiment 2, wherein said compound is a crystalline solid substantially free of amorphous Compound 24.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 24.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 25

1. Compound 25, comprising Compound 1 and L-lysine:

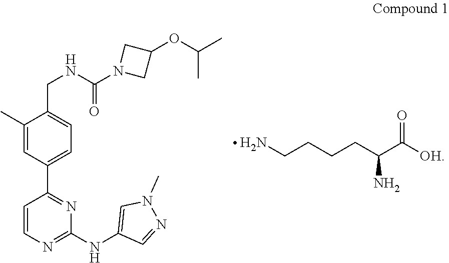 [SMILES: Cc1cc(-c2ccnc(Nc3cnn(C)c3)n2)ccc1CNC(=O)N1CC(OC(C)C)C1 ; NCCCC[C@H](N)C(=O)O]

2. The compound according to embodiment 1, wherein said compound is crystalline.

3. The compound according to embodiment 2, wherein said compound is a crystalline solid substantially free of amorphous Compound 25.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 25.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 26

1. Compound 26, comprising Compound 1 and L-phenylalanine:

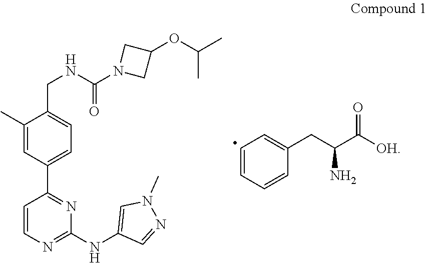 [SMILES: Cc1cc(-c2ccnc(Nc3cnn(C)c3)n2)ccc1CNC(=O)N1CC(OC(C)C)C1 ; N[C@@H](Cc1ccccc1)C(=O)O]

2. The compound according to embodiment 1, wherein said compound is crystalline.

3. The compound according to embodiment 2, wherein said compound is a crystalline solid substantially free of amorphous Compound 26.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 26.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 27

1. Compound 27, comprising Compound 1 and L-proline:

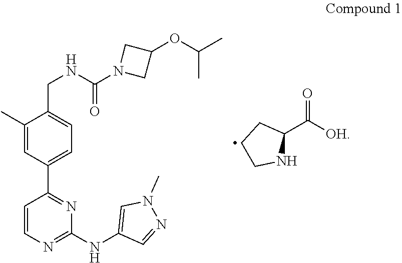 [SMILES: Cc1cc(-c2ccnc(Nc3cnn(C)c3)n2)ccc1CNC(=O)N1CC(OC(C)C)C1 ; O=C(O)[C@@H]1CCCN1]

2. The compound according to embodiment 1, wherein said compound is crystalline.

3. The compound according to embodiment 2, wherein said compound is a crystalline solid substantially free of amorphous Compound 27.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 27.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 28

1. Compound 28, comprising Compound 1 and L-serine:

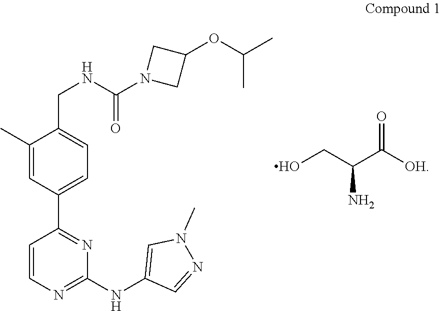

2. The compound according to embodiment 1, wherein said compound is crystalline.

3. The compound according to embodiment 2, wherein said compound is a crystalline solid substantially free of amorphous Compound 28.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 28.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 29

1. Compound 29, comprising Compound 1 and L-tyrosine:

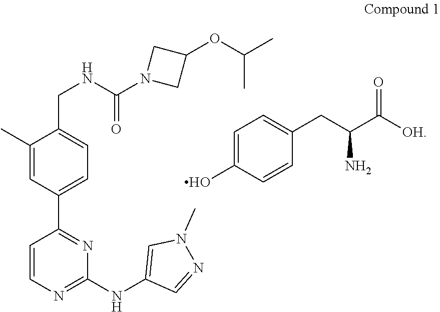

2. The compound according to embodiment 1, wherein said compound is crystalline.

3. The compound according to embodiment 2, wherein said compound is a crystalline solid substantially free of amorphous Compound 29.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 29.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 30

1. Compound 30, comprising Compound 1 and nicotinamide:

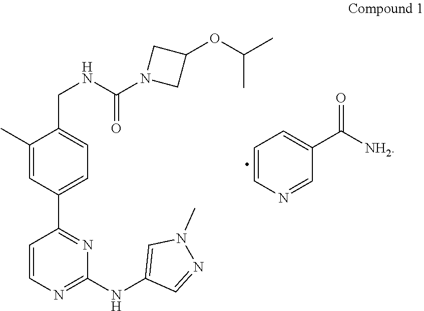 [SMILES: Cc1cc(-c2ccnc(Nc3cnn(C)c3)n2)ccc1CNC(=O)N1CC(OC(C)C)C1 ; NC(=O)c1cccnc1]

2. The compound according to embodiment 1, wherein said compound is crystalline.

3. The compound according to embodiment 2, wherein said compound is a crystalline solid substantially free of amorphous Compound 30.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 30.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 31

1. Compound 31, comprising Compound 1 and nicotinic acid:

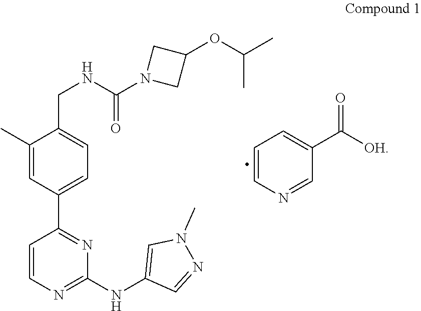 [SMILES: Cc1cc(-c2ccnc(Nc3cnn(C)c3)n2)ccc1CNC(=O)N1CC(OC(C)C)C1 ; O=C(O)c1cccnc1]

2. The compound according to embodiment 1, wherein said compound is crystalline.

3. The compound according to embodiment 2, wherein said compound is a crystalline solid substantially free of amorphous Compound 31.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 31.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

### Compound 32

1. Compound 32, comprising Compound 1 and saccharin:

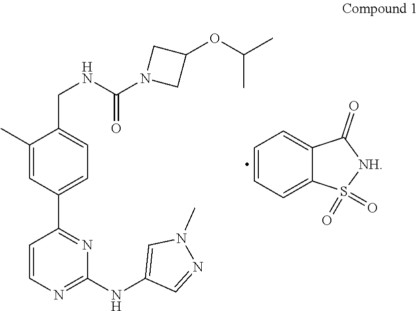 [SMILES: Cc1cc(-c2ccnc(Nc3cnn(C)c3)n2)ccc1CNC(=O)N1CC(OC(C)C)C1 ; O=C1NS(=O)(=O)c2ccccc21]

2. The compound according to embodiment 1, wherein said compound is crystalline.

3. The compound according to embodiment 2, wherein said compound is a crystalline solid substantially free of amorphous Compound 32.

4. The compound according to embodiment 1, wherein said compound is substantially free of impurities.

5. The compound according to embodiment 1, wherein said compound is of Type A.

6. The compound according to embodiment 5, having one or more peaks in its XRPD selected from those at about 7.32, about 13.26, about 15.59, about 22.83, and about 27.77 degrees 2-theta.

7. The compound according to embodiment 6, having at least two peaks in its XRPD selected from those at about 7.32, about 13.26, about 15.59, about 22.83, and about 27.77 degrees 2-theta.

8. The solid form according to embodiment 5, having a XRPD substantially similar to that depicted in FIG. 80.

9. The compound according to embodiment 1, wherein said compound is of Type B.

10. The compound according to embodiment 9, having one or more peaks in its XRPD selected from those at about 5.64, about 18.54, and about 24.31 degrees 2-theta.

11. The compound according to embodiment 10, having at least two peaks in its XRPD selected from those at about 5.64, about 18.54, and about 24.31 degrees 2-theta.

12. The compound according to embodiment 9, having an XRPD substantially similar to that depicted in FIG. 80.

13. The compound according to embodiment 1, wherein said compound is of Type C.

14. The compound according to embodiment 13, having one or more peaks in its XRPD selected from those at about 8.59, about 12.88, about 19.98, and about 25.71 degrees 2-theta.

15. The compound according to embodiment 14, having at least two peaks in its XRPD selected from those at about 8.59, about 12.88, about 19.98, and about 25.71 degrees 2-theta.

16. The compound according to embodiment 13, having an XRPD substantially similar to that depicted in FIG. 80.

17. The compound according to embodiment 1, wherein said compound is of Type D.

18. The compound according to embodiment 17, having one or more peaks in its XRPD selected from those at about 9.28, about 11.53, about 13.94, and about 19.19 degrees 2-theta.

19. The compound according to embodiment 18, having at least two peaks in its XRPD selected from those at about 9.28, about 11.53, about 13.94, and about 19.19 degrees 2-theta.

20. The compound according to embodiment 17, having an XRPD substantially similar to that depicted in FIG. 80.

21. The compound of embodiment 1, wherein said compound is amorphous.

22. The compound according to embodiment 17, wherein said compound is substantially free of crystalline Compound 32.

23. The compound according to embodiment 17, wherein said compound is substantially free of impurities.

24. A composition comprising the compound according to any one of embodiments 1-23 and a pharmaceutically acceptable carrier or excipient.

25. A method of decreasing the enzymatic activity of Bruton's tyrosine kinase comprising contacting Bruton's tyrosine kinase with an effective amount of a compound of any one of embodiments 1-23 or a composition thereof.

26. A method of treating a disorder responsive to inhibition of Bruton's tyrosine kinase comprising administering to a subject an effective amount of a compound of any one of embodiments 1-23 or a composition thereof.

27. A method of treating a disorder selected from the group consisting of autoimmune disorders, inflammatory disorders, and cancers comprising administering to a subject an effective amount of a compound of any one of embodiments 1-23 of a composition thereof.

28. The method according to embodiment 27, wherein the disorder is selected from rheumatoid arthritis, systemic lupus erythematosus, atopic dermatitis, leukemia and lymphoma.

### Compound 33

1. Compound 33, comprising Compound 1 and L-pyroglutamic acid:

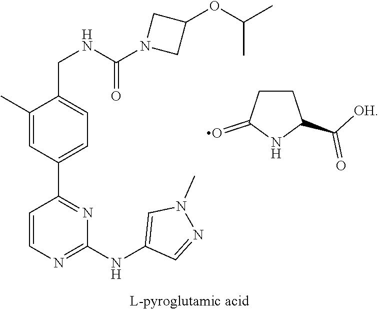

2. The compound according to embodiment 1, wherein said compound is crystalline.

3. The compound according to embodiment 2, wherein said compound is a crystalline solid substantially free of amorphous Compound 33.

4. The compound according to embodiment 1, wherein said compound is substantially free of impurities.

5. The compound of embodiment 1, wherein said compound is amorphous.

6. The compound according to embodiment 5, wherein said compound is substantially free of crystalline Compound 33.

7. The compound according to embodiment 5, wherein said compound is substantially free of impurities.

8. A composition comprising the compound according to any one of embodiments 1-7 and a pharmaceutically acceptable carrier or excipient.

9. A method of decreasing the enzymatic activity of Bruton's tyrosine kinase comprising contacting Bruton's tyrosine kinase with an effective amount of a compound of any one of embodiments 1-7 or a composition thereof.

10. A method of treating a disorder responsive to inhibition of Bruton's tyrosine kinase comprising administering to a subject an effective amount of a compound of any one of embodiments 1-7 or a composition thereof.

11. A method of treating a disorder selected from the group consisting of autoimmune disorders, inflammatory disorders, and cancers comprising administering to a subject an effective amount of a compound of any one of embodiments 1-7 of a composition thereof.

12. The method according to embodiment 11, wherein the disorder is selected from rheumatoid arthritis, systemic lupus erythematosus, atopic dermatitis, leukemia and lymphoma.

General Methods of Providing the Compounds

Compound 1 is prepared according to the methods described in detail in the '853 application, the entirety of which is hereby incorporated herein by reference.

The acid addition compounds of general formula A, which formula encompasses, inter alia, Compounds 2 through 33, and/or particular forms thereof, are prepared from Compound 1, according to the general Scheme below.

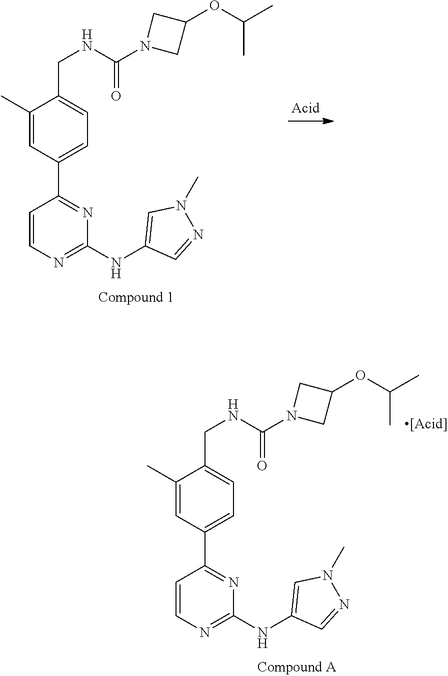

In this scheme, “Acid” represents, e.g., any of the co-formers described herein. For instance, each of Compounds 2 through 33, and forms thereof, are prepared from Compound 1 by combining Compound 1 with an appropriate acid to form the product Compound. Thus, another aspect of the present invention provides a method for preparing Compounds 2 through 33, and forms thereof.

As described generally above, in some embodiments, the present invention provides a method for preparing a Compound of the general formula A:

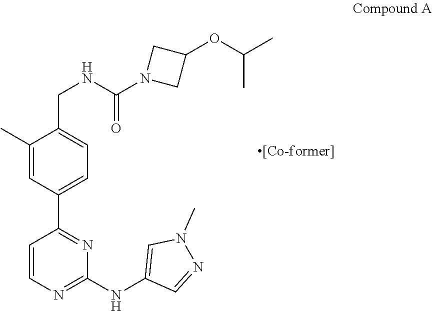

comprising steps of:

combining Compound 1:

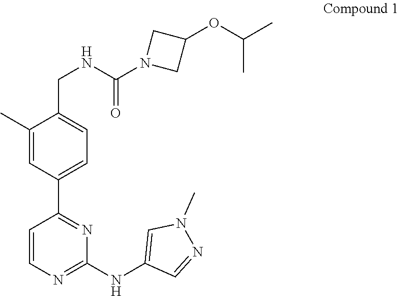 [SMILES: Cc1cc(-c2ccnc(Nc3cnn(C)c3)n2)ccc1CNC(=O)N1CC(OC(C)C)C1]

with a suitable co-former (e.g., a suitable acid) and optionally a suitable solvent under conditions suitable for forming a Compound of general formula A.

In some embodiments, Compound 1 is treated with a co-former selected from: hydrochloric acid, sulfuric acid, methanesulfonic acid, ethanedisulfonic acid, 2-hydroxyethanesulfonic acid, benzenesulfonic acid, toluenesulfonic acid, 2-naphthalene sulfonic acid, nitric acid, oxalic acid, fumaric acid, L-tartric acid, citric acid, L-malic acid, succinic acid, hippuric acid, maleic acid, glutamic acid, benzoic acid, gentisic acid, malonic acid, cinnamic acid, L-glutamide, L-lysine, L-phenylalanine, L-proline, L-serine, L-tyrosine, nicotinamide, nicotinic acid, saccharin, and L-pyroglutamic acid.

In some embodiments, a suitable co-former is hydrochloric acid.

In some embodiments, a suitable co-former is sulfuric acid.

In some embodiments, a suitable co-former is methanesulfonic acid.

In some embodiments, a suitable co-former is ethanedisulfonic acid.

In some embodiments, a suitable co-former is 2-hydroxyethanesulfonic acid.

In some embodiments, a suitable co-former is benzenesulfonic acid.

In some embodiments, a suitable co-former is toluenesulfonic acid.

In some embodiments, a suitable co-former is 2-naphthalene sulfonic acid.

In some embodiments, a suitable co-former is nitric acid.

In some embodiments, a suitable co-former is oxalic acid.

In some embodiments, a suitable co-former is fumaric acid.

In some embodiments, a suitable co-former is L-tartric acid.

In some embodiments, a suitable co-former is citric acid.

In some embodiments, a suitable co-former is L-malic acid.

In some embodiments, a suitable co-former is succinic acid.

In some embodiments, a suitable co-former is hippuric acid.

In some embodiments, a suitable co-former is maleic acid.

In some embodiments, a suitable co-former is glutamic acid.

In some embodiments, a suitable co-former is benzoic acid.

In some embodiments, a suitable co-former is gentisic acid.

In some embodiments, a suitable co-former is malonic acid.

In some embodiments, a suitable co-former is cinnamic acid.

In some embodiments, a suitable co-former is L-glutamide.

In some embodiments, a suitable co-former is L-lysine.

In some embodiments, a suitable co-former is L-phenylalanine.

In some embodiments, a suitable co-former is L-proline.

In some embodiments, a suitable co-former is L-serine.

In some embodiments, a suitable co-former is L-tyrosine.

In some embodiments, a suitable co-former is nicotinamide.

In some embodiments, a suitable co-former is nicotinic acid.

In some embodiments, a suitable co-former is saccharin.

In some embodiments, a suitable co-former is L-pyroglutamic acid.

A suitable solvent may be any solvent system (e.g., one solvent or a mixture of solvents) in which Compound 1 and/or an acid are soluble, or are at least partially soluble.

Examples of suitable solvents useful in the present invention include, but are not limited to protic solvents, aprotic solvents, polar aprotic solvent, or mixtures thereof. In certain embodiments, suitable solvents include an ether, an ester, an alcohol, a ketone, or a mixture thereof. In some embodiments, a solvent is one or more organic alcohols. In some embodiments, a solvent is chlorinated. In some embodiments, a solvent is an aromatic solvent.

In certain embodiments, a suitable solvent is methanol, ethanol, isopropanol, or acetone wherein said solvent is anhydrous or in combination with water or heptane. In some embodiments, suitable solvents include tetrahydrofuran, dimethylformamide, dimethylsulfoxide, glyme, diglyme, methyl t-butyl ether, t-butanol, n-butanol, and acetonitrile. In some embodiments, a suitable solvent is ethanol. In some embodiments, a suitable solvent is anhydrous ethanol. In some embodiments, a suitable solvent is MTBE.

In some embodiments, a suitable solvent is ethyl acetate. In some embodiments, a suitable solvent is a mixture of methanol and methylene chloride. In some embodiments, a suitable solvent is a mixture of acetonitrile and water. In certain embodiments, a suitable solvent is methyl acetate, isopropyl acetate, acetone, or tetrahydrofuran. In certain embodiments, a suitable solvent is diethylether. In certain embodiments, a suitable solvent is water. In certain embodiments, a suitable solvent is methyl ethyl ketone. In certain embodiments, a suitable solvent is toluene.

In some embodiments, the present invention provides a method for preparing a Compound of the general formula A, comprising one or more steps of removing a solvent and adding a solvent. In some embodiments, an added solvent is the same as the solvent removed. In some embodiments, an added solvent is different from a solvent removed. Means of solvent removal are known in the synthetic and chemical arts and include, but are not limited to, any of those described herein and in the Exemplification.

In some embodiments, a method for preparing a Compound of the general formula A comprises one or more steps of heating or cooling a preparation.

In some embodiments, a method for preparing a Compound of the general formula A comprises one or more steps of agitating or stirring a preparation.

In some embodiments, a method for preparing a Compound of the general formula A comprises a step of adding a suitable acid to a solution or slurry of compound 1.

In some embodiments, a method for preparing a Compound of the general formula A comprises a step of heating.

In certain embodiments, a Compound of formula A precipitates from the mixture. In another embodiment, a Compound of formula A crystallizes from the mixture. In other embodiments, a Compound of formula A crystallizes from solution following seeding of the solution (i.e., adding crystals of a Compound of formula A to the solution).

A Compound of formula A can precipitate out of the reaction mixture, or be generated by removal of part or all of the solvent through methods such as evaporation, distillation, filtration (ex. nanofiltration, ultrafiltration), reverse osmosis, absorption and reaction, by adding an anti-solvent such as heptane, by cooling or by different combinations of these methods.

As described generally above, a Compound of formula A is optionally isolated. It will be appreciated that a Compound of formula A may be isolated by any suitable physical means known to one of ordinary skill in the art. In certain embodiments, precipitated solid Compound of formula A is separated from the supernatant by filtration. In other embodiments, precipitated solid Compound of formula A is separated from the supernatant by decanting the supernatant.

In certain embodiments, a Compound of formula A is separated from the supernatant by filtration.

In certain embodiments, an isolated Compound of formula A is dried in air. In other embodiments isolated Compound of formula A is dried under reduced pressure, optionally at elevated temperature.

### Methods of Use

In certain embodiments, compounds of the present invention (e.g., any of Compounds 1-33) are for use in medicine. In some embodiments, compounds of the present invention are useful as kinase inhibitors. In certain embodiments, compounds of the present invention are selective inhibitors of Btk. In some embodiments, the present invention provides methods of decreasing Btk enzymatic activity. Such methods include contacting a Btk with an effective amount of a provided compound. Therefore, the present invention further provides methods of inhibiting Btk enzymatic activity by contacting a Btk with a compound of the present invention.

In some embodiments, the present invention provides methods of decreasing Btk enzymatic activity. In some embodiments, such methods include contacting a Btk with an effective amount of a provided compound. Therefore, the present invention further provides methods of inhibiting Btk enzymatic activity by contacting a Btk with a compound of the present invention.

Btk enzymatic activity, as used herein, refers to Btk kinase enzymatic activity. For example, where Btk enzymatic activity is decreased, PIPS binding and/or phosphorylation of PLCγ is decreased. In some embodiments, the half maximal inhibitory concentration (IC₅₀) of a provided compound against Btk is less than 1 uM. In some embodiments, the IC₅₀ of a provided compound against Btk is less than 500 nM. In some embodiments, the IC₅₀ of a provided compound against Btk is less than 100 nM. In some embodiments, the IC₅₀ of a provided compound against Btk is less than 10 nM. In some embodiments, the IC₅₀ of a provided compound against Btk is less than 1 nM. In some embodiments, the IC₅₀ of a provided compound against Btk is from 0.1 nM to 10 uM. In some embodiments, the IC₅₀ of a provided compound against Btk is from 0.1 nM to 1 uM. In some embodiments, the IC₅₀ of a provided compound against Btk is from 0.1 nM to 100 nM. In some embodiments, the IC₅₀ of a provided compound against Btk is from 0.1 nM to 10 nM.

In some embodiments, provided compounds are useful for the treatment of diseases and disorders that may be alleviated by inhibiting (i.e., decreasing) Btk enzymatic activity. By “diseases” is meant diseases or disease symptoms. Thus, the present invention provides methods of treating autoimmune disorders, inflammatory disorders, and cancers in a subject in need thereof. Such methods include administering to the subject a therapeutically effective amount of a provided compound.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barré syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjögren's syndrome, temporal arteritis, and Wegener's granulomatosis. The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present invention provides a method of treating rheumatoid arthritis or lupus.

The term “cancer” includes diseases or disorders involving abnormal cell growth and/or proliferation. In some embodiments, such cancers include as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present invention provides a method of treating leukemia or lymphoma.

The term “subject,” as used herein, refers to a mammal to whom a pharmaceutical composition is administered. Exemplary subjects include humans, as well as veterinary and laboratory animals such as horses, pigs, cattle, dogs, cats, rabbits, rats, mice, and aquatic mammals.

### Assays

To develop useful Tec kinase family inhibitors (e.g., BTK), candidate inhibitors capable of decreasing Tec kinase family enzymatic activity may be identified in vitro. The activity of the inhibitor compounds can be assayed utilizing methods known in the art and/or those methods presented herein.

Compounds that decrease Tec kinase family members' enzymatic activity may be identified and tested using a biologically active Tec kinase family member, either recombinant or naturally occurring. Tec kinases can be found in native cells, isolated in vitro, or co-expressed or expressed in a cell. Measuring the reduction in the Tec kinase family member (e.g., BTK) enzymatic activity in the presence of an inhibitor relative to the activity in the absence of the inhibitor may be performed using a variety of methods known in the art, such as the POLYGAT-LS assays described below in the Examples. Other methods for assaying the activity of Btk and other Tec kinases are known in the art. The selection of appropriate assay methods is well within the capabilities of those of skill in the art.

Once compounds are identified that are capable of reducing Tec kinase family members' enzymatic activity, the compounds may be further tested for their ability to selectively inhibit a Tec kinase family member relative to other enzymes. Inhibition by a compound of the invention is measured using standard in vitro or in vivo assays such as those well known in the art or as otherwise described herein.

Compounds may be further tested in cell models or animal models for their ability to cause a detectable changes in phenotype related to a Tec kinase family member activity. In addition to cell cultures, animal models may be used to test Tec kinase family member (e.g., BTK) inhibitors for their ability to treat autoimmune disorders, inflammatory disorders, or cancer in an animal model.

### Pharmaceutical Compositions

In another aspect, the present invention provides pharmaceutical compositions comprising any of the compounds described herein (e.g., any of Compounds 1-33) or any of the compounds described herein (e.g., any of Compounds 1-33) in combination with a pharmaceutically acceptable excipient (e.g., carrier).

The pharmaceutical compositions include optical isomers, diastereomers, or pharmaceutically acceptable salts of the inhibitors disclosed herein. A compound described herein (e.g., any of Compounds 1-33) included in the pharmaceutical composition may be covalently attached to a carrier moiety, as described above. Alternatively, a compound described herein (e.g., any of Compounds 1-33) included in the pharmaceutical composition is not covalently linked to a carrier moiety.

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compounds of the invention can be administered alone or can be coadministered to the subject. Coadministration is meant to include simultaneous or sequential administration of the compounds individually or in combination (more than one compound). The preparations can also be combined, when desired, with other active substances (e.g. to reduce metabolic degradation).

### Formulations

Compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

### Effective Dosages

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat cancer, such compositions will contain an amount of active ingredient effective to achieve the desired result (e.g. decreasing the number of cancer cells in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to Btk inhibition); presence of other diseases or other health-related problems; kind of concurrent treatment; and complications from any disease or treatment regimen. Other therapeutic regimens or agents can be used in conjunction with the methods and compounds of the invention.

For any compound described herein, the therapeutically effective amount can be initially determined from cell culture assays. Target concentrations will be those concentrations of active compound(s) that are capable of decreasing kinase enzymatic activity as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring kinase inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In some embodiments, the dosage range is 0.001% to 10% w/v. In some embodiments, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

### EXAMPLES

The examples below are meant to illustrate certain embodiments of the invention, and not to limit the scope of the invention.

### General Experimental

Abbreviations
MeOH Methanol
DMSO Dimethyl sulfoxide
NMP 1-Methyl-2-pyrrolidone
DMAc N,N-Dimethyl acetamide
EtOH Ethanol
IPA Isopropyl alcohol
ACN Acetonitrile
DCM Dichloromethane
THF Tetrahydrofuran
2-MeTHF 2-Methyltetrahydrofuran
CHCl₃ Trichloromethane
MIBK Methyl isobutyl ketone
EtOAc Ethyl acetate
IPAc Isopropyl acetate
MTBE Methyl tert-butyl ether
DSC Differential scanning calorimetry
IC Ion chromatography
NMR Nuclear magnetic resonance
TGA Thermogravimetric analysis
XRPD X-ray powder diffraction
DVS Dynamic vapor sorption

### Instruments and Methods

### A. X-Ray Powder Diffraction (XRPD)

For XRPD analysis, a PANalytical Empyrean X-ray powder diffractometer was used. The parameters used are listed in Table 34.

TABLE 34
XRPD Parameters

| Parameter | Value |
|---|---|
| X-Ray wavelength | Cu, kα, Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.013 |
| Scan speed (°/min) | About 10 |

### B. Thermogravimetric (TGA) and Differential Scanning Calorimetry (DSC)

TGA data were collected using a TA Q500/Q5000 TGA from TA Instruments. DSC was performed using a TA Q200/Q2000 DSC from TA Instruments. Detailed parameters used are listed in Table 35.

TABLE 35
TGA and DSC Parameters

| Parameters | TGA | DSC |
|---|---|---|
| Method | Ramp | Ramp |
| Sample pan | Platinum, open | Aluminum, crimped |
| Temperature | RT-desired temperature | 25° C.-desired temperature |
| Heating rate | 10° C./min | 10° C./min |
| Purge gas | N₂ | N₂ |

### C. HPLC

Agilent 1100 HPLC was utilized and detailed chromatographic conditions are listed in Table 36.

TABLE 36
Chromatographic conditions and parameters

| HPLC | Agilent 1100 with DAD detector | |
|---|---|---|
| Column | Waters Xbridge C18 150 × 4.6 mm, 5 μm | |
| Mobile phase | A: 0.1% TFA in H₂O | |
| | B: 0.1% TFA in acetonitrile | |
| | Time (min) | % B |
| Gradient table | 0.0 | 10 |
| | 1.0 | 10 |
| | 7.0 | 95 |
| | 7.1 | 10 |
| | 10.0 | 10 |

| | |
|---|---|
| Run time | 10 min |
| Post time | 0 min |
| Flow rate | 1.0 mL/min |
| Injection volume | 10 μL |
| Detector wavelength | UV at 305 nm, Reference at 400 nm |
| Column temperature | 35° C. |
| Sampler temperature | RT |
| Diluent | MeOH |

### D. Solution NMR

Solution NMR was collected on Bruker 400M NMR Spectrometer using DMSO-d₆ or MeOH-d₄.

### Example 1: 3-isopropoxy-N-(2-methyl-4-(2-((1-methyl-1H-pyrazol-4-yl)amino)pyrimidin-4-yl)benzyl)azetidine-1-carboxamide (Compound 1)

The synthesis of Compound 1 is described in detail at Example 21 of the '853 application, which is reproduced herein for ease of reference.

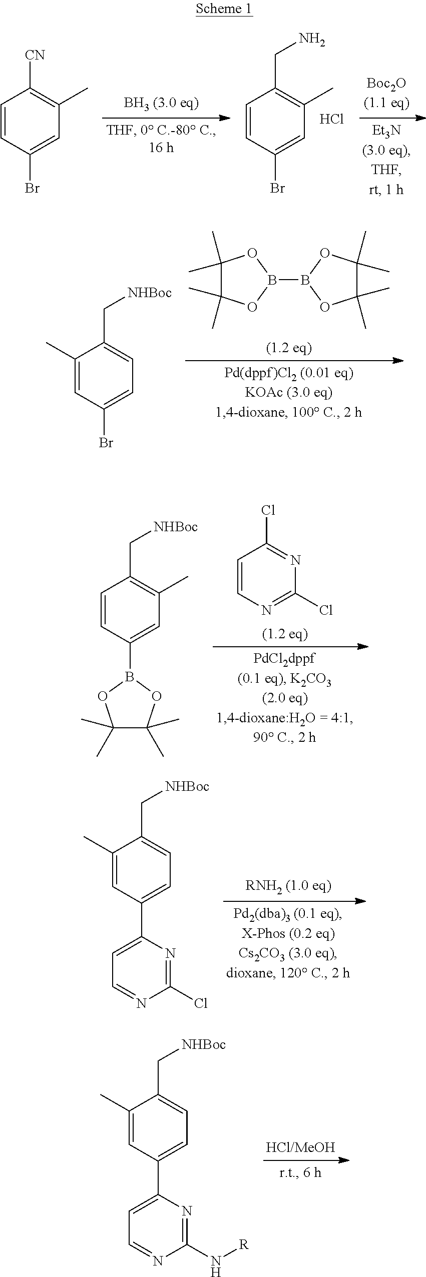

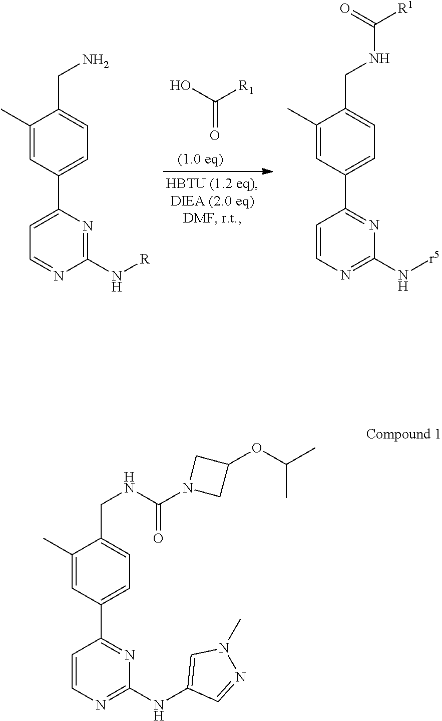

### Preparation of (4-bromo-2-methylphenyl)methanamine

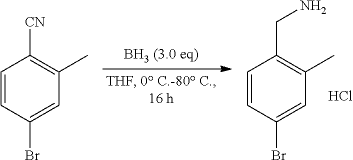

To a solution of 4-bromo-2-methylbenzonitrile (3 g, 15 mmol) in THF (20 mL), BH₃.THF (45 mL, 45 mmol) was added. The solution was stirred at 0° C. for 1 h and heated to 80° C. for 16 h. Then the mixture was quenched with MeOH. After concentrated, the residue was stirred with saturated HCl/EtOAc solution and filtered. The filter cake was rinsed with ether (20 mL×3) and dried under vacuum to afford (4-bromo-2-methylphenyl)methanamine (3.2 g, yield: 90%) as white solid. ESI-MS (M+H)⁺: 200.1

### Preparation of tert-butyl 4-bromo-2-methylbenzylcarbamate

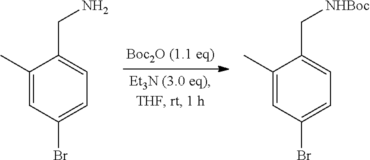

To a solution of (4-bromo-2-methylphenyl)methanamine (1.2 g, 6 mmol) in DCM (30 mL) were added TEA (1.82 g, 18 mmol) and Boc₂O (1.43 g, 6.6 mmol). The mixture was stirred at rt for 1 h. After diluted with water (50 mL), the mixture was extracted with DCM (50 mL×2). The combined organics were washed with brine (50 mL), dried (Na₂SO₄), filtered and concentrated to give crude title product (1.7 g, yield 95%) as a white solid, which was used directly in the next step without further purification. ESI-MS (M+H)⁺: 300.1.

### Preparation of tert-butyl 2-methyl-4-(4,4,5,5-tetramethyl-1,3,2-dioxaborolan-2-yl)benzylcarbamate

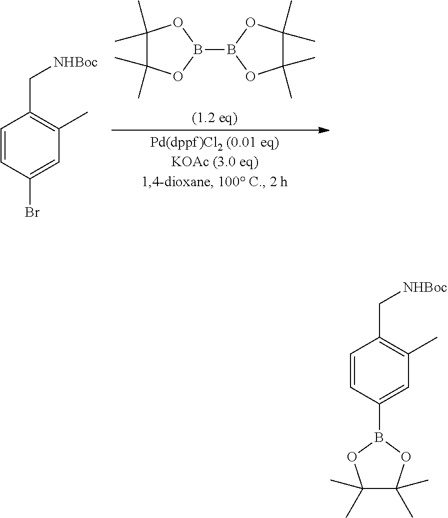

To a solution of tert-butyl 4-bromo-2-methylbenzylcarbamate (1.5 g, 5.0 mmol) in 1,4-dioxane (15 mL) were added 4,4,4′,4′,5,5,5′,5′-octamethyl-2,2′-bi(1,3,2-dioxaborolane) (1.52 g, 6.0 mmol), KOAc (1.75 g, 18 mmol) and Pd(dppf)Cl₂DCM (407 mg, 0.5 mmol) under nitrogen. The mixture was stirred at 100° C. for 2 h. After cooling down to rt, the mixture was diluted with water (50 mL) and extracted with ethyl acetate (100 mL×3). The combined organic layer was washed with brine, dried, concentrated and purified by silica gel column (petroleum ether/EtOAc=10:1) to give tert-butyl 2-methyl-4-(4,4,5,5-tetramethyl-1,3,2-dioxaborolan-2-yl)benzylcarbamate (1.2 g, yield 69%) as white solid. ESI-MS (M+H)⁺: 348.2. ¹H NMR (400 MHz, CDCl₃) δ: 7.61-7.59 (m, 2H), 7.26 (s, 1H), 4.68 (br, 1H), 4.33 (d, J=5.6 Hz, 2H), 2.32 (s, 3H), 1.45 (s, 9H), 1.34 (s, 12H).

### Preparation of tert-butyl 4-(2-chloropyrimidin-4-yl)-2-methylbenzylcarbamate

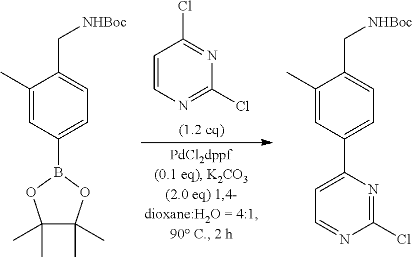

To a solution of tert-butyl 2-methyl-4-(4,4,5,5-tetramethyl-1,3,2-dioxaborolan-2-yl)benzylcarbamate (3.47 g, 10 mmol) and 2,4-dichloropyrimidine (1.79 g, 12 mmol) in 1,4-dioxane (28 mL) and H₂O (7 mL), Pd(dppf)Cl₂.DCM (815 mg, 1.0 mmol) and K₂CO₃ (2.76 g, 20 mmol) were added under N₂. The mixture was stirred at 90° C. for 2 h. After cooling to rt, the mixture was diluted with H₂O (80 mL) and extracted with EA (80 mL×2). The organic layers were dried and concentrated. The residue was purified by column chromatography (silica, petroleum ether/EtOAc=5:1 to 2:1) to give tert-butyl 4-(2-chloropyrimidin-4-yl)-2-methylbenzylcarbamate (2.67 g, yield 80%) as white solid ESI-MS (M+H)⁺: 334.1. ¹H NMR (400 MHz, CDCl₃) δ: 8.12 (d, J=5.2 Hz, 1H), 7.92 (s, 1H), 7.87 (d, J=8.0 Hz, 1H), 7.63 (d, J=5.6 Hz, 1H), 7.40 (d, J=7.6 Hz, 1H), 4.84 (br, 1H), 4.38 (d, J=5.2 Hz, 1H), 2.41 (s, 3H), 1.47 (s, 9H).

### Preparation of tert-butyl 2-methyl-4-(2-((l-methyl-1H-pyrazol-4-yl)amino)pyrimidin-4-yl)benzylcarbamate

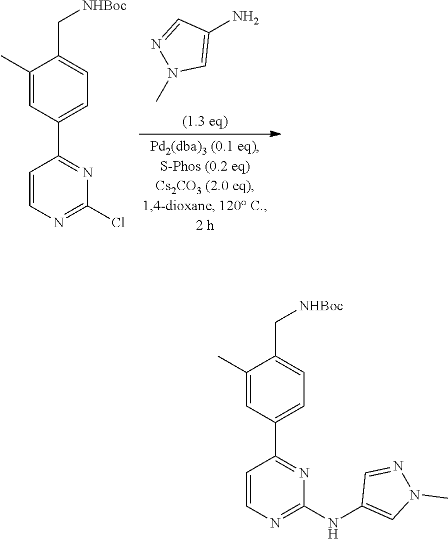

To a solution of tert-butyl 4-(2-chloropyrimidin-4-yl)-2-methylbenzylcarbamate (333 mg, 1.0 mmol) and 1-methyl-pyrazol-4-amine (126 mg, 1.3 mmol) in 1,4-dioxane (5 mL), Pd₂(dba)₃ (92 mg, 0.1 mmol), S-Phos (82 mg, 0.2 mmol) and Cs₂CO₃ (650 mg, 2.0 mmol) were added under N₂. The mixture was stirred at 120° C. for 2 h. After cooling to rt, the mixture was diluted with H₂O (40 mL) and extracted with EA (60 mL×2). The organic layers were dried and concentrated. The residue was purified by column chromatography (silica, petroleum ether/EtOAc=3:1 to 1:1) to give tert-butyl 2-methyl-4-(2-((1-methyl-1H-pyrazol-4-yl)amino)pyrimidin-4-yl)benzylcarbamate (248 mg, yield 63%) as white solid ESI-MS (M+H)⁺: 395.1. ¹H NMR (400 MHz, CD₃OD) δ: 8.38 (d, J=5.2 Hz, 1H), 7.97-7.93 (m, 3H), 7.65 (s, 1H), 7.38 (d, J=8.0 Hz, 1H), 7.20 (d, J=9.2 Hz, 1H), 4.30 (s, 2H), 3.85 (s, 3H), 2.42 (s, 3H), 1.48 (s, 9H).

### Preparation of 4-(4-(aminomethyl)-3-methylphenyl)-N-(1-methyl-1H-pyrazol-4-yl)pyrimidin-2-amine

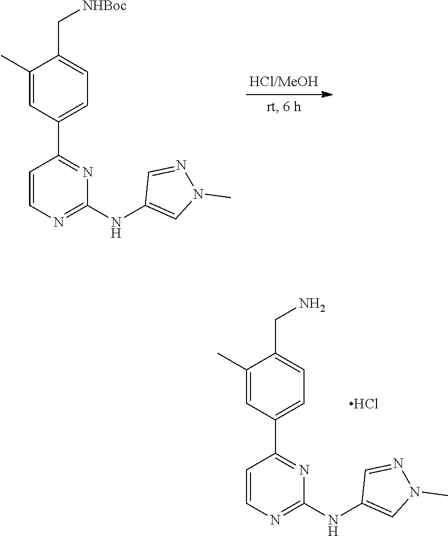

A mixture of tert-butyl 2-methyl-4-(2-((1-methyl-1H-pyrazol-4-yl)amino)pyrimidin-4-yl)benzylcarbamate (3.94 g, 10.0 mmol) in a solution of HCl in methanol (30 mL, prepared from gas HCl) was stirred at rt for 6 h. The solvent was removed and the solid was rinsed with cold diethyl ether (100 mL). The solid was dried under vacuum to give 4-(4-(aminomethyl)-3-methylphenyl)-N-(1-methyl-1H-pyrazol-4-yl)pyrimidin-2-amine (2.97 g, yield 90%) as a yellow solid ESI-MS (M+H)⁺: 295.1. ¹H NMR (400 MHz, D₂O) δ: 7.98-7.96 (m, 1H), 7.66-7.22 (m, 6H), 4.10 (s, 2H), 3.68 (s, 3H), 2.20 (s, 3H).

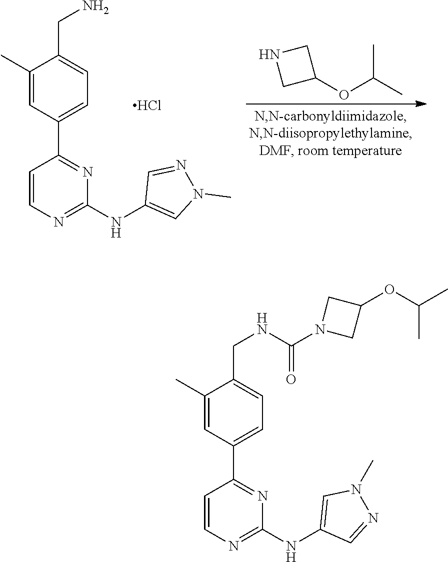

4-(4-(aminomethyl)-3-methylphenyl)-N-(1-methyl-1H-pyrazol-4-yl)pyrimidin-2-amine hydrochloride (prepared in Example 1) (200 mg, 0.7 mmol), 3-isopropoxy azetidine (113 mg, 0.747 mmol), and N,N-carbonyldiimidazole (0.110 g, 0.679 mmol) in N,N-dimethylformamide (1.58 mL, 20.4 mmol) was added N,N-diisopropylethylamine (0.473 mL, 2.72 mmol) slowly and stirred at room temperature overnight. The mixture was filtrate through celite and washed with DMF and purified by prep HPLC to give product as a solid (82 mg, yield: 30%). LCMS: Rt=1.05 min, m/z 436.3. 1H NMR (400 MHz, DMSO-d6) δ: 9.48 (s, 1H), 8.45 (d, J=5.02 Hz, 1H), 7.92 (s, 3H), 7.55 (br. s., 1H), 7.35 (d, J=8.53 Hz, 1H), 7.25 (d, J=5.27 Hz, 1H), 6.84 (s, 1H), 4.15-4.48 (m, 3H), 3.90-4.13 (m, 2H), 3.83 (s, 3H), 3.46-3.69 (m, 3H), 2.36 (s, 3H), 1.08 (d, J=6.27 Hz, 6H).

### Example 2: Solid Forms of Compound 1 (Freebase)

Studies were undertaken to identify various new forms (e.g., solid forms) of Compound 1.

For TGA studies, the temperature range was room temperature to 300° C. For DSC studies, the temperature range was room temperature to 250° C.

Dynamic Vapor Sorption (DVS) was measured via a SMS (Surface Measurement Systems) DVS Intrinsic. Parameters for DVS test were listed in Table 37.

TABLE 37
Parameters for DVS test

| Parameters | Values |
|---|---|
| Temperature | 25° C. |
| Sample size | 10-20 mg |
| Gas and flow rate | N₂, 200 mL/min |
| dm/dt | 0.002%/min |
| Min.dm/dtstability | 10 min |
| duration | |
| Max. equilibrium time | 360 min |
| RH range | 95% RH-0% RH-95% RH |
| RH step size | 10% (90% RH-0% RH-90% RH) |
| | 5% (95% RH-90% RH and 90% RH-95% RH) |

Characterization data for certain solid forms are provided herein and are briefly summarized in Table 38. Solid Forms A-E were obtained during initial screening experiments. Compound 1 Type F was obtained by heating Compound 1 Type D to 120° C. and cooling to room temperature. Compound 1 Type G was obtained by heating Compound 1 Type C to 130° C. and cooling to room temperature.

TABLE 38
Characterization summary for solid forms of Compound 1

| | Weight Loss | Endotherm | |
|---|---|---|---|
| Solid Form | (%) | (° C., peak) | Form Identity |
| Type A | 0.6 | 182.3 | Anhydrate |
| Type B | 5.9 | 107.8, 182.0 | Hydrate |
| Type C | 0.9 | 99.2, 155.5 | Anhydrate |
| Type D | 0.7 | 109.5, 152.2, 182.0 | Anhydrate |
| Type E | N/A* | N/A* | N/A* |
| Type F | 1.5 | 151.4, 181.9 | Anhydrate |
| Type G | 0.2 | 144.9, 154.8, 182.8 | Anhydrate |

N/A: Type E converted into Type B at ambient conditions and was not reproduced.

### 1. Initial Screening

The solubility of solid Form A of Compound 1 was estimated in 21 solvents at room temperature. Approximately 2 mg solids were added into a 3-mL glass vial. Solvents in Table 39 were then added step wise (100 μL per step) into the vials until the solids were dissolved or a total volume of 2 mL was reached. Results summarized in Table 39 were used to guide the solvent selection in polymorph screening.

TABLE 39
Solubility of Compound 1 Form A at Room Temprature

| | Solubility | | Solubility |
|---|---|---|---|
| Solvent | (mg/mL) | Solvent | (mg/mL) |
| MeOH | 12.5 < S < 25.0 | 2-MeTHF | 2.2 < S < 2.4 |
| EtOH | 4.4 < S < 5.5 | 1,4-Dioxane | 3.5 < S < 4.2 |
| IPA | 3.0 < S < 3.5 | NMP | S > 56.0 |
| ACN | 2.3 < S < 2.6 | DMSO | S > 38.0 |
| Acetone | 4.6 < S < 5.8 | DCM | 12.0 < S < 24.0 |
| MIBK | S < 1.4 | Toluene | S < 0.9 |
| EtOAc | 1.4 < S < 1.5 | n-Heptane | S < 1.5 |
| IPAc | S < 1.5 | DMAc | S > 54.0 |
| MTBE | S < 1.3 | H₂O | S < 1.1 |
| THF | 6.0 < S < 9.0 | CHCl₃ | 20.0 < S < 40.0 |
| Acetic acid | S > 54.0 | — | — |

Polymorph screening experiments were performed using different solution crystallization or solid transition methods. The methods utilized and crystal forms identified are summarized in Table 40. Five different solid forms were obtained from these initial screening experiments.

TABLE 40
Summary of polymorph screening experiments

| Method | Isolated Solid Forms |
|---|---|
| Anti-solvent Addition | Type A, B, D and E |
| Solid Vapor Diffusion | Type A |
| Solution Vapor Diffusion | Type B and C |
| Polymer-induced Crystallization | Type A, B and C |
| Slow Evaporation | Type A, B and D |
| Slurry at RT | Type A and B |
| Slurry at 5° C. | Type A |
| Slow Cooling | Type A, B, C and D |
| Grinding | Type A |

### Anti-Solvent Addition

A total of 24 anti-solvent addition experiments were carried out. About 15 mg of Compound 1 Type A were dissolved in 0.2-5.0 mL solvent to obtain a clear solution. The solution was magnetically stirred. Addition of 0.1 mL anti-solvent was then added stepwise until a precipitate appeared or the total amount of anti-solvent reached 15.0 mL. The precipitate was isolated for XRPD analysis. Clear solutions were transferred to agitation at 5° C. for one day, and solids were then tested by XRPD. The final clear solutions were transferred to evaporation at room temperature. Results summarized in Table 41 showed that Compound 1 Types A, B, D, and E were generated.

TABLE 41
Summary of anti-solvent addition experiments

| Solvent | Anti-solvent | Solid Form |
|---|---|---|
| MeOH | H₂O | Compound 1 Type B |
| EtOH | H₂O | Compound 1 Type B |
| Acetone | H₂O | Compound 1 Type B |
| THF | H₂O | Compound 1 Type A |
| Acetic acid | H₂O | Compound 1 Type B |
| DMSO | H₂O | Amorphous Compound 1 |
| DMF | H₂O | Compound 1 Type B |
| EtOH | n-Heptane | Compound 1 Type B |
| IPA | n-Heptane | Compound 1 Type B |
| Acetone | n-Heptane | Compound 1 Type A |
| THF | n-Heptane | Compound 1 Type E |
| Acetic acid | n-Heptane | Compound 1 Type B |
| DCM | n-Heptane | Compound 1 Type D |
| CHCl₃ | n-Heptane | Compound 1 Type D |
| MeOH | Toluene | Compound 1 Type B |
| Acetic acid | Toluene | Compound 1 Type B |
| THF | Toluene | Compound 1 Type B |
| DCM | Toluene | Compound 1 Type B |
| NMP | IPAc | N/A |
| 1,4-Dioxane | IPAc | Compound 1 Type B |
| MeOH | MIBK | Compound 1 Type B |
| DMSO | MIBK | Compound 1 Type B |
| MeOH | MTBE | Compound 1 Type B |
| CHCl₃ | MTBE | Compound 1 Type A |

N/A: no solid was obtained.

### Solid Vapor Diffusion

Solid vapor diffusion experiments were conducted using 14 different kinds of solvent. Approximately 10 mg of Compound 1 Type A were weighed into a 3-mL vial, which was placed into a 20-mL vial with 2 mL of relative solvent. The 20-mL vial was sealed with a cap and kept at room temperature allowing solvent vapor to interact with sample for 6 days. The solids were tested by XRPD, and the results summarized in Table 42 showed that no form change was observed.

TABLE 42
Summary of solid vapor diffusion experiments
using Compound 1 Type A

| Solvent | Solid Form | Solvent | Solid Form |
|---|---|---|---|
| H₂O | Compound 1 Type A | Acetone | Compound 1 Type A |
| DCM | Compound 1 Type A | DMF | Compound 1 Type A |
| EtOH | Compound 1 Type A | EtOAc | Compound 1 Type A |
| MeOH | Compound 1 Type A | 1,4-Dioxane | Compound 1 Type A |
| ACN | Compound 1 Type A | IPA | Compound 1 Type A |
| THF | Compound 1 Type A | Acetic acid | N/A |
| CHCl₃ | Compound 1 Type A | DMSO | Compound 1 Type A |

N/A: solid was dissolved during the diffusion process.

### Solution Vapor Diffusion

Approximately 15 mg of Compound 1 Type A were dissolved in 1.0 or 2.0 mL of corresponding solvent to obtain a clear solution in a 3-mL vial. This solution was then placed into a 20-mL vial with 3 mL of relative anti-solvent. The 20-mL vial was sealed with a cap and kept at room temperature allowing sufficient time for organic vapor to interact with the solution. The precipitates were isolated for XRPD analysis. The results summarized in Table 43 showed that Compound 1 Types B and C were observed.

TABLE 43
Summary of solution vapor diffusion experiments

| Solvent | Anti-solvent | Solid Form |
|---|---|---|
| MeOH | H₂O | Compound 1 Type B |
| EtOH | H₂O | Compound 1 Type B |
| THF | H₂O | Compound 1 Type B |
| Acetone | H₂O | Compound 1 Type B |
| DMF | H₂O | N/A |
| DMSO | MTBE | N/A |
| CHCl₃ | MTBE | Compound 1 Type C |
| DCM | n-Heptane | Compound 1 Type C |
| Acetic acid | n-Heptane | N/A |
| THF | n-Heptane | Compound 1 Type B |

N/A: no solid was obtained.

### Polymer-Induced Crystallization

Polymer-induced crystallization experiments were performed with two sets of polymer mixtures in five different solvents. Approximately 15 mg of Compound 1 Type A were dissolved in 1.0 or 2.0 mL of corresponding solvent to obtain a clear solution in a 3-mL vial. About 2 mg of polymer mixture were added into 3-mL glass vial. All the samples were sealed using parafilm and then transferred to evaporation at room temperature to induce precipitation. The solids were isolated for XRPD analysis. Results summarized in Table 44 showed that Compound 1 Types A, B and C were obtained.

TABLE 44
Summary of polymer-induced crystallization experiments

| | Polymer | |
|---|---|---|
| Solvent (v/v) | Mixture | Solid Form |
| MeOH | A | Compound 1 Type B |
| THF | | Compound 1 Type B |
| DCM | | Compound 1 Type C |
| Acetone | | Compound 1 Type A |
| Acetic acid/H₂O (4:1) | | N/A |
| MeOH | B | Compound 1 Type B |
| THF | | Compound 1 Type B |
| DCM | | Compound 1 Type B |
| Acetone | | Compound 1 Type A |
| Acetic acid/H₂O (4:1) | | N/A |

N/A: no solid was obtained.
Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1)
Polymer mixture B: polycaprolactone (PCL), polyethylene glycol (PEG), poly(methyl methacrylate) (PMMA) sodium alginate (SA), and hydroxyethyl cellulose (HEC) (mass ratio of 1:1:1:1:1).

### Slow Evaporation

### Slow Evaporation (Procedure 2)

Approximately 15 mg of Compound 1 Type A were dissolved in 1.0 or 2.0 mL of corresponding solvent in a 3-mL glass vial. The visually clear solutions were subjected to evaporation at room temperature to induce precipitation. The solids were isolated for XRPD analysis, and the results summarized in Table 45 indicated that Compound 1 Types A, B and D were obtained.

TABLE 45
Summary of evaporation experiments

| Solvent (v/v) | Solid Form |
|---|---|
| MeOH | Compound 1 Type B |
| EtOH | Compound 1 Type B |
| Acetone | Compound 1 Type A |
| THF | Compound 1 Type B |
| 1,4-Dioxane | Compound 1 Type B |
| DCM | Compound 1 Type B |
| MeOH/H₂O (4:1) | Compound 1 Type B |
| Acetic acid/H₂O (4:1) | N/A |
| Acetic acid/n-Heptane (4:1) | N/A |
| CHCl₃/MTBE (4:1) | Compound 1 Type D |
| THF/n-Heptane(4:l) | Compound 1 Type A |
| | Compound 1 Type B |

### Slurry at Room Temperature

Slurry conversion experiments were conducted at room temperature in different solvent systems. About 15 mg of Compound 1 Type A were suspended in 0.5 mL of solvent in a 1.5-mL glass vial. After the suspension was stirred for 3 days at room temperature, the remaining solids were isolated for XRPD analysis. Results summarized in Table 46 indicated that Compound 1 Types A and B were generated.

TABLE 46
Summary of slurry conversion experiments at room temperature

| Solvent (v/v) | Solid Form |
|---|---|
| EtOH | Compound 1 Type A |
| IPA | Compound 1 Type A |
| ACN | Compound 1 Type A |
| EtOAc | Compound 1 Type A |
| 2-MeTHF | Compound 1 Type A |
| 1,4-Dioxane | Compound 1 Type A |
| THF | Compound 1 Type A |
| Acetone | Compound 1 Type A |
| Acetone/H₂O (986:14), a_{w} = 0.197* | Compound 1 Type A |
| Acetone/H₂O (950:50), a_{w} = 0.400* | Compound 1 Type A |
| Acetone/H₂O (860:140), a_{w} = 0.596* | Compound 1 Type B |
| Acetone/H₂O (600:400), a_{w} = 0.803* | Compound 1 Type A |
| H₂O | Compound 1 Type A |
| MeOH/H₂O (1:3) | Compound 1 Type A |
| DCM/n-Heptane (1:3) | Compound 1 Type A |
| Acetic acid/n-Heptane (1:3) | N/A |
| CHCl₃/MTBE (1:3) | Compound 1 Type A |

N/A: no solid was obtained.
*the a_{w} value is calculated at 25° C.

### Slurry at 5° C.

Slurry conversion experiments were also conducted at 5° C. in different solvent systems. About 15 mg of Compound 1 Type A were suspended in 0.3 mL of solvent in a 1.5-mL glass vial. After the suspension was stirred for 3 days at 5° C., the remaining solids were isolated for XRPD analysis. Results summarized in Table 47 indicated that no new crystal form was produced.

TABLE 47
Summary of slurry conversion experiments at 5° C.

| Solvent (v/v) | Solid Form |
|---|---|
| EtOH | Compound 1 Type A |
| IPA | Compound 1 Type A |
| ACN | Compound 1 Type A |
| EtOAc | Compound 1 Type A |
| 2-MeTHF | Compound 1 Type A |
| H₂O | Compound 1 Type A |
| THF | Compound 1 Type A |
| Acetone | Compound 1 Type A |
| MeOH/H₂O (1:3) | Compound 1 Type A |
| DCM/n-Heptane (1:3) | Compound 1 Type A |
| Acetic acid/n-Heptane (1:3) | N/A |
| CHCl₃/MTBE (1:3) | Compound 1 Type A |

N/A: no solid was obtained.

### Slow Cooling

Slow cooling experiments were conducted in ten solvent systems. About 20 mg of Compound 1 Type A were suspended in 1.0 or 2.0 mL of solvent in a 3-mL glass vial at room temperature. The suspension was then heated to 50° C., equilibrated for 2 hrs and filtered to a new vial using a Nylon membrane (pore size of 0.45 μm). The filtrates were slowly cooled down to 5° C. at a rate of 0.1° C./min. The solids thus obtained were kept isothermal at 5° C. before isolation for XRPD analysis. Clear solutions were evaporated to dryness at room temperature, and then solids were tested by XRPD. Results summarized in Table 48 indicated Compound 1 Types A, B, C and D were obtained.

TABLE 48
Summary of slow cooling experiments

| Solvent (v/v) | Solid Form* |
|---|---|
| EtOH | Compound 1 Type B |
| IPA | Compound 1 Type B |
| ACN | Compound 1 Type A |
| Acetone | N/A |
| THF | N/A |
| 2-MeTHF | Compound 1 Type C |
| MeOH/H₂O (4:1) | Compound 1 Type B |
| DMF/H₂O (4:1) | N/A |
| CHCl₃/n-Heptane (4:1) | N/A |
| CHCl₃/MTBE (4:1) | Compound 1 Type D |

*no solids were generated via slow cooling, and the clear solutions were transferred to evaporation at RT.
N/A: no solid was obtained.

### Grinding

Grinding-induced phase transition experiments were performed in two conditions with or without water. About 15 mg of Compound 1 Type A were weighed into a mortar and then ground manually using a pestle for 5 minutes. The solid was analyzed by XRPD and no new crystal form was generated (Table 49).

TABLE 49
Summary of grinding experiments

| Additive | Solid Form |
|---|---|
| N/A | Compound 1 Type A |
| H₂O | Compound 1 Type A |

N/A: no additive was added.

### 2. Characterization Data for Polymorphs of Compound 1

### Compound 1 Type A

FIG. 1 provides the XRPD overlay of starting material and Compound 1 Type A reference. The figure shows that the starting sample conforms to Type A.

FIG. 2 provides the TGA/DSC curves for Compound 1 Type A. A weight loss of 0.6% up to 150° C. in TGA and the only sharp melting endotherm at 179.6° C. (onset temperature) in DSC suggested that Type A is an anhydrate.

### Compound 1 Type B

Compound 1 Type B can be prepared via solvent-mediated crystallization from multiple solvent systems. Two batches of Type B sample (807302-79-A) and (807302-61-A2) were prepared via slurry conversion of Compound 1 Type A in acetone/water (v/v, 860:140).

FIG. 3 provides XRPD patterns of two different samples of Compound 1 Type B (Samples A and A2).

FIG. 4 provides TGA/DSC curves of Compound 1 Type B.

No form change was observed after Compound 1 Type B was heated to 80° C. and cooled to room temperature.

A new form (Compound 1 Type G) was observed at 130° C., and its characterization data is provided herein.

Compound 1 Type B converted into Compound 1 Type A after heating to 170° C. and cooling to room temperature under protection of N₂. NMR studies resulted in the detection of negligible amounts of acetone, indicating Compound 1 Type B is a hydrate.

### Compound 1 Type C

Compound 1 Type C can be prepared via solvent-mediated crystallization from multiple solvent systems. For example, a sample of Compound 1 Type C sample was prepared via slow evaporation of DCM solution.

FIG. 5 provides the XRPD pattern of Compound 1 Type C.

FIG. 6 provides the TGA/DSC curves of Compound 1 Type C. As per TGA and DSC data this figure, a weight loss of 0.9% up to 150° C. was observed in TGA and DSC result showed an endotherm at 89.9° C. before melting at 150.6° C. (onset temperature).

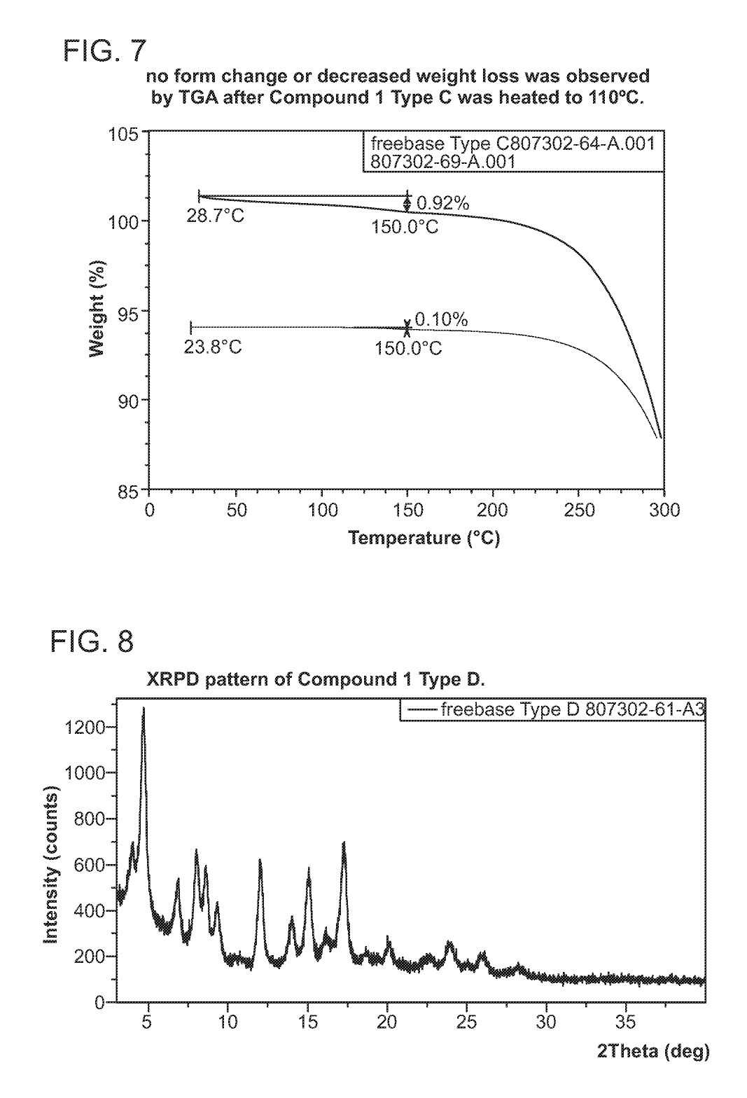

FIG. 7 shows that no form change or decreased weight loss was observed by TGA after Compound 1 Type C was heated to 110° C. Thus the first endotherm was possibly caused by removal of solvent residual, and Type C may be an anhydrate.

### Compound 1 Type D

Compound 1 Type D can be obtained via anti-solvent crystallization, slow evaporation, or cooling from solvent systems containing DCM or CHCl₃. For example, a sample of Compound 1 Type D was obtained by adding n-heptane into DCM solution.

FIG. 8 provides the XRPD pattern of Compound 1 Type D.

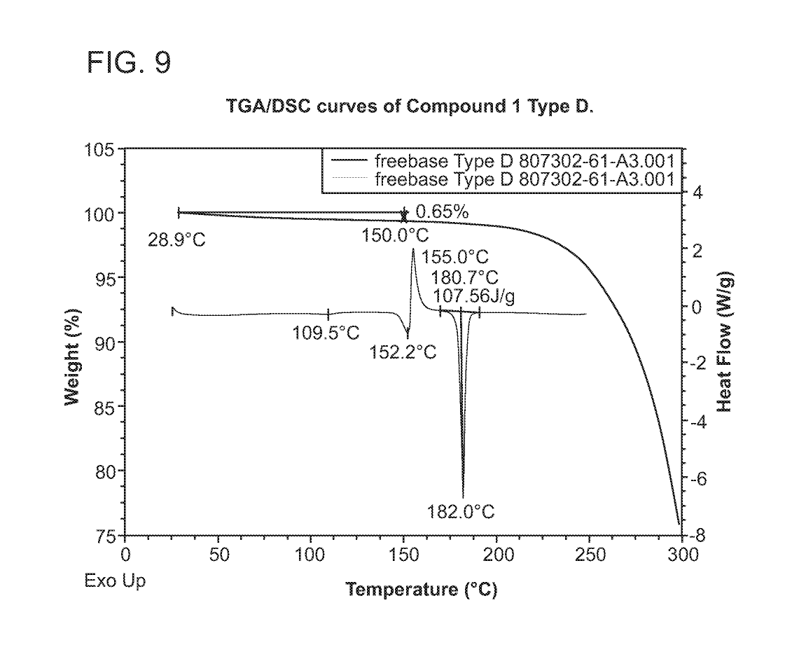

FIG. 9 provides the TGA/DSC curves of Compound 1 Type D. A limited weight loss of 0.7% was observed up to 150° C. in TGA. The DSC result showed three endotherms and one exotherm, suggesting Type D is an anhydrate.

To investigate multiple endo/exotherms detected from DSC and probe the existence of any new crystal form during the process, a sample of Compound 1 Type D was heated to different elevated temperatures by TGA, and the resulting solids were analyzed using XRPD after cooling to room temperature.

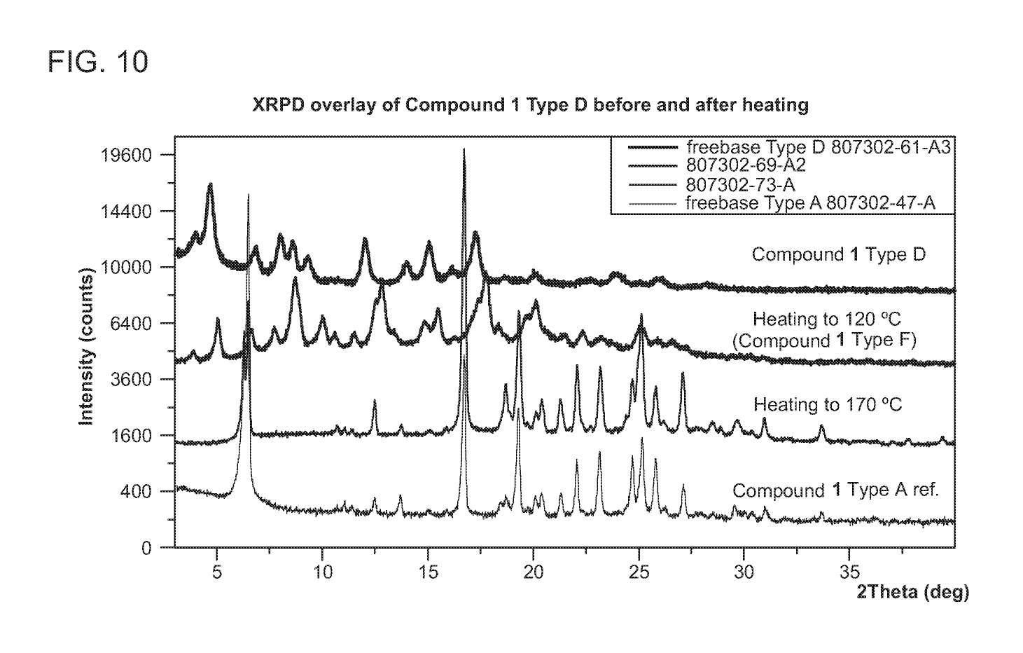

FIG. 10 provides the XRPD overlay of Compound 1 Type D before and after heating. As shown in this figure, distinctive diffraction peaks were observed after the first endotherm at 109.5° C. (peak temperature), and this new crystal form is assigned as Compound 1 Type F. The formation of Compound 1 Type A after heating sample to 170° C. suggested that the overlapped endo/exothermal signal could be caused by the melting of Compound 1 Type F followed by a recrystallization into Compound 1 Type A.

### Compound 1 Type E

Compound 1 Type E was isolated from anti-solvent addition of n-heptane into a THF solution of Compound 1 Type A at room temperature.

FIG. 11 provides the XRPD pattern of Compound 1 Type E. Sharp diffraction peaks in showed that the sample is crystalline.

FIG. 12 provides the XRPD overlay of Compound 1 Type E before and after storage. After storing at ambient conditions for 2 days, XRPD pattern re-collected conformed to Type B. XRPD pattern re-collected conformed to Type B. Several attempts to prepare Compound 1 Type E produced Compound 1 Type A or Compound 1 Type B.

### Compound 1 Type F

A sample of Compound 1 Type F was prepared by heating Compound 1 Type D to 120° C. and cooling to room temperature.

FIG. 13 provides the XRPD pattern of Compound 1 Type F.

FIG. 14 provides TGA and DSC curves of Compound 1 Type F. The limited weight loss of 1.5% up to 150° C. and no de-solvation endotherm before overlapped melting/recrystallization signal indicated that Type F is an anhydrate.

### Compound 1 Type G

A sample of Compound 1 Type G was prepared by heating Compound 1 Type C to 130° C. and cooling to room temperature.

FIG. 15 provides the XRPD pattern of Compound 1 Type G.

FIG. 16 provides the TGA/DSC curves of Compound 1 Type G. A limited weight loss of 0.2% up to 150° C. and no de-solvation endotherm before melting indicated that Type G is an anhydrate.

### 3. Investigation of Stability Relationships of Compound 1 Polymorphs

Based on the solid-state characterization data and preliminary form identity, among the six crystal forms that had been re-produced at ambient conditions, there exists a hydrate (Compound 1 Type B), and five anhydrates (Compound 1 Types A, C, D, F and G). Experiments were set up to understand the stability relationships between forms.

### Stability Relationship Between Anhydrates

To identify the most stable anhydrate at room temperature (20±2° C.) and 50° C., slurry competition experiments using mixtures of all the Compound 1 anhydrates were performed at both temperatures. In detail, suspensions with a solid load of ˜10 mg/mL were prepared using Compound 1 Type A as the starting material. After being equilibrated at room temperature and 50° C. for 0.5 hour via magnetically stirring, filtrates were transferred into a clean vial, followed by adding anhydrate mixtures to form new suspensions at both temperatures. Solids were isolated 3 days later,

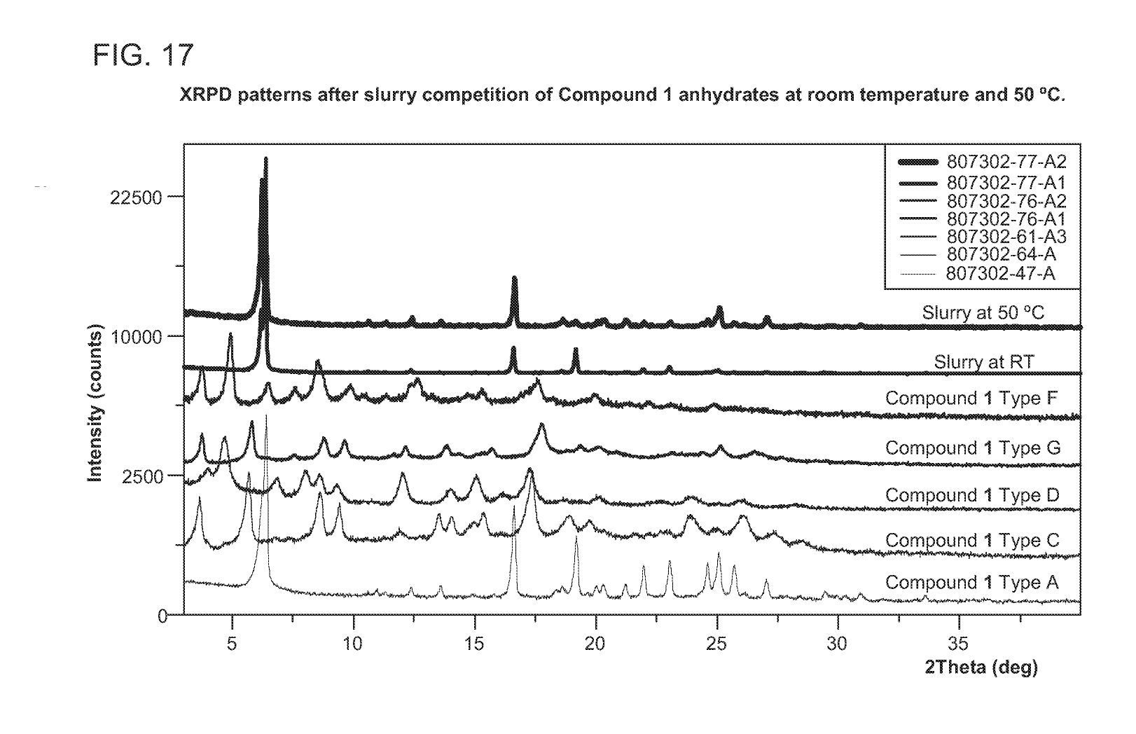

FIG. 17 provides the XRPD patterns after slurry competition of Compound 1 anhydrates at room temperature and 50° C. The XRPD patterns collected conformed to Compound 1 Type A, indicating Type A is the most stable anhydrate from room temperature to 50° C. As Compound 1 Type A possesses the highest melting point compared to all anhydrous forms, Compound 1 Type A is believed to be the thermodynamically stable anhydrate at least above room temperature.

### Critical Water Activity Determination Between Compound 1 Types A and B

Compound 1 Type A is the most thermodynamically stable anhydrate above room temperature. To study stability relationship of Compound 1 Types A and B, critical water activity was investigated through competitive slurry experiments. Acetone and water were first used to prepare a series of solvent mixtures with known water activity at room temperature (20±2° C.) and 50° C. Similar to the anhydrate experiments, Compound 1 Type A solids were suspended into each condition with an initial solid load of ˜60 mg/mL, and mixtures of Compound 1 Types A and B with a mass ratio of 1:1 were then added into each filtrate. Solids were equilibrated under each condition for about 12 hours before isolated for XRPD test. The starting mixture of Compound 1 Types A and B was characterized by XRPD and used as a control to compare the conversion trend (e.g., if it took a long time to reach equilibrium).

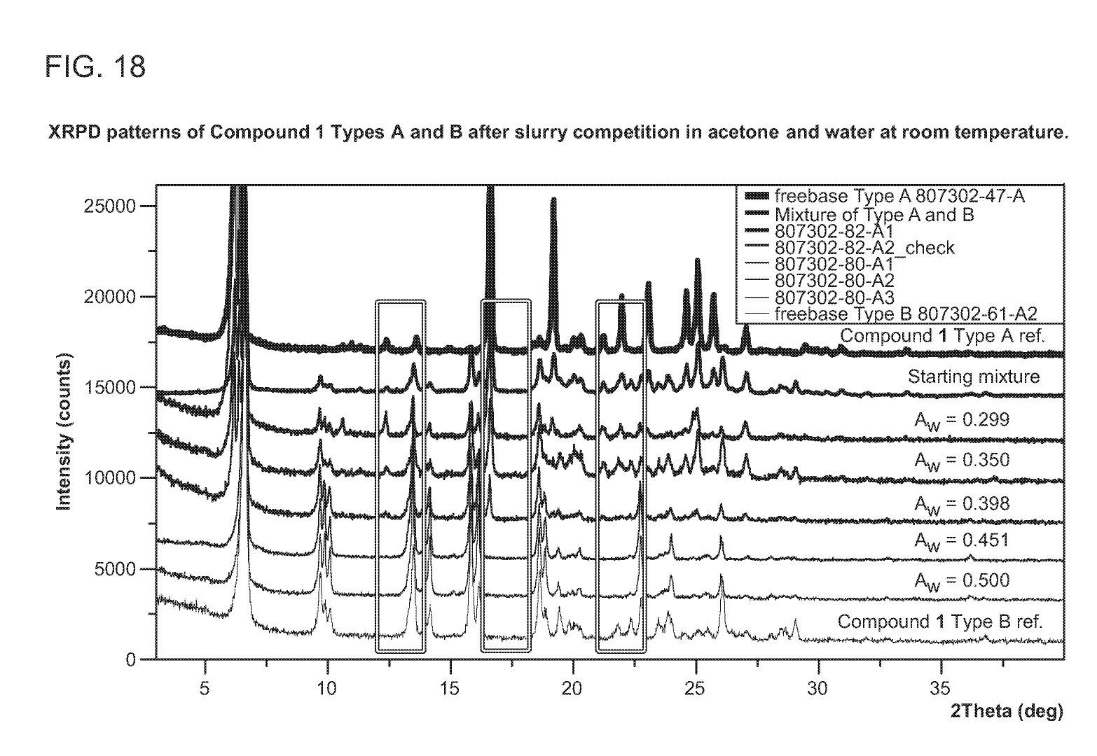

FIG. 18 provides XRPD patterns of Compound 1 Forms A and B after slurry competition in acetone and water at room temperature. The critical water activity between Compound 1 Forms Type A and B is in the range of 0.3˜0.4 at room temperature.

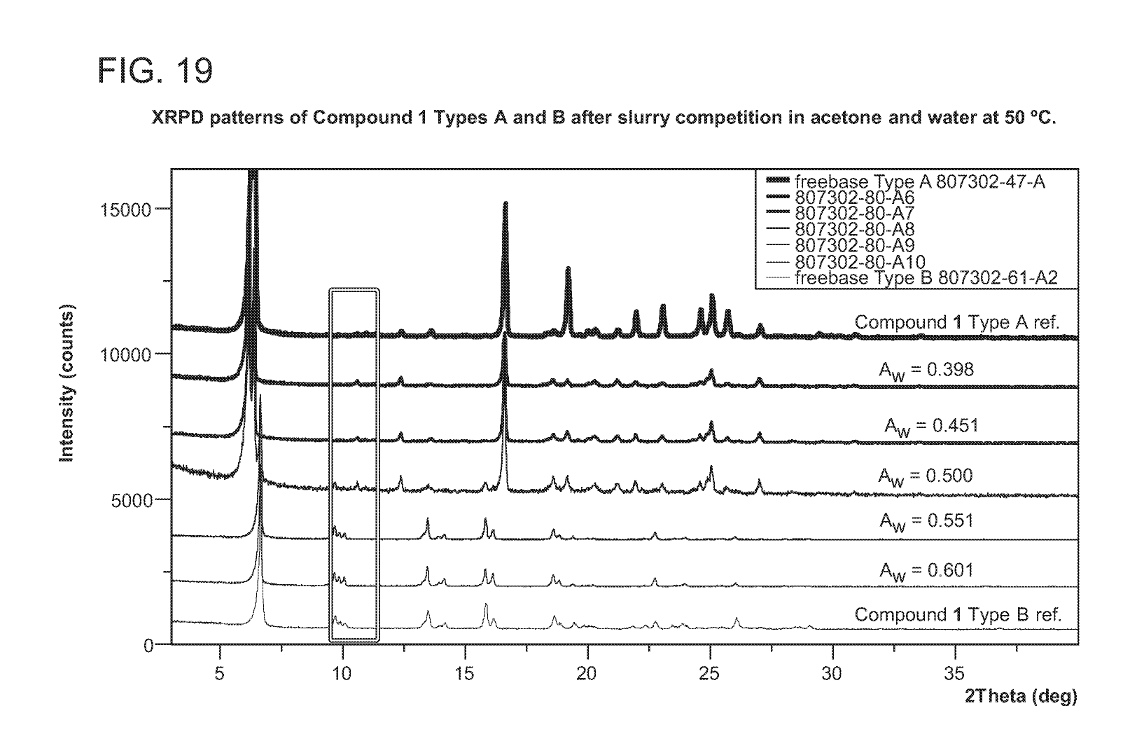

FIG. 19 provides XRPD patterns of Compound 1 Forms A and B after slurry competition in acetone and water at 50° C. The critical water activity between Compound 1 Forms Type A and B is in the range of 0.5˜0.55 at 50° C.

Critical water activity determination experiments in acetone and water are summarized in Table 50.

TABLE 50
Summary of critical water activity investigation experiments in acetone/water

| Temp. | A_{w} | Solid Form | Temp. | A_{w} | Solid Form |
|---|---|---|---|---|---|
| RT | 0.299 | Mixture of Compound 1 | 50° C. | — | — |
| (~20° C.) | | Type A and B* | | | |
| | 0.350 | Mixture of | | — | — |
| | | Compound 1 Type A and B | | | |
| | 0.398 | Mixture of Compound 1 | | 0.402 | Compound 1 Type A |
| | | Type A and B* | | | |
| | 0.451 | Compound 1 Type B | | 0.450 | Compound 1 Type A |
| | 0.500 | Compound 1 Type B | | 0.501 | Mixture of |
| | | | | | Compound 1 |
| | | | | | Type A and B |
| | 0.551 | Compound 1 Type B | | 0.551 | Compound 1 Type B |
| | 0.599 | Compound 1 Type B | | 0.599 | Compound 1 Type B |

*Increased Type A was observed at a_{w} of 0.299, while increased Type B obtained at a_{w} of 0.398.

In further studies, six more binary solvent systems were prepared with a_{w} of ˜0.5 (20° C.), including DMF, DMSO, THF, ACN, MeOH, and EtOH. As summarized in Table 51, Type B was obtained in THF/ACN/MeOH/EtOH systems, while Type A observed in DMF and DMSO systems.

TABLE 51
Summary of critical water activity investigation
experiments at room temperature

| Solvent (v/v) | A_{w} (20° C.) | Solid Form |
|---|---|---|
| DMSO/water (642:358) | 0.498 | Compound 1 Type A |
| THF/water (940:60) | 0.507 | Compound 1 Type B |
| ACN/water (967:33) | 0.548 | Compound 1 Type B |
| EtOH/water (896:104) | 0.501 | Compound 1 Type B |
| DMF/water (801:199) | 0.496 | Compound 1 Type A |
| MeOH/water (775:225) | 0.500 | Compound 1 Type B |

Further studies of form stability in DMSO and water were conducted:
A competitive slurry of Compound 1 Type A and B was performed in DMSO/water (v/v, 499:501) at 50° C., in which a_{w} equals 0.699. Compound 1 Type A was obtained after stirring overnight, suggesting the inconsistence of DMSO and water at both room temperature and 50° C.
No form change was observed after slurry of Compound 1 Type A in pure DMSO at room temperature, indicating negligible effect of DMSO solvate.

### Stability Investigation of Compound 1 Type B

To evaluate hygroscopicity and physical stability of Compound 1 Type B under different humidity, dynamic vapor diffusion (DVS) data were collected at 25° C. after pre-equilibrating the sample at ambient humidity (40% RH) until a mass change rate lower than 0.002%/min was obtained.

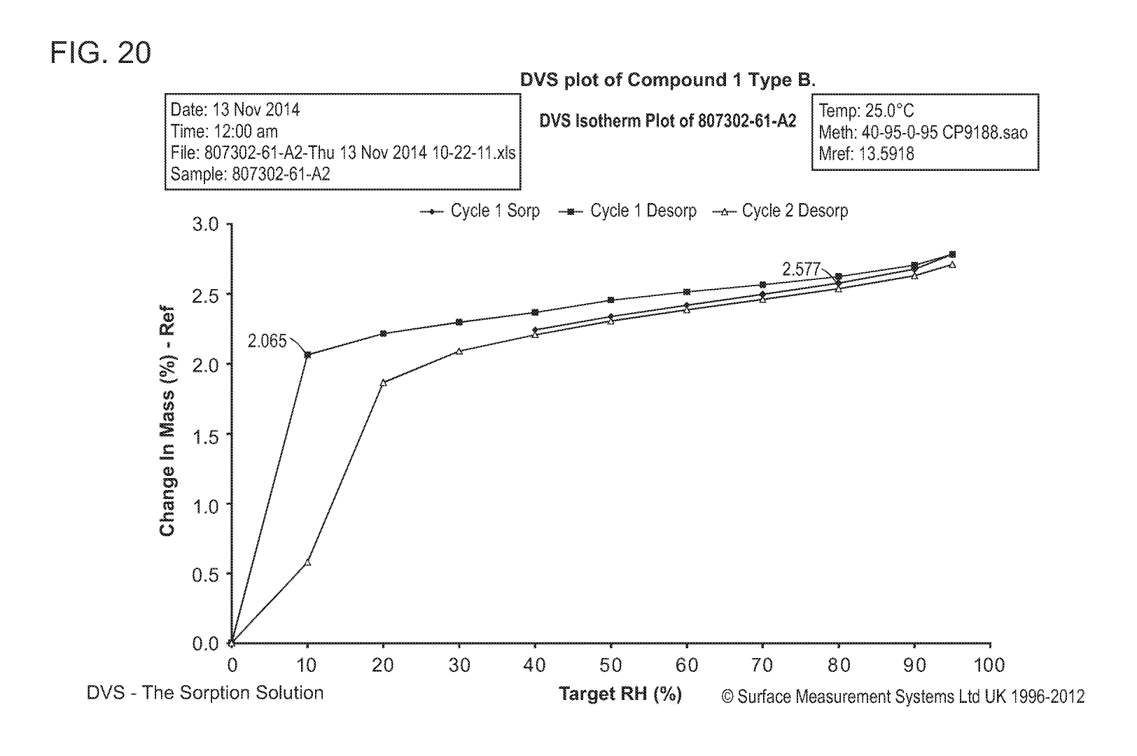

FIG. 20 provides the DVS plot of Compound 1 Type B and shows that Compound 1 Type B is slightly hygroscopic and tends to dehydrate under relative humidity (RH) less than 10%.

From XRPD studies, no form change of Compound 1 Type B was observed.

Further, Compound 1 Type B was vacuum dried at 80° C. for 15 hrs. Although this material conformed to Compound 1 Type B by the XRPD pattern, substantially decreased weight loss from 8.1% to 2.6% was observed from TGA while new phase transformation signals were observed. These data suggest that Compound 1 Type B is physically unstable at 80° C. (vacuum drying).

### Example 3: Initial Co-Former Screening

Compound 1 Type A was used as the starting material for co-former screening for the preparation of new solid forms. The mixtures of Compound 1 Type A and co-formers were stirred at different temperatures depending on the observation after mixing (Table 52):
1. Suspensions with obvious color change indicating possible reactions were stirred at room temperature.
2. Clear solutions were stirred at 4° C. to induce precipitation.
3. Suspensions with undissolved solids and no apparent color change were stirred at 50° C. to speed up the reaction.
4. Clear solutions obtained after stirring were allowed to evaporate slowly at room temperature in order to maximize the chances of identifying as many crystalline hits as possible.

TABLE 52
Temperature and experimental details of co-former screening

| | | Solvent | | | | | | | | | | | | | | | | |
|---|---|---|---|---|---|---|---|---|---|---|---|---|---|---|---|---|---|---|
| | | | | | | | | | | | | | | | | F | | |
| | | A | | B | | | C | | | D | | | E | | | MeOH/H₂O | | |
| Co-former | | Acetone | | ACN | | | THF | | | DCM | | | EtOH | | | (9:1, v/v) | | |
| 1 | hydrochloric acid | RT | P | RT | | P | RT | | P | 4° | C. | P | 4° | C. | P | 4° | C. | C |
| 2 | sulfuric acid | RT | P | RT | | P | RT | | P | RT | | P | 4° | C. | P | 4° | C. | C |
| 3 | methanesulfonic acid | RT | P | 4° | C. | P | RT | | P | 4° | C. | P | 4° | C. | P | 4° | C. | C |
| 4 | ethanedisulfonic acid | RT | P | RT | | P | RT | | P | RT | | P | RT | | P | 4° | C. | P |
| 5 | 2- | RT | P | RT | | P | RT | | P | 50° | C. | P | 4° | C. | P | 4° | C. | C |
| | hydroxyethanesulfonic | | | | | | | | | | | | | | | | | |
| | acid | | | | | | | | | | | | | | | | | |
| 6 | benzenesulfonic acid | RT | P | RT | | P | RT | | P | 4° | C. | P | 4° | C. | P | 4° | C. | C |
| 7 | toluenesulfonic acid | RT | P | 4° | C. | P | RT | | P | 4° | C. | P | 4° | C. | P | 4° | C. | C |
| 8 | 2-naphthalene sulfonic | RT | P | RT | | P | RT | | P | 4° | C. | P | 4° | C. | P | 4° | C. | C |
| | acid | | | | | | | | | | | | | | | | | |
| 9 | nitric acid | 4° C. | P | 4° | C. | P | 4° | C. | P | RT | | P | 4° | C. | P | 4° | C. | C |
| 10 | oxilic acid | RT | P | RT | | P | 4° | C. | C | 50° | C. | P | 4° | C. | C | 4° | C. | C |
| 11 | fumaric acid | 50° C. | C | 50° | C. | P | 4° | C. | P | 4° | C. | P | 4° | C. | P | 4° | C. | P |
| 12 | L-tartric acid | 50° C. | C | 50° | C. | P | 4° | C. | C | 4° | C. | P | 4° | C. | P | 4° | C. | C |
| 13 | citric acid | 50° C. | C | RT | | P | 4° | C. | C | 50° | C. | P | 4° | C. | P | 4° | C. | C |
| 14 | L-malic acid | 50° C. | P | 50° | C. | P | 4° | C. | C | 50° | C. | P | 4° | C. | P | 4° | C. | C |
| 15 | succinic acid | 50° C. | P | 50° | C. | P | 4° | C. | P | 50° | C. | P | 4° | C. | P | 4° | C. | C |
| 16 | hippuric acid | 50° C. | P | 50° | C. | P | 4° | C. | P | 50° | C. | P | 4° | C. | P | 4° | C. | C |
| 17 | maleic acid | 50° C. | C | RT | | P | 4° | C. | C | 50° | C. | P | 4° | C. | P | 4° | C. | C |
| 18 | glutamic acid | 50° C. | P | 50° | C. | P | 4° | C. | P | 4° | C. | P | 4° | C. | P | 4° | C. | P |
| 19 | benzoic acid | 50° C. | P | 50° | C. | P | 4° | C. | P | 4° | C. | P | 4° | C. | P | 4° | C. | C |
| 20 | gentisic acid | 50° C. | C | 50° | C. | P | 4° | C. | C | RT | | P | 4° | C. | P | 4° | C. | C |
| 21 | malonic acid | 50° C. | P | 50° | C. | P | 4° | C. | C | 4° | C. | P | 4° | C. | P | 4° | C. | C |
| 22 | cinnamic acid | 50° C. | P | 50° | C. | P | 4° | C. | C | 4° | C. | P | 4° | C. | P | 4° | C. | C |
| 23 | L-glutamide | 50° C. | P | 50° | C. | P | 50° | C. | P | 50° | C. | P | 50° | C. | P | 50° | C. | P |
| 24 | L-lysine | 50° C. | P | 50° | C. | P | 50° | C. | P | 50° | C. | P | 50° | C. | P | 50° | C. | P |
| 25 | L-phenylaline | 50° C. | P | 50° | C. | P | 50° | C. | P | 50° | C. | P | 50° | C. | P | 50° | C. | C |
| 26 | L-proline | 50° C. | P | 50° | C. | P | 50° | C. | P | 50° | C. | P | 50° | C. | C | 4° | C. | C |
| 27 | L-serine | 50° C. | P | 50° | C. | P | 50° | C. | P | 50° | C. | P | 50° | C. | P | 50° | C. | P |
| 28 | L-tyrosine | 50° C. | P | 50° | C. | P | 50° | C. | P | 50° | C. | P | 50° | C. | P | 50° | C. | P |
| 29 | nicotinamide | 50° C. | P | 50° | C. | P | 4° | C. | C | 50° | C. | P | 4° | C. | P | 4° | C. | C |
| 30 | nicotinic acid | 50° C. | P | 50° | C. | P | 50° | C. | P | 50° | C. | P | 50° | C. | C | 50° | C. | C |
| 31 | saccharin | 50° C. | P | 50° | C. | P | 4° | C. | P | 4° | C. | P | 50° | C. | P | 4° | C. | P |
| 32 | L-pyroglutamic Acid | 50° C. | P | 50° | C. | P | 50° | C. | C | 50° | C. | P | 4° | C. | P | 4° | C. | C |

P: precipitates observed.
C: clear solution observed and allowed to evaporate at RT.

Table 53 provides a summary of screening experiments for the identification of new solid forms that are obtained by the treatment of Compound 1 with a co-former.

TABLE 53
Solid forms obtained via co-former screening

| | | Solvent | | | | | |
|---|---|---|---|---|---|---|---|
| | | | | | | | F |
| | | A | B | C. | D | E | MeOH/H₂O |
| | | Acetone | ACN | THF | DCM | EtOH | (9:1, v/v) |
| Co-former | | New Solid Forms Obtained | | | | | |
| 1 | hydrochloric acid | Type A | Type A | Type A | — | Type A | Type A |
| 2 | sulphuric acid | Type A | Type A | Type B | Sulfate | — | Type C |
| | | | Type C | | Type C | | |
| 3 | methanesulfonic | Type A | Type A | Type A | — | Type A | — |
| | acid | | | | | | |
| 4 | ethanedisulfonic | Type A | Type A | Type A | Type A | Type A | Type A |
| | acid | | | | | | |
| 5 | 2-hydroxyethane- | Type A | Type A | Type A | Type A | — | — |
| | sulphonic acid | | | | | | |
| 6 | benzenesulfonic | Type A | Type A | Type A | — | Type B | — |
| | acid | | | | | | |
| 7 | toluenesulfonic | Type B | Type A | Type A | Type B | Type C | — |
| | acid | | | | | | |
| 8 | 2-naphthalene | Type A | Type A | Type A | Type B | Type B | Type A |
| | sulfonic acid | | | | FB Type B | | |
| 9 | nitric acid | Type A | Type A | Type B | Type A | Type A | Type C |
| 10 | oxilic acid | Type A | Type A | Type C | Oxalate | Type A | Type B |
| | | | | | Type B | Type B | |
| 11 | fumaric acid | Type A | Type A | Type A | Type A + | Type A | Type A |
| | | FB Type A | FB Type A | | Fumaric | FB Type A | FB Type A |
| | | | | | acid | Fumaric | Fumaric |
| | | | | | | acid | acid |
| 12 | L-tartric acid | Type A | Type A | Type A | — | — | Type A |
| | | FB Type A | | | | | |
| 13 | citric acid | Type B | Type A | Type B | Type A | FB Type A | Type C |
| | | | | | FB Type A | | |
| 14 | L-malic acid | — | — | Type A | — | — | FB Type A + |
| | | | | | | | FB Type C |
| 15 | succinic acid | — | — | Type A | — | — | — |
| 16 | hippuric acid | — | — | — | — | — | Type A + |
| | | | | | | | hippuric |
| | | | | | | | acid |
| 17 | maleic acid | Type B | Type A | Type B | Type A | Type B | Type B |
| | | | FB Type A | | | | FB Type C |
| 18 | glutamic acid | — | — | — | — | — | — |
| 19 | benzoic acid | — | — | — | — | — | — |
| 20 | gentisic acid | — | — | — | Type A | — | Type A |
| | | | | | | | FB Type C |
| 21 | malonic acid | — | — | — | — | — | — |
| 22 | cinnamic acid | — | — | — | — | — | — |
| 23 | L-Glutamide | — | — | — | — | — | — |
| 24 | L-Lysine | — | — | — | — | — | — |
| 25 | L-Phenylaline | — | — | — | — | — | — |
| 26 | L-Proline | — | — | — | — | — | — |
| 27 | L-Serine | — | — | — | — | — | — |
| 28 | L-Tyrosine | Type A | Type A | Type A | Type A | Type A | Type A |
| | | FB Type A | FB Type A | | | | |
| 29 | nicotinamide | — | — | — | — | — | — |
| 30 | nicotinic acid | — | — | — | — | — | — |
| 31 | saccharin | — | — | Saccharinate | Saccharinate | Saccharinate | Saccharinate |
| | | | | Type B | Type C | Type D | Type A |
| 32 | L-Pyroglutamic | — | — | — | — | — | — |
| | Acid | | | | | | |

* — indicates that a new solid form was not obtained from the experiment.
** Where a new solid form was produced along with Compound 1, Compound 1 is represented by “FB” in the entry

Crystalline solid forms obtained from these initial experiments were characterized by TGA and DSC. Stoichiometry was determined by HPLC/IC, ¹H NMR, or HPLC. The characterization results are summarized in Table 54.

TABLE 54
Summary of characterization of crystalline solid forms

| | | | | | Molar |
|---|---|---|---|---|---|
| | | | | | ratio |
| | | Safety | Wt | Endotherm | (acid/ |
| Solid Form | | Class | Loss/% | (° C., onset) | base) |
| HCl | Type A | I | 2.0 | 128.5 | 0.92 |
| (Compound 2) | | | | | |
| Sulfate | Type A | I | 1.9 | 169.5 | 0.91 |
| (Compound 3) | Type B | | 1.0 | 179.0 | 0.99 |
| | Type C | | 3.4 | 131.3, 166.8 | 0.89 |
| Mesylate | Type A | II | 1.8 | 171.1 | 0.78 |
| (Compound 4) | | | | | |
| Edisylate | Type A | II | 4.0 | 63.4, 197.6 | 0.98 |
| (Compound 5) | | | | | |
| Isethionate | Type A | II | 1.8 | 69.8*, 163.5 | 0.98 |
| (Compound 6) | | | | | |
| Besylate | Type A | II | 1.5 | 168.4 | 0.76 |
| (Compound 7) | Type B | | 3.3 | 71.1*, 165.9* | 0.87 |
| Tosylate | Type A | II | 0.8 | 159.2 | 0.89 |
| (Compound 8) | Type B | | 0.9 | 162.4 | 0.92 |
| | Type C | | 1.4 | 64.1, 171.2 | 0.91 |
| Napsylate | Type A | II | 2.1 | 75.7*, 174.0 | 1.00 |
| (Compound 9) | Type B | | 1.2 | 177.8 | 0.99 |
| Nitrate | Type A | III | 0.5 | N/A | 1.02 |
| (Compound 10) | Type B | | 0.6 | N/A | 0.98 |
| | Type C | | 1.5 | 107.6* | 1.07 |
| Oxalate | Type A | III | 3.2 | 91.7*, 139.7*, | 1.72 |
| (Compound 11) | | | | 162.2* | |
| | Type B | | 2.0 | 97.8 | 1.56 |
| | Type C | | 3.9 | 75.7, 116.0, | 1.51 |
| | | | | 136.5 | |
| Fumarate | Type A | I | 12.7 | 77.9 | 0.93 |
| (Compound 12) | | | | | |
| L-tartrate | Type A | I | 9.5 | 74.5 | 0.98 |
| (Compound 13) | | | | | |
| Citrate | Type A | I | 0.7 | 139.4 | 2.00 |
| (Compound 14) | Type B | | 4.5 | 100.1 | 1.09 |
| L-malate | Type A | I | 8.5 | 61.2 | 1.06 |
| (Compound 15) | | | | | |
| Succinate | Type A | I | 7.0 | 67.5 | 0.99 |
| (Compound 16) | | | | | |
| Maleate | Type A | I | 0.4 | 149.6 | 1.06 |
| (Compound 18) | Type B | | 1.3 | 140.4 | 0.90 |
| Gentisate | Type A | II | 3.7 | 112.9 | 1.04 |
| (Compound 21) | | | | | |
| Saccharinate | Type A | I | 2.9 | 150.5 | 0.89 |
| (Compound 32) | Type B | | 13.2 | 69.9*, 76.2*, | 0.83 |
| | | | | 86.3*, 152.7 | |
| | Type C | | 1.3 | 59.3*, 126.5 | 0.80 |
| | Type D | | 3.3 | 153.3 | 0.93 |

*peak temperature

### Example 4: Compound 2

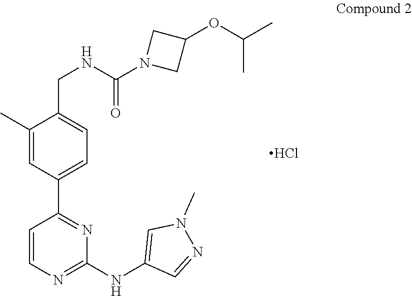 [SMILES: Cc1cc(-c2ccnc(Nc3cnn(C)c3)n2)ccc1CNC(=O)N1CC(OC(C)C)C1 ; Cl]

### Compound 2 Type A

From the initial screen using hydrochloric acid, one solid form (Compound 2 Type A) was obtained.

FIG. 21 provides the XRPD pattern of Compound 2 Type A.

FIG. 22 provides the TGA and DSC curves of Compound 2 Type A. DSC/TGA data (FIG. 7-2) identified an endotherm at 128.5° C. (onset temperature) and a weight loss of 2.0% up to 100° C.

Based on the solid-state characterization results, Compound 2 Type A was scaled up to about one gram using the following preparation procedure.
1. Weigh 1.0 g of Compound 1 into a 20-mL glass vial.
2. Add 15 mL of THF and 191 μL of conc. HCl (37.5%) to the vial.
3. Stir the mixture with a magnetic stir bar (800 rpm) at RT for 20 hrs.
4. Vacuum filter and dry the wet cake at 50° C. for 5 hrs.
5. Collect solids (1.0 g obtained with yield of ˜92.3%).

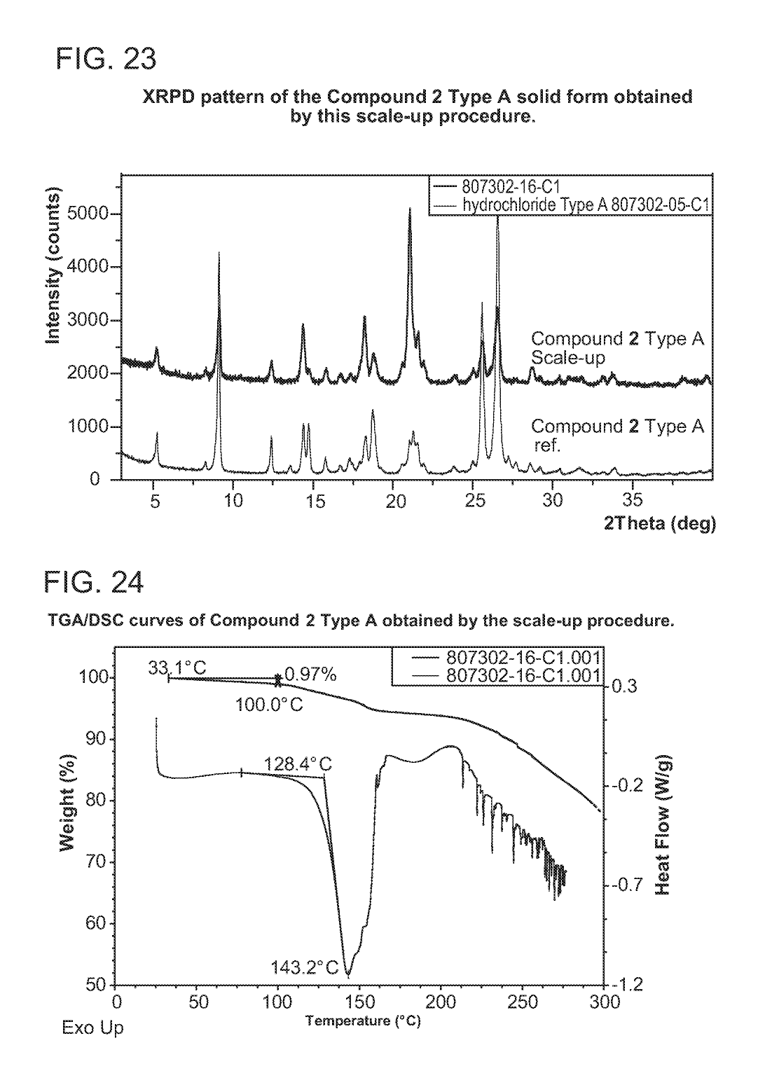

FIG. 23 provides the XRPD pattern of the Compound 2 Type A solid form obtained by this scale-up procedure.

FIG. 24 provides the TGA/DSC curves of Compound 2 Type A obtained by the scale-up procedure. TGA/DSC data showed a weight loss of 1.0% up to 100° C. and an endotherm at 128.4° C. (onset temperature) possibly associated with melting/decomposition.

### Stoichiometry of Compound 2 Type A

The starting material Compound 2 Type A was suspended in different solvent systems, and the remaining solids with improved crystallinity were isolated for further investigation. Results summarized in Table 55 indicate that the stoichiometry of Compound 2 Type A is 1:1.

TABLE 55
Summary of molar ratio results for Compound 2 Type A batches

| | Molar Ratio |
|---|---|
| Preparation Method | (acid/base) |
| Reaction crystallization | 0.88 |
| Slurry in EtOH at 50° C. | 0.93 |
| Slurry in MeOH/THF (v/v, 1:5) at 50° C. | 0.96 |
| Slurry in DMAc/toluene (v/v, 1:5) at 50° C. | 0.96 |

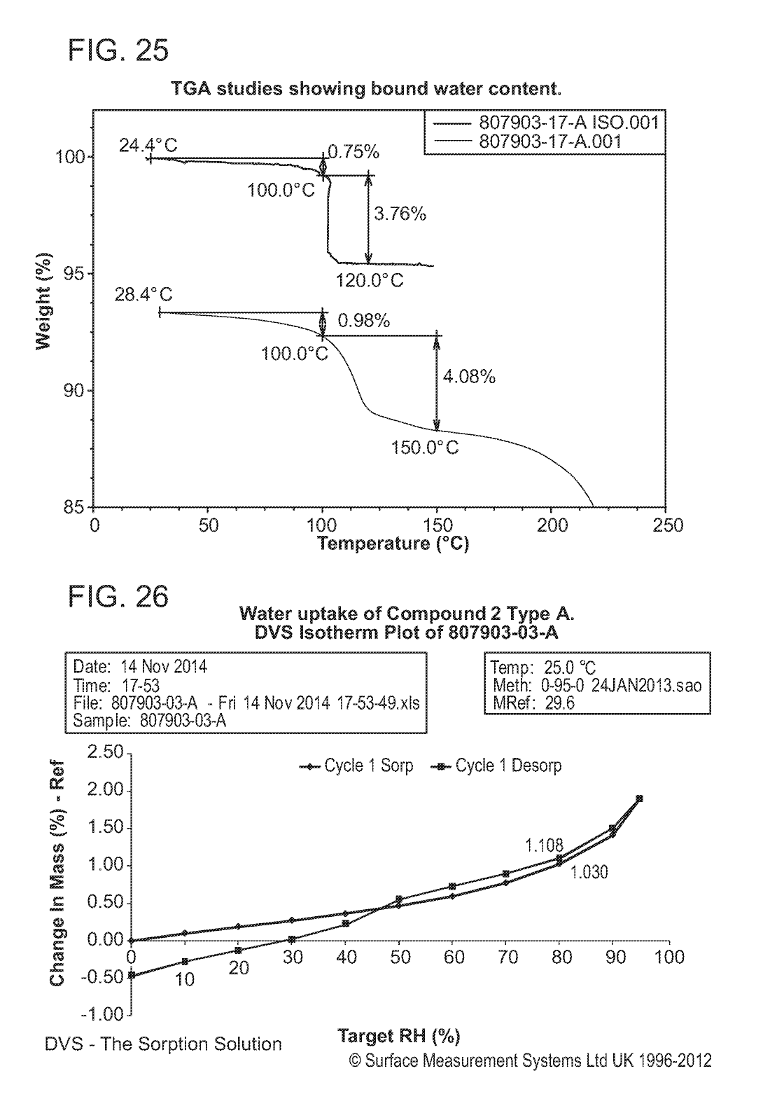

To further investigate the weight loss and thermal change observed in the temperature range of 100˜150° C., Compound 2 Type A—obtained via slurry of starting Compound 2 Type A in EtOH at 50° C.—was further characterized by Karl-Fisher titration (KF) and stepwise isothermal TGA. Stepwise isothermal TGA was applied aiming to separate the unbound and bound water. Result from Karl Fisher testing indicated a water content of 4.3% (Table 56), consistent with the TGA results of bound water content was 3.8% from two-step weight loss before 150° C. (FIG. 25). These results are further supported by the observation of yellow gel after dehydration. This suggests that Compound 2 Type A is a monohydrate (calculated water content of a monohydrate is 3.7%).

TABLE 56
Water content of Compound 2 Type A

| | | Averaged Water |
|---|---|---|
| Mass/mg | Water Content/% | Content/% |
| 39.49 | 4.29 | 4.3 |
| 41.41 | 4.39 | |

To evaluate the hygroscopicity and physical stability of Compound 2 Type A under increased humidity, DVS isotherm data was collected at 25° C. with the starting sample pre-dried at 0% RH until a mass change rate lower than 0.002%/min. Compound 2 Type A appears to be slightly hygroscopic, with a water uptake of 1.0% at 25° C./80% RH (FIG. 26). After the DVS test, no form change was observed by XRPD.

### Screening Experiments Using Compound 2 Type A

The solubility of starting Compound 2 Type A was estimated in 21 solvents at room temperature (RT, 20±3° C.). Approximately 2 mg solids were added into a 3-mL glass vial. Solvents in Table 4-6 were then added stepwise (100 μL per step) into the vials until the solids were dissolved or a total volume of 2 mL was reached. Results are summarized in Table 57, which were used to guide the solvent selection in polymorph screening.

TABLE 57
Solubility of Compound 2 Type A at room temperature

| Solvent | Solubility (mg/mL) | Solvent | Solubility (mg/mL) |
|---|---|---|---|
| MeOH | S >34.0 | 2-MeTHF | S <1.0 |
| EtOH | 4.4 < S < 5.5 | 1,4-dioxane | S <1.0 |
| IPA | 1.7 < S < 1.8 | NMP | 14.7 < S < 22.0 |
| ACN | S <1.0 | DMSO | 14.0 < S < 21.0 |
| Acetone | S <0.9 | CHCl₃ | S <1.1 |
| MIBK | S <1.0 | Toluene | S <1.1 |
| EtOAc | S <1.0 | n-heptane | S <0.9 |
| IPAc | S <1.2 | DMAc | 22.0 < S < 44.0 |
| MTBE | S <1.0 | H₂O | N/A |
| THF | S <0.9 | DCM | S <1.1 |

N/A: disproportionation occurred along with obvious color change of solids from orange to faint yellow.

Polymorph screening experiments were performed using different solution crystallization or solid transition methods. The methods utilized and crystal forms identified are summarized in Table 58.

TABLE 58
Summary of polymorph screening experiments

| | No. of | |
|---|---|---|
| Method | Experiments | Isolated Crystal Forms |
| Anti-solvent Addition | 24 | Compound 2 Type A |
| Slow Evaporation | 11 | Compound 2 Type A, B |
| Slow Cooling | 9 | Compound 2 Type A |
| Slurry Conversion at RT/50° C. | 29 | Compound 2 Type A |
| Solid Vapor Diffusion | 13 | Compound 2 Type A |
| Solution Vapor Diffusion | 13 | Compound 2 Type A |
| Polymer-induced Crystallization | 8 | Compound 2 Type |
| | | A, B, C |
| Grinding | 2 | Compound 2 Type A |
| Total | 109 | Compound 2 Type |
| | | A, B, C |

### Compound 2 Type B

In screening experiments, Compound 2 Type B was obtained by crystallization from solvent systems containing methanol (Table 59). Slow evaporation experiments were performed under 11 conditions. Briefly, ˜15 mg of Compound 2 Type A sample were dissolved in 0.3-4.0 mL of corresponding solvent in a 3-mL glass vial. The visually clear solutions were subjected to evaporation at room temperature to induce precipitation. The solids were isolated for XRPD analysis to identify any new solid forms.

TABLE 59
Summary of slow evaporation experiments

| Solvent (v/v) | Solid Form |
|---|---|
| MeOH | Compound 2 Type A + |
| | Compound 2 Type B |
| EtOH | Compound 2 Type A |
| IPA | Compound 2 Type A |
| DCM | Compound 2 Type A |
| CHCl₃ | Compound 2 Type A |
| MeOH/MIBK (4:1) | Compound 2 Type B |
| MeOH/toluene (4:1) | Compound 2 Type A + |
| | Compound 2 Type B |
| MeOH/EtOAc (4:1) | Compound 2 Type A |
| MeOH/1,4-dioxane (4:1) | Compound 2 Type A |
| MeOH/CHCl₃ (4:1) | Compound 2 Type B |
| MeOH/ACN (4:1) | Compound 2 Type A + |
| | Compound 2 Type B |

A sample of Compound 2 Type B reference was generated via evaporation of a solution in MeOH/MIBK (v/v, 4:1) at room temperature. By XRPD, Compound 2 Type B converted into Compound 2 Type A during air drying at ambient conditions. As the sample was still wet after form transformation, Compound 2 Type B is suspected to be a metastable form at ambient conditions.

FIG. 27 provides an overlay of XRPD data Compound 2 Type B before and after storage and as compared to Compound 2 Type A.

### Compound 2 Type C

Polymer-induced crystallization experiments were performed with two sets of polymer mixtures in four different solvents. Approximate 15 mg of Compound 2 Type A were dissolved in 0.3-2.5 mL of an appropriate solvent to obtain a clear solution in a 3-mL vial. About 2 mg of polymer mixture were added into 3-mL glass vial. All the samples were sealed using parafilm and then transferred to evaporation at room temperature to induce precipitation. The solids were isolated for XRPD analysis. Results are summarized in Table 60.

TABLE 60
Summary of polymer-induced crystallization experiments

| Solvent (v/v) | Polymer Mixture | Solid Form |
|---|---|---|
| EtOH | A | Compound 2 Type A |
| CHCl₃ | | amorphous |
| MeOH/toluene (4:1) | | Compound 2 Type A |
| MeOH/EtOAc (4:1) | | Compound 2 Type A |
| EtOH | B | Compound 2 Type A + |
| | | Compound 2 Type C |
| CHCl₃ | | amorphous |
| MeOH/toluene (4:1) | | Compound 2 Type A + |
| | | Compound 2 Type B |
| MeOH/EtOAc (4:1) | | Compound 2 Type A |

Polymer mixture A: polyvinyl pyrrolidone (PVP), polyvinyl alcohol (PVA), polyvinylchloride (PVC), polyvinyl acetate (PVAC), hypromellose (HPMC), methyl cellulose (MC) (mass ratio of 1:1:1:1:1:1)
Polymer mixture B: polycaprolactone (PCL), polyethylene glycol (PEG), poly(methyl methacrylate) (PMMA) sodium alginate (SA), and hydroxyethyl cellulose (HEC) (mass ratio of 1:1:1:1:1).

Compound 2 Type C was crystallized from a solution in the ethanol by addition of polymer mixture B at ambient conditions. Based on XRPD comparison with Compound 2 Type A, new X-ray diffraction peaks assigned to a possible new form pattern, assigned as Compound 2 Type C (FIG. 28).

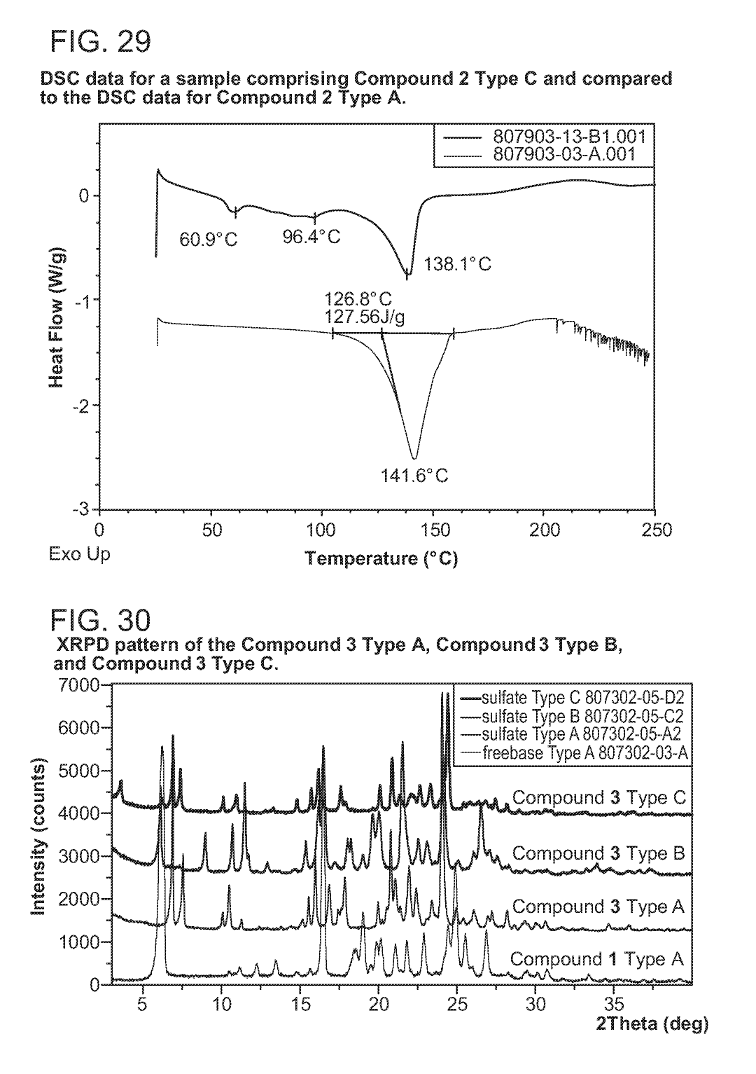

FIG. 29 provides DSC data for a sample comprising Compound 2 Type C and compared to the DSC data for Compound 2 Type A. This mixture demonstrated two endotherms at 60.9° C. and 96.4° C. (peak temperature) by DSC before the dehydration/melting of Compound 2 Type A. The new diffraction peaks associated with Type C disappeared after heating to 100° C. This suggests that Type C could have converted into Type A or amorphous material after heating.

Subsequent experiments for the re-preparation of Compound 2 Type C did afford the desired product.

### Example 5: Compound 3

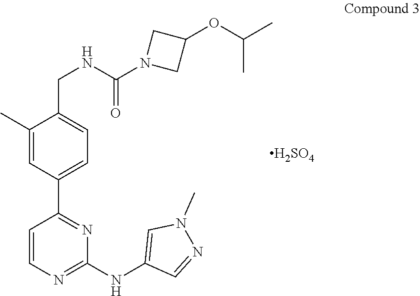 [SMILES: Cc1cc(-c2ccnc(Nc3cnn(C)c3)n2)ccc1CNC(=O)N1CC(OC(C)C)C1 ; "·H2SO4"]

Three sulfate solid forms of Compound 3 Type A, Compound 3 Type B, and Compound 3 Type C were obtained from screening.

### Compound 3 Type A

FIG. 30 provides the XRPD pattern of the Compound 3 Type A, Compound 3 Type B, and Compound 3 Type C.

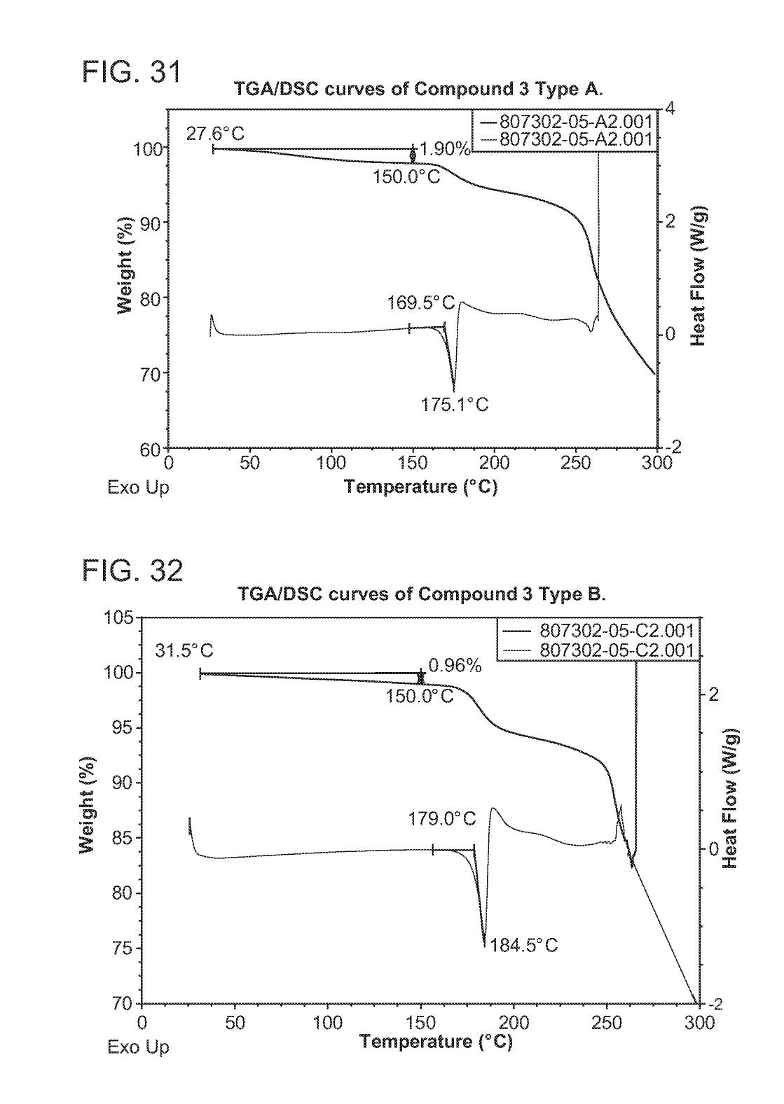

FIG. 31 provides the TGA/DSC curves of Compound 3 Type A. The figure shows an endotherm (possibly melting/decomposition) at 169.5° C. (onset temperature) and a weight loss of 1.9% up to 150° C.

### Compound 3 Type B

FIG. 32 provides the TGA/DSC curves of Compound 3 Type B. The data showed an endotherm (possibly melting/decomposition) at 179.0° C. (onset temperature) and a weight loss of 1.0% up to 150° C.

Based on the solid-state characterization results, Compound 3 Type B was scaled up to about one gram using the following preparation procedure.
1. Weigh 1.0 g of freebase into a 20-mL glass vial.
2. Add 20 mL of THF and 125 μL of conc. H₂SO₄ (98%) to the vial.
3. Stir the mixture with a magnetic stir bar (800 rpm) at RT for 50 hrs.
4. Vacuum filter and dry the wet cake at 50° C. for 5 hrs.
5. Collect solids (1.2 g obtained with yield of ˜98.0%).

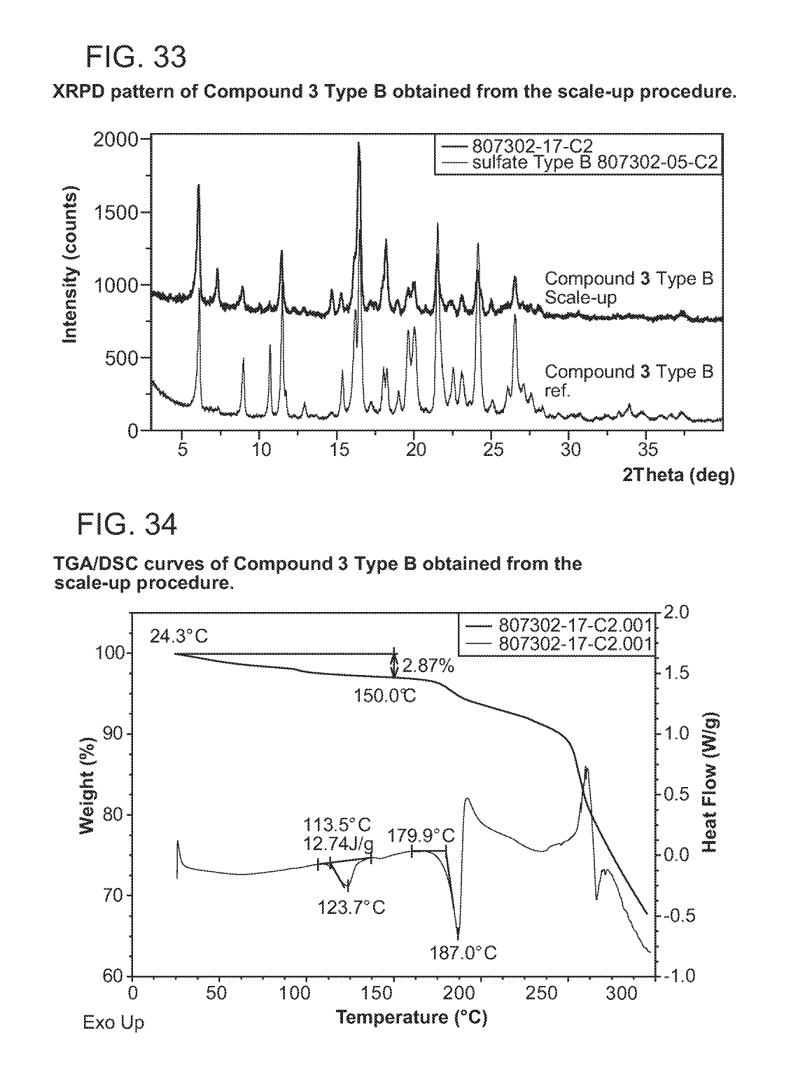

FIG. 33 provides the XRPD pattern of Compound 3 Type B obtained from the scale-up procedure.

FIG. 34 provides the TGA/DSC curves of Compound 3 Type B. The data showed a weight loss of 2.9% up to 150° C. and an endotherm at 113.5° C. (onset temperature) followed by another endotherm (possibly melting/decomposition) at 179.9° C. (onset temperature). HPLC/IC result determined a stoichiometry of 1.03 (acid/base) for the scaled up sulfate Type B (807302-17-C2).

### Compound 3 Type C

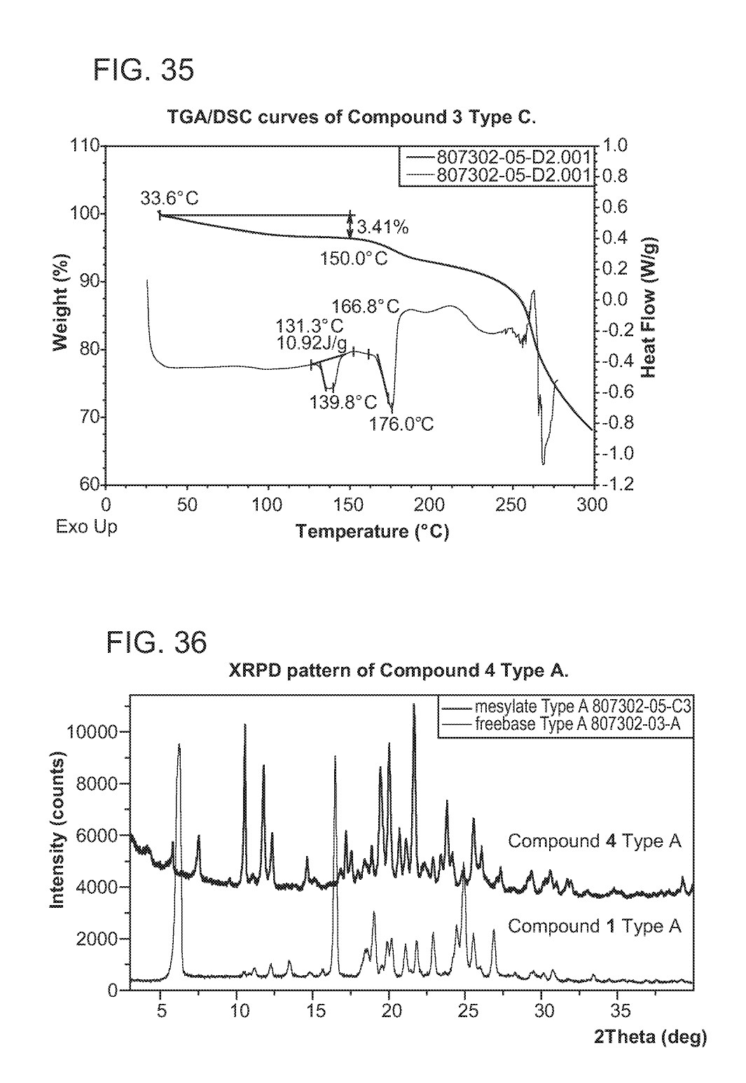

FIG. 35 provides the TGA/DSC curves of Compound 3 Type C. The data show an endotherm at 131.3° C. followed by another endotherm (possibly melting/decomposition) at 166.8° C. (onset temperature). A weight loss of 3.4% up to 150° C. was observed.

### Example 6: Compound 4

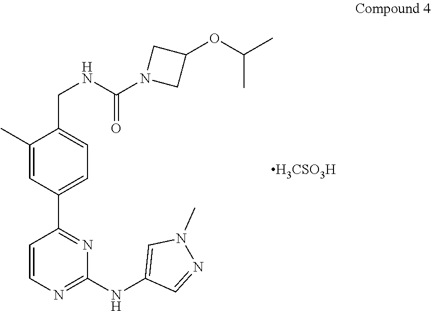 [SMILES: Cc1cc(-c2ccnc(Nc3cnn(C)c3)n2)ccc1CNC(=O)N1CC(OC(C)C)C1 ; "·H3CSO3H"]

### Compound 4 Type A

FIG. 36 provides the XRPD pattern of Compound 4 Type A.

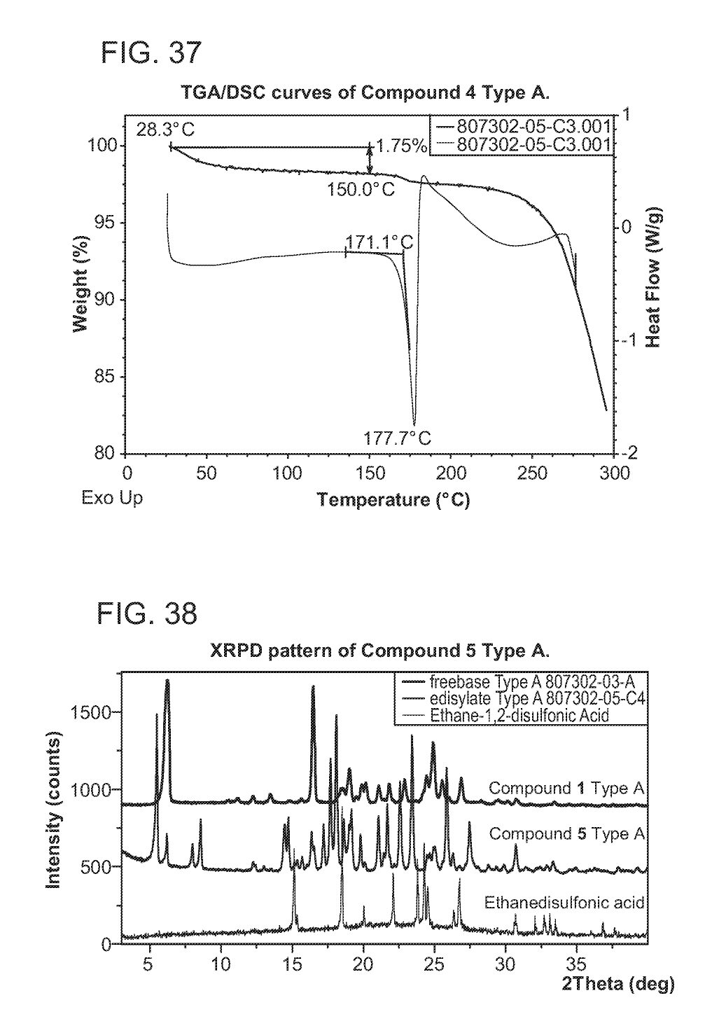

FIG. 37 provides the TGA/DSC curves of Compound 4 Type A. DSC/TGA data (FIG. 7-8) showed an endotherm (possibly melting/decomposition) at 171.1° C. (onset temperature) and a weight loss of 1.8% up to 150° C.

### Example 7: Compound 5

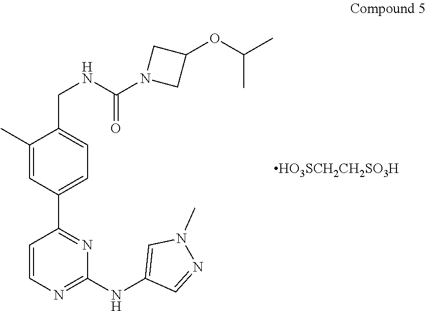

FIG. 38 provides the XRPD pattern of Compound 5 Type A.

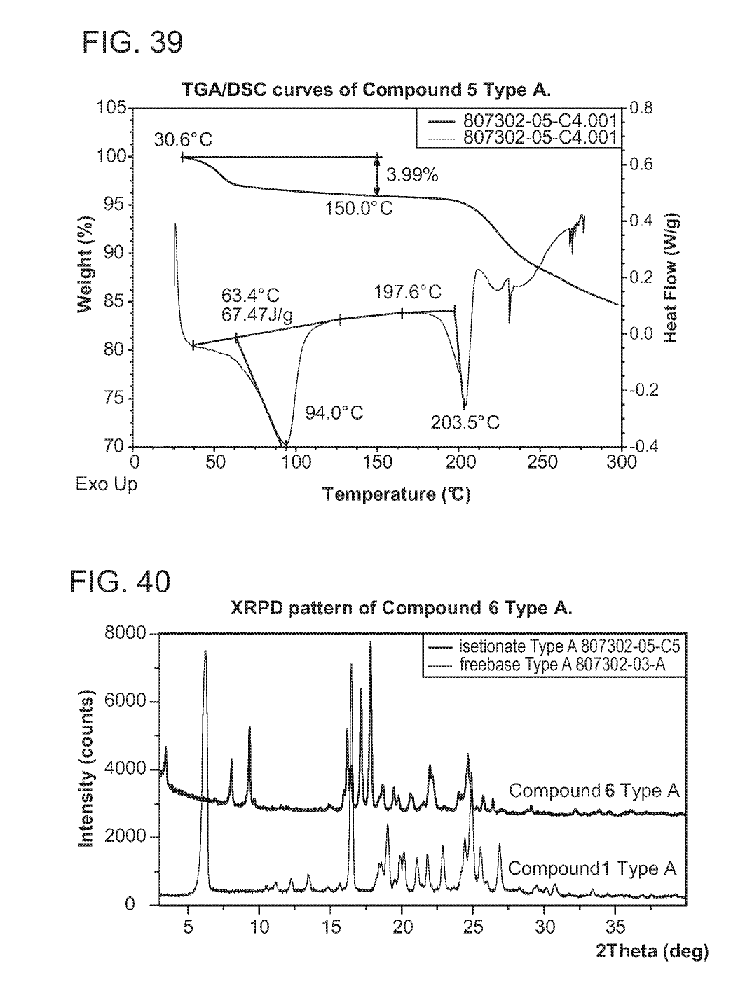

FIG. 39 provides the TGA/DSC curves of Compound 5 Type A. DSC/TGA data showed an endotherm at 63.4° C. followed by another endotherm (possibly melting/decomposition) at 197.6° C. (onset temperature). A weight loss of 4.0% up to 150° C. was observed.

¹H NMR data indicated a stoichiometry of 0.98 (acid/base) Compound 5 Type A.

### Example 8: Compound 6

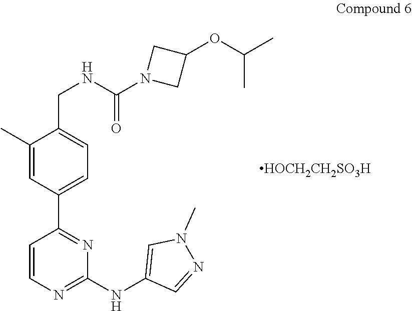 [SMILES: Cc1cc(-c2ccnc(Nc3cnn(C)c3)n2)ccc1CNC(=O)N1CC(OC(C)C)C1 ; "·HOCH2CH2SO3H"]

FIG. 40 provides the XRPD pattern of Compound 6 Type A.

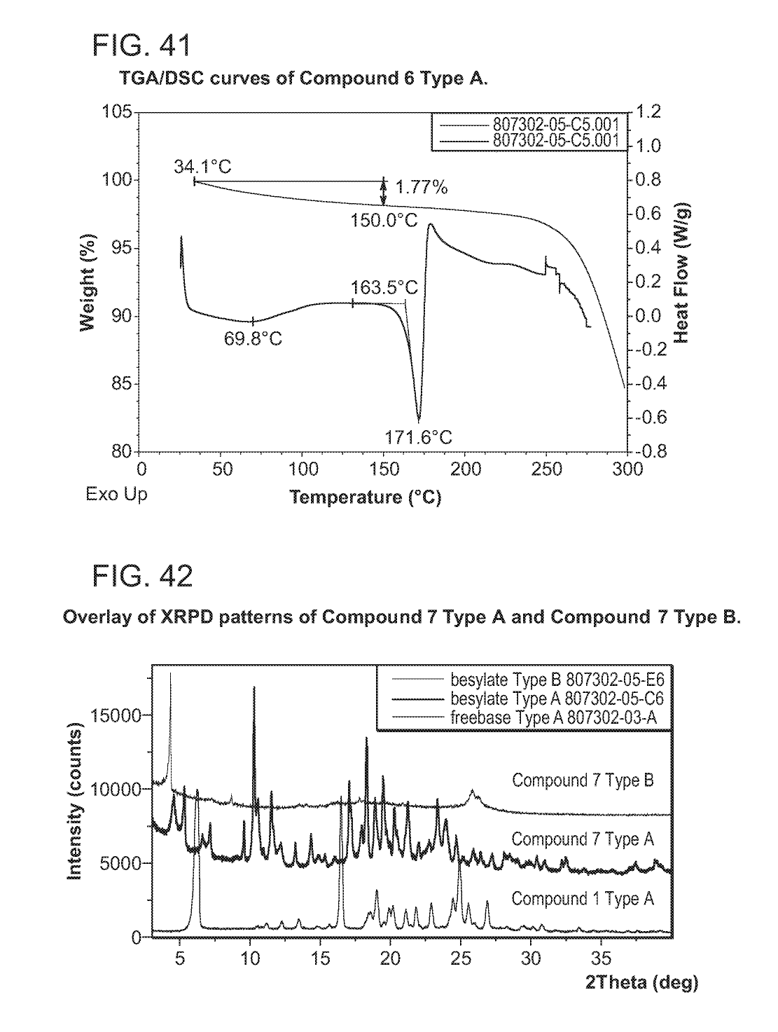

FIG. 41 provides the TGA/DSC curves of Compound 6 Type A. DSC/TGA data showed an endotherm (possibly melting/decomposition) at 163.5° C. (onset temperature) and a weight loss of 1.8% up to 150° C.

¹H NMR indicated a stoichiometry of 0.98 (acid/base) for Compound 6 Type A.

### Example 9: Compound 7

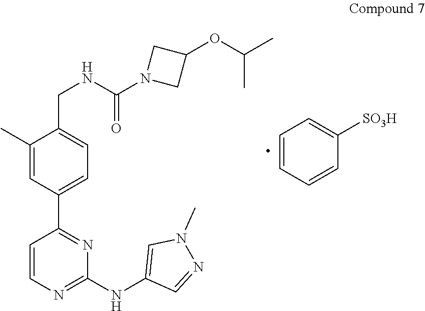 [SMILES: Cc1cc(-c2ccnc(Nc3cnn(C)c3)n2)ccc1CNC(=O)N1CC(OC(C)C)C1 ; O=S(=O)(O)c1ccccc1]

FIG. 42 provides an overlay of XRPD patterns of Compound 7 Type A and Compound 7 Type B.

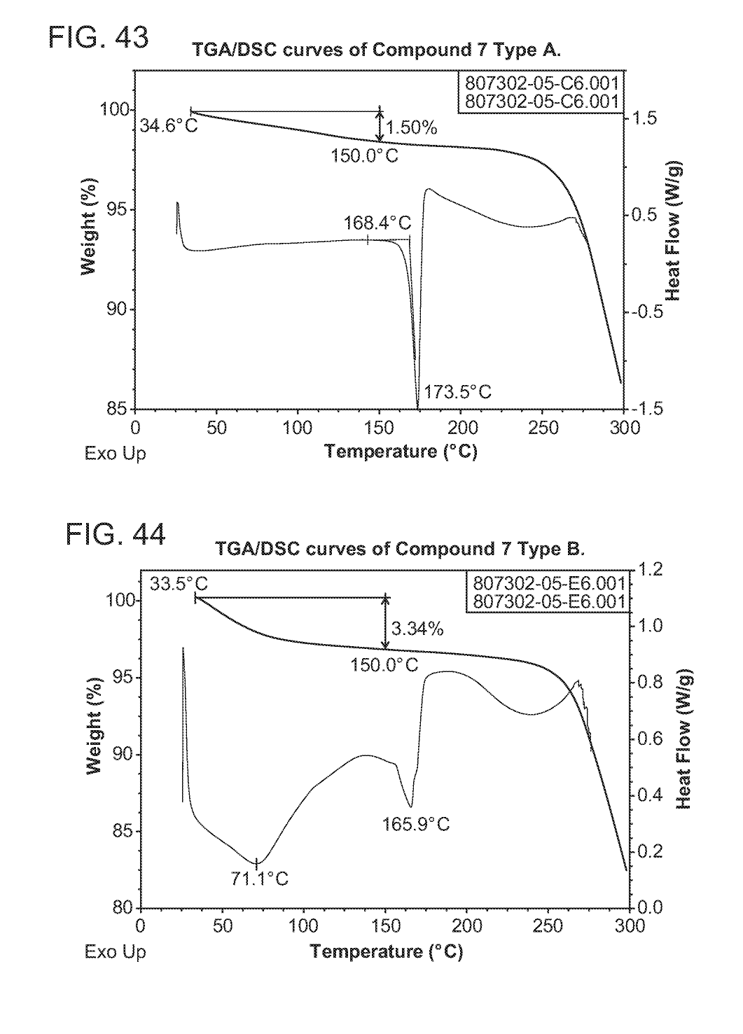

FIG. 43 provides TGA/DSC curves of Compound 7 Type A. The data showed an endotherm (possibly melting/decomposition) at 168.4° C. (onset temperature) and a weight loss of 1.5% up to 150° C.

¹H NMR data indicated a stoichiometry of 0.76 (acid/base) for Compound 7 Form A.

FIG. 44 provides TGA/DSC curves of Compound 7 Type B. The DSC/TGA data showed an endotherm (possibly melting/decomposition) at 165.9° C. (peak temperature) and a weight loss of 3.3% up to 150° C.

¹H NMR data indicated a stoichiometry of 0.87 (acid/base) for Compound 7 Form B.

### Example 10: Compound 8

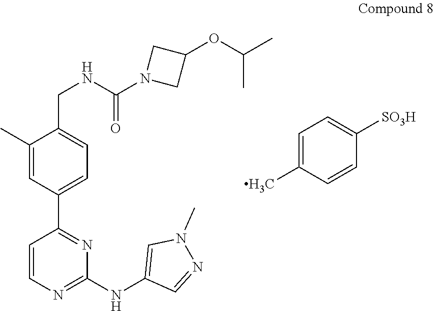

FIG. 45 provides an overlay of XRPD patterns of Compound 8 Types A-C, Compound 1 Type A, and toluenesulfonic acid.

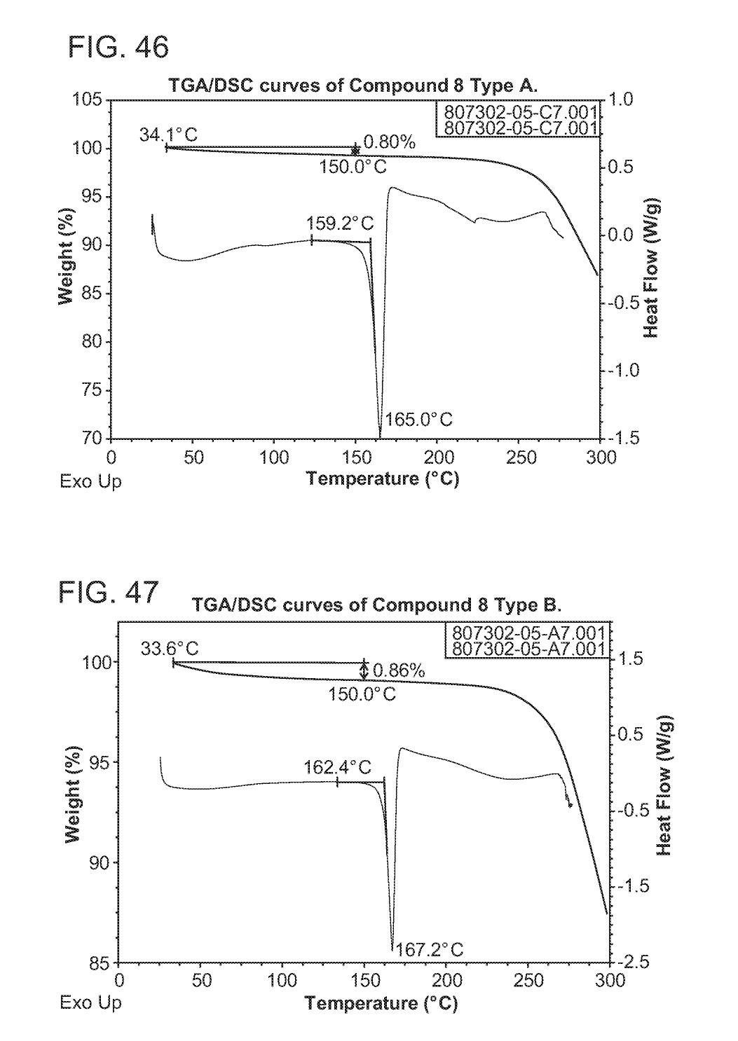

FIG. 46 provides TGA/DSC curves of Compound 8 Type A. The data showed an endotherm (possibly melting/decomposition) at 159.2° C. (onset temperature) and a weight loss of 0.8% up to 150° C. ¹H NMR data indicated a stoichiometry of 0.89 (acid/base) for Compound 8 Type A.

FIG. 47 provides TGA/DSC curves of Compound 8 Type B. The data showed an endotherm (possibly melting/decomposition) at 162.4° C. (onset temperature) and a weight loss of 0.9% up to 150° C. ¹H NMR data indicated a stoichiometry of 0.92 (acid/base) for Compound 8 Type B.

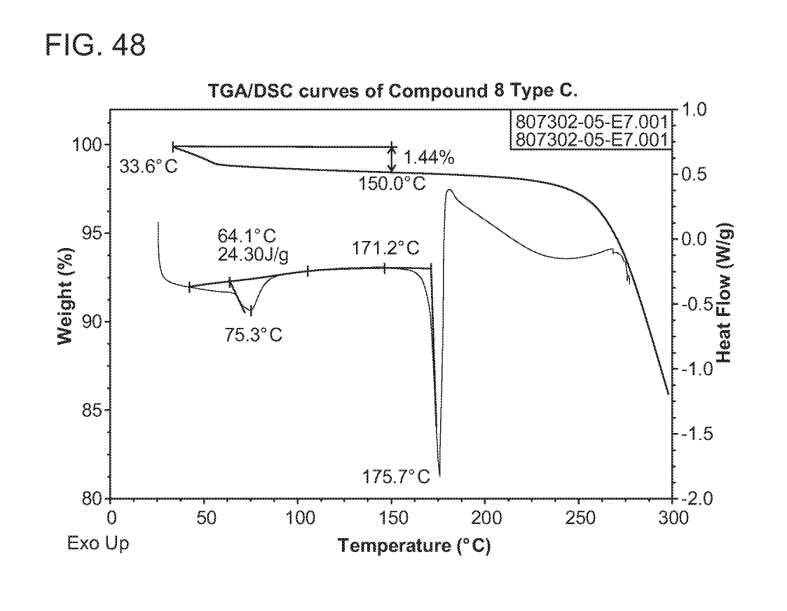

FIG. 48 provides TGA/DSC curves of Compound 8 Type C. The data showed an endotherm at 64.1° C. followed by another endotherm (possibly melting/decomposition) at 171.2° C. (onset temperature). A weight loss of 1.4% up to 150° C. was observed. ¹H NMR data indicated a stoichiometry of 0.91 (acid/base) for Compound 8 Type C.

### Example 11: Compound 9

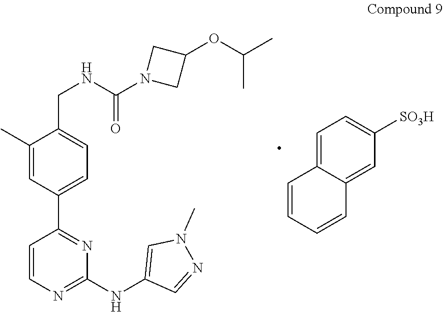 [SMILES: Cc1cc(-c2ccnc(Nc3cnn(C)c3)n2)ccc1CNC(=O)N1CC(OC(C)C)C1 ; O=S(=O)(O)c1ccc2ccccc2c1]

FIG. 49 provides an overlay of XRPD patterns of Compound 9 Type A and Type B.

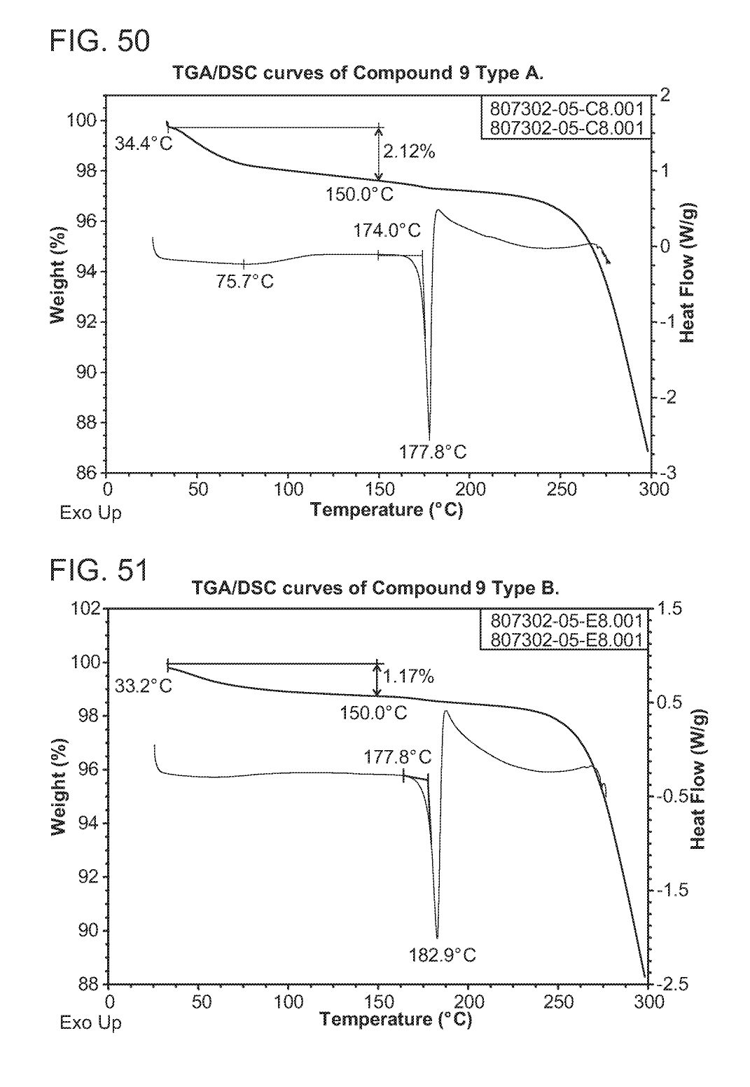

FIG. 50 provides TGA/DSC curves of Compound 9 Type A. DSC/TGA data showed an endotherm (possibly melting/decomposition) at 174.0° C. (onset temperature) and a weight loss of 2.1% up to 150° C. ¹H NMR data indicated a stoichiometry of 1.00 (acid/base) for Compound 9 Type A.

FIG. 51 provides TGA/DSC curves of Compound 9 Type B. DSC/TGA data of showed an endotherm (possibly melting/decomposition) at 177.8° C. (onset temperature) and a weight loss of 1.2% up to 150° C. ¹H NMR data shown in FIG. 7-32 indicated a stoichiometry of 0.99 (acid/base) for Compound 9 Type B.

### Example 12: Compound 10

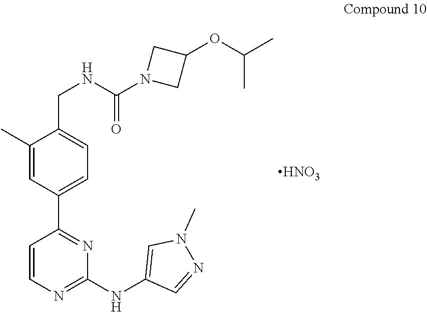 [SMILES: Cc1cc(-c2ccnc(Nc3cnn(C)c3)n2)ccc1CNC(=O)N1CC(OC(C)C)C1 ; "·HNO3"]

FIG. 52 provides an overlay of XRPD patterns of Compound 10 Type A, Type B, and Type C with Compound 1 Type A.

### Compound 10 Type A

FIG. 53 provides TGA/DSC curves of Compound 10 Type A. DSC/TGA data showed an exotherm at 129.9° C. (onset temperature) apparently associated with decomposition upon heating, and a weight loss of 0.5% up to 100° C.

### Compound 10 Type B

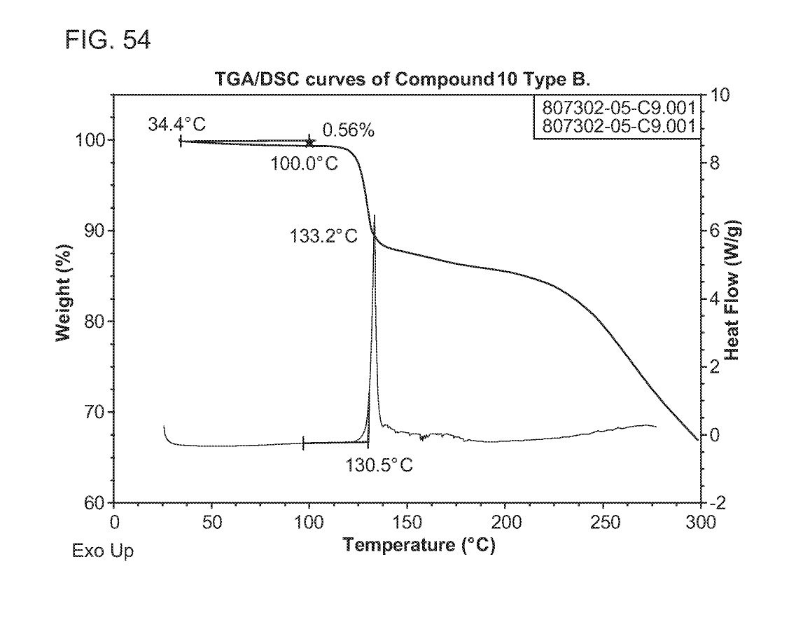

FIG. 54 provides TGA/DSC curves of Compound 10 Type B. DSC/TGA data showed an exotherm at 130.5° C. (onset temperature) apparently associated with decomposition upon heating, and a weight loss of 0.6% up to 100° C.

### Compound 10 Type C

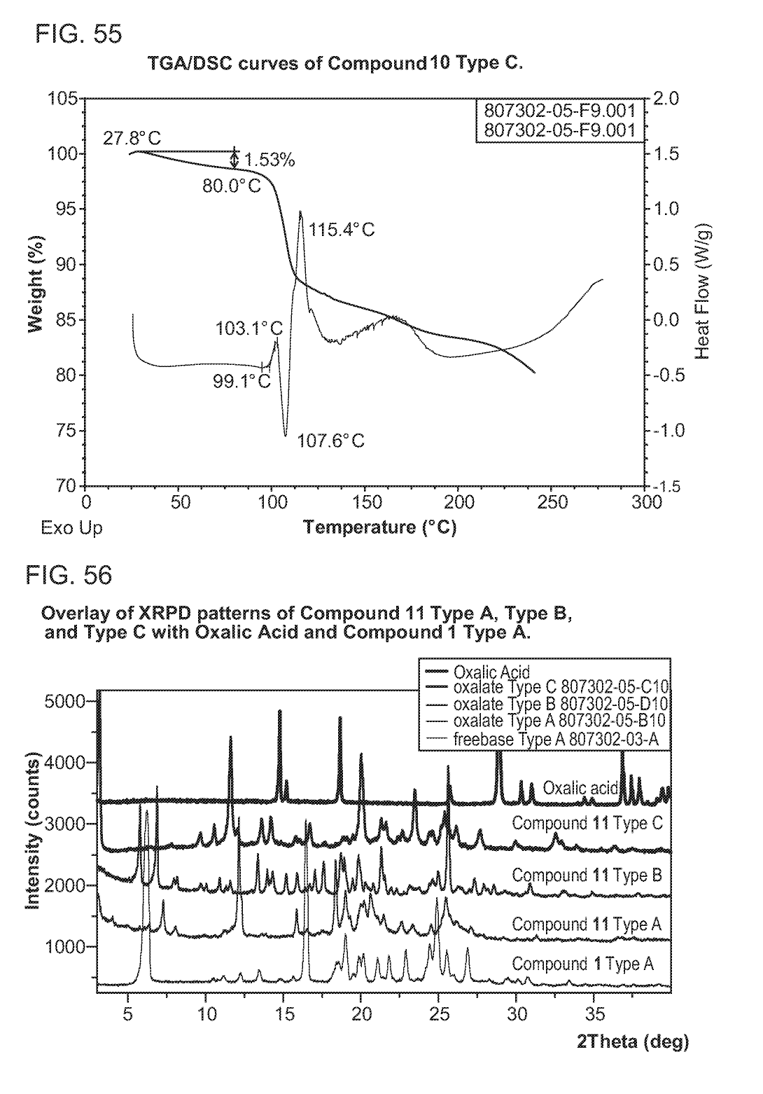

FIG. 55 provides TGA/DSC curves of Compound 10 Type C. DSC/TGA data showed multiple thermal events before the final exotherm apparently associated with decomposition. A weight loss of 1.5% up to 80° C. was observed.

### Example 13: Compound 11

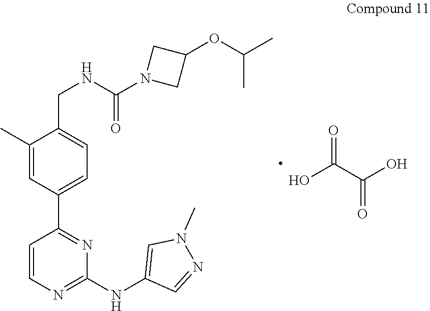 [SMILES: Cc1cc(-c2ccnc(Nc3cnn(C)c3)n2)ccc1CNC(=O)N1CC(OC(C)C)C1 ; O=C(O)C(=O)O]

FIG. 56 provides an overlay of XRPD patterns of Compound 11 Type A, Type B, and Type C with Oxalic Acid and Compound 1 Type A.

### Compound 11 Type A

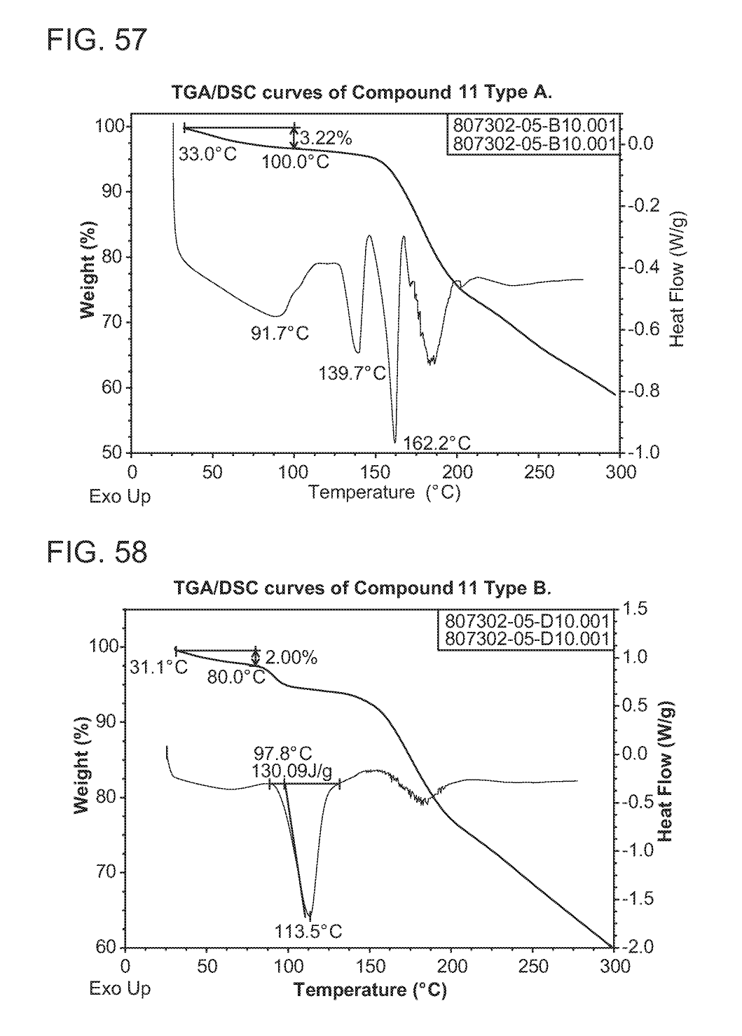

FIG. 57 provides TGA/DSC curves of Compound 11 Type A. DSC/TGA data showed multiple overlapped thermal events before the final apparent decomposition. A weight loss of 3.2% up to 100° C. was observed.

### Compound 11 Type B

FIG. 58 provides TGA/DSC curves of Compound 11 Type B. DSC/TGA data showed an endotherm at 97.8° C. (onset temperature) and a weight loss of 2.0% up to 80° C.

### Compound 11 Type C

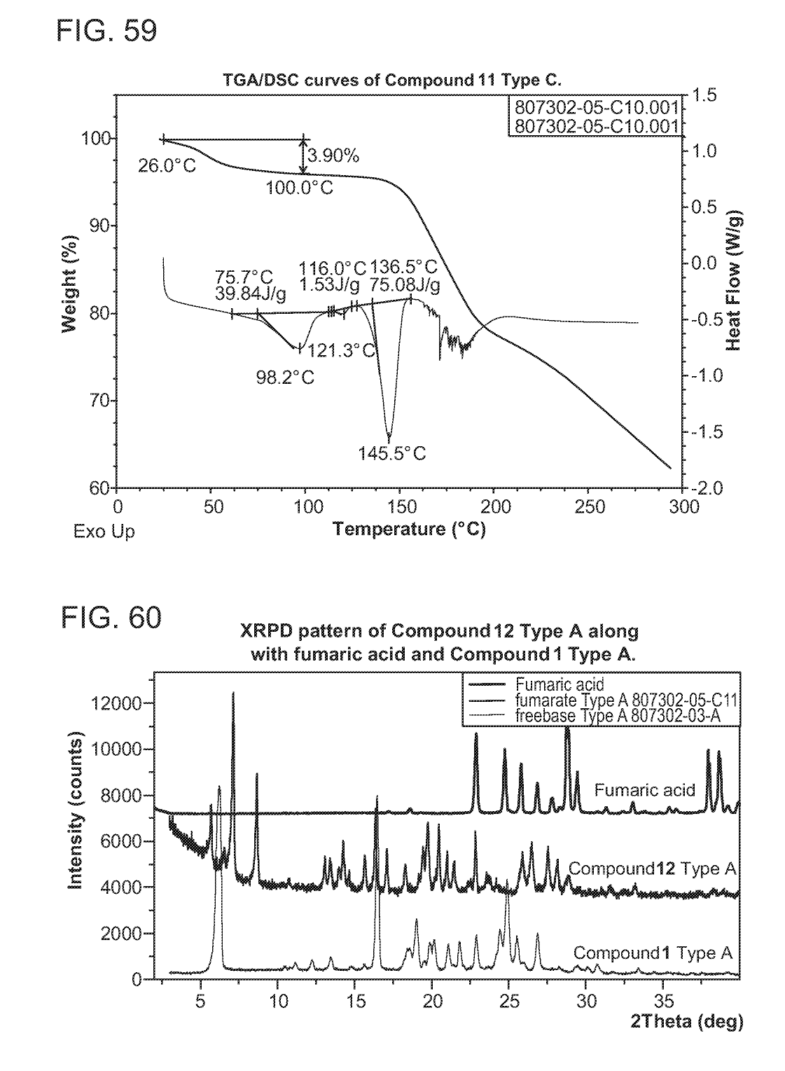

FIG. 59 provides TGA/DSC curves of Compound 11 Type C. DSC/TGA data showed multiple endotherms before apparent decomposition. A weight loss of 3.9% up to 100° C. was observed.

### Example 14: Compound 12

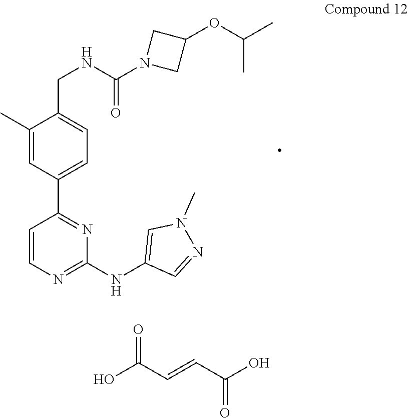 [SMILES: Cc1cc(-c2ccnc(Nc3cnn(C)c3)n2)ccc1CNC(=O)N1CC(OC(C)C)C1 ; O=C(O)/C=C/C(=O)O]

### Compound 12 Type A

FIG. 60 provides the XRPD pattern of Compound 12 Type A along with fumaric acid and Compound 1 Type A.

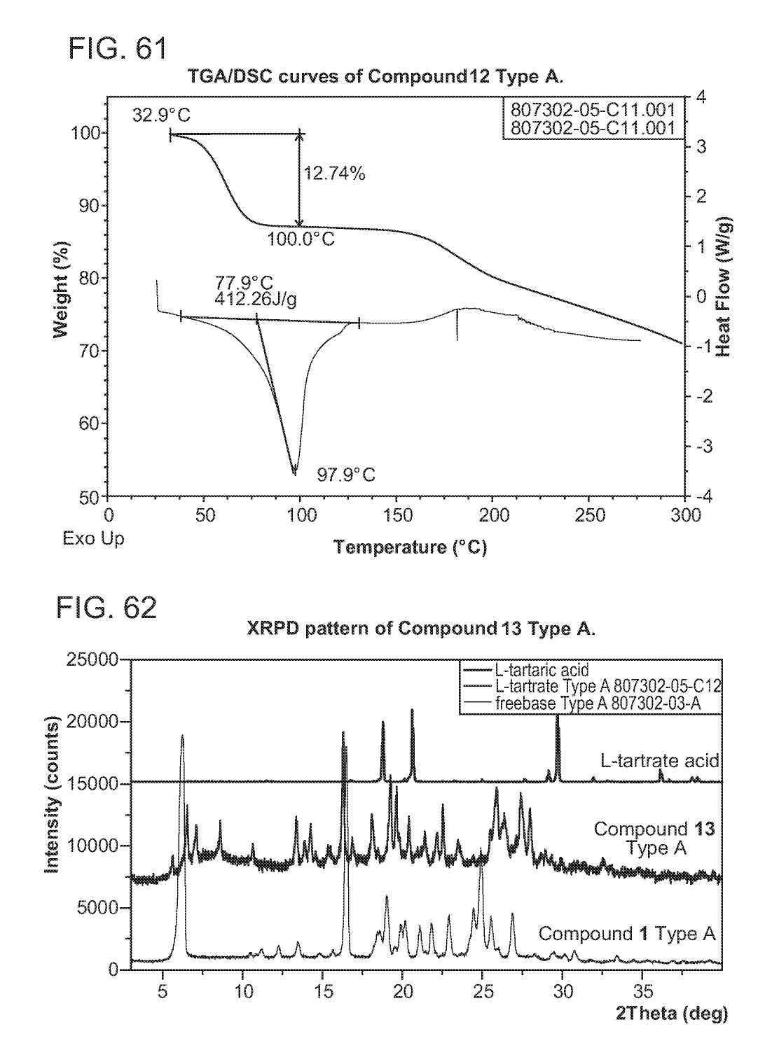

FIG. 61 provides TGA/DSC curves of Compound 12 Type A. DSC/TGA data showed an endotherm at 77.9° C. (onset temperature) and a weight loss of 12.7% up to 100° C. ¹H NMR data indicated a stoichiometry of 0.93 (acid/base) for Compound 12 Type A.

### Example 15: Compound 13

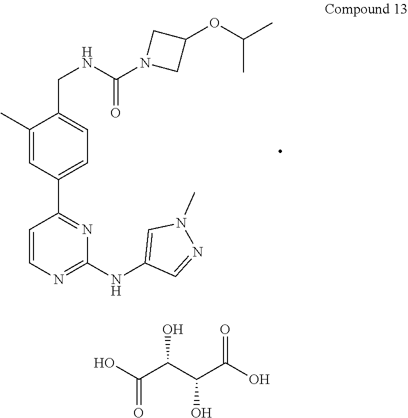 [SMILES: Cc1cc(-c2ccnc(Nc3cnn(C)c3)n2)ccc1CNC(=O)N1CC(OC(C)C)C1 ; O=C(O)[C@H](O)[C@@H](O)C(=O)O]

### Compound 13 Type A

FIG. 62 provides the XRPD pattern of Compound 13 Type A.

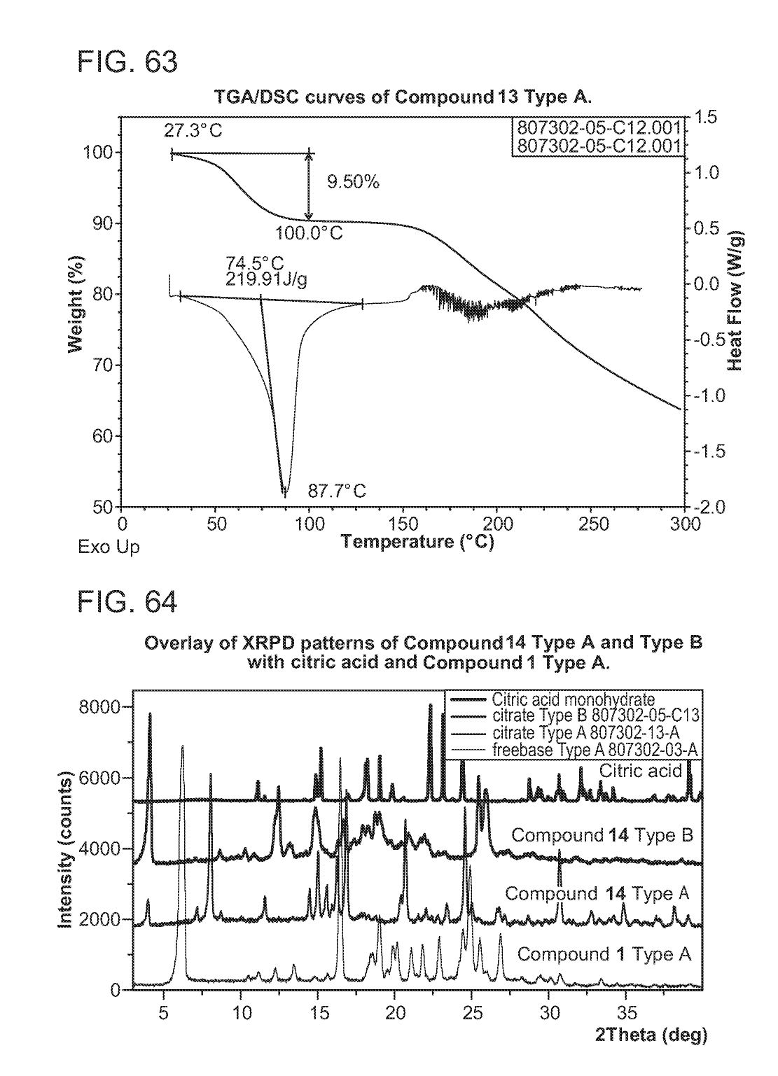

FIG. 63 provides TGA/DSC curves of Compound 13 Type A. DSC/TGA data showed an endotherm at 74.5° C. (onset temperature) and a weight loss of 9.5% up to 100° C. ¹H NMR data indicated a stoichiometry of 0.98 (acid/base) for Compound 13 Type A.

### Example 16: Compound 14

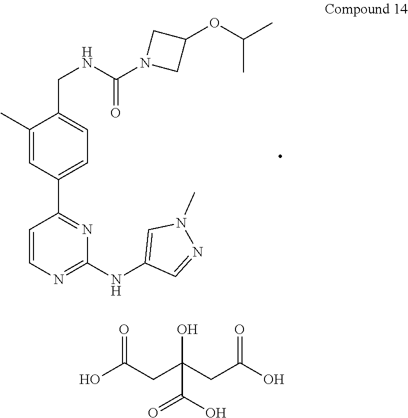 [SMILES: Cc1cc(-c2ccnc(Nc3cnn(C)c3)n2)ccc1CNC(=O)N1CC(OC(C)C)C1 ; O=C(O)CC(O)(CC(=O)O)C(=O)O]

FIG. 64 provides an overlay of XRPD patterns of Compound 14 Type A and Type B with citric acid and Compound 1 Type A.

### Compound 14 Type A

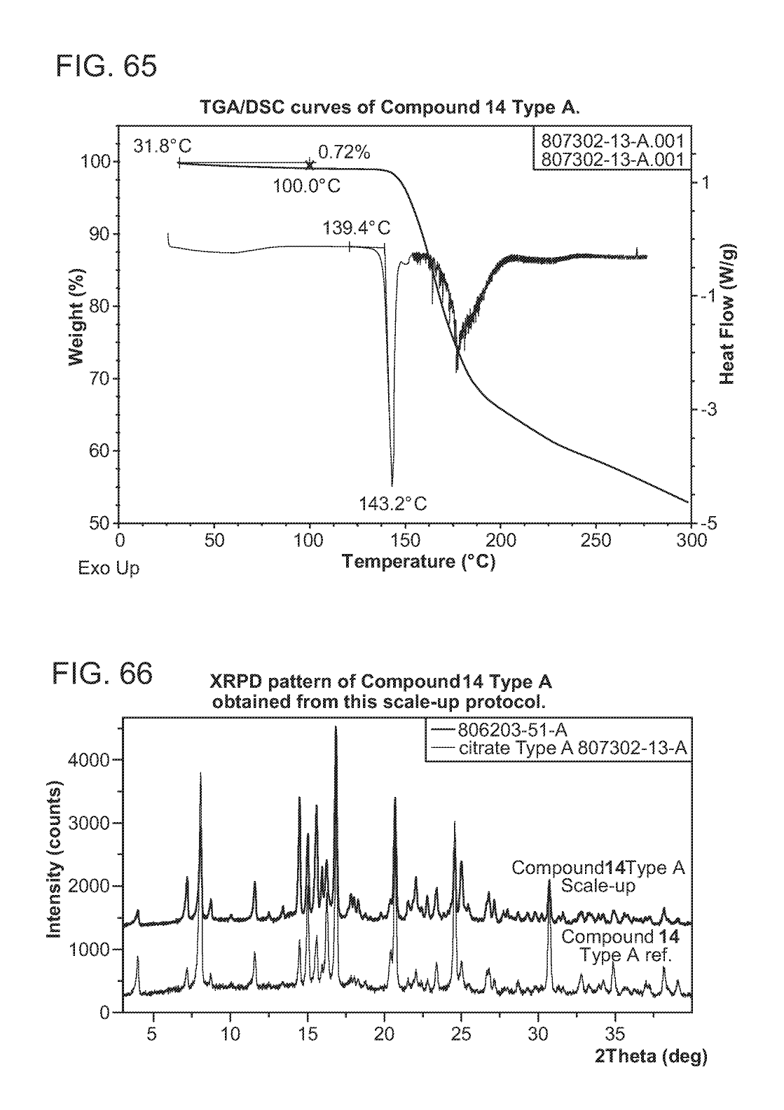

FIG. 65 provides TGA/DSC curves of Compound 14 Type A. The data showed an endotherm (possibly melting/decomposition) at 139.4° C. (onset temperature) and a weight loss of 0.7% up to 100° C. ¹H NMR data indicated a stoichiometry of 2.00 (acid/base) for Compound 14 Type A.

Based on the solid-state characterization results, Compound 14 Type A was scaled up to about one gram using the following preparation procedure.
1. Add 55 mL of ACN into a 250-mL vessel.
2. Keep stirring the ACN at RT (750 rpm) with a magnetic stir bar.
3. Add 0.98 g of citric acid and 1.0 g of freebase into the vessel.
4. Keep stirring at RT for 2 days.
5. Vacuum filter and dry the wet cake under vacuum at 50° C.
6. Collect solids (1.7 g obtained with yield of ˜90.3%).

FIG. 66 provides the XRPD pattern of Compound 14 Type A obtained from this scale-up protocol.

FIG. 67 provides TGA/DSC curves of Compound 14 Type A. TGA data showed a weight loss of 2.2% up to 100° C. and DSC showed an apparent melting endotherm at 140.4° C. (onset temperature). ¹H NMR data indicated a stoichiometry of 1.94 (acid/base) for the scaled up Compound 14 Type A.

### Compound 14 Type B

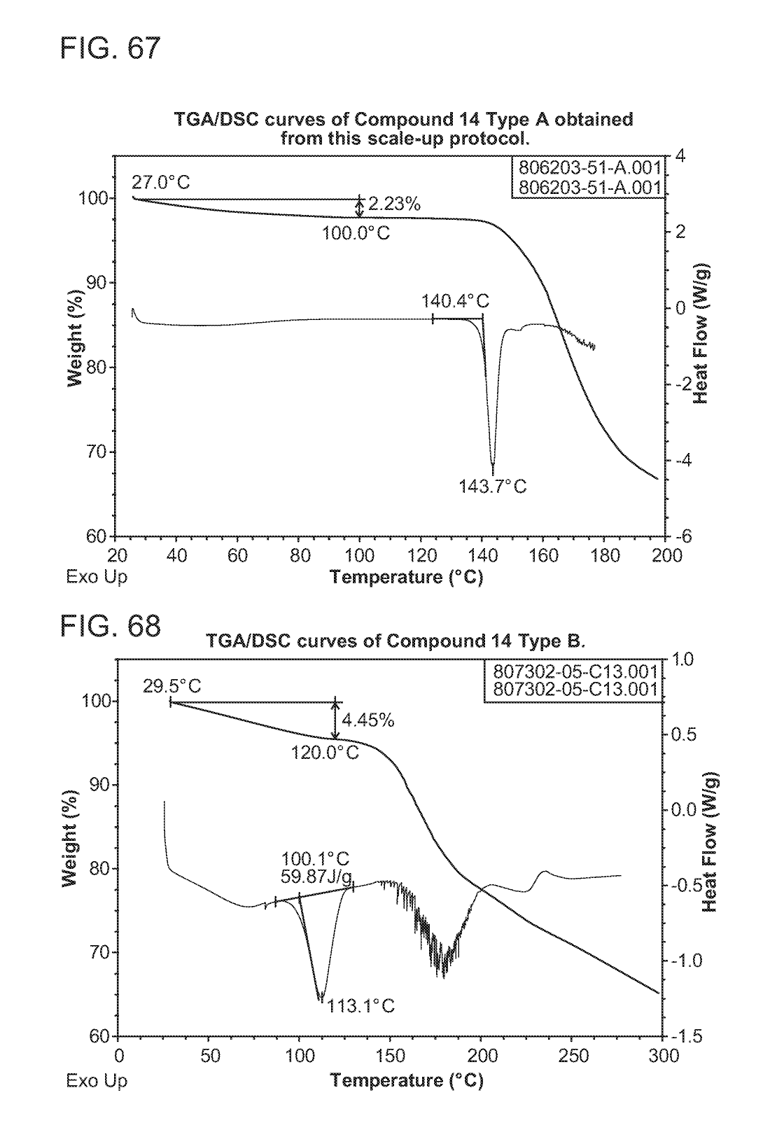

FIG. 68 provides TGA/DSC curves of Compound 14 Type B. DSC/TGA data of showed an endotherm at 100.1° C. (onset temperature) and a weight loss of 4.5% up to 120° C. ¹H NMR data indicated a stoichiometry of 1.09 (acid/base) for Compound 14 Type B.

### Example 17: Compound 15

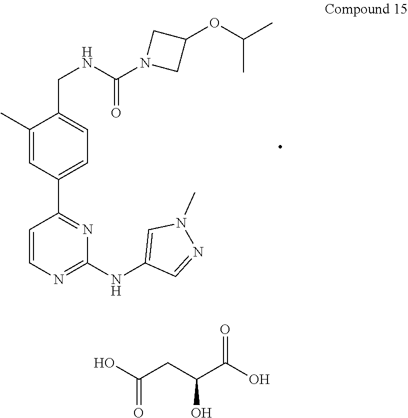 [SMILES: Cc1cc(-c2ccnc(Nc3cnn(C)c3)n2)ccc1CNC(=O)N1CC(OC(C)C)C1 ; O=C(O)C[C@H](O)C(=O)O]

FIG. 69 provides the XRPD pattern of Compound 15 Type A.

FIG. 70 provides TGA/DSC curves of Compound 15 Type A. DSC/TGA data showed an endotherm at 61.2° C. (onset temperature) and a weight loss of 8.5% up to 100° C. ¹H NMR data indicated a stoichiometry of 1.06 (acid/base) for Compound 15 Type A.

### Example 18: Compound 16

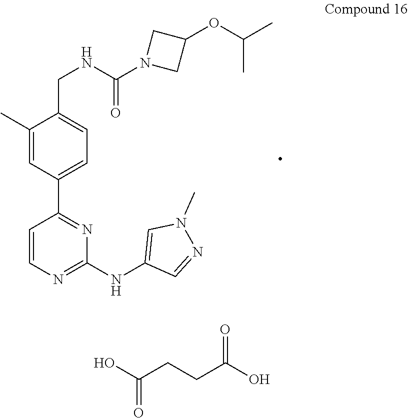 [SMILES: Cc1cc(-c2ccnc(Nc3cnn(C)c3)n2)ccc1CNC(=O)N1CC(OC(C)C)C1 ; O=C(O)CCC(=O)O]

FIG. 71 provides the XRPD pattern of Compound 16 Type A.

FIG. 72 provides TGA/DSC curves of Compound 16 Type A. DSC/TGA data showed an endotherm at 67.5° C. (onset temperature) and a weight loss of 7.0% up to 100° C. ¹H NMR data indicated a stoichiometry of 0.99 (acid/base) for Compound 16 Type A.

### Example 19: Compound 18

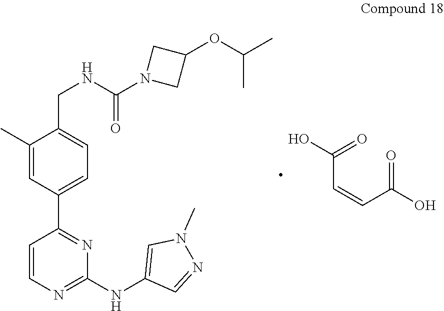 [SMILES: Cc1cc(-c2ccnc(Nc3cnn(C)c3)n2)ccc1CNC(=O)N1CC(OC(C)C)C1 ; O=C(O)/C=C\C(=O)O]

FIG. 73 provides an overlay of XRPD patterns of Compound 18 Type A and B with maleic acid and Compound 1 Type A.

### Compound 18 Type A

FIG. 74 provides TGA/DSC curves of Compound 18 Type A. DSC/TGA data showed an endotherm (possibly melting/decomposition) at 149.6° C. (onset temperature) and a weight loss of 0.4% up to 100° C. ¹H NMR data indicated a stoichiometry of 1.06 (acid/base) for Compound 18 Type A.

Based on the solid-state characterization results, Compound 18 Type A was scaled up to about one gram using the following preparation procedure.
1. Add 55 mL of DCM and 1.1 g of freebase into a 100-mL vessel.
2. Heat the suspension to 40° C. with agitation to obtain a clear solution.
3. Add 0.30 g of maleic acid to the vessel.
4. Stir at RT for 2 days (750 rpm).
5. Vacuum filter and dry the wet cake under vacuum at 50° C.
6. Collect solids (˜0.68 g)
   Another two batches of Compound 18 Type A were obtained with a similar procedure. A total of 1.9 g of freebase was used and 1.1 g of Compound 18 Type A was obtained.
   (yield of ˜45.7%).

FIG. 75 provides XRPD pattern of Compound 18 Type A as obtained from the scale-up procedure.

FIG. 76 provides TGA/DSC curves of Compound 18 Type A. TGA/DSC data showed a weight loss of 1.8% up to 100° C. and an apparent melting/decomposition endotherm at 144.5° C. (onset temperature). ¹H NMR data indicated a stoichiometry of 1.61 (acid/base) for the scaled up Compound 18 Type A.

### Compound 18 Type B

FIG. 77 provides TGA/DSC curves of Compound 18 Type B that showed an endotherm (possibly melting/decomposition) at 140.4° C. (onset temperature) and a weight loss of 1.3% up to 100° C.

### Example 20: Compound 21

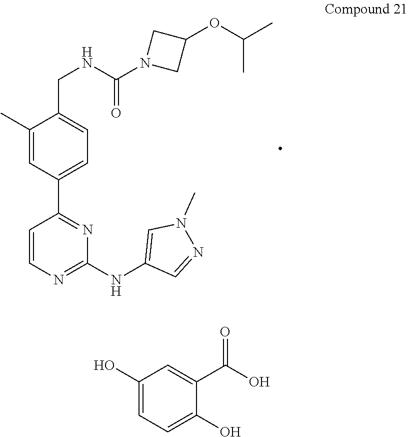 [SMILES: Cc1cc(-c2ccnc(Nc3cnn(C)c3)n2)ccc1CNC(=O)N1CC(OC(C)C)C1 ; O=C(O)c1cc(O)ccc1O]

FIG. 78 provides the XRPD pattern of Compound 21 Type A.

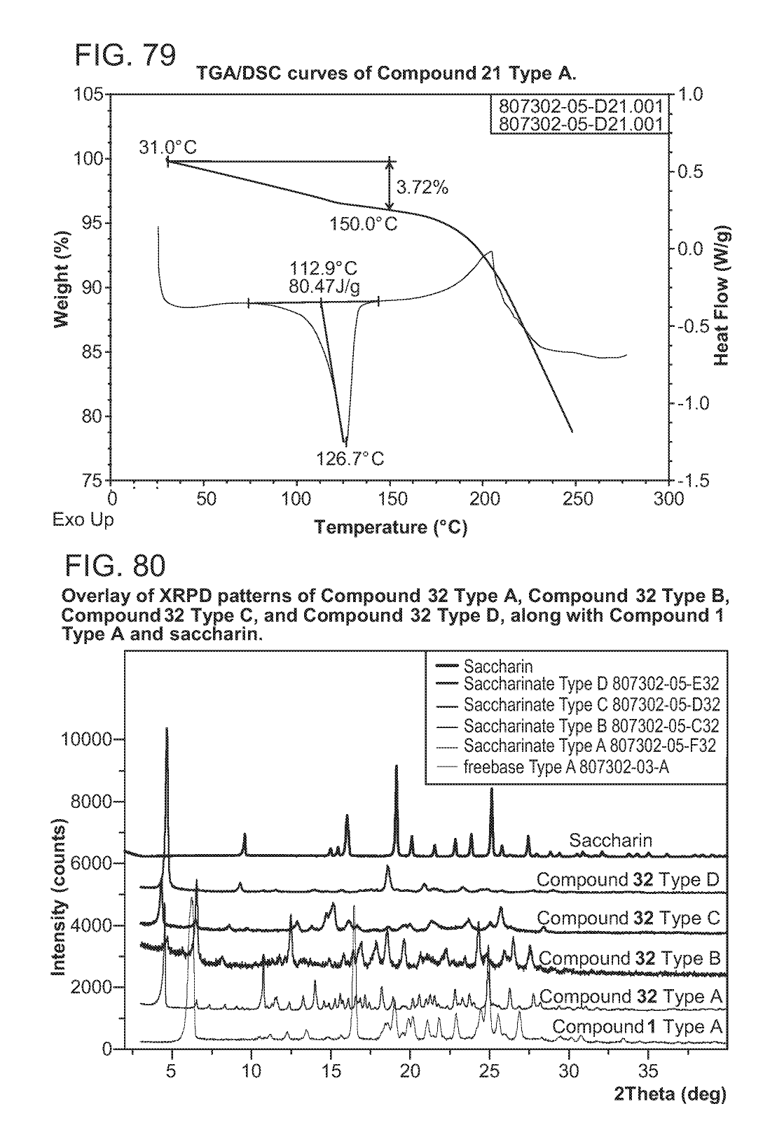

FIG. 79 provides the TGA/DSC curves of Compound 21 Type A and showed an endotherm at 112.9° C. (onset temperature) and a weight loss of 3.7% up to 150° C.

¹H NMR data indicated a stoichiometry of 1.04 (acid/base) for Compound 21 Type A.

### Example 21: Compound 32

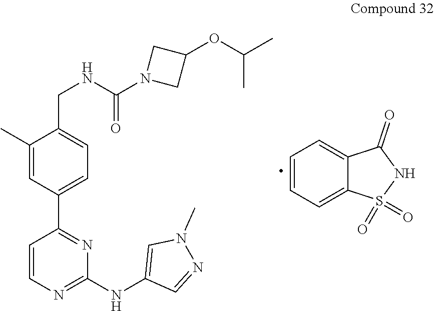 [SMILES: Cc1cc(-c2ccnc(Nc3cnn(C)c3)n2)ccc1CNC(=O)N1CC(OC(C)C)C1 ; O=C1NS(=O)(=O)c2ccccc21]

FIG. 80 provides an overlay of XRPD patterns of Compound 32 Type A, Compound 32 Type B, Compound 32 Type C, and Compound 32 Type D, along with Compound 1 Type A and saccharin.

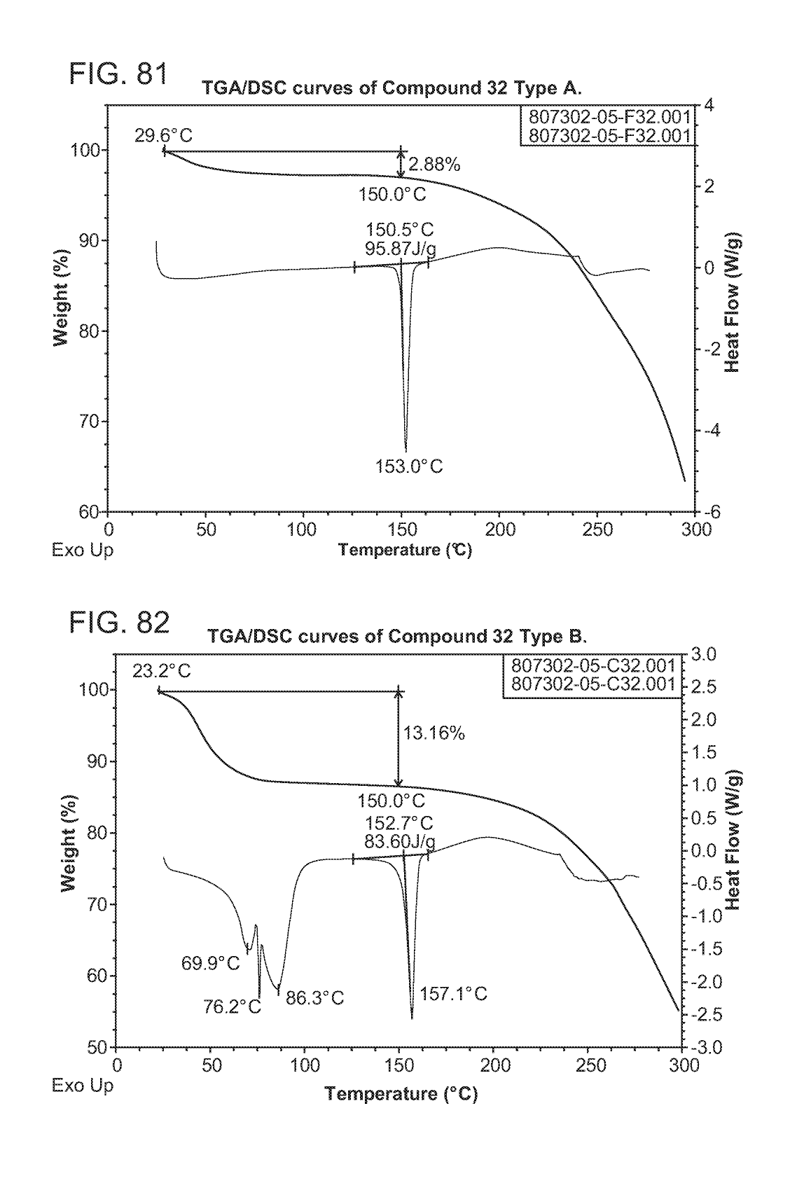

FIG. 81 provides TGA/DSC curves of Compound 32 Type A that showed an endotherm apparently associated with melting at 150.5° C. (onset temperature) and a weight loss of 2.9% up to 150° C.

FIG. 82 provides TGA/DSC curves of Compound 32 Type B that showed multiple endotherms before a possibly melting endotherm at 152.7° C. (onset temperature). A weight loss of 13.2% up to 150° C. was observed.

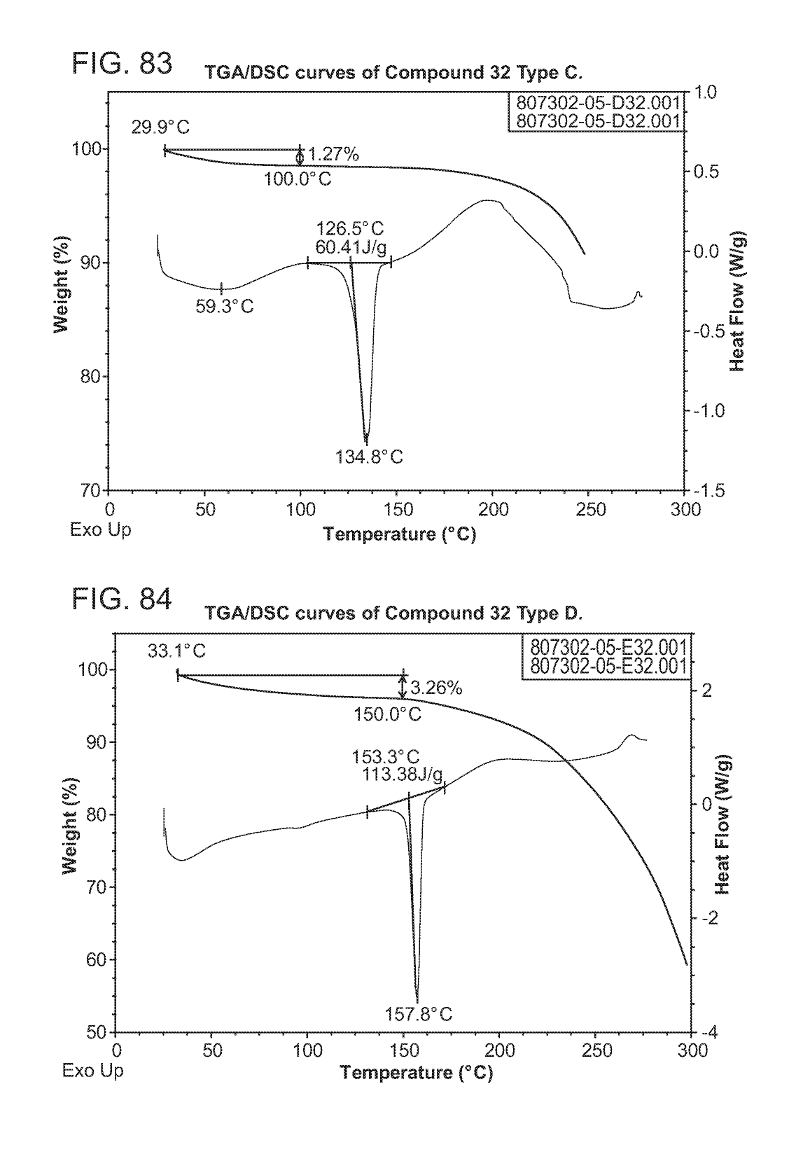

FIG. 83 provides TGA/DSC curves of Compound 32 Type C that showed an apparently melting endotherm at 126.5° C. (onset temperature) and a weight loss of 1.3% up to 100° C.

FIG. 84 provides TGA/DSC curves of Compound 32 Type D that showed a seemingly melting endotherm at 153.3° C. (onset temperature) and a weight loss of 3.3% up to 150° C.

### Example 22: Protocol for Human B Cell Stimulation

Human B cells are purified from 150 ml of blood. Briefly, the blood can be diluted 1/2 with PBS and centrifuged through a Ficoll density gradient. The B cells can be isolated from the mononuclear cells by negative selection using the B cell isolation kit II from Milenyi (Auburn, Calif.). 50,000 B cells per well can then be stimulated with 10 ug/ml of goat F(ab′)2 anti-human IgM antibodies (Jackson ImmunoResearch Laboratories, West Grove, Pa.) in a 96-well plate. Compounds can be diluted in DMSO and added to the cells. Final concentration of DMSO is 0.5%. Proliferation can be measured after 3 days using Promega CellTiter-Glo (Madison, Wis.).

### Example 23: In Vitro BTK Kinase Assay: BTK-POLYGAT-LS ASSAY

The purpose of the BTK in vitro assay is to determine compound potency against BTK through the measurement of IC₅₀. Compound inhibition can be measured after monitoring the amount of phosphorylation of a fluorescein-labeled polyGAT peptide (Invitrogen PV3611) in the presence of active BTK enzyme (Upstate 14-552), ATP, and inhibitor. The BTK kinase reaction can be done in a black 96 well plate (costar 3694). For a typical assay, a 24 μL aliquot of a ATP/peptide master mix (final concentration; ATP 10 μM, polyGAT 100 nM) in kinase buffer (10 mM Tris-HCl pH 7.5, 10 mM MgCl₂, 200 μM Na₃PO₄, 5 mM DTT, 0.01% Triton X-100, and 0.2 mg/ml casein) can be added to each well. Next, 1 μL of a 4-fold, 40× compound titration in 100% DMSO solvent can be added, followed by addition of 15 uL of BTK enzyme mix in 1× kinase buffer (with a final concentration of 0.25 nM). The assay can be incubated for 30 minutes before being stopped with 28 μL of a 50 mM EDTA solution. Aliquots (5 μL) of the kinase reaction can be transferred to a low volume white 384 well plate (Corning 3674), and 5 μL of a 2× detection buffer (Invitrogen PV3574, with 4 nM Tb—PY20 antibody, Invitrogen PV3552) can be added. The plate can be covered and incubated for 45 minutes at room temperature. Time resolved fluorescence (TRF) on Molecular Devices M5 (332 nm excitation; 488 nm emission; 518 nm fluorescein emission) can be measured. IC₅₀ values can be calculated using a four parameter fit with 100% enzyme activity determined from the DMSO control and 0% activity from the EDTA control.

### Example 24: In Vitro Inhibition of BTK Activity in Mouse Whole Blood

Anti-rabbit MSD plates (Meso Scale Discovery, Rockville, Md.) can be coated with 35 uL/well of rabbit anti-BTK C82B8 (Cell Signaling Technology, Danvers, Mass.) diluted 1:50 in PBS. Plates can be incubated for 2 hours±1 hour at room temp, shaking (setting 3-5) or ON at 4° C. Plates can be blocked with MSD Blocker A (Meso Scale Discovery, Rockville, Md.) using 3% MSD Blocker A in TBST. Coated plates can be first washed 3× with 250 uL/well TBST followed by addition of 200 uL/well 3% Blocker A/TBST. Plates can be blocked for >2 hour at room temperature, shaking or ON at 4° C.

Whole blood can be collected from DBA/1 mice in 16×100 sodium heparin tubes (Becton Dickinson, Cat No. 367874). Blood from multiple DBA/1 mice can be pooled. 96 uL of whole blood per well can be aliquotted into a 96-round bottom plate changing tips each time. 4 uL diluted test compound can be added to each sample, mixed, and incubated for 30 min at 37° C.

For serial dilutions of test compound, 1000× plate can be produced with serial dilutions of test compound in 100% DMSO. Ten dilutions, done 1:3, starting at 10 mM can be created by: adding 15 uL of test compound at 10 mM in 100% DMSO to well A1; adding 10 uL 100% DMSO to wells A2-A12; diluting 5 uL from well A1 to well A2 and mixing; continuing 1:3 serial dilutions, changing tips between transfers, to well A10. Wells A11 and A12 can contain 100% DMSO without test compound.

For dilution 1, a 1:40 plate can be created. Using a 12-well multi-channel pipette, each concentration of test compound or DMSO can be diluted 1:40 by adding 2 uL from each well of 1000× stock plate to 78 uL water and mixing.

For dilution 2, test compound or DMSO can be added to whole blood by diluting 1:25. Using a 12-well multi-channel pipette, 4 uL from 1:40 plate (B) can be added to 96 uL whole blood and mixed.

Lysing buffer used to lyse whole blood can be prepared as follows. A 10× Lysis buffer can be prepared using 1500 mM NaCl; 200 mM Tris, pH 7.5; 10 mM EDTA; 10 mM EGTA; and 10% Triton-X-100. The 10× Lysis buffer is diluted to 1× in dH₂O, and complete lysing buffer (+/− phosphatase inhibitors) can be prepared as follows:

| | +PPi (mL) | −PPi (mL) |
|---|---|---|
| 1X Lysis buffer | 10 | 10 |
| 500 mM PMSF in DMSO | 0.02 | 0.02 |
| Phosphatase Inhibitor 3 | 0.1 | |
| Phosphatase Inhibitor 2 | 0.1 | |
| Protease Inhibitor (cOmplete) (1 tablet for 10 mL) | 1 tablet | 1 tablet |
| PhosStop (1 tablet for 10 mL) | 1 tablet | |
| Sodium Orthovanadate (Na₃VO₄) (50 uM final) | 0.1 | |
| Sodium Fluoride (NaF) (10 mM final) | 0.005 | |
| 1% Deoxycholate (0.25% final) | 2.5 | 2.5 |

100 uL of complete lysing buffer (+/− phosphatase inhibitors) can be added to each well, and mixed well by pipetting up and down a few times. Wells 1-10 and 12 can receive 1× Lysis buffer containing phosphatase inhibitors (+PPi) and well 11 can receive 1× Lysis buffer without phosphatase inhibitors (−PPi). Samples can be incubated for 1 hour on ice or at 4° C. Samples can be mixed again at half time point for complete lysing.

Blocking buffer can be washed off blocked MSD plates with 250 uL TBST per well 3 times. 100-150 uL of whole blood lysates can be added to each well of the coated and blocked MSD plates followed by incubation overnight in a cold room with shaking.

The plates can then washed 4 times with 250 μL TBST per well. Biotinylated phospho-tyrosine mouse mAb (pY100, Cell Signaling Technology, Danvers, Mass.) can be diluted 1:125 in 1% Blocker A. Mouse anti-BTK mAb (Fitzgerald Industries International, Acton, Mass.) can be diluted 1:900 in 1% Blocker A. 35 μL of diluted pY100 or diluted anti-BTK mAb can be added to each well and incubated for 2 hours at room temperature, shaking.

Plates can be then washed 3 times with 250 uL TBST/well. 35 uL of 1:500 Streptavidin-Sulfo-Tag labeled antibody in 3% Blocker A can be added to each well. For anti-BTK, 35 uL of 1:500 anti-mouse-Tag labeled antibody in 3% Blocker A can be added to each well. Plates can be incubated for 1 hour at room temperature, shaking.

To develop and read the plates, 1× Read Buffer in dH₂O can be prepared from 4× stock. Plates can be washed 3 times with 250 uL TBST/well. 150 uL of 1×MSD Read Buffer is added to each well. Plates can be read in a SECTOR Imager 6000 (Meso Scale Discovery, Rockville, Md.).

### Materials

| ITEM | VENDOR | CATALOG NO. |
|---|---|---|
| Anti-rabbit MSD plates | MSD | L45RA-1 |
| Rabbit anti-BTK (C82B8) | Cell Signaling | 3533S |
| PBS | Media Prep | |
| MSD Blocker A | MSD | R93BA-4 |
| TBST (1xTBS/0.1% Tween20) | Media Prep | |
| 10X Lysing Buffer | Media Prep | |
| PMSF in DMSO (500 mM) | Media Prep | |
| Phosphatase Cocktail Inhibitor 3 | Sigma Aldrich | P0044-5ML |
| Phosphatase Cocktail Inhibitor 2 | Sigma Aldrich | P5726-1ML |
| cOmplete Mini | Roche | 11 836 153 001 |
| PhosStop Inhibitor | Roche | 04 906 837 001 |
| Sodium Orthovanadate 100 mM | Media Prep | |
| Sodium Fluoride 1M | Media Prep | |
| 1% Deoxycholate | Media Prep | |
| pTyr 100 ms mAb biotinylated | Cell Signaling | 9417S |
| Streptavidin Sulfo-Tag | MSD | R32AD-1 |
| MSD Read Buffer 4X | MSD | R92TC-1 |
| Costar 96-round bottom | Costar/Fisher | 3799 |
| Mouse anti-BTK (7F12H4) | Fitzgerald | 10R-1929 |
| Anti-mouse Sulfo-Tag | MSD | R32AC-5 |

### Example 25: PK/PD Correlation in DBA1 Mice

Mice can be dosed orally (PO) with test compound in CMC-Tween and killed by CO₂ asphyxiation at various times after dosing. Heparinized whole blood can be immediately collected by cardiac puncture and split into two samples. One sample can be used to quantify the amount of test compound present and the other is lysed in MSD lysis buffer in the presence of phosphatase inhibitors. Heparinized whole blood from cardiac punctures of vehicle (CMC-Tween) dosed mice can be lysed either in the presence (high control) or absence (low control) of phosphatase inhibitors. Lysed whole blood samples can be analyzed for phospho-BTK as described above. The percent inhibition of phospho-BTK in each whole blood sample from dosed mice can be calculated as follows: (1-((pBTK(x+PPi)−pBTK(vehicle −PPi))/(pBTK(vehicle+PPi))))*100, where pBTK(x+PPi) is the ECL signal for whole blood from each test compound-treated mouse, pBTK(vehicle−PPi) is the average ECL signal of whole blood from vehicle-treated mice lysed in the absence of phosphatase inhibitors (low control) and pBTK(vehicle+PPi) is the average ECL signal of whole blood from vehicle-treated mice lysed in the presence of phosphatase inhibitors (high control).

### Example 26: In Vitro PD Assay in Human Whole Blood

Human heparinized venous blood can be purchased from Bioreclamation, Inc. or SeraCare Life Sciences and shipped overnight. Whole blood can be aliquoted into 96-well plate and “spiked” with serial dilutions of test compound in DMSO or with DMSO without drug. The final concentration of DMSO in all wells can be 0.1%. The plate can be incubated at 37° C. for 30 min. Lysis buffer containing protease and phosphatase inhibitors can be added to the drug-containing samples and one of the DMSO-only samples (+PPi, high control), while lysis buffer containing protease inhibitors can be added to the other DMSO-only samples (−PPi, low control). All of the lysed whole blood samples can be subjected to the total BTK capture and phosphotyrosine detection method described in Example 24. ECL values can be graphed in Prism and a best-fit curve with restrictions on the maximum and minimum defined by the +PPi high and −PPi low controls can be used to estimate the test compound concentration that results in 50% inhibition of ECL signal by interpolation.

### Example 27: Preparation of Single Crystals of Compound 1

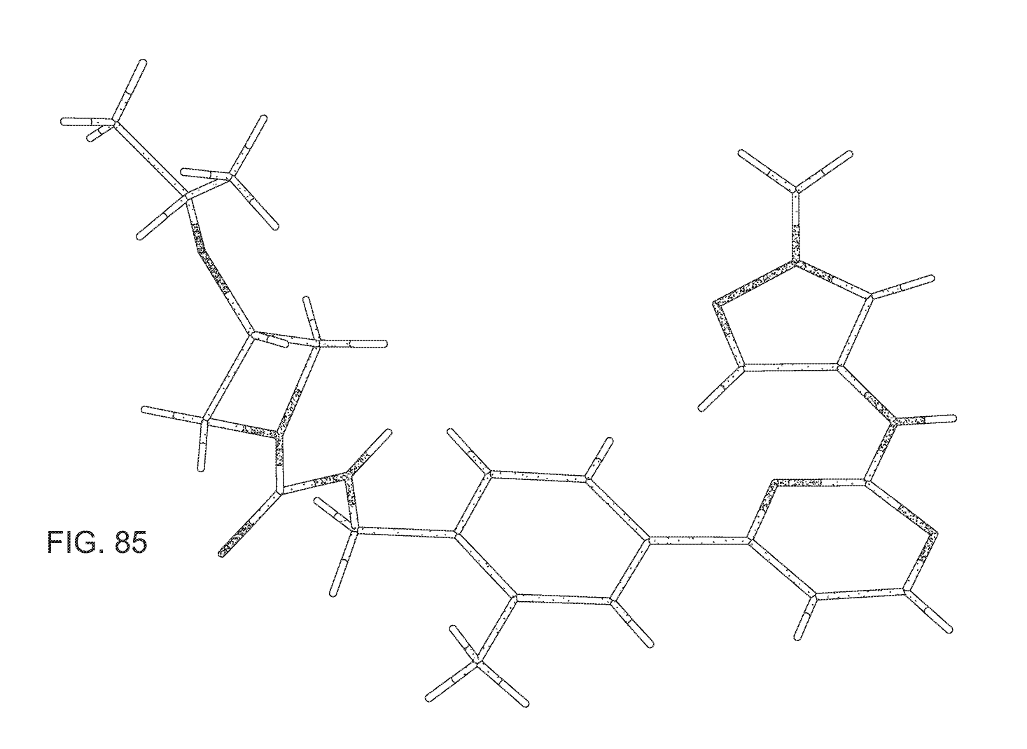

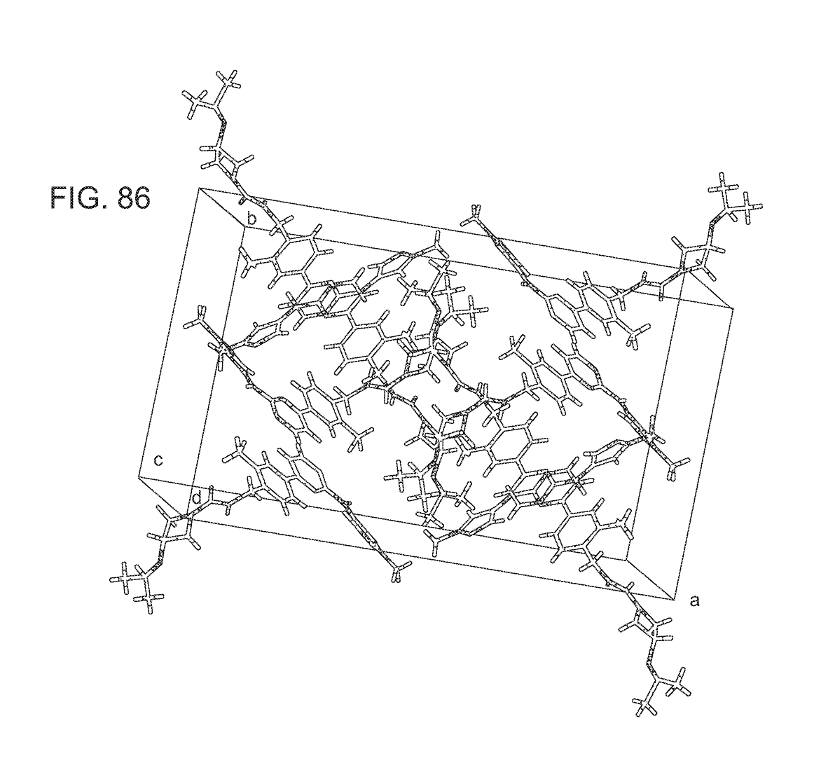

9.9 mg of Compound 1 was weighed into a 3 mL vial with the addition of 1.0 mL Acetone/H₂O (1/10, v/v) mixture solvent. An additional 0.6 mL of acetone was added into the system. The solution was vortexed and sonicated for 3 minutes. Then the solution was filtered with a 0.45 μm filter to another two 4 mL vials. Seeds of Compound 1 were added in the vials that contained the filtrate. The vials were covered with caps with one hole, and then the vials were kept at room temperature in a fume hood for slow evaporation. After four days, needle-like crystals were obtained. The chemical structure and unit cell based on the XRPD pattern of the resultant crystals of Compound 1 are shown in FIG. 85 and FIG. 86, respectively. The details of crystal data and structure refinement are listed in Table 61.

TABLE 61

| | |
|---|---|
| Empirical formula | C₂₃H₂₉N₇O₂ |
| Formula weight | 435.53 |
| Temperature | 298(2)K |
| Wavelength | 0.71073 A |
| Crystal system, space group | Monoclinic, C2/c |
| Unit cell dimensions | a = 28.029(4) Å alpha = 90 deg. |
| | b = 16.780(2) Å beta = 96.033(5) deg. |
| | c = 9.7571(14) Å gamma = 90 deg. |
| Volume/Å³ | 4563.5(11) |
| Z, Calculated density/Mg/m³ | 8, 1.268 Mg/m³ |
| Absorption coefficient | 0.085 mm⁻¹ |
| F(000) | 1856 |
| Crystal size | 0.23 × 0.18 × 0.14 |
| Theta range for data collection | 2.47 to 24.70 deg |
| Limiting indices | −32 ≤ h ≤ 32, |
| | −19 ≤ k ≤ 19, |
| | −11 ≤ l ≤ 11 |
| Reflections collected/unique | 28559/3518 [R(int) = 0.1151] |
| Completeness | 90.4% |
| Refinement method | Semi-empirical from equivalents |
| Data/restraints/parameters | 3518/0/294 |
| Goodness-of-fit on F² | 1.185 |
| Final R indices [I > 2sigma(I)] | R1 = 0.1646, wR2 = 0.3757 |
| Largest diff. peak and hole | 0.743 and −0.383 e · A⁻³ |

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound comprising Compound 1 and an acid:

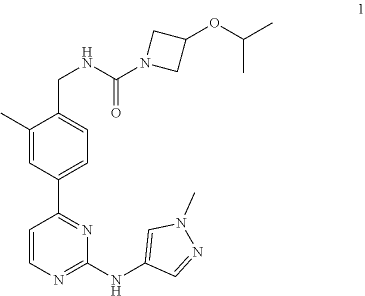 [SMILES: Cc1cc(-c2ccnc(Nc3cnn(C)c3)n2)ccc1CNC(=O)N1CC(OC(C)C)C1]

2. The compound of claim 1, wherein the compound is selected from:
(1) Compound 2 comprising Compound 1 and hydrochloric acid: 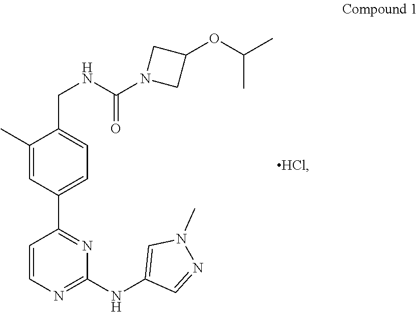 [SMILES: Cc1cc(-c2ccnc(Nc3cnn(C)c3)n2)ccc1CNC(=O)N1CC(OC(C)C)C1]
(2) Compound 3 comprising Compound 1 and sulfuric acid, 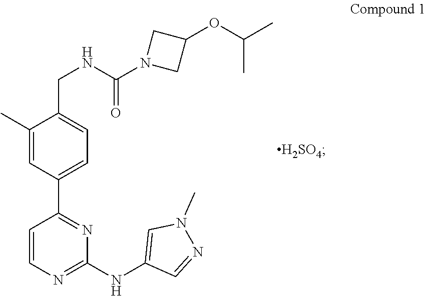 [SMILES: Cc1cc(-c2ccnc(Nc3cnn(C)c3)n2)ccc1CNC(=O)N1CC(OC(C)C)C1]
(3) Compound 4 comprising Compound 1 and methansulfonic acid: 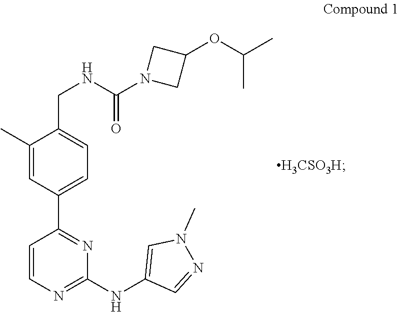 [SMILES: Cc1cc(-c2ccnc(Nc3cnn(C)c3)n2)ccc1CNC(=O)N1CC(OC(C)C)C1]
(4) Compound 5 comprising Compound 1 and ethanedisulfonic acid: 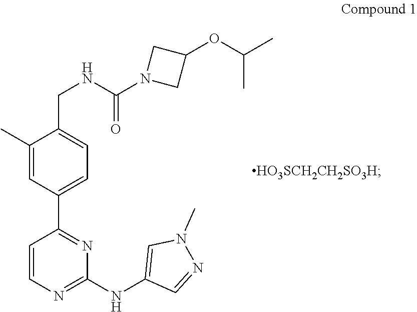 [SMILES: Cc1cc(-c2ccnc(Nc3cnn(C)c3)n2)ccc1CNC(=O)N1CC(OC(C)C)C1 ; "·HO3SCH2CH2SO3H;"]
(5) Compound 6 comprising Compound 1 and 2-hydroxyethanesulfonic acid: 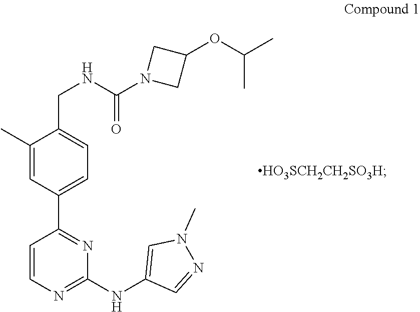 [SMILES: Cc1cc(-c2ccnc(Nc3cnn(C)c3)n2)ccc1CNC(=O)N1CC(OC(C)C)C1 ; "·HO3SCH2CH2SO3H;"]
(6) Compound 7 comprising Compound 1 and benzenesulfonic acid: 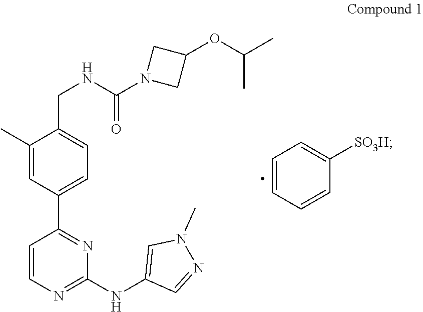 [SMILES: Cc1cc(-c2ccnc(Nc3cnn(C)c3)n2)ccc1CNC(=O)N1CC(OC(C)C)C1 ; O=S(=O)(O)c1ccccc1]
(7) Compound 8 comprising Compound 1 and toluenesulfonic acid: 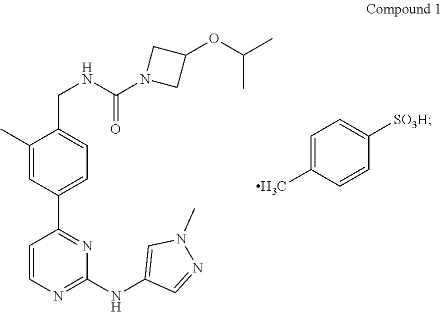
(8) Compound 9 comprising Compound 1 and 2-naphthalenesulfonic acid: 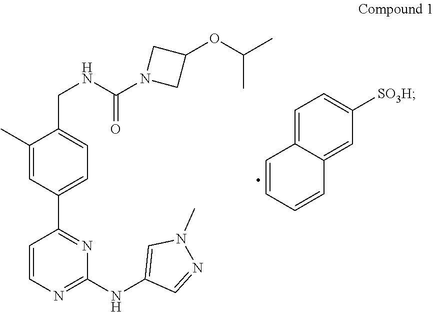 [SMILES: Cc1cc(-c2ccnc(Nc3cnn(C)c3)n2)ccc1CNC(=O)N1CC(OC(C)C)C1 ; O=S(=O)(O)c1ccc2ccccc2c1]
(9) Compound 10 comprising Compound 1 and nitric acid: 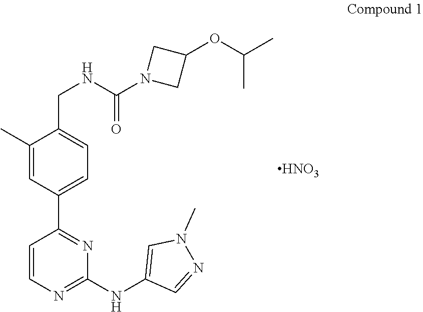 [SMILES: Cc1cc(-c2ccnc(Nc3cnn(C)c3)n2)ccc1CNC(=O)N1CC(OC(C)C)C1 ; "·HNO3"]
(10) Compound 11 comprising Compound 1 and oxalic acid: 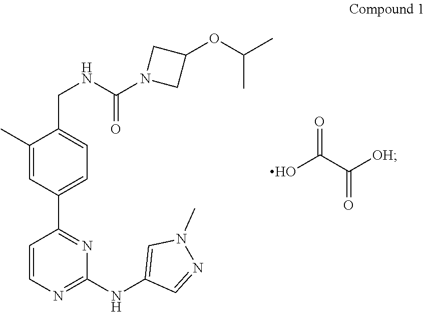
(11) Compound 12 comprising Compound 1 and fumaric acid: 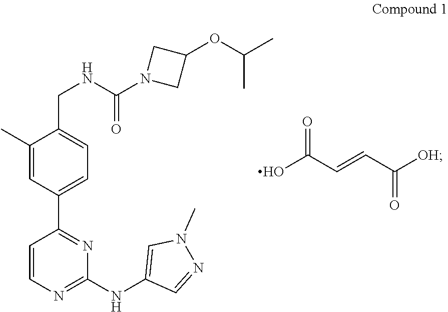
(12) Compound 13 comprising Compound 1 and L-tartaric acid: 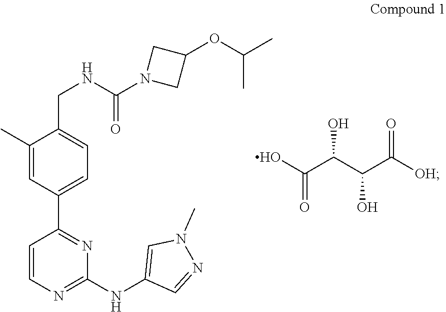
(13) Compound 14 comprising Compound 1 and citric acid: 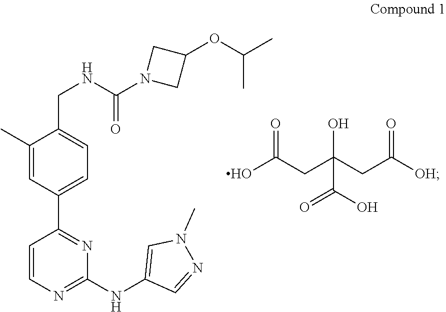
(14) Compound 15 comprising Compound 1 and L-malic acid: 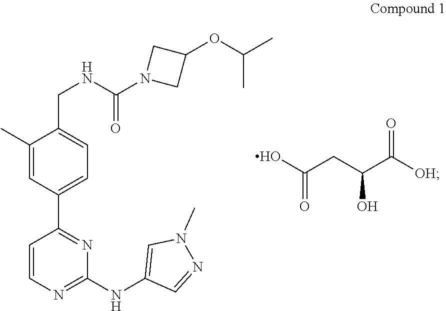
(15) Compound 16 comprising Compound 1 and succinic acid: 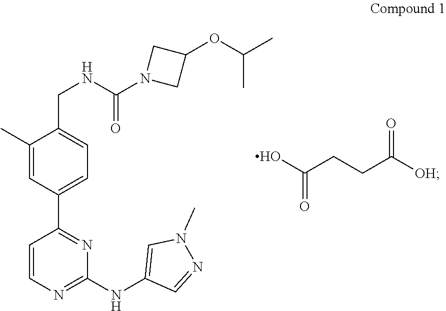
(16) Compound 17 comprising Compound 1 and hippuric acid: 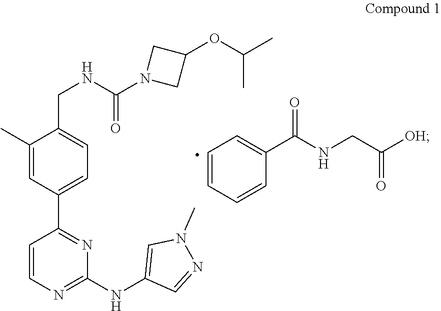
(17) Compound 18 comprising Compound 1 and maleic acid: 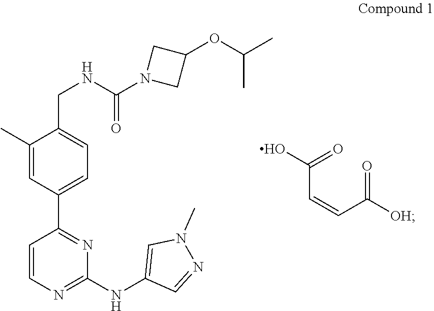
(18) Compound 19 comprising Compound 1 and glutamic acid: 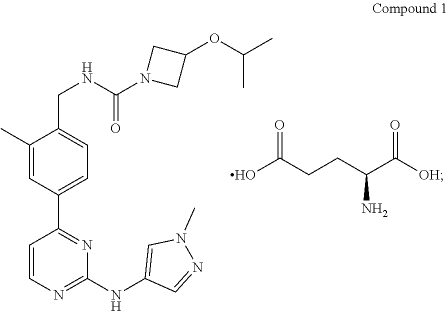 [SMILES: Cc1cc(-c2ccnc(Nc3cnn(C)c3)n2)ccc1CNC(=O)N1CC(OC(C)C)C1 ; N[C@@H](CCC(=O)O)C(=O)O]
(19) Compound 20 comprising Compound 1 and benzoic acid: 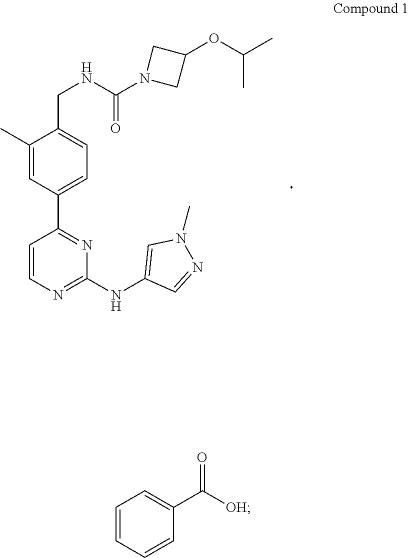 [SMILES: Cc1cc(-c2ccnc(Nc3cnn(C)c3)n2)ccc1CNC(=O)N1CC(OC(C)C)C1 ; O=C(O)c1ccccc1]
(20) Compound 21 comprising Compound 1 and gentisic acid: 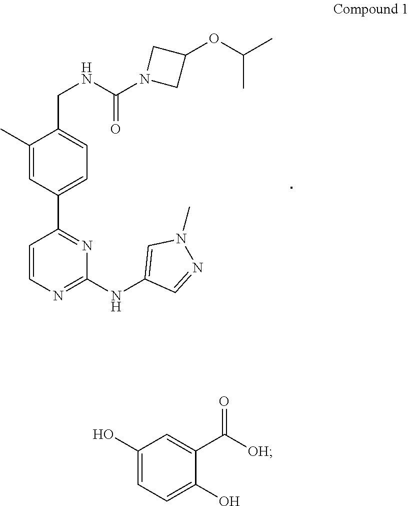 [SMILES: Cc1cc(-c2ccnc(Nc3cnn(C)c3)n2)ccc1CNC(=O)N1CC(OC(C)C)C1 ; O=C(O)c1cc(O)ccc1O]
(21) Compound 22 comprising Compound 1 and malonic acid: 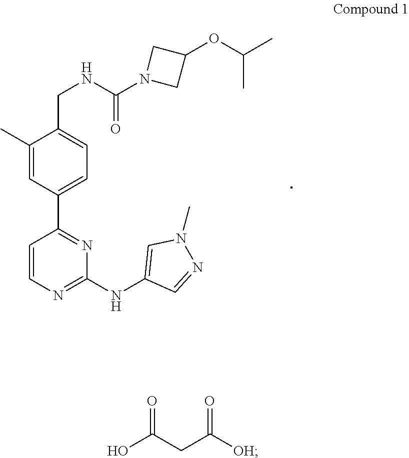 [SMILES: Cc1cc(-c2ccnc(Nc3cnn(C)c3)n2)ccc1CNC(=O)N1CC(OC(C)C)C1 ; O=C(O)CC(=O)O]
(22) Compound 23 comprising Compound 1 and cinnamic acid: 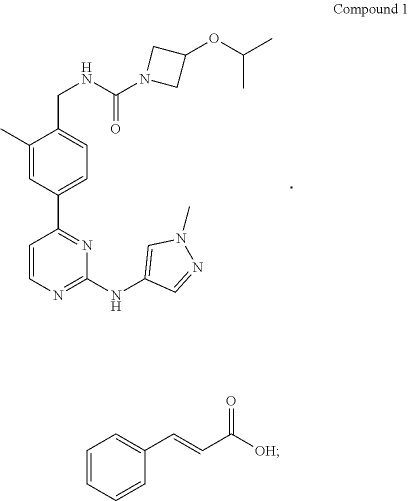 [SMILES: Cc1cc(-c2ccnc(Nc3cnn(C)c3)n2)ccc1CNC(=O)N1CC(OC(C)C)C1 ; O=C(O)/C=C/c1ccccc1]
(23) Compound 24 comprising Compound 1 and L-glutamine: 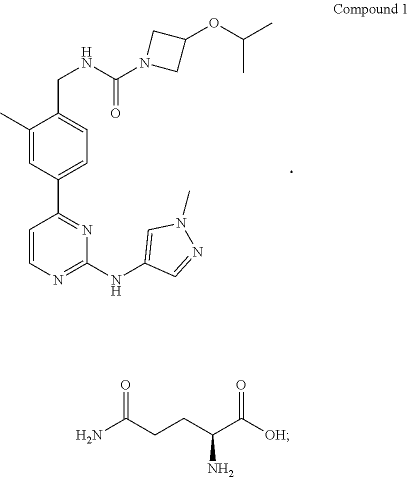 [SMILES: Cc1cc(-c2ccnc(Nc3cnn(C)c3)n2)ccc1CNC(=O)N1CC(OC(C)C)C1 ; NC(=O)CC[C@H](N)C(=O)O]
(24) Compound 25 comprising Compound 1 and L-lysine: 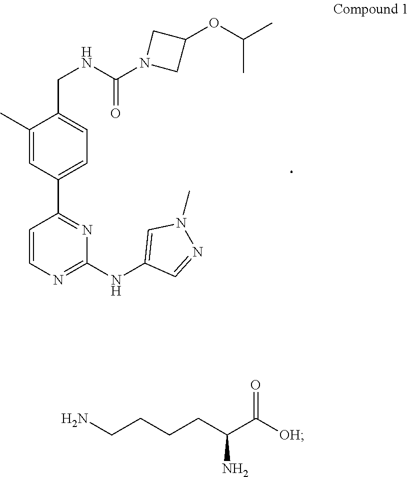 [SMILES: Cc1cc(-c2ccnc(Nc3cnn(C)c3)n2)ccc1CNC(=O)N1CC(OC(C)C)C1 ; NCCCC[C@H](N)C(=O)O]
(25) Compound 26 comprising Compound 1 and L-phenylalanine: 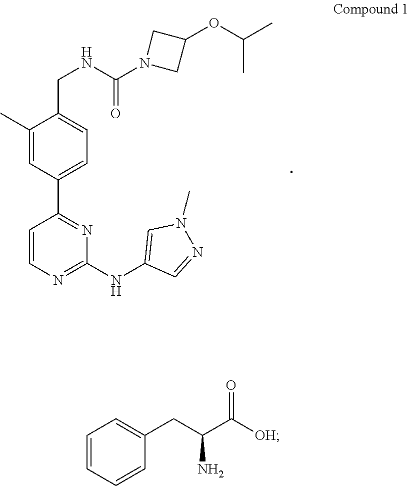 [SMILES: Cc1cc(-c2ccnc(Nc3cnn(C)c3)n2)ccc1CNC(=O)N1CC(OC(C)C)C1 ; N[C@@H](Cc1ccccc1)C(=O)O]
(26) Compound 27 comprising Compound 1 and L-proline: 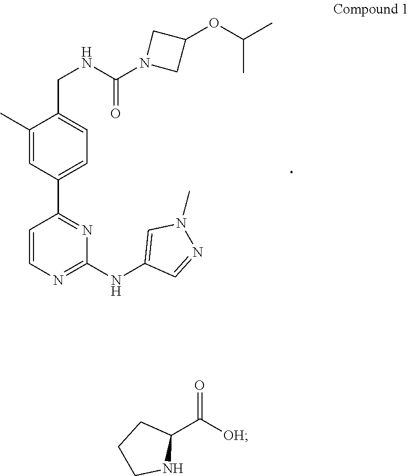 [SMILES: Cc1cc(-c2ccnc(Nc3cnn(C)c3)n2)ccc1CNC(=O)N1CC(OC(C)C)C1 ; O=C(O)[C@@H]1CCCN1]
(27) Compound 28 comprising Compound 1 and L-serine: 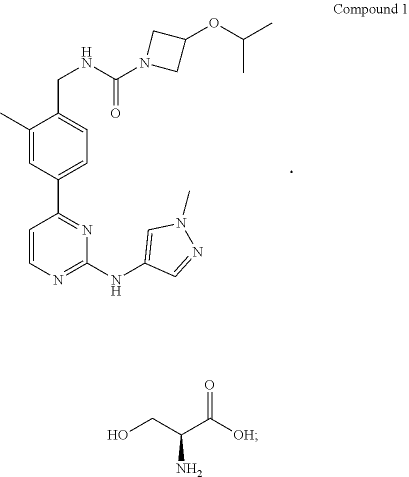 [SMILES: Cc1cc(-c2ccnc(Nc3cnn(C)c3)n2)ccc1CNC(=O)N1CC(OC(C)C)C1 ; N[C@@H](CO)C(=O)O]
(28) Compound 29 comprising Compound 1 and L-tyrosine: 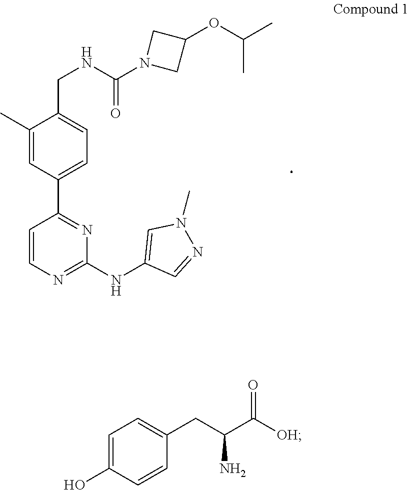 [SMILES: Cc1cc(-c2ccnc(Nc3cnn(C)c3)n2)ccc1CNC(=O)N1CC(OC(C)C)C1 ; N[C@@H](Cc1ccc(O)cc1)C(=O)O]
(29) Compound 30 comprising Compound 1 and nicotinamide: 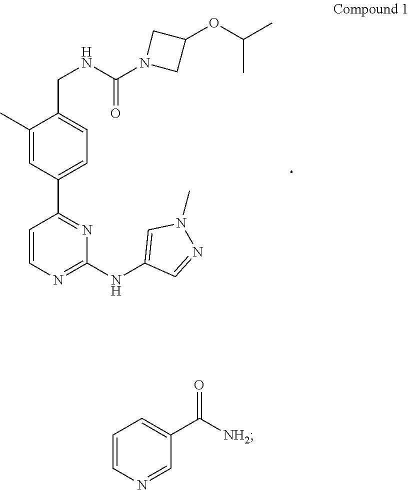 [SMILES: Cc1cc(-c2ccnc(Nc3cnn(C)c3)n2)ccc1CNC(=O)N1CC(OC(C)C)C1 ; NC(=O)c1cccnc1]
(30) Compound 31 comprising Compound 1 and nicotinic acid: 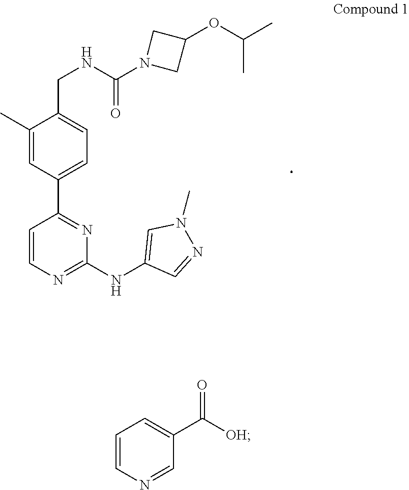 [SMILES: Cc1cc(-c2ccnc(Nc3cnn(C)c3)n2)ccc1CNC(=O)N1CC(OC(C)C)C1 ; O=C(O)c1cccnc1]
(31) Compound 32 comprising Compound 1 and saccharin: 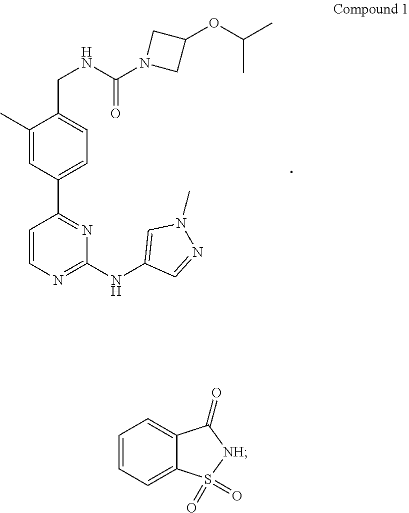 [SMILES: Cc1cc(-c2ccnc(Nc3cnn(C)c3)n2)ccc1CNC(=O)N1CC(OC(C)C)C1 ; O=C1NS(=O)(=O)c2ccccc21]
and
(32) Compound 33 comprising Compound 1 and L-pyroglutamic acid:

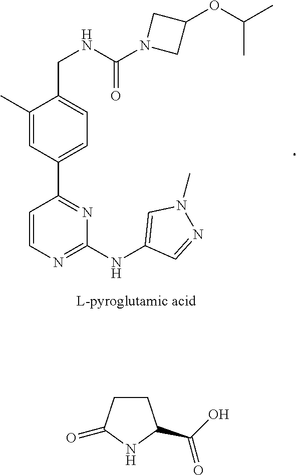

3-33. (canceled)

34. The compound according to claim 1, wherein the solid form of the compound has an acid:base ratio of about 1:1.

35. The compound according to claim 1, wherein said compound is crystalline.

36. The compound of claim 1, wherein said compound is amorphous.

37. The compound according to claim 1, wherein said compound is substantially free of impurities.

38. A composition comprising the compound according to claim 1 and a pharmaceutically acceptable carrier or excipient.

39. A method of decreasing the enzymatic activity of Bruton's tyrosine kinase comprising contacting Bruton's tyrosine kinase with an effective amount of a compound of claim 1 or a composition thereof.

40. A method of treating a disorder responsive to inhibition of Bruton's tyrosine kinase comprising administering to a subject an effective amount of a compound of claim 1 or a composition thereof.

41. A method of treating a disorder selected from the group consisting of autoimmune disorders, inflammatory disorders, and cancers comprising administering to a subject an effective amount of a compound of claim 1 of a composition thereof.

42. The method according to claim 41, wherein the disorder is selected from rheumatoid arthritis, systemic lupus erythematosus, atopic dermatitis, leukemia and lymphoma.

43. Compound 1: 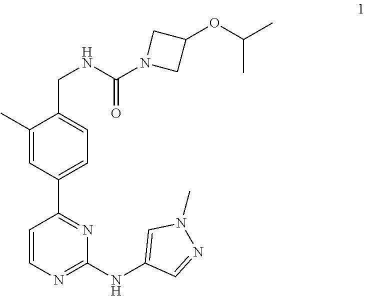 [SMILES: Cc1cc(-c2ccnc(Nc3cnn(C)c3)n2)ccc1CNC(=O)N1CC(OC(C)C)C1]
wherein said compound is crystalline.

44. The compound of claim 43, wherein the compound is of Type A, Type B, Type C, Type D, Type E, Type F or Type G polymorphic form of the compound.

45. A composition comprising Compound 1 according to claim 43 and a pharmaceutically acceptable carrier or excipient.

46. A method of decreasing the enzymatic activity of Bruton's tyrosine kinase comprising contacting Bruton's tyrosine kinase with an effective amount of Compound 1 according to claim 43 or a composition thereof.

47. A method of treating a disorder responsive to inhibition of Bruton's tyrosine kinase comprising administering to a subject an effective amount of Compound 1 according to claim 43 or a composition thereof.

48. A method of treating a disorder selected from the group consisting of autoimmune disorders, inflammatory disorders, and cancers comprising administering to a subject an effective amount of Compound 1 according to claim 43 or a composition thereof.

49. The method according to claim 48, wherein the disorder is selected from rheumatoid arthritis, systemic lupus erythematosus, atopic dermatitis, leukemia and lymphoma.
